SEQUENCE LISTING (107 sequences)

SEQ ID NO: 1 - DNA, 1026 bp, Synechococcus elongatus
atgttcggtcttatcggtcatctcaccagtttggagcaggcccgcgacgtttctcgcaggatgggctacgacgaatacgccgatcaaggattggagttttggagtagcgctcctcctcaaatcgttgatgaaatcacagtcaccagtgccacaggcaaggtgattcacggtcgctacatcgaatcgtgtttcttgccggaaatgctggcggcgcgccgcttcaaaacagccacgcgcaaagttctcaatgccatgtcccatgcccaaaaacacggcatcgacatctcggccttggggggctttacctcgattattttcgagaatttcgatttggccagtttgcggcaagtgcgcgacactaccttggagtttgaacggttcaccaccggcaatactcacacggcctacgtaatctgtagacaggtggaagccgctgctaaaacgctgggcatcgacattacccaagcgacagtagcggttgtcggcgcgactggcgatatcggtagcgctgtctgccgctggctcgacctcaaactgggtgtcggtgatttgatcctgacggcgcgcaatcaggagcgtttggataacctgcaggctgaactcggccggggcaagattctgcccttggaagccgctctgccggaagctgactttatcgtgtgggtcgccagtatgcctcagggcgtagtgatcgacccagcaaccctgaagcaaccctgcgtcctaatcgacgggggctaccccaaaaacttgggcagcaaagtccaaggtgagggcatctatgtcctcaatggcggggtagttgaacattgcttcgacatcgactggcagatcatgtccgctgcagagatggcgcggcccgagcgccagatgtttgcctgctttgccgaggcgatgctcttggaatttgaaggctggcatactaacttctcctggggccgcaaccaaatcacgatcgagaagatggaagcgatcggtgaggcatcggtgcgccacggcttccaacccttggcattggcaatttga

SEQ ID NO: 2 - protein, 341 aa, Synechococcus elongatus
MFGLIGHLTSLEQARDVSRRMGYDEYADQGLEFWSSAPPQIVDEITVTSATGKVIHGRYIESCFLPEMLAARRFKTATRKVLNAMSHAQKHGIDISALGGFTSIIFENFDLASLRQVRDTTLEFERFTTGNTHTAYVICRQVEAAAKTLGIDITQATVAVVGATGDIGSAVCRWLDLKLGVGDLILTARNQERLDNLQAELGRGKILPLEAALPEADFIVWVASMPQGVVIDPATLKQPCVLIDGGYPKNLGSKVQGEGIYVLNGGVVEHCFDIDWQIMSAAEMARPERQMFACFAEAMLLEFEGWHTNFSWGRNQITIEKMEAIGEASVRHGFQPLALAI

SEQ ID NO: 3 - DNA, 1023 bp, Synechocystis sp.
atgtttggtcttattggtcatctcacgagtttagaacacgcccaagcggttgctgaagatttaggctatcctgagtacgccaaccaaggcctggatttttggtgttcggctcctccccaagtggttgataattttcaggtgaaaagtgtgacggggcaggtgattgaaggcaaatatgtggagtcttgctttttgccggaaatgttaacccaacggcggatcaaagcggccattcgtaaaatcctcaatgctatggccctggcccaaaaggtgggcttggatattacggccctgggaggcttttcttcaatcgtatttgaagaatttaacctcaagcaaaataatcaagtccgcaatgtggaactagattttcagcggttcaccactggtaatacccacaccgcttatgtgatctgccgtcaggtcgagtctggagctaaacagttgggtattgatctaagtcaggcaacggtagcggtttgtggcgccacgggagatattggtagcgccgtatgtcgttggttagatagcaaacatcaagttaaggaattattgctaattgcccgtaaccgccaaagattggaaaatctccaagaggaattgggtcggggcaaaattatggatttggaaacagccctgccccaggcagatattattgtttgggtggctagtatgcccaagggggtagaaattgcgggggaaatgctgaaaaagccctgtttgattgtggatgggggctatcccaagaatttagacaccagggtgaaagcggatggggtgcatattctcaagggggggattgtagaacattcccttgatattacctgggaaattatgaagattgtggagatggatattccctcccggcaaatgttcgcctgttttgcggaggccattttgctagagtttgagggctggcgcactaatttttcctggggccgcaaccaaatttccgttaataaaatggaggcgattggtgaagcttctgtcaagcatggcttttgccctttagtagctctttag

SEQ ID NO: 4 - protein, 340 aa, Synechocystis sp.
MFGLIGHLTSLEHAQAVAEDLGYPEYANQGLDFWCSAPPQVVDNFQVKSVTGQVIEGKYVESCFLPEMLTQRRIKAAIRKILNAMALAQKVGLDITALGGFSSIVFEEFNLKQNNQVRNVELDFQRFTTGNTHTAYVICRQVESGAKQLGIDLSQATVAVCGATGDIGSAVCRWLDSKHQVKELLLIARNRQRLENLQEELGRGKIMDLETALPQADIIVWVASMPKGVEIAGEMLKKPCLIVDGGYPKNLDTRVKADGVHILKGGIVEHSLDITWEIMKIVEMDIPSRQMFACFAEAILLEFEGWRTNFSWGRNQISVNKMEAIGEASVKHGFCPLVAL

SEQ ID NO: 5 - DNA, 243 bp, Synechococcus elongatus PCC 7942
atgagccaagaagacatcttcagcaaagtcaaagacattgtggctgagcagctgagtgtggatgtggctgaagtcaagccagaatccagcttccaaaacgatctgggagcggactcgctggacaccgtggaactggtgatggctctggaagaggctttcgatatcgaaatccccgatgaagccgctgaaggcattgcgaccgttcaagacgccgtcgatttcatcgctagcaaagctgcctag

SEQ ID NO: 6 - protein, 80 aa, Synechococcus elongatus PCC 7942
MSQEDIFSKVKDIVAEQLSVDVAEVKPESSFQNDLGADSLDTVELVMALEEAFDIEIPDEAAEGIATVQDAVDFIASKAA

SEQ ID NO: 7 - DNA, 261 bp, Acinetobacter sp. ADP1
atgtcgaacctggcggatgagatcaaacaaatgatcattgacgtcctcgctctcgaggatatccaaatccaggatattgatgaaacggcaccgctgttcggggatggtttgggcctggatagtattgacgcgctcgaactcggcctggccttgaaaaagcgctaccacatccatttgaatgccgaatctgacgaaactaagcagcactttcggtccattcagagcctggtgaccctggtggaggcccaacagaaagcttag

SEQ ID NO: 8 - protein, 86 aa, Acinetobacter sp. ADP1
MSNLADEIKQMIIDVLALEDIQIQDIDETAPLFGDGLGLDSIDALELGLALKKRYHIHLNAESDETKQHFRSIQSLVTLVEAQQKA

SEQ ID NO: 9 - DNA, 246 bp, Acinetobacter sp. ADP1
atgttgagtcaggaacacatcctctccacactccgcgaatggatggaggacttgtttgaaatcgagcctgaaaccattcaactggattctaacctgtactcggacctggatgtggatagcattgatgcggtcgatctgattgtcaagatcaaagagctcacgggcaaacaggtgaaaccggaagacttcaagaatgtccggactgtccatgatgttgtgaccgtgatccaaaacatgacggcttag

SEQ ID NO: 10 - protein, 81 aa, Acinetobacter sp. ADP1
MLSQEHILSTLREWMEDLFEIEPETIQLDSNLYSDLDVDSIDAVDLIVKIKELTGKQVKPEDFKNVRTVHDVVTVIQNMTA

SEQ ID NO: 11 - DNA, 345 bp, Acinetobacter sp. ADP1
atggtcgtctacacgtggccgaaatgtcgttgcattaactttcagaaaatccaatacagcatcaaactgacagcgatcaaaacgcctcgagcaatgcgccgcattcccgtgtctgatattgaacaacgggtgaagcaggccgtggcagaacagctcggcatcaaagccgaagaaatcaagaatgaggcttcgttcatggatgacttgggtgccgacagtctggatctcgtcgagctggtgatgagctttgagaatgattttgatatcaccattccggatgaagactcgaacgagatcactaccgttcaatccgcgattgactacgtgaccaagaagctgggttag

SEQ ID NO: 12 - protein, 114 aa, Acinetobacter sp. ADP1
MVVYTWPKCRCINFQKIQYSIKLTAIKTPRAMRRIPVSDIEQRVKQAVAEQLGIKAEEIKNEASFMDDLGADSLDLVELVMSFENDFDITIPDEDSNEITTVQSAIDYVTKKLG

SEQ ID NO: 13 - DNA, 246 bp, Spinacia oleracea
gcaaagaaggaaacaattgacaaagtgtgcgacattgtaaaggagaaactggctttaggagctgatgttgtggtcacagctgattccgagtttagtaaactcggtgctgattcattggacacggttgagatagtgatgaacctcgaggaagagttcggtatcaatgtggatgaagataaagctcaagatatatcaaccatccaacaagccgccgacgttattgagagtcttcttgagaagaaatag

SEQ ID NO: 14 - protein, 81 aa, Spinacia oleracea
AKKETIDKVCDIVKEKLALGADVVVTADSEFSKLGADSLDTVEIVMNLEEEFGINVDEDKAQDISTIQQAADVIESLLEKK

SEQ ID NO: 15 - protein, 7 aa, Artificial Sequence
  Heptapeptide retention motif
FYXDWWN

SEQ ID NO: 16 - DNA, 1752 bp, Escherichia coli
atgttaacggcatgtatatcatttggggttgcgatgacgacgaacacgcattttagaggtgaagaattgaaaaaagtgtggctcaatcggtatccggcggatgtcccaactgaaatcaaccctgatcgatatcagtccctcgtggacatgtttgaacagagcgtggcacgctacgccgatcagcccgccttcgtgaatatgggcgaggttatgacgtttcggaaattggaagaacgctctcgggcgtttgcggcttatttgcagcagggcctgggcctgaagaaaggtgatcgggtcgccttgatgatgcccaacctcttgcaatacccggtcgccctgtttggaatcctgcgtgctggcatgattgtcgtgaatgtgaatcctctctacacccctcgtgaactcgaacaccagctgaacgatagtggcgcttccgctattgttatcgtgtctaatttcgctcatacgctggagaaggtcgtggacaagacagccgttcaacacgtcattctgacccgcatgggtgatcaactgagtacggcaaaaggtacggtcgtcaattttgtcgtcaaatatatcaaacgtctggtccccaagtaccatctgccagacgcgatttccttccggagtgctttgcataacggatatcgaatgcaatacgtgaaacccgaactggtgcctgaggacctcgcatttctgcagtacacaggtggcaccaccggggtggccaagggtgctatgctgacacatcgaaatatgctcgccaacctcgagcaggtcaacgccacctacggtccgctgttgcacccaggcaaggagctggttgtgacggctttgcccctgtatcatatttttgctctgacgatcaactgcctgctgtttattgagttgggtggtcagaacctcctgatcaccaatccacgcgatattccgggcctcgttaaagaactcgcgaaatacccctttactgcgatcacgggtgttaatactctctttaacgcgctgctcaacaataaggagttccaacagttggacttcagcagcctgcatctctctgccggcggtggcatgcctgtgcaacaagttgttgcggagcgatgggtgaaattgacggggcagtatctgttggaggggtacgggttgaccgaatgcgcacctctggtgtcggtgaacccctacgatattgactaccacagcggatcgatcggcctgccggtgccgtcgacagaagcgaaactggttgacgacgatgataacgaggtgcccccaggccaaccgggggagttgtgtgttaagggaccgcaagtcatgctcgggtactggcagcggccggatgccactgatgaaattatcaagaatggttggctccacaccggggacattgcagttatggatgaagagggattcctgcgcatcgtcgatcgcaaaaaagacatgatcctcgtgtccggctttaatgtctatccaaatgaaatcgaggatgtcgttatgcagcaccctggggtgcaggaggttgccgctgttggcgtgcctagcgggagtagcggcgaagcggtcaaaattttcgttgtcaagaaggaccccagtttgaccgaagagtcgttggtcacgttctgtcgccgccaactgactggatataaagtccccaaactcgtcgaatttcgggatgaattgcccaagtcgaacgtcggcaagatcctccgccgcgagttgcgcgatgaagcacgcggtaaggttgacaataaggcttag

SEQ ID NO: 17 - protein, 583 aa, Escherichia coli
MLTACISFGVAMTTNTHFRGEELKKVWLNRYPADVPTEINPDRYQSLVDMFEQSVARYADQPAFVNMGEVMTFRKLEERSRAFAAYLQQGLGLKKGDRVALMMPNLLQYPVALFGILRAGMIVVNVNPLYTPRELEHQLNDSGASAIVIVSNFAHTLEKVVDKTAVQHVILTRMGDQLSTAKGTVVNFVVKYIKRLVPKYHLPDAISFRSALHNGYRMQYVKPELVPEDLAFLQYTGGTTGVAKGAMLTHRNMLANLEQVNATYGPLLHPGKELVVTALPLYHIFALTINCLLFIELGGQNLLITNPRDIPGLVKELAKYPFTAITGVNTLFNALLNNKEFQQLDFSSLHLSAGGGMPVQQVVAERWVKLTGQYLLEGYGLTECAPLVSVNPYDIDYHSGSIGLPVPSTEAKLVDDDDNEVPPGQPGELCVKGPQVMLGYWQRPDATDEIIKNGWLHTGDIAVMDEEGFLRIVDRKKDMILVSGFNVYPNEIEDVVMQHPGVQEVAAVGVPSGSSGEAVKIFVVKKDPSLTEESLVTFCRRQLTGYKVPKLVEFRDELPKSNVGKILRRELRDEARGKVDNKA

SEQ ID NO: 18 - DNA, 2103 bp, Saccharomyces cerevisiae S288c
atggttgctcaatataccgttccagttgggaaagccgccaatgagcatgaaactgctccaagaagaaattatcaatgccgcgagaagccgctcgtcagaccgcctaacacaaagtgttccactgtttatgagtttgttctagagtgctttcagaagaacaaaaattcaaatgctatgggttggagggatgttaaggaaattcatgaagaatccaaatcggttatgaaaaaagttgatggcaaggagacttcagtggaaaagaaatggatgtattatgaactatcgcattatcattataattcatttgaccaattgaccgatatcatgcatgaaattggtcgtgggttggtgaaaataggattaaagcctaatgatgatgacaaattacatctttacgcagccacttctcacaagtggatgaagatgttcttaggagcgcagtctcaaggtattcctgtcgtcactgcctacgatactttgggagagaaagggctaattcattctttggtgcaaacggggtctaaggccatttttaccgataactctttattaccatccttgatcaaaccagtgcaagccgctcaagacgtaaaatacataattcatttcgattccatcagttctgaggacaggaggcaaagtggtaagatctatcaatctgctcatgatgccatcaacagaattaaagaagttagacctgatatcaagacctttagctttgacgacatcttgaagctaggtaaagaatcctgtaacgaaatcgatgttcatccacctggcaaggatgatctttgttgcatcatgtatacgtctggttctacaggtgagccaaagggtgttgtcttgaaacattcaaatgttgtcgcaggtgttggtggtgcaagtttgaatgttttgaagtttgtgggcaataccgaccgtgttatctgttttttgccactagctcatatttttgaattggttttcgaactattgtccttttattggggggcctgcattggttatgccaccgtaaaaactttaactagcagctctgtgagaaattgtcaaggtgatttgcaagaattcaagcccacaatcatggttggtgtcgccgctgtttgggaaacagtgagaaaagggatcttaaaccaaattgataatttgcccttcctcaccaagaaaatcttctggaccgcgtataataccaagttgaacatgcaacgtctccacatccctggtggcggcgccttaggaaacttggttttcaaaaaaatcagaactgccacaggtggccaattaagatatttgttaaacggtggttctccaatcagtcgggatgctcaggaattcatcacaaatttaatctgccctatgcttattggttacggtttaaccgagacatgcgctagtaccaccatcttggatcctgctaattttgaactcggcgtcgctggtgacctaacaggttgtgttaccgtcaaactagttgatgttgaagaattaggttattttgctaaaaacaaccaaggtgaagtttggatcacaggtgccaatgtcacgcctgaatattataagaatgaggaagaaacttctcaagctttaacaagcgatggttggttcaagaccggtgacatcggtgaatgggaagcaaatggccatttgaaaataattgacaggaagaaaaacttggtcaaaacaatgaacggtgaatatatcgcactcgagaaattagagtccgtttacagatctaacgaatatgttgctaacatttgtgtttatgccgaccaatctaagactaagccagttggtattattgtaccaaatcatgctccattaacgaagcttgctaaaaagttgggaattatggaacaaaaagacagttcaattaatatcgaaaattatttggaggatgcaaaattgattaaagctgtttattctgatcttttgaagacaggtaaagaccaaggtttggttggcattgaattactagcaggcatagtgttctttgacggcgaatggactccacaaaacggttttgttacgtccgctcagaaattgaaaagaaaagacattttgaatgctgtcaaagataaagttgacgccgtttatagttcgtcttaa

SEQ ID NO: 19 - protein, 700 aa, Saccharomyces cerevisiae S288c
MVAQYTVPVGKAANEHETAPRRNYQCREKPLVRPPNTKCSTVYEFVLECFQKNKNSNAMGWRDVKEIHEESKSVMKKVDGKETSVEKKWMYYELSHYHYNSFDQLTDIMHEIGRGLVKIGLKPNDDDKLHLYAATSHKWMKMFLGAQSQGIPVVTAYDTLGEKGLIHSLVQTGSKAIFTDNSLLPSLIKPVQAAQDVKYIIHFDSISSEDRRQSGKIYQSAHDAINRIKEVRPDIKTFSFDDILKLGKESCNEIDVHPPGKDDLCCIMYTSGSTGEPKGVVLKHSNVVAGVGGASLNVLKFVGNTDRVICFLPLAHIFELVFELLSFYWGACIGYATVKTLTSSSVRNCQGDLQEFKPTIMVGVAAVWETVRKGILNQIDNLPFLTKKIFWTAYNTKLNMQRLHIPGGGALGNLVFKKIRTATGGQLRYLLNGGSPISRDAQEFITNLICPMLIGYGLTETCASTTILDPANFELGVAGDLTGCVTVKLVDVEELGYFAKNNQGEVWITGANVTPEYYKNEEETSQALTSDGWFKTGDIGEWEANGHLKIIDRKKNLVKTMNGEYIALEKLESVYRSNEYVANICVYADQSKTKPVGIIVPNHAPLTKLAKKLGIMEQKDSSINIENYLEDAKLIKAVYSDLLKTGKDQGLVGIELLAGIVFFDGEWTPQNGFVTSAQKLKRKDILNAVKDKVDAVYSSS

SEQ ID NO: 20 - DNA, 2235 bp, Saccharomyces cerevisiae S288c
atggccgctccagattatgcacttaccgatttaattgaatcggatcctcgtttcgaaagtttgaagacaagattagccggttacaccaaaggctctgatgaatatattgaagagctatactctcaattaccactgaccagctatcccaggtacaaaacatttttaaagaaacaggcggttgccatttcgaatccggataatgaagctggttttagctcgatttataggagttctctttcttctgaaaatctagtgagctgtgtggataaaaacttaagaactgcatacgatcacttcatgttttctgcaaggagatggcctcaacgtgactgtttaggttcaaggccaattgataaagccacaggcacctgggaggaaacattccgtttcgagtcgtactccacggtatctaaaagatgtcataatatcggaagtggtatattgtctttggtaaacacgaaaaggaaacgtcctttggaagccaatgattttgttgttgctatcttatcacacaacaaccctgaatggatcctaacagatttggcctgtcaggcctattctctaactaacacggctttgtacgaaacattaggtccaaacacctccgagtacatattgaatttaaccgaggcccccattctgatttttgcaaaatcaaatatgtatcatgtattgaagatggtgcctgatatgaaatttgttaatactttggtttgtatggatgaattaactcatgacgagctccgtatgctaaatgaatcgttgctacccgttaagtgcaactctctcaatgaaaaaatcacatttttttcattggagcaggtagaacaagttggttgctttaacaaaattcctgcaattccacctaccccagattccttgtatactatttcgtttacttctggtactacaggtttacctaaaggtgtggaaatgtctcacagaaacattgcgtctgggatagcatttgctttttctaccttcagaataccgccagataaaagaaaccaacagttatatgatatgtgttttttgccattggctcatatttttgaaagaatggttattgcgtatgatctagccatcgggtttggaataggcttcttacataaaccagacccaactgtattggtagaggatttgaagattttgaaaccttacgcggttgccctggttcctagaatattaacacggtttgaagccggtataaaaaacgctttggataaatcgactgtccagaggaacgtagcaaatactatattggattctaaatcggccagatttaccgcaagaggtggtccagataaatcgattatgaattttctagtttatcatcgcgtattgattgataaaatcagagactctttaggtttgtccaataactcgtttataattaccggatcagctcccatatctaaagataccttactatttttaagaagtgccttggatattggtataagacagggctacggcttaactgaaacttttgctggtgtctgtttaagcgaaccgtttgaaaaagatgtcggatcttgtggtgccataggtatttctgcagaatgtagattgaagtctgttccagaaatgggttaccatgccgacaaggatttaaaaggtgaactgcaaattcgtggcccacaggtttttgaaagatattttaaaaatccgaatgaaacttcaaaagccgttgaccaagatggttggttttccacgggagatgttgcatttatcgatggaaaaggtcgcatcagcgtcattgatcgagtcaagaactttttcaagctagcacatggtgaatatattgctccagagaaaatcgaaaatatttatttatcatcatgcccctatatcacgcaaatatttgtctttggagatcctttaaagacatttttagttggcatcgttggtgttgatgttgatgcagcgcaaccgattttagctgcaaagcacccagaggtgaaaacgtggactaaggaagtgctagtagaaaacttaaatcgtaataaaaagctaaggaaggaatttttaaacaaaattaataaatgcaccgatgggctacaaggattcgaaaaattgcataacatcaaagtcggacttgagcctttaactctcgaggatgatgttgtgacgccaacttttaaaataaagcgtgccaaagcatcaaaattcttcaaagatacattagaccaactatacgccgaaggttcactagtcaagacagaaaagctttag

SEQ ID NO: 21 - protein, 744 aa, Saccharomyces cerevisiae S288c
MAAPDYALTDLIESDPRFESLKTRLAGYTKGSDEYIEELYSQLPLTSYPRYKTFLKKQAVAISNPDNEAGFSSIYRSSLSSENLVSCVDKNLRTAYDHFMFSARRWPQRDCLGSRPIDKATGTWEETFRFESYSTVSKRCHNIGSGILSLVNTKRKRPLEANDFVVAILSHNNPEWILTDLACQAYSLTNTALYETLGPNTSEYILNLTEAPILIFAKSNMYHVLKMVPDMKFVNTLVCMDELTHDELRMLNESLLPVKCNSLNEKITFFSLEQVEQVGCFNKIPAIPPTPDSLYTISFTSGTTGLPKGVEMSHRNIASGIAFAFSTFRIPPDKRNQQLYDMCFLPLAHIFERMVIAYDLAIGFGIGFLHKPDPTVLVEDLKILKPYAVALVPRILTRFEAGIKNALDKSTVQRNVANTILDSKSARFTARGGPDKSIMNFLVYHRVLIDKIRDSLGLSNNSFIITGSAPISKDTLLFLRSALDIGIRQGYGLTETFAGVCLSEPFEKDVGSCGAIGISAECRLKSVPEMGYHADKDLKGELQIRGPQVFERYFKNPNETSKAVDQDGWFSTGDVAFIDGKGRISVIDRVKNFFKLAHGEYIAPEKIENIYLSSCPYITQIFVFGDPLKTFLVGIVGVDVDAAQPILAAKHPEVKTWTKEVLVENLNRNKKLRKEFLNKINKCTDGLQGFEKLHNIKVGLEPLTLEDDVVTPTFKIKRAKASKFFKDTLDQLYAEGSLVKTEKL

SEQ ID NO: 22 - DNA, 2081 bp, Artificial Sequence
  S. cerevisiae FadD homolog (Faa3p)  - codon optimized
atgtctgaacaacactcggtggccgtcggtaaagccgctaacgaacatgaaactgccccccgacgtaacgtgcgcgtgaaaaaacgccccttgattcgccctctcaatagcagcgcgtcgacgttgtatgagtttgccctggaatgctttaacaaggggggcaaacgcgatggcatggcgtggcgagacgtcatcgagattcacgaaacgaagaagactatcgtgcgtaaggtcgacggaaaggataaaagcattgaaaagacctggctgtactacgaaatgagcccgtacaaaatgatgacgtatcaggaactcatttgggtgatgcatgatatgggtcgcgggctcgccaagattggcatcaagcccaacggtgaacacaaatttcatattttcgcgtcgacctcccacaaatggatgaaaatctttctcggctgcatctcgcaaggcattcctgtggtcaccgcttatgataccctcggcgaaagtggtctcattcattctatggtggaaacagagagtgctgctatctttacagataaccaattgctggcgaaaatgatcgtgcctctgcagtctgctaaagatatcaagtttctcattcacaacgagccaatcgaccccaatgatcgacgccagaatggaaaactctataaagctgctaaggacgcgatcaacaagattcgcgaggttcggcctgatatcaagatttactcgttcgaagaagtggttaaaatcggcaagaagagtaaagatgaagtgaaactgcatccgcccgaacccaaggatctcgcgtgtatcatgtacaccagtggatctatcagcgcgcccaaaggggtggtcctgacccattataatatcgtcagtgggattgcaggcgttgggcataacgtctttggctggatcggctccaccgatcgtgtcctgagctttttgcctctcgcacacattttcgaactcgtttttgaattcgaagcgttctactggaatggtattctgggatacggcagcgtgaaaaccttgacgaatacgagcacccgcaactgtaaaggtgatctggtggagtttaaaccgaccatcatgattggtgttgcggccgtttgggagacggtccgcaaagcgatcctggagaaaatcagtgatttgacaccggtgctgcagaagattttctggtcggcttacagcatgaaagagaaaagtgtgccatgcacgggatttttgtctcgtatggtctttaaaaaggttcgacaagctaccggtggtcacctcaagtatattatgaatggcggctccgctatctctattgacgcccaaaaattctttagtatcgtcttgtgcccgatgatcattggttatggcttgactgaaacagtggcaaacgcctgtgttctcgagccggaccattttgagtatggcatcgttggggacctggtggggtcggtcacggcaaaattggttgacgtgaaggatctggggtactatgccaaaaataatcagggggaactcctgttgaagggagcgcccgtctgcagcgaatactacaagaatccgattgagacagctgtgagcttcacatacgacggttggtttcgtaccggcgatatcgtcgagtggacgccaaagggtcagctcaaaattattgatcggcgcaagaacctggtcaagactttgaatggcgagtatattgcgctggaaaagctggagagcgtttaccgctcgaacagttacgtcaagaatatctgtgtgtacgccgatgagtcccgagtgaaacccgttggtattgtggtcccaaaccctggaccgctgtctaagtttgctgtcaagctgcgcattatgaagaagggggaagacattgagaattatattcacgataaggcgctccggaacgcagtgttcaaagagatgatcgccactgcaaaatcgcagggcctggtcggcattgagctgttgtgtggtatcgttttcttcgacgaggaatggactcccgaaaatggcttcgtgactagcgcccaaaagttgaaacggcgcgagattttggcagccgtcaaatccgaggttgaacgcgtctataaagaaaatag

SEQ ID NO: 23 - protein, 694 aa, Saccharomyces cerevisiae S288c
MSEQHSVAVGKAANEHETAPRRNVRVKKRPLIRPLNSSASTLYEFALECFNKGGKRDGMAWRDVIEIHETKKTIVRKVDGKDKSIEKTWLYYEMSPYKMMTYQELIWVMHDMGRGLAKIGIKPNGEHKFHIFASTSHKWMKIFLGCISQGIPVVTAYDTLGESGLIHSMVETESAAIFTDNQLLAKMIVPLQSAKDIKFLIHNEPIDPNDRRQNGKLYKAAKDAINKIREVRPDIKIYSFEEVVKIGKKSKDEVKLHPPEPKDLACIMYTSGSISAPKGVVLTHYNIVSGIAGVGHNVFGWIGSTDRVLSFLPLAHIFELVFEFEAFYWNGILGYGSVKTLTNTSTRNCKGDLVEFKPTIMIGVAAVWETVRKAILEKISDLTPVLQKIFWSAYSMKEKSVPCTGFLSRMVFKKVRQATGGHLKYIMNGGSAISIDAQKFFSIVLCPMIIGYGLTETVANACVLEPDHFEYGIVGDLVGSVTAKLVDVKDLGYYAKNNQGELLLKGAPVCSEYYKNPIETAVSFTYDGWFRTGDIVEWTPKGQLKIIDRRKNLVKTLNGEYIALEKLESVYRSNSYVKNICVYADESRVKPVGIVVPNPGPLSKFAVKLRIMKKGEDIENYIHDKALRNAVFKEMIATAKSQGLVGIELLCGIVFFDEEWTPENGFVTSAQKLKRREILAAVKSEVERVYKENS

SEQ ID NO: 24 - DNA, 1632 bp, Synechococcus elongatus PCC 7942
atgaatatccacactgtcgcgacgcaagcctttagcgaccaaaagcccggtacctccggcctgcgcaagcaagttcctgtcttccaaaaacggcactatctcgaaaactttgtccagtcgatcttcgatagccttgagggttatcagggccagacgttagtgctggggggtgatggccgctactacaatcgcacagccatccaaaccattctgaaaatggcggcggccaatggttggggccgcgttttagttggacaaggcggtattctctccacgccagcagtctccaacctaatccgccagaacggagccttcggcggcatcatcctctcggctagccacaacccagggggccctgagggcgatttcggcatcaagtacaacatcagcaacggtggccctgcacccgaaaaagtcaccgatgccatctatgcctgcagcctcaaaattgaggcctaccgcattctcgaagccggtgacgttgacctcgatcgactcggtagtcaacaactgggcgagatgaccgttgaggtgatcgactcggtcgccgactacagccgcttgatgcaatccctgtttgacttcgatcgcattcgcgatcgcctgaggggggggctacggattgcgatcgactcgatgcatgccgtcaccggtccctacgccaccacgatttttgagaaggagctaggcgcggcggcaggcactgtttttaatggcaagccgctggaagactttggcgggggtcacccagacccgaatttggtctacgcccacgacttggttgaactgttgtttggcgatcgcgccccagattttggcgcggcctccgatggcgatggcgatcgcaacatgatcttgggcaatcacttttttgtgacccctagcgacagcttggcgattctcgcagccaatgccagcctagtgccggcctaccgcaatggactgtctgggattgcgcgatccatgcccaccagtgcggcggccgatcgcgtcgcccaagccctcaacctgccctgctacgaaaccccaacgggttggaagtttttcggcaatctgctcgatgccgatcgcgtcaccctctgcggcgaagaaagctttggcacaggctccaaccatgtgcgcgagaaggatggcctgtgggccgtgctgttctggctgaatattctggcggtgcgcgagcaatccgtggccgaaattgtccaagaacactggcgcacctacggccgcaactactactctcgccacgactacgaaggggtggagagcgatcgagccagtacgctggtggacaaactgcgatcgcagctacccagcctgaccggacagaaactgggagcctacaccgttgcctacgccgacgacttccgctacgaagatccggtcgatggcagcatcagcgaacagcagggcattcgtattggctttgaagacggctcacgtatggtcttccgcttgtctggtactggtacggcaggagccaccctgcgcctctacctcgagcgcttcgaaggggacaccaccaaacagggtctcgatccccaagttgccctggcagatttgattgcaatcgccgatgaagtcgcccagatcacaaccttgacgggcttcgatcaaccgacagtgatcacctga

SEQ ID NO: 25 - DNA, 1704 bp, Synechocystis sp. PCC 6803
gtgtctaagcccctgatcgccgccctccattttttacaatttttgtatatgacaagcagaattaatcccctcgccggccagcatccccccgccgacagccttttggatgtggccaaacttttagacgactattaccgtcagcaaccggacccggaaaatcccgcccagttagtgagctttggtacctctggccatcggggttctgccctcaacggtacttttaatgaagcccatattttggcggtgacccaggcagtggtggactatcgccaagcccagggcattacggggcccctttatatggggatggatagccatgctctgtcggaaccagcccagaaaacggcgttggaagtgttggccgctaaccaagtagaaacttttttaaccaccgccacggatttaacccgtttcacccccactccggcggtatcctacgccattttgacccacaaccagggacgtaaagaaggtttagcggacggcattattattaccccttcccacaatccccccactgatggaggctttaaatataatcccccctccggtggcccggcggaaccggaagcgacccaatggattcagaaccgggccaatgagttgctgaaaaatggcaataaaacagttaaacggctggattacgagcaggcattaaaagccaccaccacccatgcccatgattttgtcactccctatgtggccggtctggcggacatcattgacttggatgtaattcgttcagcgggcttgcgcttgggagttgaccccctggggggagccaatgtgggctattgggaacccattgccgctaaatacaatttgaacatcagcttggttaatcccggggtagatcccacgtttaaatttatgaccctggattgggacggcaaaatccgcatggattgttcttccccctacgccatggccagtttggtgaaaatcaaagaccattacgacattgcctttggcaacgacaccgacggcgatcgccatggcattgtcacccccagcgtgggtttgatgaatcccaatcattttctttccgtggccatttggtatttgtttagtcagcggcaacagtggtcagggctgtcggcgatcggcaaaaccctagtcagcagcagcatgattgaccgggtgggggccatgattaatcgccaagtttacgaagtgcccgtgggctttaaatggtttgtcagcggtttgctagatggttcctttggctttgggggtgaagaaagtgccggggcttcgtttttgaaaaaaaatggcaccgtttggaccaccgacaaagatggcaccattatggatttattggcggcggaaatcaccgctaaaaccggcaaagatcccggcctccattaccaggatttgaccgctaagttaggtaatcccatttaccaacgcattgatgcccccgccactccggcccaaaaagaccgcttgaaaaaactgtcccccgatgacgttacagctacctccttagctggggatgccattactgctaaattaaccaaagcccctggcaaccaagcggcgatcggtgggttgaaggtgaccactgcggaaggttggtttgcggcccggccctccggcacggaaaatgtttacaaaatctatgccgaaagtttcaaagacgaagcccatctccaggctattttcacggaggcggaagccattgttacctcggctttgggctaa

SEQ ID NO: 26 - DNA, 1659 bp, Synechococcus sp. WH8102
atgaccacctcggcccccgcggaaccgaccctgcgcctggtgcgcctggacgcacctttcacggatcagaaacccggcacatccggtttgcgcaaaagcagccagcagttcgagcaagcgaactatctggagagctttgtggaagccgtattccgcaccttgcccggtgttcaagggggcacgctggtgttgggaggtgacggccgttacggcaaccgccgtgccatcgacgtgatcctgcgcatgggcgcggcccacggcctcagcaaggtgatcgtcaccaccggcggcatcctctccaccccggcggcctcgaacctgattcgccagcgtcaggccatcggcggcatcatcctctcggcaagccacaaccctggcggccccaatggagacttcggcgtcaaggtgaatggcgccaacggtggcccgaccccggcctcgttcaccgatgcggtgttcgagtgcaccaagaccttggagcaatacacgatcgttgatgccgcggccatcgccatcgatacccccggcagctacagcatcggcgccatgcaggtggaggtgatcgacggcgtcgacgacttcgtggctctgatgcaacagctgttcgactttgatcggatccgggagctgatccgcagcgacttcccgctggcgtttgatgcgatgcatgcggtcactggcccctacgccactcgcctgttggaagagatcctcggcgctcctgccggcagcgtccgcaacggcgttcctctggaggacttcggcggcggccaccccgaccccaacctcacctacgcccacgagctggccgaacttctgctcgacggggaggagttccgcttcggggccgcctgcgacggcgatggtgaccgcaacatgatcctggggcagcactgcttcgtaaaccccagcgacagcctggcggtgctcacagccaacgccacggtggcaccggcctatgccgatggtttggctggcgtggcccgctcgatgcccaccagctctgccgtggatgtggtggccaaggaactgggcatcgactgctacgagacccccaccggctggaagttcttcggcaatctgctggatgccggcaaaatcacgctctgcggtgaagagagcttcggcaccggcagcaaccacgtgcgtgaaaaggatggcctctgggctgttctgttctggctgcagatcctggccgagcgccgctgcagcgtcgccgagatcatggctgagcattggaagcgcttcggccgccactactactctcgccacgactacgaagccgtcgccagcgacgcagcccatgggctgttccaccgcctcgagggcatgctccctggtctggtggggcagagcttcgctggccgcagcgtcagcgcagccgacaacttcagctacaccgatcccgttgatggctctgtgaccaagggccagggcctgcgcatcctgctggaggatggcagccgcgtgatggtgcgcctctcgggcaccggcaccaagggcgccacgatccgcgtctatctggagagttatgtaccgagcagcggtgatctcaaccaggatccccaggtcgctctggccgacatgatcagcgccatcaatgaactggcggagatcaagcagcgcaccggcatggatcggcccaccgtgatcacctga

SEQ ID NO: 27 - DNA, 1293 bp, Synechococcus elongatus PCC 7942
gtgaaaaacgtgctggcgatcattctcggtggaggcgcaggcagtcgtctctatccactaaccaaacagcgcgccaaaccagcggtccccctggcgggcaaataccgcttgatcgatattcccgtcagcaattgcatcaacgctgacatcaacaaaatctatgtgctgacgcagtttaactctgcctcgctcaaccgccacctcagtcagacctacaacctctccagcggctttggcaatggctttgttgaggtgctagcagctcagattacgccggagaaccccaactggttccaaggcaccgccgatgcggttcgccagtatctctggctaatcaaagagtgggatgtggatgagtacctgatcctgtcgggggatcatctctaccgcatggactatagccagttcattcagcggcaccgagacaccaatgccgacatcacactctcggtcttgccgatcgatgaaaagcgcgcctctgattttggcctgatgaagctagatggcagcggccgggtggtcgagttcagcgaaaagcccaaaggggatgaactcagggcgatgcaagtcgataccacgatcctcgggcttgaccctgtcgctgctgctgcccagcccttcattgcctcgatgggcatctacgtcttcaagcgggatgttctgatcgatttgctcagccatcatcccgagcaaaccgactttggcaaggaagtgattcccgctgcagccacccgctacaacacccaagcctttctgttcaacgactactgggaagacatcggcacgatcgcctcattctacgaggccaatctggcgctgactcagcaacctagcccacccttcagcttctacgacgagcaggcgccgatttacacccgcgctcgctacctgccgccaaccaagctgctcgattgccaggtgacccagtcgatcattggcgagggctgcattctcaagcaatgcaccgttcagaattccgtcttagggattcgctcccgcattgaggccgactgcgtgatccaggacgccttgttgatgggcgctgacttctacgaaacctcggagctacggcaccagaatcgggccaatggcaaagtgccgatgggaatcggcagtggcagcaccatccgtcgcgccatcgtcgacaaaaatgcccacattggccagaacgttcagatcgtcaacaaagaccatgtggaagaggccgatcgcgaagatctgggctttatgatccgcagcggcattgtcgttgtggtcaaaggggcggttattcccgacaacacggtgatctaa

SEQ ID NO: 28 - DNA, 1320 bp, Synechocystis sp. PCC 6803
gtgtgttgttggcaatcgagaggtctgcttgtgaaacgtgtcttagcgattatcctgggcggtggggccgggacccgcctctatcctttaaccaaactcagagccaaacccgcagttcccttggccggaaagtatcgcctcatcgatattcccgtcagtaattgcatcaactcagaaatcgttaaaatttacgtccttacccagtttaattccgcctcccttaaccgtcacatcagccgggcctataatttttccggcttccaagaaggatttgtggaagtcctcgccgcccaacaaaccaaagataatcctgattggtttcagggcactgctgatgcggtacggcaatacctctggttgtttagggaatgggacgtagatgaatatcttattctgtccggcgaccatctctaccgcatggattacgcccaatttgttaaaagacaccgggaaaccaatgccgacataaccctttccgttgtgcccgtggatgacagaaaggcacccgagctgggcttaatgaaaatcgacgcccagggcagaattactgacttttctgaaaagccccagggggaagccctccgggccatgcaggtggacaccagcgttttgggcctaagtgcggagaaggctaagcttaatccttacattgcctccatgggcatttacgttttcaagaaggaagtattgcacaacctcctggaaaaatatgaaggggcaacggactttggcaaagaaatcattcctgattcagccagtgatcacaatctgcaagcctatctctttgatgactattgggaagacattggtaccattgaagccttctatgaggctaatttagccctgaccaaacaacctagtcccgactttagtttttataacgaaaaagcccccatctataccaggggtcgttatcttccccccaccaaaatgttgaattccaccgtgacggaatccatgatcggggaaggttgcatgattaagcaatgtcgcatccaccactcagttttaggcattcgcagtcgcattgaatctgattgcaccattgaggatactttggtgatgggcaatgatttctacgaatcttcatcagaacgagacaccctcaaagcccggggggaaattgccgctggcataggttccggcaccactatccgccgagccatcatcgacaaaaatgcccgcatcggcaaaaacgtcatgattgtcaacaaggaaaatgtccaggaggctaaccgggaagagttaggtttttacatccgcaatggcatcgtagtagtgattaaaaatgtcacgatcgccgacggcacggtaatctag

SEQ ID NO: 29 - DNA, 1290 bp, Synechococcus sp. PCC 7002
gtgaaacgagtcctaggaatcatacttggcggcggcgcaggtactcgcctatatccgctaacaaaactcagagctaagcccgcagtacctctagcaggcaaatatcgtctcattgatattcctgttagcaattgcattaattctgaaattcataaaatctacattttaacccaatttaattcagcatctttaaatcgtcacattagtcgaacctacaactttaccggcttcaccgaaggctttaccgaagtactcgcagcccaacaaactaaagaaaatcccgattggttccaaggcaccgccgacgctgtccgacagtacagttggcttctagaagactgggatgtcgatgaatacatcattctctccggtgatcacctctaccgtatggattaccgtgaatttatccagcgccaccgtgacactggggcagacatcaccctgtctgtggttcccgtgggcgaaaaagtagcccccgcctttgggttgatgaaaattgatgccaatggtcgtgtcgtggactttagtgaaaagcccactggtgaagcccttaaggcgatgcaggtggatacccagtccttgggtctcgatccagagcaggcgaaagaaaagccctacattgcgtcgatggggatctacgtctttaagaaacaagtactcctcgatctactcaaagaaggcaaagataaaaccgatttcgggaaagaaattattcctgatgcggccaaggactacaacgttcaggcctatctctttgatgattattgggctgacattgggaccatcgaagcgttctatgaagcaaaccttggcttgacgaagcagccgatcccaccctttagtttctatgacgaaaaggctcccatctacacccgggcgcgctacttaccgccgacgaaggtgctcaacgctgacgtgacagaatcgatgatcagcgaaggttgcatcattaaaaactgccgcattcaccactcagttcttggcattcgcacccgtgtcgaagcggactgcactatcgaagatacgatgatcatgggcgcagattattatcagccctatgagaagcgccaggattgtctccgtcgtggcaagcctcccattgggattggtgaagggacaacgattcgccgggcgatcatcgataaaaatgcacgcatcggtaaaaacgtgatgatcgtcaataaggaaaatgtggaggagtcaaaccgtgaggagcttggctactacattcgcagcggcattacagtggtgctaaagaacgccgttattcccgacggtacggtcatttaa

SEQ ID NO: 30 - DNA, 1296 bp, Synechococcus sp. WH8102
atgaagcgggttttggccatcattctcggcggcggtgccgggactcgtctctacccgctcaccaagatgcgcgccaagccggccgtccccttggccggtaagtatcgactgattgatatccccatcagcaactgcatcaactcgaacatcaacaagatgtacgtgatgacgcagttcaacagtgcgtctctcaatcgtcacctcagccagacgttcaacctgagcgcatccttcggtcagggattcgtcgaggtgcttgctgcccagcagacgcctgacagtccatcctggtttgaaggcactgccgacgctgtgcggaagtaccagtggctgttccaggaatgggatgtcgatgaatacctgatcctgtccggtgaccagctgtaccggatggattacagcctgttcgttgaacatcaccgcagcactggtgctgacctcaccgttgcagcccttcctgtggacccgaaacaggccgaggcgttcggcttgatgcgcacggatggtgacggagacatcaaggagttccgcgaaaagcccaagggtgattctttgcttgagatggcggttgacaccagccgatttggactcagtgcgaattcggccaaggagcgtccctacctggcgtcgatggggatttatgtcttcagcagagacactctgttcgacctgctcgattccaatcctggttataaggacttcggcaaggaagtcattcctgaggccctcaagcgtggcgacaagctgaagagctatgtctttgacgattattgggaagatatcggaacgatcggagcgttctacgaggccaacctggcgctcacccagcaacccacaccccccttcagcttctacgacgagaagttcccgatctacactcgtccccgctatttacccccgagcaaactggttgatgctcagatcaccaattcgatcgttggcgaaggctcaattttgaagtcatgcagcattcatcactgcgttttgggtgttcgcagtcgcattgaaaccgatgtggtgctgcaagacaccttggtgatgggcgctgacttctttgaatccagtgatgagcgtgccgtgcttcgcgagcgtggtggtattccggtcggggtgggccaaggtacgactgtgaagcgcgccatcctcgataaaaacgctcgcatcggatccaacgtcaccatcgtcaacaaggatcacgtcgaggaagctgatcgttccgatcagggcttctatattcgtaatggcattgttgttgttgtcaagaacgccaccatccaggacggaactgtgatctga

SEQ ID NO: 31 - DNA, 1296 bp, Synechococcus sp. RCC 307
atgaaacgggttctcgcaatcattctcggtggcggtgcgggtacgcggctctatccgctgaccaaaatgcgggccaaaccagccgtgccgctggcgggtaagtaccgcctcatcgacatccccgttagcaactgcatcaacagcgggatcaacaagatctatgtgctgacgcagttcaacagcgcatcactgaatcgccacatcgctcaaaccttcaacctctcctcggggtttgatcaagggtttgttgaagttctggcggcccagcagaccccagatagccccagttggtttgaaggaacagccgatgctgttcgtaaatacgaatggctgctgcaggagtgggacatcgacgaagtgctgatcctttcgggtgaccagctctaccggatggactatgcccattttgtggctcagcaccgcgccagcggcgctgacctcaccgtggccgccctcccggttgatcgcgagcaagcccagagctttggcttgatgcacaccggtgcagaagcctccatcaccaagttccgcgaaaagcccaaaggcgaggcactcgatgagatgtcctgcgataccgccagcatgggcttgagcgctgaggaagcccatcgccggccgttcctggcttccatgggcatctacgtgttcaagcgggacgtgctcttccgcttactggctgaaaaccccggtgccactgacttcggtaaggagatcatccccaaggcactcgacgatggcttcaaactccgctcctatctcttcgacgattactgggaagacatcggaaccatccgtgctttctatgaagcgaatctggcgctgacgacccagccgcgtccgcccttctctttctacgacaagcgtttcccgatctacacacgtcatcgctacctgccgccctccaagcttcaagatgcgcaggtcaccgactccattgttggtgaggggtccattttgaaggcttgcagtattcaccactgcgtcttgggtgtgcgcagccgcattgaagacgaggttgccttgcaagacaccctggtgatgggcaacgacttctatgagtccggcgaagagcgggccatcctgcgggaacgtggtggcatccccatgggtgtgggccgaggaaccacggtgaaaaaggccatcctcgataagaacgtccgcatcggcagcaacgtcagcatcatcaacaaagacaacgttgaggaagccgaccgcgctgagcagggcttctacatccgtggcgggattgtggtgatcaccaaaaacgcttcgattcccgacgggatggtgatctga

SEQ ID NO: 32 - DNA, 1287 bp, Trichodesmium erythraeum IMS 101
gtgaaaaacgtactaagtataattctaggcggtggcgcaggtacccgtttatatcccttaacaaaactacgggccaagcctgcagtgcccctagcaggaaaatatcgtttaatagatattcctataagtaattgcataaactcagaaatccagaaaatttatgttttgacccaatttaactcagcttctctaaaccgccatatcactcgtacctataacttctcaggtttcagtgatggttttgtcgaagttctagcagctcaacaaactaaagataatccagagtggtttcaaggaacagcagatgctgtccgtaaatatatatggttattcaaagagtgggatattgattattatctaattctctctggagaccatctctaccgtatggactaccgagactttgtccaacgccatatcgacaccaaggcagatatcaccctttctgtcttgcctattgatgaagcacgggcctccgagtttggcgtcatgaaaattgataactcaggtcgaattgttgaatttagtgaaaaaccgaaaggtaatgcccttaaagctatggcagttgatacttctattttaggagtcagtccagaaatagctacaaaacaaccttatattgcttctatgggaatttatgtatttaataaagatgcaatgatcaaacttatagaagattcagaggatacagattttggtaaggaaattttacccaagtcggctcaatcttataatcttcaagcctacccattccaaggttactgggaagacatcggaaccatcaaatcattttatgaagctaatttggctttgactcaacagcctcagccaccctttagcttttatgatgaacaagcccctatctatacccgctctcgttatttacctccgagcaaacttttggactgtgagattacagagtcaattgtgggagaaggttgtattcttaaaaaatgtcggattgaccattgtgtcttaggagtgcgatcgcgtatagaagctaattgtataattcaagattctctgctaatgggttcagatttctatgaatctcctacagaacgtcgatatggcctaaaaaaaggttctgtacctttgggtattggtgctgaaacgaaaattcgtggagcaattattgacaaaaatgcccgcattggttgtaatgtccaaataatcaataaggacaatgtagaagaagcccaacgtgaggaggaagggtttatcattcgcagtggtattgttgttgttttgaaaaatgctactattcccgatggtacagtgatttag

SEQ ID NO: 33 - DNA, 1290 bp, Anabaena variabilis
gtgaaaaaagtcttagcaattattcttggtggtggtgcgggtactcgcctttacccactaaccaaactccgcgctaaaccggcagtaccagtggcagggaaataccgcctaatagatatccctgtcagtaactgcattaattcggaaatttttaaaatctacgtattaacacaatttaactcagcttctctcaatcgccacattgcccgtacctacaactttagtggttttagcgagggttttgtggaagtgctggccgcccagcagacaccagagaaccctaactggttccaaggtacagccgatgctgtacgtcagtatctctggatgttacaagagtgggacgtagatgaatttttgatcctgtcaggagatcacctgtaccggatggattatcgcctatttatccagcgccatcgagaaaccaatgcggatatcacactttccgtaattcccattgacgatcgccgcgcctcggattttggtttaatgaagatcgataactctggacgagtcatcgattttagcgaaaaacccaaaggcgaagccttaaccaaaatgcgtgttgataccaccgttttaggcttgacaccagaacaggcagcatcacagccttacatcgcctcgatggggatttacgtatttaaaaaagatgttttgatcaaactgttgaaggaatctttagaacgtactgatttcggcaaagaaattattcctgatgcctccaaagatcacaacgttcaagcttacttattcgatgactactgggaagatattgggacaatcgaagctttttataatgctaatttagcattgactcagcagcccatgccgccctttagcttctacgacgaagaagcaccaatttatacccgcgcacgttacttaccacccacaaaactattagattgccacgttacagaatcaatcattggcgaaggctgtattctgaaaaactgtcgcattcaacactcagtattgggagtgcgatcgcgtattgaaaccggctgcgtcatcgaagaatctttactcatgggtgccgacttctaccaagcttcagtggaacgccagtgcagcattgacaaaggagacatccccgtaggcatcggcccagataccattattcgccgtgccatcatcgataaaaatgcccgcatcggtcacgatgtcaaaattatcaataaagacaacgtgcaggaagccgaccgcgaaagtcaaggattttacatccgcagtggcattgtcgtcgttctcaaaaatgccgtcattaccgatggcacaataatttag

SEQ ID NO: 34 - DNA, 1290 bp, Nostoc sp. PCC 7120
gtgaaaaaagtcttagcaattattcttggtggtggtgcgggtactcgcctttacccactaaccaaactccgcgctaaaccggcagtaccagtggcagggaaataccgcctaatagatatccctgtcagtaactgcattaattcggaaatttttaaaatctacgtattaacacaatttaactcagcttctctcaatcgccacattgcccgtacctacaactttagtggttttagcgagggttttgtggaagtgctggccgcccagcagacaccagagaaccctaactggttccaaggtacagccgatgctgtacgtcagtatctctggatgttacaagagtgggacgtagatgaatttttgatcctgtcgggggatcacctgtaccggatggactatcgcctatttatccagcgccatcgagaaaccaatgcggatatcacactttccgtaattcccattgatgatcgccgcgcctcggattttggtttaatgaaaatcgataactctggacgagtcattgatttcagtgaaaaacccaagggcgaagccttaaccaaaatgcgtgttgataccacggttttaggcttgacaccagaacaggcggcatcacagccttacattgcctcgatggggatttacgtatttaaaaaagacgttttgatcaagctgttgaaggaagctttagaacgtactgatttcggcaaagaaattattcctgatgccgccaaagatcacaacgttcaagcttacctattcgatgactactgggaagatattgggacaatcgaagctttttataacgccaatttagcgttaactcagcagcccatgccgccctttagcttctacgatgaagaagcacctatttatacccgcgctcgttacttaccacccacaaaactattagattgccacgttacagaatcaatcattggcgaaggctgtattctgaaaaactgtcgcattcaacactcagtattgggagtgcgatcgcgtattgaaactggctgcatgatcgaagaatctttactcatgggtgccgacttctaccaagcttcagtggaacgccagtgcagcatcgataaaggagacatccctgtaggcatcggtccagatacaatcattcgccgtgccatcatcgataaaaatgcccgcatcggtcacgatgtcaaaattatcaataaagacaacgtgcaagaagccgaccgcgaaagtcaaggattttacatccgcagtggcattgtcgtcgtcctcaaaaatgccgttattacagatggcacaatcatttag

SEQ ID NO: 35 - DNA, 1398 bp, Synechococcus elongatus PCC 7942
atgcggattctgttcgtggctgccgaatgtgctcccttcgccaaagtgggaggcatgggagatgtggttggttccctgcccaaagtgctgaaagctctgggccatgatgtccgaatcttcatgccgtactacggctttctgaacagtaagctcgatattcccgctgaaccgatctggtggggctacgcgatgtttaatcacttcgcggtttacgaaacgcagctgcccggttcagatgtgccgctctacttaatggggcatccagcttttgatccgcatcgcatctactcaggagaagacgaagactggcgcttcacgttttttgccaatggggctgctgaattttcttggaactactggaaaccacaagtcattcactgccacgattggcacactgggatgattccggtttggatgcaccagtccccggatatctcgactgtcttcaccattcataacttggcctaccaagggccgtggcgctggaagctcgagaaaatcacctggtgcccttggtacatgcagggcgacagcaccatggcggcggccttgctctatgccgatcgcgtcaacacggtatcgcccacctatgcccagcagattcaaacaccgacctacggtgaaaagctggagggtcttctctcatttatcagtggcaagctaagcggcatccttaacgggattgatgttgatagctacaaccctgcaacggatacgcggattgtggccaactacgatcgcgacactcttgataaacgactgaacaataagctggcgctccaaaaggagatggggcttgaggtcaatcccgatcgcttcctgattggctttgtggctcgtctagtcgagcagaagggcattgacttgctgctgcaaattcttgatcgctttctgtcttacagcgatgcccaatttgttgtcttaggaacgggcgagcgctactacgaaacccagctctgggagttggcgacccgctatccgggccggatgtccacttatctgatgtacgacgaggggctgtcgcgacgcatttatgccggtagcgacgccttcttggtgccctctcgttttgaaccttgcggtatcacgcaaatgctggcactgcgctacggcagtgtgccgattgtgcgccgtacgggggggttggtcgatacggtcttccaccacgatccgcgtcatgccgagggcaatggctattgcttcgatcgctacgagccgctggacctctatacctgtctggtgcgggcttgggagagttaccagtaccagccccaatggcaaaagctacagcaacggggtatggccgttgatctgagctggaaacaatcggcgatcgcctacgaacagctctacgctgaagcgattgggctaccgatcgatgtcttacaggaggcctag

SEQ ID NO: 36 - DNA, 1434 bp, Synechocystis sp. PCC 6803
atgaagattttatttgtggcggcggaagtatcccccctagcaaaggtaggtggcatgggggatgtggtgggttccctgcctaaagttctgcatcagttgggccatgatgtccgtgtcttcatgccctactacggtttcatcggcgacaagattgatgtgcccaaggagccggtctggaaaggggaagccatgttccagcagtttgctgtttaccagtcctatctaccggacaccaaaattcctctctacttgttcggccatccagctttcgactcccgaaggatctatggcggagatgacgaggcgtggcggttcacttttttttctaacggggcagctgaatttgcctggaaccattggaagccggaaattatccattgccatgattggcacactggcatgatccctgtttggatgcatcagtccccagacatcgccaccgttttcaccatccataatcttgcttaccaagggccctggcggggcttgcttgaaactatgacttggtgtccttggtacatgcagggagacaatgtgatggcggcggcgattcaatttgccaatcgggtgactaccgtttctcccacctatgcccaacagatccaaaccccggcctatggggaaaagctggaagggttattgtcctacctgagtggtaatttagtcggtattctcaacggtattgatacggagatttacaacccggcggaagaccgctttatcagcaatgttttcgatgcggacagtttggacaagcgggtgaaaaataaaattgccatccaggaggaaacggggttagaaattaatcgtaatgccatggtggtgggtatagtggctcgcttggtggaacaaaaggggattgatttggtgattcagatccttgaccgcttcatgtcctacaccgattcccagttaattatcctcggcactggcgatcgccattacgaaacccaactttggcagatggcttcccgatttcctgggcggatggcggtgcaattactccacaacgatgccctttcccgtcgagtctatgccggggcggatgtgtttttaatgccttctcgctttgagccctgtgggctgagtcaattgatggccatgcgttatggctgtatccccattgtgcggcggacagggggtttggtggatacggtatccttctacgatcctatcaatgaagccggcaccggctattgctttgaccgttatgaacccctggattgctttacggccatggtgcgggcctgggagggtttccgtttcaaggcagattggcaaaaattacagcaacgggccatgcgggcagactttagttggtaccgttccgccggggaatatatcaaagtttataagggcgtggtggggaaaccggaggaattaagccccatggaagaggaaaaaatcgctgagttaactgcttcctatcgctaa

SEQ ID NO: 37 - DNA, 1437 bp, Synechococcus sp. PCC 7002
atgcgtattttgtttgtttctgccgaggctgctcccatcgctaaagctggaggcatgggagatgtggtgggatcactgcctaaagttttacggcagttaggacatgacgcgagaattttcttaccctattacggctttctcaacgacaaactcgacatccctgcagaacccgtttggtggggcagtgcgatgttcaatacttttgccgtttatgaaactgtgttgcccaacaccgatgtccccctttatctgtttggccatcccgcctttgatggacggcatatttatggtgggcaggatgaattttggcgctttaccttttttgccaatggggccgctgaatttatgtggaaccactggaaaccccagatcgcccactgtcacgactggcacacgggcatgattccggtatggatgcaccaatcgccggatatcagtacggtgtttacgatccacaacttagcctaccaagggccttggcggggtttcctggagcgcaatacttggtgtccctggtatatggatggtgataacgtgatggcttcggcgctgatgtttgccgatcaggtgaacaccgtatctcccacctatgcccaacaaatccaaaccaaagtctatggtgaaaaattagagggtttgttgtcttggatcagtggcaaaagtcgcggcatcgtgaatggtattgacgtagaactttataatccttctaacgatcaagccctggtgaagcaattttctacgactaatcttgaggatcgggccgccaacaaagtgattatccaagaagaaacggggctagaggtcaactccaaggcttttttgatggcgatggtcacccgcttagtggaacaaaagggcattgatctgctgctaaatatcctggagcagtttatggcatacactgacgcccagctcattatcctcggcactggcgatcgccactacgaaacccaactctggcagactgcctaccgctttaaggggcggatgtccgtgcaactgctctataatgatgccctctcccgccggatttacgctggatccgatgtctttttgatgccgtcacgctttgagccctgtggcattagtcaaatgatggcgatgcgctacggttctgtaccgattgtgcggcgcaccgggggtttggtggatacggtctctttccatgatccgattcaccaaaccgggacaggctttagttttgaccgctacgaaccgctggatatgtacacctgcatggtgcgggcttgggaaagtttccgctacaaaaaagactgggctgaactacaaagacgaggcatgagccatgactttagttggtacaaatctgccggggaatatctcaagatgtaccgccaaagcattaaagaagctccggaattaacgaccgatgaagccgaaaaaatcacctatttagtgaaaaaacacgccatttaa

SEQ ID NO: 38 - DNA, 1542 bp, Synechococcus sp. WH8102
atgcgcatcctcttcgctgccgcggaatgcgccccgatgatcaaggtcggtggcatgggggatgtggtgggatcgctgcctccggctctggccaagcttggccacgacgtgcggctgatcatgccgggctactccaagctctggaccaagctgacgatctcggacgaacccatctggcgcgcccagacgatgggtacggaattcgcggtttacgagacgaagcatccaggcaatgggatgaccatctacctggtgggacatccggtgttcgatcccgagcggatctatggcggtgaagatgaggactggcgcttcaccttctttgccagtgccgccgctgaattcgcctggaatgtctggaagccgaatgttcttcactgccacgactggcacaccggcatgattccggtctggatgcaccaggacccggagatcagcacggtcttcaccatccacaacctcaagtaccagggcccctggcgttggaagctggatcgcatcacctggtgcccctggtacatgcagggagatcacaccatggcggcggcacttctgtacgccgaccgggtcaacgccgtctcccccacctacgccgaggaaatccgtacggcggagtacggcgaaaagctggatggtttgctcaatttcgtctccggcaagctgcgcggcatcctcaatggcattgacctcgaggcctggaacccccagaccgatggggctctgccggccaccttcagcgccgacgacctctccggtaaagcggtctgcaagcgggtgttgcaggagcgcatgggtcttgaggtgcgtgacgacgcctttgtcctcggcatggtcagccgactcgtcgatcagaagggcgtcgatctgcttctgcaggtggcggaccgtttgctcgcctacaccgacacgcagatcgtggtgctcggcaccggtgaccgtggcctggaatccggcctgtggcagctggcctcccgccatgccggccgttgcgccgtcttcctcacctacgacgacgacctctcccgactgatctatgccggcagtgacgccttcctgatgcccagtcgcttcgagccctgcggcatcagccagctgtacgccatgcgttacggctccgttcctgtggtgcgcaaggtgggcggcctggtggacaccgttcctccccacagtccagctgatgccagcgggaccggcttctgcttcgatcgttttgagccggtcgacttctacaccgcattggtgcgtgcctgggaggcctaccgccatcgcgacagctggcaggagttgcagaagcgcggcatgcagcaggactacagctgggaccgttcggccatcgattacgacgtcatgtaccgcgatgtctgcggtctgaaggaacccacccctgatgccgcgatggtggaacagttctcccagggacaggctgcggatccctcccgcccagaggatgatgcgatcaatgctgctcccgaggcggtcaccgcgccgtccggccccagccgcaacccccttaatcgtctcttcggccgcagggccgactga

SEQ ID NO: 39 - DNA, 1524 bp, Synechococcus sp RCC 307
atgcgcatcctctttgctgcggccgaatgcgcaccgatggtgaaagtcggcggcatgggagatgtggtgggatctctgcctccagccctcgctgagttgggtcacgacgtgcgcgtgatcatgcccggctacggcaagctctggtcccagcttgatgtgcccagcgagccgatctggcgtgcccaaaccatgggcaccgattttgctgtctatgagacccgtcaccccaagaccgggctcacgatctatttggtgggccatccggtttttgatggtgagcgcatctatggaggtgaagacgaggactggcgcttcaccttcttcgctagcgccacctccgaatttgcctggaacgcttggaagccccaggtgctgcattgccatgactggcacaccggcatgattccggtgtggatgcaccaagaccccgagatcagcacggtcttcaccatccacaacctcaaatatcaaggtccctggcgctggaagctcgagcgcatgacctggtgcccctggtacatgcagggcgaccacaccatggcggcagccttgctgtatgccgaccgcgtcaatgcggtttcacccacctacgcccaagagatccgcacgccggaatacggcgaacaactggaggggttgctgaactacatcagcggcaagctgcgaggcatcctcaatggcatcgatgtggaggcttggaatcccgccactgattcgcggattccggccacctacagcactgctgacctcagtggcaaagccgtctgcaagcgggctctgcaagagcgcatggggcttcaggtgaaccccgacacctttgtgatcggtttggtgagccgtttggtggaccaaaaaggcgtcgacctgctgctgcaggttgccgaacgcttccttgcctacaccgatacgcagatcgttgtgttgggcaccggggatcgccatttggaatcgggcctgtggcaaatggcgagtcagcacagcggccgcttcgcttccttcctcacctacgacgatgatctctcccggctgatctacgccggcagtgatgccttcttgatgccctcgcgctttgagccctgcggcatcagccagttgctctcgatgcgctacggcaccatcccggtggtgcgccgcgtcggtggactggtcgacaccgtgcctccctatgttcccgccacccaagagggcaatggcttctgcttcgaccgctatgaagcgatcgacctttacaccgccttggtgcgcgcctgggaggcctaccgccatcaagacagctggcagcaattgatgaagcgggtgatgcaggttgatttcagctgggctcgttccgccttggaatacgaccgcatgtatcgcgatgtttgcggaatgaaggagcccacgccggaagccgatgcggtggcggccttctccattccccagccgcctgaacagcaggccgcacgtgctgccgctgaagccgctgaccccaacccccaacggcgctttaatccccttggattgctgcgccgaaacggcggttga

SEQ ID NO: 40 - DNA, 1383 bp, Trichodesmium erythraeum IMS 101
atgcgaattttatttgtgtctgctgaagcgactcctttagcaaaagttggtggtatggcagatgtagtgggtgccttacccaaagtactacggaaaatgggtcacgatgttcgtatcttcatgccttattatggctttttaggcgacaagatggaagttcctgaggaacctatctgggaaggaacggccatgtatcaaaactttaagatttatgagacggtactaccaaaaagtgacgtgccattgtacctatttggtcacccggctttttggccacgtcatatttactatggagatgatgaggactggagattcactctatttgctaatggggcggccgagttttgctggaatggctggaaaccagagatagttcattgtaatgactggcacactggcatgattccagtttggatgcacgaaactccagacattaaaaccgtatttactattcataacctagcttatcaaggaccttggcgctggtacttggaaagaattacttggtgtccttggtacatggaagggcataatacaatggcagcagcagttcagtttgcagatcgggtaactactgtttctccaacctatgctagtcagatccaaacacctgcctacggagaaaatctagatggtttaatgtcttttattacggggaaactacacggtatcctcaatggtattgatatgaacttttataatccagctaatgacagatatattcctcaaacttatgatgtcaataccctggaaaaacgggttgacaataaaattgctcttcaagaagaagtaggttttgaagttaacaaaaatagctttctcatgggaatggtctcccgactggtagaacaaaaaggacttgatttaatgctgcaagtcttagatcggtttatggcttatactgatactcagtttattttgttgggtacaggcgatcgcttctatgaaacccaaatgtggcaaatagcaagtcgttatcctggtcggatgagtgtccaacttttacataatgatgccctttcccgacgaatatatgcaggtactgatgctttcttaatgcccagtcgatttgagccttgtggtattagtcagttattggcaatgcgttatggtagtatacctattgtccgtcgcacaggtgggttagttgatactgtctctttctatgatcctattaataatgtaggtactggctattcttttgatcgctatgaaccactagacctgcttactgcaatggtccgagcctatgaaggtttccggttcaaagatcaatggcaggagttacagaagcgtggcatgagagagaactttagctgggataagtcagctcaaggttatatcaaaatgtacaaatcaatgctcggattacctgaagaataa

SEQ ID NO: 41 - DNA, 1419 bp, Anabaena variabilis
atgcggattctatttgtggcagcagaagcagcacccatcgcaaaagtaggagggatgggtgatgttgtcggtgcattacctaaggtcttgagaaaaatggggcatgatgtgcgtatcttcttgccctattacggctttttgccagacaaaatggaaattcccaaagatccaatctggaagggatacgccatgtttcaggactttacagttcacgaagcagttctgcctggtactgatgttcccttgtatttatttggacatccagccttcaacccccggcgaatttattcgggagatgatgaagactggcggttcaccttgttttccaatggtgcggcggaattttgttggaattactggaaaccagaaattattcactgtcacgattggcacacaggcatgattcctgtgtggatgaaccaatcaccagatatcaccacagtcttcactatccacaacctagcttaccaagggccttggcgttggtatctagataaaattacttggtgtccttggtatatgcagggacacaacacaatggcggcggctgtccagtttgctgacagagtaaataccgtttctcctacatacgccgagcaaatcaagaccccggcttacggtgagaaaatagaaggcttgctgtctttcatcagtggtaaattatctgggattgttaacggtatagatacggaagtttatgacccagctaatgataaatttattgctcaaacttttactgctgatactttagataaacgcaaagccaacaaaattgctttacaagaagaagtagggttagaagttaacagcaatgcctttttaattggcatggtgacaaggttagtcgagcagaagggtttagatttagtcatccaaatgctcgatcgctttatggcttatactgatgctcagttcgtcttgttaggaacaggcgatcgctactacgaaactcaaatgtggcaattagcatcccgctaccccggacgtatggccacctatctcctatacaatgatgccctatcccgccgcatctacgccggttctgatgcctttttaatgcccagccgctttgaaccatgcggtattagccagatgatggctttacgctacggttccatccccatcgttcgccgcactgggggtttagttgacaccgtatcccaccacgaccccgtaaacgaagccggtacaggctactgctttgaccgctacgaacccctagacttattcacctgcatgattcgcgcctgggaaggcttccgctacaaaccccaatggcaagaactacaaaagcgtggtatgagtcaagacttcagctggtacaaatccgctaaggaatacgacagactctatcgctcaatatacggtttgccagaagcagaagagacacagccagagttaattctggcaaatcagtag

SEQ ID NO: 42 - DNA, 1419 bp, Nostoc sp. PCC 7120
atgcggattctatttgtggcagcagaagcagcacccattgcaaaagtaggagggatgggtgatgttgtcggtgcattacctaaggtcttgagaaaaatggggcatgatgtacgtatcttcttgccctattacggctttttgccagacaaaatggagattcccaaagatccaatatggaagggatacgccatgtttcaggactttacagttcacgaagcagttctgcctggtactgatgttcccttgtatttatttggacatccagcctttaccccccggcggatttattcgggagatgatgaagactggcgcttcaccttgttttccaatggtgcggctgagttttgctggaattactggaaacccgacattattcactgtcatgattggcacacgggcatgattcctgtgtggatgaaccaatcaccagatatcaccacagtcttcactatccacaatctggcttaccaagggccttggcgttggtatttagataaaattacttggtgtccttggtatatgcagggacacaacacaatggcggcggctgtccagtttgcggacagggtaaatacagtttctcccacatacgccgagcaaatcaagaccccggcttacggtgagaaaatagaaggtttgctgtctttcatcagtggtaaattatctgggattgttaacggtatagatacggaagtttacgacccagctaatgataaatatattgctcaaacgttcactgccgatactttagataaacgcaaagccaacaaaattgctttacaagaagaagtaggattagaagttaacagcaatgcctttttaattggcatggtgacaaggttagtcgagcagaagggcttagatttagtcatccaaatgctcgatcgctttatggcttatactgatgctcagttcgtcttgttgggaacaggcgatcgctactacgaaacccaaatgtggcaattagcatcccgctaccccggtcgtatggctacttacctcctgtataacgatgccctatctcgccgcatctacgctggtactgatgcctttttgatgcccagtcgctttgaaccatgcggtattagtcaaatgatggctttacgctacggttccattcccatcgtccgccgcactggaggcttggttgacaccgtatcccaccacgaccccatcaacgaagcaggtacaggctactgcttcgaccgctacgaacccctcgacttatttacctgcatgattcgcgcctgggaaggcttccgctacaaaccacaatggcaagaactacaaaaacgcggtatgagtcaagacttcagctggtacaaatccgctaaggaatacgacaaactctatcgctcaatgtacggtttgccagacccagaagagacacagccggagttaattctgacaaatcagtag

SEQ ID NO: 43 - DNA, 1953 bp, Synechococcus elongatus PCC 7942 0918
atggtgactggaaccgccctcgcgcaaccccgcgccattacgccccacgaacagcagcttttggccaaactgaaaagctatcgcgatatccaaagcttgtcgcaaatttggggacgtgctgccagtcaatttggatcgatgccggctttggttgcaccccatgccaaaccagcgatcaccctcagttatcaagaattggcgattcagatccaagcgtttgcagccggactgctcgcgctgggagtgcctacctccacagccgatgactttccgcctcgcttggcgcagtttgcggataacagcccccgctggttgattgctgaccaaggcacgttgctggcaggggctgccaatgcggtgcgcggcgcccaagctgaagtatcggagctgctctacgtcttagaggacagcggttcgatcggcttgattgtcgaagacgcggcgctgctgaagaaactacagcctggtttagcgtcactatcgctgcagtttgtgatcgtgctcagcgatgaagtagtcgagatcgacagcctgcgcgtcgttggttttagtgacgtgctggagatggggcgatcgctgccggcaccggagccaattttgcagctcgatcgcttagccactttgatctatacctcgggcaccacaggcccaccgaagggcgtgatgctttctcacggcaacctgctgcaccaagtcacaacattaggtgtggttgtgcagccgcaacctggcgacaccgtgctgagtattttgccgacttggcactcctacgagcgagcttgtgaatatttcctgctctcccagggctgcacacaggtctacacgacgctgcgcaatgtcaaacaagacatccggcagtatcggccgcagttcatggtcagtgtgctgcgcctctgggaatcgatctacgagggcgtgcagaagcagtttcgcgagcaaccggcgaagaaacgtcgcttgatcgataccttctttggcttgagtcaacgctatgttttggcacggcgccgctggcaaggactggatttgctggcactgaaccaatccccagcccagcgcctcgctgagggtgtccggatgttggcgctagcaccgttgcataagctgggcgatcgcctcgtctacggcaaagtacgagaagccacgggtggccgaattcggcaggtgatcagtggcggtggctcactggcactgcacctcgataccttcttcgaaattgttggtgttgatttgctggtgggttatggcttgacagaaacctcaccagtgctgacggggcgacggccttggcacaacctacggggttcggccggtcagccgattccaggtacggcgattcggatcgtcgatcctgaaacgaaggaaaaccgacccagtggcgatcgcggcttggtgctggcgaaagggccgcaaatcatgcagggctacttcaataaacccgaggcgaccgcgaaagcgatcgatgccgaaggttggtttgacaccggcgacttaggctacatcgtcggtgaaggcaacttggtgctaacggggcgcgctaaggacacgatcgtgctgaccaatggcgaaaacattgaaccccagccgattgaagatgcctgcctacgaagttcctatatcagccaaatcatgttggtgggacaagaccgcaagagtttgggggcgttgattgtgcccaatcaagaggcgatcgcactctgggccagcgaacagggcatcagccaaaccgatctgcagggagtggtacagaagctgattcgcgaggaactgaaccgcgaagtgcgcgatcgcccgggctaccgcatcgacgatcgcattggaccattccgcctcatcgaagaaccgttcagcatggaaaatggccagctaacccaaaccctgaaaatccgtcgcaacgttgtcgcggaacactacgcggctatgatcgacgggatgtttgaatcggcgagttaa

SEQ ID NO: 44 - protein, 650 aa, Synechococcus elongatus PCC 7942 0918
MVTGTALAQPRAITPHEQQLLAKLKSYRDIQSLSQIWGRAASQFGSMPALVAPHAKPAITLSYQELAIQIQAFAAGLLALGVPTSTADDFPPRLAQFADNSPRWLIADQGTLLAGAANAVRGAQAEVSELLYVLEDSGSIGLIVEDAALLKKLQPGLASLSLQFVIVLSDEVVEIDSLRVVGFSDVLEMGRSLPAPEPILQLDRLATLIYTSGTTGPPKGVMLSHGNLLHQVTTLGVVVQPQPGDTVLSILPTWHSYERACEYFLLSQGCTQVYTTLRNVKQDIRQYRPQFMVSVLRLWESIYEGVQKQFREQPAKKRRLIDTFFGLSQRYVLARRRWQGLDLLALNQSPAQRLAEGVRMLALAPLHKLGDRLVYGKVREATGGRIRQVISGGGSLALHLDTFFEIVGVDLLVGYGLTETSPVLTGRRPWHNLRGSAGQPIPGTAIRIVDPETKENRPSGDRGLVLAKGPQIMQGYFNKPEATAKAIDAEGWFDTGDLGYIVGEGNLVLTGRAKDTIVLTNGENIEPQPIEDACLRSSYISQIMLVGQDRKSLGALIVPNQEAIALWASEQGISQTDLQGVVQKLIREELNREVRDRPGYRIDDRIGPFRLIEEPFSMENGQLTQTLKIRRNVVAEHYAAMIDGMFESAS

SEQ ID NO: 45 - protein, 325 aa, Synechococcus sp. PCC7002
MPKTERRTFLLDFEKPLSELESRIHQIRDLAAENNVDVSEQIQQLEARADQLREEIFSTLTPAQRLQLARHPRRPSTLDYVQMMADEWFELHGDRGGSDDPALIGGVARFDGQPVMMLGHQKGRDTKDNVARNFGMPAPGGYRKAMRLMDHANRFGMPILTFIDTPGAWAGLEAEKLGQGEAIAFNLREMFSLDVPIICTVIGEGGSGGALGIGVGDRVLMLKNSVYTVATPEACAAILWKDAGKSEQAAAALKITAEDLKSLEIIDEIVPEPASCAHADPIGAAQLLKAAIQDNLQALLKLTPERRRELRYQRFRKIGVFLESS

SEQ ID NO: 46 - protein, 165 aa, Synechococcus sp. PCC 7002
MAINLQEIQELLSTIGQTNVTEFELKTDDFELRVSKGTVVAAPQTMVMSEAIAQPAMSTPVVSQATATPEASQAETPAPSVSIDDKWVAITSPMVGTFYRAPAPGEDPFVAVGDRVGNGQTVCIIEAMKLMNEIEAEVSGEVVKIAVEDGEPIEFGQTLMWVNPT

SEQ ID NO: 47 - protein, 448 aa, Synechococcus sp. PCC 7002
MQFSKILIANRGEVALRIIHTCQELGIATVAVHSTVDRQALHVQLADESICIGPPQSSKSYLNIPNIIAAALSSNADAIHPGYGFLAENAKFAEICADHQITFIGPSPEAMIAMGDKSTAKKTMQAAKVPTVPGSAGLVASEEQALEIAQQIGYPVMIKATAGGGGRGMRLVPSAEELPRLYRAAQGEAEAAFGNGGVYIEKFIERPRHIEFQILADQYGNVIHLGERDCSIQRRHQKLLEEAPSAILTPRLRDKMGKAAVKAAKSIDYVGAGTVEFLVDKNGDFYFMEMNTRIQVEHPVTEMVTGLDLIAEQIKVAQGDRLSLNQNQVNLNGHAIECRINAEDPDHDFRPTPGKISGYLPPGGPGVRMDSHVYTDYEISPYYDSLIGKLIVWGPDRDTAIRRMKRALRECAITGVSTTISFHQKILNHPAFLAADVDTNFIQQHMLP

SEQ ID NO: 48 - protein, 319 aa, Synechococcus sp. PCC 7002
MSLFDWFAANRQNSETQLQPQQEREIADGLWTKCKSCDALTYTKDLRNNQMVCKECGFHNRVGSRERVRQLIDEGTWTEISQNVAPTDPLKFRDKKAYSDRLKDYQEKTNLTDAVITGTGLIDGLPLALAVMDFGFMGGSMGSVVGEKICRLVEHGTAEGLPVVVVCASGGARMQEGMLSLMQMAKISGALERHRTKKLLYIPVLTNPTTGGVTASFAMLGDLILAEPKATIGFAGRRVIEQTLREKLPDDFQTSEYLLQHGFVDAIVPRTELKKTLAQMISLHQPFHPILPELQLAPHVEKEKVYEPIASTSTNDFYK

SEQ ID NO: 49 - protein, 2311 aa, Triticum aestivum
MGSTHLPIVGLNASTTPSLSTIRPVNSAGAAFQPSAPSRTSKKKSRRVQSLRDGGDGGVSDPNQSIRQGLAGIIDLPKEGTSAPEVDISHGSEEPRGSYQMNGILNEAHNGRHASLSKVVEFCMALGGKTPIHSVLVANNGRAAAKFMRSVRTWANETFGSEKAIQLIAMATPEDMRINAEHIRIADQFVEVPGGTNNNNYANVQLIVEIAVRTGVSAVWPGWGHASENPELPDALNANGIVFLGPPSSSMNALGDKVGSALIAQAAGVPTLPWGGSQVEIPLEVCLDSIPAEMYRKACVSTTEEALASCQMIGYPAMIKASWGGGGKGIRKVNNDDDVRALFKQVQGEVPGSPIFIMRLASQSRHLEVQLLCDQYGNVAALHSRDCSVQRRHQKIIEEGPVTVAPRETVKELEQAARRLAKAVGYVGAATVEYLYSMETGEYYFLELNPRLQVEHPVTEWIAEVNLPAAQVAVGMGIPLWQVPEIRRFYGMDNGGGYDIWRETAALATPFNFDEVDSQWPKGHCVAVRITSEDPDDGFKPTGGKVKEISFKSKPNVWAYFSVKSGGGIHEFADSQFGHVFAYGVSRAAAITNMSLALKEIQIRGEIHSNVDYTVDLLNASDFKENRIHTGWLDNRIAMRVQAERPPWYISVVGGALYKTITSNTDTVSEYVSYLVKGQIPPKHISLVHSTVSLNIEESKYTIETIRSGQGSYRLRMNGSVIEANVQTLCDGGLLMQLDGNSHVIYAEEEAGGTRLLIDGKTYLLQNDHDPSRLLAETPCKLLRFLVADGAHVEADVPYAEVEVMKMCMPLLSPAAGVINVLLSEGQPMQAGDLIARLDLDDPSAVKRAEPFNGSFPEMSLPIAASGQVHKRCATSLNAARMVLAGYDHPINKVVQDLVSCLDAPELPFLQWEELMSVLATRLPRLLKSELEGKYSEYKLNVGHGKSKDFPSKMLREIIEENLAHGSEKEIATNERLVEPLMSLLKSYEGGRESHAHFIVKSLFEDYLSVEELFSDGIQSDVIERLRQQHSKDLQKVVDIVLSHQGVRNKTKLILTLMEKLVYPNPAVYKDQLTRFSSLNHKRYYKLALKASELLEQTKLSELRTSIARSLSELEMFTEERTAISEIMGDLVTAPLPVEDALVSLFDCSDQTLQQRVIETYISRLYQPHLVKDSIQLKYQESGVIALWEFAEAHSEKRLGAMVIVKSLESVSAAIGAALKGTSRYASSEGNIMHIALLGADNQMHGTEDSGDNDQAQVRIDKLSATLEQNTVTADLRAAGVKVISCIVQRDGALMPMRHTFLLSDEKLCYGEEPVLRHVEPPLSALLELGKLKVKGYNEVKYTPSRDRQWNIYTLRNTENPKMLHRVFFRTLVRQPGASNKFTSGNISDVEVGGAEESLSFTSSSILRSLMTAIEELELHAIRTGHSHMFLCILKERKLLDLVPVSGNKVVDIGQDEATACLLLKEMALQIHELVGARMHHLSVCQWEVKLKLDSDGPASGTWRVVTTNVTSHTCTVDIYREVEDTESQKLVYHSAPSSSGPLHGVALNTPYQPLSVIDLKRCSARNNRTTYCYDFPLAFETAVQKSWSNISSDNNRCYVKATELVFAHKNGSWGTPVIPMERPAGLNDIGMVAWILDMSTPEYPNGRQIVVIANDITFRAGSFGPREDAFFETVTNLACERRLPLIYLAANSGARIGIADEVKSCFRVGWSDDGSPERGFQYIYLTEEDHARISASVIAHKMQLDNGEIRWVIDSVVGKEDGLGVENIHGSAAIASAYSRAYEETFTLTFVTGRTVGIGAYLARLGIRCIQRTDQPIILTGFSALNKLLGREVYSSHMQLGGPKIMATNGVVHLTVSDDLEGVSNILRWLSYVPANIGGPLPITKSLDPPDRPVAYIPENTCDPRAAISGIDDSQGKWLGGMFDKDSFVETFEGWAKSVVTGRAKLGGIPVGVIAVETQTMMQLIPADPGQLDSHERSVPRAGQVWFPDSATKTAQAMLDFNREGLPLFILANWRGFSGGQRDLFEGILQAGSTIVENLRAYNQPAFVYIPKAAELRGGAWVVIDSKINPDRIEFYAERTAKGNVLEPQGLIEIKFRSEELQECMGRLDPELINLKAKLQGVKHENGSLPESESLQKSIEARKKQLLPLYTQIAVRFAELHDTSLRMAAKGVIKKVVDWEDSRSFFYKRLRRRISEDVLAKEIRGVSGKQFSHQSAIELIQKWYLASKGAETGSTEWDDDDAFVAWRENPENYQEYIKEPRAQRVSQLLSDVADSSPDLEALPQGLSMLLEKMDPAKREIVEDFEINLVK

SEQ ID NO: 50 - DNA, 978 bp, Synechococcus sp. PCC 7002
atgccgaaaacggagcgccggacgtttctgcttgattttgaaaaacctctttcggaattagaatcacgcatccatcaaattcgtgatcttgctgcggagaataatgttgatgtttcagaacagattcagcagctagaggcgcgggcagaccagctccgggaagaaatttttagtaccctcaccccggcccaacggctgcaattggcacggcatccccggcgtcccagcacccttgattatgttcaaatgatggcggacgaatggtttgaactccatggcgatcgcggtggatctgatgatccggctctcattggcggggtggcccgcttcgatggtcaaccggtgatgatgctagggcaccaaaaaggacgggatacgaaggataatgtcgcccgcaattttggcatgccagctcctgggggctaccgtaaggcgatgcggctgatggaccatgccaaccgttttgggatgccgattttaacgtttattgatactcctggggcttgggcgggtttagaagcggaaaagttgggccaaggggaggcgatcgcctttaacctccgggaaatgtttagcctcgatgtgccgattatttgcacggtcattggcgaaggcggttccggtggggccttagggattggcgtgggcgatcgcgtcttgatgttaaaaaattccgtttacacagtggcgaccccagaggcttgtgccgccattctctggaaagatgccgggaaatcagagcaggccgccgccgccctcaagattacagcagaggatctgaaaagccttgagattatcgatgaaattgtcccagagccagcctcctgcgcccacgccgatcccattggggccgcccaactcctgaaagcagcgatccaagataacctccaagccttgctgaagctgacgccagaacgccgccgtgaattgcgctaccagcggttccggaaaattggtgtgtttttagaaagttcctaa

SEQ ID NO: 51 - DNA, 498 bp, Synechococcus sp.  PCC 7002
atggctattaatttacaagagatccaagaacttctatccaccatcggccaaaccaatgtcaccgagtttgaactcaaaaccgatgattttgaactccgtgtgagcaaaggtactgttgtggctgctccccagacgatggtgatgtccgaggcgatcgcccaaccagcaatgtccactcccgttgtttctcaagcaactgcaaccccagaagcctcccaagcggaaaccccggctcccagtgtgagcattgatgataagtgggtcgccattacctcccccatggtgggaacgttttaccgcgcgccggcccctggtgaagatcccttcgttgccgttggcgatcgcgttggcaatggtcaaaccgtttgcatcatcgaagcgatgaaattaatgaatgagattgaggcagaagtcagcggtgaagttgttaaaattgccgttgaagacggtgaacccattgaatttggtcagaccctaatgtgggtcaacccaacctaa

SEQ ID NO: 52 - DNA, 1347 bp, Synechococcus sp. PCC 7002
atgcagttttcaaagattctcatcgccaatcgcggagaagttgccctacgcattatccacacctgtcaggagctcggcattgccacagttgccgtccactccaccgtagatcgccaagccctccacgttcagctcgccgatgagagcatttgcattggcccgccccagagcagcaaaagctatctcaacattcccaatattatcgctgcggccctcagcagtaacgccgacgcaatccacccaggctacggtttcctcgctgaaaatgccaagtttgcagaaatttgtgccgaccaccaaatcaccttcattggcccttccccagaagcaatgatcgccatgggggacaaatccaccgccaaaaaaacgatgcaggcggcaaaagtccctaccgtacccggtagtgctgggttggtggcctccgaagaacaagccctagaaatcgcccaacaaattggctaccctgtgatgatcaaagccacggcgggtggtggtggccgggggatgcgccttgtgcccagcgctgaggagttaccccgtttgtaccgagcggcccagggggaagcagaagcagcctttgggaatggcggcgtttacatcgaaaaatttattgaacggccccgtcacatcgaatttcagatcctcgcggatcagtacggcaatgtaattcacctcggcgaacgggattgttcgatccaacggcggcaccaaaaactcctcgaagaagctcccagcgcgatcctcacccccagactgcgggacaaaatggggaaagcggcagtaaaagcggcgaaatccattgattatgtcggggcggggacggtggaattcctcgtggataagaatggggatttctactttatggaaatgaatacccgcattcaggtggaacacccggtcacagagatggtgacgggactagatctgatcgccgagcaaattaaagttgcccaaggcgatcgcctcagtttgaatcaaaatcaagtgaacttgaatggtcatgccatcgagtgccggattaatgccgaagatcccgaccatgatttccgaccgaccccaggcaaaatcagtggctatcttccccccggtggccctggggtacggatggattcccacgtttacaccgactatgaaatttctccttactacgattctttgatcggtaaattaatcgtttggggaccagaccgagacaccgccattcgccgcatgaagcgggcactccgagaatgtgccattactggagtatcgaccaccattagcttccaccaaaagattttgaatcatccggcttttttggcggccgatgtcgatacaaactttatccagcagcacatgttgccctag

SEQ ID NO: 53 - DNA, 960 bp, Synechococcus sp. PCC 7002
atgtctctttttgattggtttgccgcaaatcgccaaaattctgaaacccagctccagccccaacaggagcgcgagattgccgatggcctctggacgaaatgcaaatcctgcgatgctctcacctacactaaagacctccgcaacaatcaaatggtctgtaaagagtgtggcttccataaccgggtcggcagtcgggaacgggtacgccaattgattgacgaaggcacctggacagaaattagtcagaatgtcgcgccgaccgaccccctgaaattccgcgacaaaaaagcctatagcgatcgcctcaaagattaccaagagaaaacgaacctcaccgatgctgtaatcactggcacaggactgattgacggtttaccccttgctttggcagtgatggactttggctttatgggcggcagcatgggatccgttgtcggcgaaaaaatttgtcgcctcgtagaacatggcaccgccgaaggtttacccgtggtggttgtttgtgcttctggtggagcaagaatgcaagagggcatgctcagtctgatgcagatggcgaaaatctctggtgccctcgaacgccatcgcaccaaaaaattactctacatccctgttttgactaatcccaccaccgggggcgtcaccgctagctttgcgatgttgggcgatttgattcttgccgaacccaaagcaaccatcggttttgctggacgccgcgtcattgaacaaacattgcgcgaaaaacttcctgacgattttcagacatctgaatatttactccaacatgggtttgtggatgcgattgtgccccgcactgaattgaaaaaaaccctcgcccaaatgattagtctccatcagccctttcacccgattctgccagagctacaattggctccccatgtggaaaaagaaaaagtttacgaacccattgcctctacttcaaccaacgacttttacaagtag

SEQ ID NO: 54 - DNA, 6936 bp, Triticum aestivum
atgggatccacacatttgcccattgtcggccttaatgcctcgacaacaccatcgctatccactattcgcccggtaaattcagccggtgctgcattccaaccatctgccccttctagaacctccaagaagaaaagtcgtcgtgttcagtcattaagggatggaggcgatggaggcgtgtcagaccctaaccagtctattcgccaaggtcttgccggcatcattgacctcccaaaggagggcacatcagctccggaagtggatatttcacatgggtccgaagaacccaggggctcctaccaaatgaatgggatactgaatgaagcacataatgggaggcatgcttcgctgtctaaggttgtcgaattttgtatggcattgggcggcaaaacaccaattcacagtgtattagttgcgaacaatggaagggcagcagctaagttcatgcggagtgtccgaacatgggctaatgaaacatttgggtcagagaaggcaattcagttgatagctatggctactccagaagacatgaggataaatgcagagcacattagaattgctgatcaatttgttgaagtacccggtggaacaaacaataacaactatgcaaatgtccaactcatagtggagatagcagtgagaaccggtgtttctgctgtttggcctggttggggccatgcatctgagaatcctgaacttccagatgcactaaatgcaaacggaattgtttttcttgggccaccatcatcatcaatgaacgcactaggtgacaaggttggttcagctctcattgctcaagcagcaggggttccgactcttccttggggtggatcacaggtggaaattccattagaagtttgtttggactcgatacctgcggagatgtataggaaagcttgtgttagtactacggaggaagcacttgcgagttgtcagatgattgggtatccagccatgattaaagcatcatggggtggtggtggtaaagggatccgaaaggttaataacgacgatgatgtcagagcactgtttaagcaagtgcaaggtgaagttcctggctccccaatatttatcatgagacttgcatctcagagtcgacatcttgaagttcagttgctttgtgatcaatatggcaatgtagctgcgcttcacagtcgtgactgcagtgtgcaacggcgacaccaaaagattattgaggaaggaccagttactgttgctcctcgcgagacagtgaaagagctagagcaagcagcaaggaggcttgctaaggctgtgggttatgttggtgctgctactgttgaatatctctacagcatggagactggtgaatactattttctggaacttaatccacggttgcaggttgagcatccagtcaccgagtggatagctgaagtaaacttgcctgcagctcaagttgcagttggaatgggtatacccctttggcaggttccagagatcagacgtttctatggaatggacaatggaggaggctatgacatttggagggaaacagcagctcttgctactccatttaacttcgatgaagtggattctcaatggccaaagggtcattgtgtagcagttaggataaccagtgaggatccagatgacggattcaagcctaccggtggaaaagtaaaggagatcagttttaaaagcaagccaaatgtttgggcctatttctctgttaagtccggtggaggcattcatgaatttgctgattctcagtttggacatgtttttgcatatggagtgtctagagcagcagcaataaccaacatgtctcttgcgctaaaagagattcaaattcgtggagaaattcattcaaatgttgattacacagttgatctcttgaatgcctcagacttcaaagaaaacaggattcatactggctggctggataacagaatagcaatgcgagtccaagctgagagacctccgtggtatatttcagtggttggaggagctctatataaaacaataacgagcaacacagacactgtttctgaatatgttagctatctcgtcaagggtcagattccaccgaagcatatatcccttgtccattcaactgtttctttgaatatagaggaaagcaaatatacaattgaaactataaggagcggacagggtagctacagattgcgaatgaatggatcagttattgaagcaaatgtccaaacattatgtgatggtggacttttaatgcagttggatggaaacagccatgtaatttatgctgaagaagaggccggtggtacacggcttctaattgatggaaagacatacttgttacagaatgatcacgatccttcaaggttattagctgagacaccctgcaaacttcttcgtttcttggttgccgatggtgctcatgttgaagctgatgtaccatatgcggaagttgaggttatgaagatgtgcatgcccctcttgtcacctgctgctggtgtcattaatgttttgttgtctgagggccagcctatgcaggctggtgatcttatagcaagacttgatcttgatgacccttctgctgtgaagagagctgagccatttaacggatctttcccagaaatgagccttcctattgctgcttctggccaagttcacaaaagatgtgccacaagcttgaatgctgctcggatggtccttgcaggatatgatcacccgatcaacaaagttgtacaagatctggtatcctgtctagatgctcctgagcttcctttcctacaatgggaagagcttatgtctgttttagcaactagacttccaaggcttcttaagagcgagttggagggtaaatacagtgaatataagttaaatgttggccatgggaagagcaaggatttcccttccaagatgctaagagagataatcgaggaaaatcttgcacatggttctgagaaggaaattgctacaaatgagaggcttgttgagcctcttatgagcctactgaagtcatatgagggtggcagagaaagccatgcacactttattgtgaagtcccttttcgaggactatctctcggttgaggaactattcagtgatggcattcagtctgatgtgattgaacgcctgcgccaacaacatagtaaagatctccagaaggttgtagacattgtgttgtctcaccagggtgtgagaaacaaaactaagctgatactaacactcatggagaaactggtctatccaaaccctgctgtctacaaggatcagttgactcgcttttcctccctcaatcacaaaagatattataagttggcccttaaagctagcgagcttcttgaacaaaccaagcttagtgagctccgcacaagcattgcaaggagcctttcagaacttgagatgtttactgaagaaaggacggccattagtgagatcatgggagatttagtgactgccccactgccagttgaagatgcactggtttctttgtttgattgtagtgatcaaactcttcagcagagggtgatcgagacgtacatatctcgattataccagcctcatcttgtcaaggatagtatccagctgaaatatcaggaatctggtgttattgctttatgggaattcgctgaagcgcattcagagaagagattgggtgctatggttattgtgaagtcgttagaatctgtatcagcagcaattggagctgcactaaagggtacatcacgctatgcaagctctgagggtaacataatgcatattgctttattgggtgctgataatcaaatgcatggaactgaagacagtggtgataacgatcaagctcaagtcaggatagacaaactttctgcgacactggaacaaaatactgtcacagctgatctccgtgctgctggtgtgaaggttattagttgcattgttcaaagggatggagcactcatgcctatgcgccataccttcctcttgtcggatgaaaagctttgttatggggaagagccggttctccggcatgtggagcctcctctttctgctcttcttgagttgggtaagttgaaagtgaaaggatacaatgaggtgaagtatacaccgtcacgtgatcgtcagtggaacatatacacacttagaaatacagagaaccccaaaatgttgcacagggtgtttttccgaactcttgtcaggcaacccggtgcttccaacaaattcacatcaggcaacatcagtgatgttgaagtgggaggagctgaggaatctctttcatttacatcgagcagcatattaagatcgctgatgactgctatagaagagttggagcttcacgcgattaggacaggtcactctcatatgtttttgtgcatattgaaagagcgaaagcttcttgatcttgttcccgtttcagggaacaaagttgtggatattggccaagatgaagctactgcatgcttgcttctgaaagaaatggctctacagatacatgaacttgtgggtgcaaggatgcatcatctttctgtatgccaatgggaggtgaaacttaagttggacagcgatgggcctgccagtggtacctggagagttgtaacaaccaatgttactagtcacacctgcactgtggatatctaccgtgaggttgaagatacagaatcacagaaactagtataccactctgctccatcgtcatctggtcctttgcatggcgttgcactgaatactccatatcagcctttgagtgttattgatctgaaacgttgctccgctagaaacaacagaactacatactgctatgattttccgttggcatttgaaactgcagtgcagaagtcatggtctaacatttctagtgacaataaccgatgttatgttaaagcaacggagctggtgtttgctcacaagaatgggtcatggggcactcctgtaattcctatggagcgtcctgctgggctcaatgacattggtatggtagcttggatcttggacatgtccactcctgaatatcccaatggcaggcagattgttgtcatcgcaaatgatattacttttagagctggatcgtttggtccaagggaagatgcattttttgaaactgttaccaacctagcttgtgagaggaggcttcctctcatctacttggcagcaaactctggtgctcggatcggcatagcagatgaagtaaaatcttgcttccgtgttggatggtctgatgatggcagccctgaacgtgggtttcaatatatttatctgactgaagaagaccatgctcgtattagcgcttctgttatagcgcacaagatgcagcttgataatggtgaaattaggtgggttattgattctgttgtagggaaggaggatgggctaggtgtggagaacatacatggaagtgctgctattgccagtgcctattctagggcctatgaggagacatttacgcttacatttgtgactggaaggactgttggaataggagcatatcttgctcgacttggcatacggtgcattcagcgtactgaccagcccattatcctaactgggttttctgccttgaacaagcttcttggccgggaagtgtacagctcccacatgcagttgggtggccccaaaattatggcgacaaacggtgttgtccatctgacagtttcagatgaccttgaaggtgtatctaatatattgaggtggctcagctatgttcctgccaacattggtggacctcttcctattacaaaatctttggacccacctgacagacccgttgcttacatccctgagaatacatgcgatcctcgtgctgccatcagtggcattgatgatagccaagggaaatggttggggggcatgttcgacaaagacagttttgtggagacatttgaaggatgggcgaagtcagttgttactggcagagcgaaactcggagggattccggtgggtgttatagctgtggagacacagactatgatgcagctcatccctgctgatccaggccagcttgattcccatgagcgatctgttcctcgtgctgggcaagtctggtttccagattcagctactaagacagcgcaggcaatgctggacttcaaccgtgaaggattacctctgttcatccttgctaactggagaggcttctctggtggacaaagagatctttttgaaggaatccttcaggctgggtcaacaattgttgagaaccttagggcatacaatcagcctgcctttgtatatatccccaaggctgcagagctacgtggaggggcttgggtcgtgattgatagcaagataaatccagatcgcattgagttctatgctgagaggactgcaaagggcaatgttctcgaacctcaagggttgatcgagatcaagttcaggtcagaggaactccaagagtgcatgggtaggcttgatccagaattgataaatctgaaggcaaagctccagggagtaaagcatgaaaatggaagtctacctgagtcagaatcccttcagaagagcatagaagcccggaagaaacagttgttgcctttgtatactcaaattgcggtacggttcgctgaattgcatgacacttcccttagaatggctgctaagggtgtgattaagaaggttgtagactgggaagattctaggtcgttcttctacaagagattacggaggaggatatccgaggatgttcttgcgaaggaaattagaggtgtaagtggcaagcagttttctcaccaatcggcaatcgagctgatccagaaatggtacttggcctctaagggagctgaaacaggaagcactgaatgggatgatgacgatgcttttgttgcctggagggaaaaccctgaaaactaccaggagtatatcaaagaacccagggctcaaagggtatctcagttgctctcagatgttgcagactccagtccagatctagaagccttgccacagggtctttctatgctactagagaagatggatcctgcaaagagggaaattgttgaagactttgaaataaaccttgtaaagtaa

SEQ ID NO: 55 - protein, 2235 aa, Saccharomyces cerevisiae
MEFSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLSDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVSVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 56 - DNA, 6708 bp, Artificial Sequence
  Codon-optimized S. cerevisiae acetyl Coa carboxylase (ACC1)
atggaattctccgaggaaagtttgttcgaaagcagtccgcagaaaatggaatatgaaattacgaattattcggaacgccacacggagctccccgggcacttcatcggactcaacaccgtggataagctcgaagaaagtcccctccgcgattttgtgaaaagccacggcggccataccgtgatctcgaagattctgattgccaataacggaattgccgctgtcaaggagatccgcagcgtccggaagtgggcgtacgaaacttttggcgatgaccgtacagtccagtttgttgctatggcgactccggaagacttggaggcgaatgcggaatacattcgaatggccgatcaatacatcgaagtccccggaggaacgaacaacaacaattatgcgaacgtcgatttgatcgtggatatcgcagaacgcgcggacgtggatgctgtttgggccggatggggccacgcttcggaaaaccctctgttgccggaaaaactcagccagtctaaacggaaagtcattttcatcggccctccgggcaacgcaatgcgctcgttgggtgataagatcagctcgaccattgtggctcagagcgctaaagtcccatgtattccctggtcgggtaccggcgtggatacggtccatgttgatgagaaaactggactggtcagcgtcgatgatgatatctaccaaaagggctgttgcaccagcccggaagatggcctgcaaaaggcgaagcgcatcgggttcccagtcatgatcaaggcatccgaaggcggaggcggtaagggtatccgccaggttgagcgtgaagaagattttatcgcactgtatcatcaagcggctaacgaaatcccgggctcgccaattttcattatgaaactggctggtcgggcgcgtcatctcgaagtgcaactcctcgctgaccagtacggtacgaacatctctttgttcggtcgggattgttcggtccagcgtcgtcaccagaagatcattgaagaagcccctgttaccatcgcaaaggccgagacgtttcatgagatggagaaagcggccgtccgcctcggcaagctggtcggttacgttagcgcaggcaccgtggaatacctctattcccacgacgatggtaagttttactttctcgaactgaatcctcgcctgcaggttgaacacccgaccacagagatggtgtcgggggtcaatctgccggctgcgcagttgcagattgcaatgggcattccgatgcatcgaatcagcgacatccgaaccctgtacggcatgaacccgcacagtgcgagcgaaatcgactttgagttcaagacccaagacgccacgaagaaacagcgacgcccaattccgaagggccattgcaccgcgtgtcgcattacctcggaggaccccaatgatggttttaagccctcgggcggtactctgcacgagctcaacttccgctcctcctcgaacgtctggggctatttcagcgtcggaaataatggtaacattcatagtttttccgattcccaatttggccatatcttcgcctttggcgaaaaccgacaagctagccgcaaacacatggtcgtggcgttgaaggagctgagtatccgaggggactttcgcacgacggtggaatatctgatcaaactgctcgaaacggaggactttgaggataacacaattaccaccggatggttggacgacctgattacgcacaaaatgaccgccgagaaacccgaccccaccttggcagtgatttgtggcgcggcaacgaaggcctttttggcctctgaagaggcacgccacaagtacattgagagtctccaaaagggtcaggtgctgagtaaagatctgctgcaaaccatgtttcctgtcgactttattcatgaggggaaacgctacaaattcacggttgctaagtctggtaatgatcggtacacattgtttatcaatggatcgaagtgcgatattatcttgcgacaactctccgacggcggcctcctgattgctatcggcgggaaaagtcataccatctattggaaagaagaggtcgccgccacccgactgagcgttgattcgatgactactctgctcgaagttgaaaacgatccaacgcaactgcgcactccctctccgggtaagctcgtgaagtttctcgtcgagaatggcgaacacattattaagggccagccgtatgcggaaatcgaggtgatgaagatgcagatgcccctggtcagccaagagaacggtattgtgcaactgctgaaacagcccggcagcaccatcgtcgctggcgatatcatggctatcatgaccctcgatgatccttccaaagtcaaacatgccctgcccttcgaaggcatgctccccgattttggctcccccgtgattgagggcaccaaaccagcttacaagtttaaatcgctggtttccaccctcgagaacatcttgaagggctacgataatcaggtcattatgaatgccagcctccagcagctcattgaggtcctccgtaaccccaagctgccctacagtgaatggaagctccacatcagtgcgctccactcgcgactgcccgcgaagctcgatgagcagatggaagagctcgtcgctcgcagcctgcgtcgcggcgcagtctttccggcacggcaactgtcgaagctcatcgatatggctgtcaaaaaccccgaatacaaccccgataaactcttgggtgctgtcgttgagccgctcgccgatatcgcgcacaagtacagtaatggcctggaggcgcacgaacacagtatctttgttcacttcctggaagaatactatgaggttgagaaactgttcaatgggcctaatgtccgggaagagaatattatcctgaagctccgtgatgaaaatccgaaagatttggataaagtcgccttgacggtgctcagtcatagcaaggtgagtgccaagaacaatctcatcctggcgatcttgaaacactaccaacctttgtgcaagctgagttccaaggtgtcggctatttttagtacgcccctgcagcacatcgtggaactcgaaagtaaagccaccgccaaggtggctctgcaggcccgggagattctgatccagggtgctctgccgagcgtgaaagagcggacggaacaaatcgaacacatcctgaagagttcggtcgtgaaggttgcatatggcagcagtaaccctaaacgctcggaaccggacctcaatatcctgaaggatctgatcgatagtaattatgttgtttttgatgtcctgctccaatttctgactcaccaagatccggttgttactgcggctgccgcgcaagtttacattcgacgcgcctatcgcgcctacacaatcggcgatattcgagtccatgagggcgtgaccgttccaatcgttgaatggaaattccagttgccatcggcggctttttctacattcccaacagtcaagagtaagatgggcatgaatcgtgccgtttcggtcagtgatttgtcctatgtcgcaaactcgcaatctagtcctctgcgagagggcatcctgatggcagtggatcatttggatgatgtcgatgagatcctctcgcaaagtctcgaggtcattcctcgccaccaatcgtcgtccaatggcccagctcccgatcgatccggttcttccgccagcttgtcgaatgtcgccaacgtctgtgtggcgtcgactgaggggttcgaaagcgaagaagaaattttggtccgcttgcgggaaattttggacctcaacaagcaggaactgattaatgcctctattcgccgcattacgtttatgttcggtttcaaggatggctcgtacccaaaatactatacgttcaacggcccgaactacaatgagaacgagactatccgacatattgaacctgccctcgctttccaactggaactggggcggctctcgaatttcaatattaagcctatttttaccgacaaccgtaacatccacgtttacgaggctgtcagcaaaacaagcccgctggataagcgattcttcacccggggcattatccgcacaggccacatccgtgacgatatcagtatccaagaatacctgactagcgaagctaaccgcttgatgagcgacattttggataatctggaagtgactgatacttccaacagcgacttgaatcacatttttatcaacttcattgccgtgttcgatatctcgccggaagatgtggaagccgcgtttggaggctttctggaacggtttggcaaacggctgctgcgcttgcgggtgtctagcgcggagattcggattatcatcaaagatccgcaaacgggggctcctgtgccactgcgcgcgctgattaataacgtctcgggttacgtgatcaagaccgagatgtacacagaggttaaaaacgctaaaggcgagtgggtcttcaagagcttgggcaaacccggcagcatgcatctccgccccatcgccacgccgtatccggtcaaggagtggctgcagcccaagcgatacaaggcgcacttgatggggacgacatatgtttacgattttcctgaactgttccgtcaagcaagcagctcccagtggaaaaacttttccgcagatgtgaaattgactgatgatttcttcatctcgaatgagctcatcgaagatgagaatggcgagctgaccgaagttgagcgagaacctggtgccaatgcgattgggatggtcgcctttaaaatcacggtcaaaactcccgagtaccctcggggtcgccagttcgtcgttgtggctaacgatatcacctttaagattggatcgtttggcccgcaggaggatgagttctttaacaaggtcactgaatacgcccgaaaacgaggcattccgcggatttacttggcagccaatagcggtgcgcgcatcggcatggctgaagaaatcgttccgctgtttcaggttgcctggaacgacgcggccaaccccgacaaggggttccagtacttgtatctgacttccgaaggcatggagacgttgaagaaatttgataaggagaatagtgtcttgactgagcggaccgttattaacggcgaggagcggtttgtcattaagactatcatcggcagcgaagatggcctcggcgtcgaatgtttgcgcgggtccggcctgatcgcaggggcaacctcgcgagcctatcacgatatctttaccattactttggtcacgtgtcgttcggttggcattggagcatacctcgtgcgcctcggtcagcgcgccatccaagtggaaggccaacctatcattttgactggcgcgcctgctatcaataagatgctgggccgtgaagtctacacatcgaacctccaactgggcggtacccaaattatgtataacaatggcgtcagccatctgacagccgtcgatgacctggctggcgttgaaaagattgttgagtggatgagctatgtgcccgccaaacggaacatgccagtccccattttggaaaccaaggatacctgggatcgcccagtggatttcactccgactaatgatgaaacctacgatgtccgctggatgatcgaagggcgcgaaactgagtcgggcttcgagtacggactgtttgataagggtagtttctttgagactctcagtggttgggccaaaggcgttgtcgtcggtcgggcacgtctgggcggcatcccgctgggagttattggtgttgagacacgtacggtggaaaatctgatcccggctgatccggccaaccccaatagtgcggaaacgctgattcaagagcccgggcaagtgtggcacccgaatagtgcctttaagacggcgcaggctattaatgattttaacaacggcgaacaactgcctatgatgattctggcgaattggcgggggtttagtggtgggcagcgcgacatgttcaacgaagtgctcaagtacggctccttcatcgtggacgccctggtcgactataaacaaccaattatcatctatattccccctaccggcgagctgcgaggcggtagctgggtcgtggtggaccctactattaatgcagatcaaatggagatgtacgccgacgtgaatgctcgagcgggcgtgctggaaccacaagggatggttggcatcaaattccgccgcgaaaaactgttggatactatgaatcgactggatgataaatatcgcgagctgcgcagccaactgtcgaacaagtctctggccccggaagtccatcaacagatttctaaacagctggcagatcgcgaacgtgaactcttgccgatctacggccaaatcagcctccaatttgccgacctgcatgatcgcagcagccgcatggttgcgaaaggtgtcatcagcaaagagctcgagtggacggaagctcggcggtttttcttttggcggctgcgccgacgcctgaatgaagaatacttgattaagcgtctgagccaccaggtcggcgaggctagtcggttggaaaagatcgcccgcattcggagttggtatccggcatcggttgaccacgaggacgatcgccaggtcgctacctggatcgaagagaactacaaaaccttggatgataagctgaaaggactgaagctggagtctttcgcccaagatctcgccaagaagatccgtagcgatcatgacaatgcaatcgacggtttgagcgaggttatcaagatgttgtctaccgacgacaaggagaagctgctcaaaacgctgaagtag

SEQ ID NO: 57 - DNA, 6702 bp, Artificial Sequence
  Synthetic construct Saccharomyces cerevisiae clone FLH148869.01X ACC1
atgagcgaagaaagcttattcgagtcttctccacagaagatggagtacgaaattacaaactactcagaaagacatacagaacttccaggtcatttcattggcctcaatacagtagataaactagaggagtccccgttaagggactttgttaagagtcacggtggtcacacggtcatatccaagatcctgatagcaaataatggtattgccgccgtgaaagaaattagatccgtcagaaaatgggcatacgagacgttcggcgatgacagaaccgtccaattcgtcgccatggccaccccagaagatctggaggccaacgcagaatatatccgtatggccgatcaatacattgaagtgccaggtggtactaataataacaactacgctaacgtagacttgatcgtagacatcgccgaaagagcagacgtagacgccgtatgggctggctggggtcacgcctccgagaatccactattgcctgaaaaattgtcccagtctaagaggaaagtcatctttattgggcctccaggtaacgccatgaggtctttaggtgataaaatctcctctaccattgtcgctcaaagtgctaaagtcccatgtattccatggtctggtaccggtgttgacaccgttcacgtggacgagaaaaccggtctggtctctgtcgacgatgacatctatcaaaagggttgttgtacctctcctgaagatggtttacaaaaggccaagcgtattggttttcctgtcatgattaaggcatccgaaggtggtggtggtaaaggtatcagacaagttgaacgtgaagaagatttcatcgctttataccaccaggcagccaacgaaattccaggctcccccattttcatcatgaagttggccggtagagcgcgtcacttggaagttcaactgctagcagatcagtacggtacaaatatttccttgttcggtagagactgttccgttcagagacgtcatcaaaaaattatcgaagaagcaccagttacaattgccaaggctgaaacatttcacgagatggaaaaggctgccgtcagactggggaaactagtcggttatgtctctgccggtaccgtggagtatctatattctcatgatgatggaaaattctactttttagaattgaacccaagattacaagtcgagcatccaacaacggaaatggtctccggtgttaacttacctgcagctcaattacaaatcgctatgggtatccctatgcatagaataagtgacattagaactttatatggtatgaatcctcattctgcctcagaaatcgatttcgaattcaaaactcaagatgccaccaagaaacaaagaagacctattccaaagggtcattgtaccgcttgtcgtatcacatcagaagatccaaacgatggattcaagccatcgggtggtactttgcatgaactaaacttccgttcttcctctaatgtttggggttacttctccgtgggtaacaatggtaatattcactccttttcggactctcagttcggccatatttttgcttttggtgaaaatagacaagcttccaggaaacacatggttgttgccctgaaggaattgtccattaggggtgatttcagaactactgtggaatacttgatcaaacttttggaaactgaagatttcgaggataacactattaccaccggttggttggacgatttgattactcataaaatgaccgctgaaaagcctgatccaactcttgccgtcatttgcggtgccgctacaaaggctttcttagcatctgaagaagcccgccacaagtatatcgaatccttacaaaagggacaagttctatctaaagacctactgcaaactatgttccctgtagattttatccatgagggtaaaagatacaagttcaccgtagctaaatccggtaatgaccgttacacattatttatcaatggttctaaatgtgatatcatactgcgtcaactatctgatggtggtcttttgattgccataggcggtaaatcgcataccatctattggaaagaagaagttgctgctacaagattatccgttgactctatgactactttgttggaagttgaaaacgatccaacccagttgcgtactccatcccctggtaaattggttaaattcttggtggaaaatggtgaacacattatcaagggccaaccatatgcagaaattgaagttatgaaaatgcaaatgcctttggtttctcaagaaaatggtatcgtccagttattaaagcaacctggttctaccattgttgcaggtgatatcatggctattatgactcttgacgatccatccaaggtcaagcacgctctaccatttgaaggtatgctgccagattttggttctccagttatcgaaggaaccaaacctgcctataaattcaagtcattagtgtctactttggaaaacattttgaagggttatgacaaccaagttattatgaacgcttccttgcaacaattgatagaggttttgagaaatccaaaactgccttactcagaatggaaactacacatctctgctttacattcaagattgcctgctaagctagatgaacaaatggaagagttagttgcacgttctttgagacgtggtgctgttttcccagctagacaattaagtaaattgattgatatggccgtgaagaatcctgaatacaaccccgacaaattgctgggcgccgtcgtggaaccattggcggatattgctcataagtactctaacgggttagaagcccatgaacattctatatttgtccatttcttggaagaatattacgaagttgaaaagttattcaatggtccaaatgttcgtgaggaaaatatcattctgaaattgcgtgatgaaaaccctaaagatctagataaagttgcgctaactgttttgtctcattcgaaagtttcagcgaagaataacctgatcctagctatcttgaaacattatcaaccattgtgcaagttatcttctaaagtttctgccattttctctactcctctacaacatattgttgaactagaatctaaggctaccgctaaggtcgctctacaagcaagagaaattttgattcaaggcgctttaccttcggtcaaggaaagaactgaacaaattgaacatatcttaaaatcctctgttgtgaaggttgcctatggctcatccaatccaaagcgctctgaaccagatttgaatatcttgaaggacttgatcgattctaattacgttgtgttcgatgttttacttcaattcctaacccatcaagacccagttgtgactgctgcagctgctcaagtctatattcgtcgtgcttatcgtgcttacaccataggagatattagagttcacgaaggtgtcacagttccaattgttgaatggaaattccaactaccttcagctgcgttctccacctttccaactgttaaatctaaaatgggtatgaacagggctgtttctgtttcagatttgtcatatgttgcaaacagtcagtcatctccgttaagagaaggtattttgatggctgtggatcatttagatgatgttgatgaaattttgtcacaaagtttggaagttattcctcgtcaccaatcttcttctaacggacctgctcctgatcgttctggtagctccgcatcgttgagtaatgttgctaatgtttgtgttgcttctacagaaggtttcgaatctgaagaggaaattttggtaaggttgagagaaattttggatttgaataagcaggaattaatcaatgcttctatccgtcgtatcacatttatgttcggttttaaagatgggtcttatccaaagtattatacttttaacggtccaaattataacgaaaatgaaacaattcgtcacattgagccggctttggccttccaactggaattaggaagattgtccaacttcaacattaaaccaattttcactgataatagaaacatccatgtctacgaagctgttagtaagacttctccattggataagagattctttacaagaggtattattagaacgggtcatatccgtgatgacatttctattcaagaatatctgacttctgaagctaacagattgatgagtgatatattggataatttagaagtcaccgacacttcaaattctgatttgaatcatatcttcatcaacttcattgcggtgtttgatatctctccagaagatgtcgaagccgccttcggtggtttcttagaaagatttggtaagagattgttgagattgcgtgtttcttctgccgaaattagaatcatcatcaaagatcctcaaacaggtgccccagtaccattgcgtgccttgatcaataacgtttctggttatgttatcaaaacagaaatgtacaccgaagtcaagaacgcaaaaggtgaatgggtatttaagtctttgggtaaacctggatccatgcatttaagacctattgctactccttaccctgttaaggaatggttgcaaccaaaacgttataaggcacacttgatgggtaccacatatgtctatgacttcccagaattattccgccaagcatcgtcatcccaatggaaaaatttctctgcagatgttaagttaacagatgatttctttatttccaacgagttgattgaagatgaaaacggcgaattaactgaggtggaaagagaacctggtgccaacgctattggtatggttgcctttaagattactgtaaagactcctgaatatccaagaggccgtcaatttgttgttgttgctaacgatatcacattcaagatcggttcctttggtccacaagaagacgaattcttcaataaggttactgaatatgctagaaagcgtggtatcccaagaatttacttggctgcaaactcaggtgccagaattggtatggctgaagagattgttccactatttcaagttgcatggaatgatgctgccaatccggacaagggcttccaatacttatacttaacaagtgaaggtatggaaactttaaagaaatttgacaaagaaaattctgttctcactgaacgtactgttataaacggtgaagaaagatttgtcatcaagacaattattggttctgaagatgggttaggtgtcgaatgtctacgtggatctggtttaattgctggtgcaacgtcaagggcttaccacgatatcttcactatcaccttagtcacttgtagatccgtcggtatcggtgcttatttggttcgtttgggtcaaagagctattcaggtcgaaggccagccaattattttaactggtgctcctgcaatcaacaaaatgctgggtagagaagtttatacttctaacttacaattgggtggtactcaaatcatgtataacaacggtgtttcacatttgactgctgttgacgatttagctggtgtagagaagattgttgaatggatgtcttatgttccagccaagcgtaatatgccagttcctatcttggaaactaaagacacatgggatagaccagttgatttcactccaactaatgatgaaacttacgatgtaagatggatgattgaaggtcgtgagactgaaagtggatttgaatatggtttgtttgataaagggtctttctttgaaactttgtcaggatgggccaaaggtgttgtcgttggtagagcccgtcttggtggtattccactgggtgttattggtgttgaaacaagaactgtcgagaacttgattcctgctgatccagctaatccaaatagtgctgaaacattaattcaagaacctggtcaagtttggcatccaaactccgccttcaagactgctcaagctatcaatgactttaacaacggtgaacaattgccaatgatgattttggccaactggagaggtttctctggtggtcaacgtgatatgttcaacgaagtcttgaagtatggttcgtttattgttgacgcattggtggattacaaacaaccaattattatctatatcccacctaccggtgaactaagaggtggttcatgggttgttgtcgatccaactatcaacgctgaccaaatggaaatgtatgccgacgtcaacgctagagctggtgttttggaaccacaaggtatggttggtatcaagttccgtagagaaaaattgctggacaccatgaacagattggatgacaagtacagagaattgagatctcaattatccaacaagagtttggctccagaagtacatcagcaaatatccaagcaattagctgatcgtgagagagaactattgccaatttacggacaaatcagtcttcaatttgctgatttgcacgataggtcttcacgtatggtggccaagggtgttatttctaaggaactggaatggaccgaggcacgtcgtttcttcttctggagattgagaagaagattgaacgaagaatatttgattaaaaggttgagccatcaggtaggcgaagcatcaagattagaaaagatcgcaagaattagatcgtggtaccctgcttcagtggaccatgaagatgataggcaagtcgcaacatggattgaagaaaactacaaaactttggacgataaactaaagggtttgaaattagagtcattcgctcaagacttagctaaaaagatcagaagcgaccatgacaatgctattgatggattatctgaagttatcaagatgttatctaccgatgataaagaaaaattgttgaagactttgaaatag

SEQ ID NO: 58 - protein, 458 aa, Acinetobacter baylii sp.
MRPLHPIDFIFLSLEKRQQPMHVGGLFLFQIPDNAPDTFIQDLVNDIRISKSIPVPPFNNKLNGLFWDEDEEFDLDHHFRHIALPHPGRIRELLIYISQEHSTLLDRAKPLWTCNIIEGIEGNRFAMYFKIHHAMVDGVAGMRLIEKSLSHDVTEKSIVPPWCVEGKRAKRLREPKTGKIKKIMSGIKSQLQATPTVIQELSQTVFKDIGRNPDHVSSFQAPCSILNQRVSSSRRFAAQSFDLDRFRNIAKSLNVTINDVVLAVCSGALRAYLMSHNSLPSKPLIAMVPASIRNDDSDVSNRITMILANLATHKDDPLQRLEIIRRSVQNSKQRFKRMTSDQILNYSAVVYGPAGLNIISGMMPKRQAFNLVISNVPGPREPLYWNGAKLDALYPASIVLDGQALNITMTSYLDKLEVGLIACRNALPRMQNLLTHLEEEIQLFEGVIAKQEDIKTAN

SEQ ID NO: 59 - protein, 446 aa, Streptomyces coelicolor
MTPDPLAPLDLAFWNIESAEHPMHLGALGVFEADSPTAGALAADLLAARAPAVPGLRMRIRDTWQPPMALRRPFAFGGATREPDPRFDPLDHVRLHAPATDFHARAGRLMERPLERGRPPWEAHVLPGADGGSFAVLFKFHHALADGLRALTLAAGVLDPMDLPAPRPRPEQPPRGLLPDVRALPDRLRGALSDAGRALDIGAAAALSTLDVRSSPALTAASSGTRRTAGVSVDLDDVHHVRKTTGGTVNDVLIAVVAGALRRWLDERGDGSEGVAPRALIPVSRRRPRSAHPQGNRLSGYLMRLPVGDPDPLARLGTVRAAMDRNKDAGPGRGAGAVALLADHVPALGHRLGGPLVSGAARLWFDLLVTSVPLPSLGLRLGGHPLTEVYPLAPLARGHSLAVAVSTYRGRVHYGLLADAKAVPDLDRLAVAVAEEVETLLTACRP

SEQ ID NO: 60 - protein, 457 aa, Alcanivorax borkumensis
MKALSPVDQLFLWLEKRQQPMHVGGLQLFSFPEGAGPKYVSELAQQMRDYCHPVAPFNQRLTRRLGQYYWTRDKQFDIDHHFRHEALPKPGRIRELLSLVSAEHSNLLDRERPMWEAHLIEGIRGRQFALYYKIHHSVMDGISAMRIASKTLSTDPSEREMAPAWAFNTKKRSRSLPSNPVDMASSMARLTASISKQAATVPGLAREVYKVTQKAKKDENYVSIFQAPDTILNNTITGSRRFAAQSFPLPRLKVIAKAYNCTINTVVLSMCGHALREYLISQHALPDEPLIAMVPMSLRQDDSTGGNQIGMILANLGTHICDPANRLRVIHDSVEEAKSRFSQMSPEEILNFTALTMAPTGLNLLTGLAPKWRAFNVVISNIPGPKEPLYWNGAQLQGVYPVSIALDRIALNITLTSYVDQMEFGLIACRRTLPSMQRLLDYLEQSIRELEIGAGIK

SEQ ID NO: 61 - protein, 460 aa, Acinetobacter baylii sp.
MEFRPLHPIDFIFLSLEKRQQPMHVGGLFLFQIPDNAPDTFIQDLVNDIRISKSIPVPPFNNKLNGLFWDEDEEFDLDHHFRHIALPHPGRIRELLIYISQEHSTLLDRAKPLWTCNIIEGIEGNRFAMYFKIHHAMVDGVAGMRLIEKSLSHDVTEKSIVPPWCVEGKRAKRLREPKTGKIKKIMSGIKSQLQATPTVIQELSQTVFKDIGRNPDHVSSFQAPCSILNQRVSSSRRFAAQSFDLDRFRNIAKSLNVTINDVVLAVCSGALRAYLMSHNSLPSKPLIAMVPASIRNDDSDVSNRITMILANLATHKDDPLQRLEIIRRSVQNSKQRFKRMTSDQILNYSAVVYGPAGLNIISGMMPKRQAFNLVISNVPGPREPLYWNGAKLDALYPASIVLDGQALNITMTSYLDKLEVGLIACRNALPRMQNLLTHLEEEIQLFEGVIAKQEDIKTAN

SEQ ID NO: 62 - DNA, 1377 bp, Artificial Sequence
  Codon-optimized Acinetobacter baylii sp. atfA
atgcggcccttgcaccccattgacttcatctttctgagtttggagaaacggcaacagcccatgcatgtcggtggcttgtttctcttccaaatccccgataacgccccggacacctttattcaggatctggtcaatgatatccggatctcgaaatcgatccccgtgccgccgtttaataataaactgaacggcctcttttgggacgaagacgaggaatttgatctggatcaccattttcggcacatcgctttgccccacccgggtcggattcgcgaactcctgatctatattagccaagaacacagcacgttgttggaccgggccaaaccgctctggacgtgcaatatcatcgaaggcatcgaaggcaaccgctttgcgatgtacttcaagattcatcacgcgatggttgacggtgtcgctggcatgcgcctgatcgaaaaatcgctgagccatgatgtgaccgaaaagagtatcgtccccccctggtgcgtggaaggtaagcgcgccaagcgcctccgcgaaccgaaaacgggcaagattaagaaaatcatgagcggtatcaagtcgcagctgcaggctaccccgaccgtgatccaggagctgtcgcaaaccgtgtttaaggatattggtcggaacccggatcatgtcagtagtttccaagctccctgttcgatcttgaatcagcgcgttagcagcagccgccggttcgctgctcaaagttttgatctcgatcggtttcggaatattgccaagtcgctgaacgtcaccatcaatgatgtggttctcgcggtttgttcgggtgccctccgcgcgtatctgatgagccataacagtctccccagtaagccgctgattgctatggttcccgcgtcgattcggaatgacgacagcgatgtgagcaaccggattaccatgatcctggctaacctcgcgacccacaaagatgatccgttgcaacgcctggagattatccgccgcagtgtgcagaacagtaaacagcgcttcaaacggatgaccagtgatcaaattctgaattacagcgctgtggtctatggtcccgccggcttgaatattatcagtggtatgatgcccaaacgccaagcgtttaacttggtgatcagtaatgtgccgggtccgcgcgaacccttgtattggaacggtgctaaactcgatgccctctaccccgccagtatcgtgctcgatggccaggctctcaatattaccatgaccagctatctcgataaactcgaggtgggtttgattgcgtgccgcaacgcgctgccccgcatgcagaacttgctgacccacctggaagaggaaatccagctcttcgagggcgtgattgcgaagcaggaagatattaaaacggccaactag

SEQ ID NO: 63 - DNA, 1376 bp, Acinetobacter sp.
atgcgcccattacatccgattgattttatattcctgtcactagaaaaaagacaacagcctatgcatgtaggtggtttatttttgtttcagattcctgataacgccccagacacctttattcaagatctggtgaatgatatccggatatcaaaatcaatccctgttccaccattcaacaataaactgaatgggcttttttgggatgaagatgaagagtttgatttagatcatcattttcgtcatattgcactgcctcatcctggtcgtattcgtgaattgcttatttatatttcacaagagcacagtacgctgctagatcgggcaaagcccttgtggacctgcaatattattgaaggaattgaaggcaatcgttttgccatgtacttcaaaattcaccatgcgatggtcgatggcgttgctggtatgcggttaattgaaaaatcactctcccatgatgtaacagaaaaaagtatcgtgccaccttggtgtgttgagggaaaacgtgcaaagcgcttaagagaacctaaaacaggtaaaattaagaaaatcatgtctggtattaagagtcagcttcaggcgacacccacagtcattcaagagctttctcagacagtatttaaagatattggacgtaatcctgatcatgtttcaagctttcaggcgccttgttctattttgaatcagcgtgtgagctcatcgcgacgttttgcagcacagtcttttgacctagatcgttttcgtaatattgccaaatcgttgaatgtgaccattaatgatgttgtactagcggtatgttctggtgcattacgtgcgtatttgatgagtcataatagtttgccttcaaaaccattaattgccatggttccagcctctattcgcaatgacgattcagatgtcagcaaccgtattacgatgattctggcaaatttggcaacccacaaagatgatcctttacaacgtcttgaaattatccgccgtagtgttcaaaactcaaagcaacgcttcaaacgtatgaccagcgatcagattctaaattatagtgctgtcgtatatggccctgcaggactcaacataatttctggcatgatgccaaaacgccaagccttcaatctggttatttccaatgtgcctggcccaagagagccactttactggaatggtgccaaacttgatgcactctacccagcttcaattgtattagacggtcaagcattgaatattacaatgaccagttatttagataaacttgaagttggtttgattgcatgccgtaatgcattgccaagaatgcagaatttactgacacatttagaagaagaaattcaactatttgaaggcgtaattgcaaagcaggaagatattaaaacagccaatta

SEQ ID NO: 64 - DNA, 1341 bp, Artificial Sequence
  Codon-optimized Streptomyces coelicolor DGAT
atgacgcctgacccgttggctcccttggacttggctttctggaatatcgaaagtgccgagcacccgatgcacttgggggcactgggggtctttgaggcggatagtccaaccgctggtgcactcgccgcggatctcctggctgcccgcgctcccgcagtgcccgggctgcgcatgcggattcgcgatacatggcagccgcctatggcgctccgtcgcccttttgcttttggcggtgctacacgcgagcccgacccgcggtttgatccactcgatcatgtgcggctccatgccccagcgacggatttccacgcacgcgcaggtcggttgatggagcgccctctggaacgaggccgtcctccttgggaagcccatgtcctgccaggggctgacggtggatcgtttgcggtcttgtttaagttccatcatgccctggccgacggtctgcgggcgctgacgctggcggcgggcgtgctcgatccgatggatctccccgctccacggccccgcccagagcagcccccccgtggtctcctgccggatgtccgcgcgctgccggatcggctgcgaggggctctgtctgacgcgggccgcgcgttggacatcggcgccgccgcagccctcagcaccctggatgtgcggagcagtcccgctctgactgcggcgtcctcgggcacgcgacgtaccgccggcgtgtccgtggatctcgacgacgtgcaccatgttcgcaaaacgacaggcggtaccgttaacgatgttttgatcgccgttgttgccggggccctgcgacgctggctggatgaacgaggcgatgggtcggaaggcgtcgccccgcgcgccctcattcccgtcagccggcggcgacctcggagcgcacacccgcaaggcaaccgattgagtggctacctgatgcgcttgccggtcggcgacccggaccctctcgcacggttgggaaccgtccgtgccgcgatggatcgaaataaggatgcggggcccggccgcggagctggcgcagttgctctcttggcagaccacgttcctgccctgggccaccgcctgggtggacccctcgtctcgggcgctgctcgactgtggttcgatctgttggtcacgagcgtcccgttgccctctttgggtttgcgcctcggtgggcatccgctgaccgaagtgtacccactggcccccctggcccgtggccactccttggcggtggcggtgagcacttatcgcggtcgggttcattacggtctcctcgctgatgctaaagccgttcctgatctggatcgtctggcagtggccgtcgccgaggaggttgaaaccttgctcactgcgtgccgcccctag

SEQ ID NO: 65 - DNA, 1374 bp, Artificial Sequence
  Codon-optimized Alcanivorax borkumensis DGAT
atgaaagctttgagccccgttgatcagctgtttctgtggttggaaaaacggcagcaacccatgcatgtgggtgggttgcagctgttctcctttcccgaaggcgcggggccgaaatatgtctcggaactggcccaacagatgcgcgattattgtcaccctgtcgccccgttcaaccaacgtctgacacggcgcctggggcaatactactggacacgtgataagcaatttgacattgaccatcattttcggcacgaggccctgcccaaaccgggtcggattcgcgagttgctcagcttggtgagtgcggaacactccaacttgttggatcgtgaacgacccatgtgggaagcgcacctgatcgaaggaatccgcgggcgccaatttgccttgtattacaaaattcatcactccgtcatggacggtatctccgctatgcggattgcctctaagaccttgtccacggaccccagtgagcgggagatggcccccgcttgggcgtttaatactaagaagcgatcgcgcagcctgccaagcaatcccgtggatatggcgagctcgatggctcgactcactgcaagtatttcgaaacaagctgccaccgtgcccggcctggcacgagaggtctacaaggtgacccaaaaagctaaaaaggatgaaaattacgttagtattttccaagcaccagacaccatcctcaataatacgattacgggcagtcgacgcttcgccgctcagtcgttccctctcccccgtctgaaggttatcgctaaggcttacaactgcactattaacacggttgtgctctcgatgtgcggccacgccctgcgcgaatacctcatcagtcaacatgccctgccggatgaacccctgatcgcgatggtccctatgagcctgcgccaagatgatagcaccggaggcaaccagatcggaatgattttggcgaatctgggcacgcatatctgcgatcctgccaatcgcctgcgtgtcatccatgatagcgtggaggaggcgaaaagccgttttagccaaatgtctccggaggagattctgaactttacagcactcactatggcgccgaccggtctgaacttgctcaccggtttggctcccaaatggcgcgcatttaacgtcgttatctctaacatcccagggccaaaggaaccactgtactggaatggggcacagctccagggtgtgtatccggtctccatcgccttggatcggattgccctgaacattacactgacgtcttatgttgatcagatggagttcggcttgattgcgtgtcgccggaccctcccgtcgatgcaacgactcctcgactatctcgaacagagtatccgcgaactggagattggcgcgggcatcaaatag

SEQ ID NO: 66 - DNA, 1383 bp, Artificial Sequence
  Codon-optimized Acinetobacter baylii sp. DGATd
atggaattccggcccttgcaccccattgacttcatctttctgagtttggagaaacggcaacagcccatgcatgtcggtggcttgtttctcttccaaatccccgataacgccccggacacctttattcaggatctggtcaatgatatccggatctcgaaatcgatccccgtgccgccgtttaataataaactgaacggcctcttttgggacgaagacgaggaatttgatctggatcaccattttcggcacatcgctttgccccacccgggtcggattcgcgaactcctgatctatattagccaagaacacagcacgttgttggaccgggccaaaccgctctggacgtgcaatatcatcgaaggcatcgaaggcaaccgctttgcgatgtacttcaagattcatcacgcgatggttgacggtgtcgctggcatgcgcctgatcgaaaaatcgctgagccatgatgtgaccgaaaagagtatcgtccccccctggtgcgtggaaggtaagcgcgccaagcgcctccgcgaaccgaaaacgggcaagattaagaaaatcatgagcggtatcaagtcgcagctgcaggctaccccgaccgtgatccaggagctgtcgcaaaccgtgtttaaggatattggtcggaacccggatcatgtcagtagtttccaagctccctgttcgatcttgaatcagcgcgttagcagcagccgccggttcgctgctcaaagttttgatctcgatcggtttcggaatattgccaagtcgctgaacgtcaccatcaatgatgtggttctcgcggtttgttcgggtgccctccgcgcgtatctgatgagccataacagtctccccagtaagccgctgattgctatggttcccgcgtcgattcggaatgacgacagcgatgtgagcaaccggattaccatgatcctggctaacctcgcgacccacaaagatgatccgttgcaacgcctggagattatccgccgcagtgtgcagaacagtaaacagcgcttcaaacggatgaccagtgatcaaattctgaattacagcgctgtggtctatggtcccgccggcttgaatattatcagtggtatgatgcccaaacgccaagcgtttaacttggtgatcagtaatgtgccgggtccgcgcgaacccttgtattggaacggtgctaaactcgatgccctctaccccgccagtatcgtgctcgatggccaggctctcaatattaccatgaccagctatctcgataaactcgaggtgggtttgattgcgtgccgcaacgcgctgccccgcatgcagaacttgctgacccacctggaagaggaaatccagctcttcgagggcgtgattgcgaagcaggaagatattaaaacggccaactag

SEQ ID NO: 67 - DNA, 2535 bp, Synechococcus elongatus PCC 7942
atgagtgattccaccgcccaactcagctacgaccccaccacgagctacctcgagcccagtggcttggtctgtgaggatgaacggacttctgtgactcccgagaccttgaaacgggcttacgaggcccatctctactacagccagggcaaaacctcagcgatcgccaccctgcgtgatcactacatggcactggcctacatggtccgcgatcgcctcctgcaacggtggctagcttcactgtcgacctatcaacaacagcacgtcaaagtggtctgttacctgtccgctgagtttttgatgggtcggcacctcgaaaactgcctgatcaacctgcatcttcacgaccgcgttcagcaagttttggatgaactgggtctcgattttgagcaactgctagagaaagaggaagaacccgggctaggcaacggtggcctcggtcgcctcgcagcttgtttcctcgactccatggctaccctcgacattcctgccgtcggctatggcattcgctatgagttcggtatcttccaccaagaactccacaacggctggcagatcgaaatccccgataactggctgcgctttggcaacccttgggagctagagcggcgcgaacaggccgtggaaattaagttgggcggccacacggaggcctaccacgatgcgcgaggccgctactgcgtctcttggatccccgatcgcgtcattcgcgccatcccctacgacacccccgtaccgggctacgacaccaataacgtcagcatgttgcggctctggaaggctgagggcaccacggaactcaaccttgaggctttcaactcaggcaactacgacgatgcggttgccgacaaaatgtcgtcggaaacgatctcgaaggtgctctatcccaacgacaacaccccccaagggcgggaactgcggctggagcagcagtatttcttcgtctcggcttcgctccaagacatcatccgtcgccacttgatgaaccacggtcatcttgagcggctgcatgaggcgatcgcagtccagcttaacgacacccatcccagcgtggcggtgccggagttgatgcgcctcctgatcgatgagcatcacctgacttgggacaatgcttggacgattacacagcgcaccttcgcctacaccaaccacacgctgctacctgaagccttggaacgctggcccgtgggcatgttccagcgcactttaccgcgcttgatggagattatctacgaaatcaactggcgcttcttggccaatgtgcgggcctggtatcccggtgacgacacgagagctcgccgcctctccctgattgaggaaggagctgagccccaggtgcgcatggctcacctcgcctgcgtgggcagtcatgccatcaacggtgtggcagccctgcatacgcaactgctcaagcaagaaaccctgcgagatttctacgagctttggcccgagaaattcttcaacatgaccaacggtgtgacgccccgccgctggctgctgcaaagtaatcctcgcctagccaacctgatcagcgatcgcattggcaatgactggattcatgatctcaggcaactgcgacggctggaagacagcgtgaacgatcgcgagtttttacagcgctgggcagaggtcaagcaccaaaataaggtcgatctgagccgctacatctaccagcagactcgcatagaagtcgatccgcactctctctttgatgtgcaagtcaaacggattcacgaatacaaacgccagctcctcgctgtcatgcatatcgtgacgctctacaactggctgaagcacaatccccagctcaacctggtgccgcgcacttttatctttgcgggcaaagcggccccgggttactaccgtgccaagcaaatcgtcaaactgatcaatgcggtcgggagcatcatcaaccatgatcccgatgtccaagggcgactgaaggtcgtcttcctacctaacttcaacgtttccttggggcagcgcatttatccagctgccgatttgtcggagcaaatctcaactgcagggaaagaagcgtccggcaccggcaacatgaagttcaccatgaatggcgcgctgacaatcggaacctacgatggtgccaacatcgagatccgcgaggaagtcggccccgaaaacttcttcctgtttggcctgcgagccgaagatatcgcccgacgccaaagtcggggctatcgacctgtggagttctggagcagcaatgcggaactgcgggcagtcctcgatcgctttagcagtggtcacttcacaccggatcagcccaacctcttccaagacttggtcagcgatctgctgcagcgggatgagtacatgttgatggcggactatcagtcctacatcgactgccagcgcgaagctgctgctgcctaccgcgattccgatcgctggtggcggatgtcgctactcaacaccgcgagatcgggcaagttctcctccgatcgcacgatcgctgactacagcgaacagatctgggaggtcaaaccagtccccgtcagcctaagcactagcttttag

SEQ ID NO: 68 - protein, 844 aa, Synechococcus elongatus PCC 7942
MSDSTAQLSYDPTTSYLEPSGLVCEDERTSVTPETLKRAYEAHLYYSQGKTSAIATLRDHYMALAYMVRDRLLQRWLASLSTYQQQHVKVVCYLSAEFLMGRHLENCLINLHLHDRVQQVLDELGLDFEQLLEKEEEPGLGNGGLGRLAACFLDSMATLDIPAVGYGIRYEFGIFHQELHNGWQIEIPDNWLRFGNPWELERREQAVEIKLGGHTEAYHDARGRYCVSWIPDRVIRAIPYDTPVPGYDTNNVSMLRLWKAEGTTELNLEAFNSGNYDDAVADKMSSETISKVLYPNDNTPQGRELRLEQQYFFVSASLQDIIRRHLMNHGHLERLHEAIAVQLNDTHPSVAVPELMRLLIDEHHLTWDNAWTITQRTFAYTNHTLLPEALERWPVGMFQRTLPRLMEIIYEINWRFLANVRAWYPGDDTRARRLSLIEEGAEPQVRMAHLACVGSHAINGVAALHTQLLKQETLRDFYELWPEKFFNMTNGVTPRRWLLQSNPRLANLISDRIGNDWIHDLRQLRRLEDSVNDREFLQRWAEVKHQNKVDLSRYIYQQTRIEVDPHSLFDVQVKRIHEYKRQLLAVMHIVTLYNWLKHNPQLNLVPRTFIFAGKAAPGYYRAKQIVKLINAVGSIINHDPDVQGRLKVVFLPNFNVSLGQRIYPAADLSEQISTAGKEASGTGNMKFTMNGALTIGTYDGANIEIREEVGPENFFLFGLRAEDIARRQSRGYRPVEFWSSNAELRAVLDRFSSGHFTPDQPNLFQDLVSDLLQRDEYMLMADYQSYIDCQREAAAAYRDSDRWWRMSLLNTARSGKFSSDRTIADYSEQIWEVKPVPVSLSTSF

SEQ ID NO: 69 - DNA, 2085 bp, Synechococcus elongatus PCC 7942
atgactgtttcatcccgtcgccctgaatcgaccgtggctgttgaccccggccaaagctatcccctcggggcaaccgtctatcccaccggcgtcaacttctcgctctacaccaagtacgcgacgggcgttgaattactgctgtttgatgaccctgagggtgcccagcctcaacggacagtgcgcctcgatccgcacctcaatcgcacctctttctactggcatgtttttattccgggcattcgctccggtcaggtttatgcttaccgcgtctttggcccctacgcacctgatcgcggcctctgttttaaccccaacaaagtgctgctggatccctacgctcgcggggttgtcggctggcagcactacagtcgcgaagcggctattaaacccagtaataactgcgttcaagccctgcgtagcgtggttgttgaccccagcgactacgactgggaaggcgatcgccatccacgcacaccctacgctcgcacagtaatctatgagctgcatgttggcggcttcaccaagcatcccaattccggcgtcgcccctgaaaaacgtggcacctacgctggtctaatcgaaaaaattccctacctgcaatccctcggcgtcacggccgttgagttgctgccggtgcaccagttcgatcgccaagatgcccccttaggacgcgagaactactggggctacagcaccatggctttttttgcgccccacgcagcctacagctctcgccatgatccacttggtccagttgatgagttccgcgacctcgtcaaggcgctccaccaagcagggattgaggtgattctcgacgtggtgttcaaccacactgctgaagggaatgaagacggtccaacgctgtctttcaaaggtctagcgaattcaacctactatctgctggatgaacaggcgggctatcgcaactacaccggctgcggcaacaccgtcaaagctaacaattcgatcgtgcgatcgctgattctcgattgcctgcgttattgggtctcggaaatgcacgtcgatggcttccgctttgaccttgcgtcggtgctgagtcgtgatgccaatggcaaccccctatcggatccgcccttgctttgggcgattgattccgatccggttttggccggtacgaagctcattgctgaagcttgggacgcagccggcttatatcaggttggtacctttattggcgatcgctttgggacttggaacggtcccttccgggacgatattcggcgtttttggcgtggagatcagggctgtacttacgccctcagtcaacgcctgctgggtagccccgatgtctacagcacagaccaatggtatgccggacgcaccattaacttcatcacctgccatgacggctttacgctgcgagatctagtcagctatagccagaagcacaactttgccaatggagagaacaatcgggacgggaccaatgacaactacagctggaactacggcattgaaggcgagaccgatgaccccacgattctgagcttacgggaacggcagcagcgcaatttgctcgccacgttattcctcgcccagggcacaccgatgctgacgatgggcgatgaggtcaaacgcagtcagcagggtaacaataacgcctactgccaagacaatgagatcagctggtttgattggtcgctgtgcgatcgccatgccgatttcttggtgttcagtcgccgcctgattgaactttcccagtcgctggtgatgttccaacagaacgaactgctgcagaacgaaccccatccgcgtcgtccctatgccatctggcatggcgtcaaactcaaacaacccgattgggcgctgtggtcccacagtctggccgtcagtctctgccatcctcgccagcaggaatggctttacctagcctttaatgcttactgggaagacctgcgcttccagttgccgaggcctcctcgcggccgcgtttggtatcgcttgctcgatacttcactgccgaatcttgaagcttgtcatctgccggatgaggcaaaaccctgcctacggcgcgattacatcgtcccagcgcgatcgctcttactgttgatggctcgtgcttaa

SEQ ID NO: 70 - protein, 694 aa, Synechococcus elongatus PCC 7942
MTVSSRRPESTVAVDPGQSYPLGATVYPTGVNFSLYTKYATGVELLLFDDPEGAQPQRTVRLDPHLNRTSFYWHVFIPGIRSGQVYAYRVFGPYAPDRGLCFNPNKVLLDPYARGVVGWQHYSREAAIKPSNNCVQALRSVVVDPSDYDWEGDRHPRTPYARTVIYELHVGGFTKHPNSGVAPEKRGTYAGLIEKIPYLQSLGVTAVELLPVHQFDRQDAPLGRENYWGYSTMAFFAPHAAYSSRHDPLGPVDEFRDLVKALHQAGIEVILDVVFNHTAEGNEDGPTLSFKGLANSTYYLLDEQAGYRNYTGCGNTVKANNSIVRSLILDCLRYWVSEMHVDGFRFDLASVLSRDANGNPLSDPPLLWAIDSDPVLAGTKLIAEAWDAAGLYQVGTFIGDRFGTWNGPFRDDIRRFWRGDQGCTYALSQRLLGSPDVYSTDQWYAGRTINFITCHDGFTLRDLVSYSQKHNFANGENNRDGTNDNYSWNYGIEGETDDPTILSLRERQQRNLLATLFLAQGTPMLTMGDEVKRSQQGNNNAYCQDNEISWFDWSLCDRHADFLVFSRRLIELSQSLVMFQQNELLQNEPHPRRPYAIWHGVKLKQPDWALWSHSLAVSLCHPRQQEWLYLAFNAYWEDLRFQLPRPPRGRVWYRLLDTSLPNLEACHLPDEAKPCLRRDYIVPARSLLLLMARA

SEQ ID NO: 71 - DNA, 1500 bp, Synechococcus elongatus PCC 7942
gtgtttacacgagccgccggcattttgttacatcccacttcgttgccggggccattcggcagcggcgaccttggtccggcctcgcggcagtttcttgactggttggcaacggcgggacaacaactgtggcaagtgttgccccttgggccgacaggctatggctattcgccttacctctgctattccgccttggctggcaatcccgctctgatcagccctgaactcttggcagaagatggctggctccaagaatcggactgggcagactgtcctgcttttccgagcgatcgcgtcgattttgccagcgtcttgccctatcgcgatcaactgctgcgccgtgcctacagccaattcctgcaaagagcggcttccagcgatcgccaactctttcaagctttctgtgaacaggaagcccattggctggatgactacgccctgttcatggcgattaagctggctagccaaggtcagccttggacagaatggccggaagcgctgcgtcagcggcaacctcaagccttggctaaagcccgcgatcgctggggcggcgaaattggcttccagcagtttctgcagtggcaatttcgcgagcagtggttggccctgcgggaagaagcccaagcccgccatatttcgctgattggcgatattccgatctacgtcgctcatgacagtgcggacgtttgggccaatcctcagttctttgccctcgatcctgaaacgggcgcagttgatcagcaggccggtgtgccgcctgactatttctccgaaaccggccaactctggggcaatcccgtctacaactgggctgcgctgcaggcggatggctatcgctggtggttgcaacggctgcaacagctcctcagcttagtggactacattcgcatcgaccacttccgcggtttagaggcgttttggtcggttcccgctggtgaagaaacggcgatcgacggagagtgggtcaaagccccaggcgctgatctgctgagcacgattcgccaaaaactgggagcgctaccgattctggcagaggatctcggtgtgattacgccggaggtggaagcgctgcgcgatcgctttgagctgccgggcatgaagattctgcagttcgcctttgactctggggccggcaatgcctatctaccgcacaactactggggtcgtcgctgggtggcttacaccggcacccacgacaatgacacgaccgtcggctggttcctgtcccgcaatgacagcgatcgccaaacggtgctggattatctgggcgcagagtcgggctgggaaattgagtggaagctgatccgcttggcttggagctcgacggcagattgggcgatcgcaccgctccaagatgtcttcgggctggatagcagcgcccgcatgaatcgaccggggcaagccaccggcaactgggactggcgcttcagtgccgactggctgacgggcgatcgtgcccaacgcctgcggcgactctcgcagctctatggacgctgtagatga

SEQ ID NO: 72 - protein, 499 aa, Synechococcus elongatus PCC 7942
MFTRAAGILLHPTSLPGPFGSGDLGPASRQFLDWLATAGQQLWQVLPLGPTGYGYSPYLCYSALAGNPALISPELLAEDGWLQESDWADCPAFPSDRVDFASVLPYRDQLLRRAYSQFLQRAASSDRQLFQAFCEQEAHWLDDYALFMAIKLASQGQPWTEWPEALRQRQPQALAKARDRWGGEIGFQQFLQWQFREQWLALREEAQARHISLIGDIPIYVAHDSADVWANPQFFALDPETGAVDQQAGVPPDYFSETGQLWGNPVYNWAALQADGYRWWLQRLQQLLSLVDYIRIDHFRGLEAFWSVPAGEETAIDGEWVKAPGADLLSTIRQKLGALPILAEDLGVITPEVEALRDRFELPGMKILQFAFDSGAGNAYLPHNYWGRRWVAYTGTHDNDTTVGWFLSRNDSDRQTVLDYLGAESGWEIEWKLIRLAWSSTADWAIAPLQDVFGLDSSARMNRPGQATGNWDWRFSADWLTGDRAQRLRRLSQLYGRCR

SEQ ID NO: 73 - protein, 543 aa, Synechococcus elongatus PCC 7942
MNIHTVATQAFSDQKPGTSGLRKQVPVFQKRHYLENFVQSIFDSLEGYQGQTLVLGGDGRYYNRTAIQTILKMAAANGWGRVLVGQGGILSTPAVSNLIRQNGAFGGIILSASHNPGGPEGDFGIKYNISNGGPAPEKVTDAIYACSLKIEAYRILEAGDVDLDRLGSQQLGEMTVEVIDSVADYSRLMQSLFDFDRIRDRLRGGLRIAIDSMHAVTGPYATTIFEKELGAAAGTVFNGKPLEDFGGGHPDPNLVYAHDLVELLFGDRAPDFGAASDGDGDRNMILGNHFFVTPSDSLAILAANASLVPAYRNGLSGIARSMPTSAAADRVAQALNLPCYETPTGWKFFGNLLDADRVTLCGEESFGTGSNHVREKDGLWAVLFWLNILAVREQSVAEIVQEHWRTYGRNYYSRHDYEGVESDRASTLVDKLRSQLPSLTGQKLGAYTVAYADDFRYEDPVDGSISEQQGIRIGFEDGSRMVFRLSGTGTAGATLRLYLERFEGDTTKQGLDPQVALADLIAIADEVAQITTLTGFDQPTVIT

SEQ ID NO: 74 - protein, 567 aa, Synechocystis sp. PCC 6803
MSKPLIAALHFLQFLYMTSRINPLAGQHPPADSLLDVAKLLDDYYRQQPDPENPAQLVSFGTSGHRGSALNGTFNEAHILAVTQAVVDYRQAQGITGPLYMGMDSHALSEPAQKTALEVLAANQVETFLTTATDLTRFTPTPAVSYAILTHNQGRKEGLADGIIITPSHNPPTDGGFKYNPPSGGPAEPEATQWIQNRANELLKNGNKTVKRLDYEQALKATTTHAHDFVTPYVAGLADIIDLDVIRSAGLRLGVDPLGGANVGYWEPIAAKYNLNISLVNPGVDPTFKFMTLDWDGKIRMDCSSPYAMASLVKIKDHYDIAFGNDTDGDRHGIVTPSVGLMNPNHFLSVAIWYLFSQRQQWSGLSAIGKTLVSSSMIDRVGAMINRQVYEVPVGFKWFVSGLLDGSFGFGGEESAGASFLKKNGTVWTTDKDGTIMDLLAAEITAKTGKDPGLHYQDLTAKLGNPIYQRIDAPATPAQKDRLKKLSPDDVTATSLAGDAITAKLTKAPGNQAAIGGLKVTTAEGWFAARPSGTENVYKIYAESFKDEAHLQAIFTEAEAIVTSALG

SEQ ID NO: 75 - DNA, 1632 bp, Synechococcus elongatus PCC 7942; identical to SEQ ID NO: 24

SEQ ID NO: 76 - protein, 543 aa, Synechococcus elongatus PCC 7942; identical to SEQ ID NO: 73

SEQ ID NO: 77 - protein, 552 aa, Synechococcus sp. WH8102
MTTSAPAEPTLRLVRLDAPFTDQKPGTSGLRKSSQQFEQANYLESFVEAVFRTLPGVQGGTLVLGGDGRYGNRRAIDVILRMGAAHGLSKVIVTTGGILSTPAASNLIRQRQAIGGIILSASHNPGGPNGDFGVKVNGANGGPTPASFTDAVFECTKTLEQYTIVDAAAIAIDTPGSYSIGAMQVEVIDGVDDFVALMQQLFDFDRIRELIRSDFPLAFDAMHAVTGPYATRLLEEILGAPAGSVRNGVPLEDFGGGHPDPNLTYAHELAELLLDGEEFRFGAACDGDGDRNMILGQHCFVNPSDSLAVLTANATVAPAYADGLAGVARSMPTSSAVDVVAKELGIDCYETPTGWKFFGNLLDAGKITLCGEESFGTGSNHVREKDGLWAVLFWLQILAERRCSVAEIMAEHWKRFGRHYYSRHDYEAVASDAAHGLFHRLEGMLPGLVGQSFAGRSVSAADNFSYTDPVDGSVTKGQGLRILLEDGSRVMVRLSGTGTKGATIRVYLESYVPSSGDLNQDPQVALADMISAINELAEIKQRTGMDRPTVIT

SEQ ID NO: 78 - DNA, 1662 bp, Synechococcus sp. RCC 307
gtgacgctttcctcacccagcactgagttctccgtgcagcagatcaagctgccagaagcgtttcaagaccagaagcctggcacctcgggactgcgcaagagcacccaacaatttgaacagcctcattacctcgaaagttttatcgaggcgatcttccgcaccctccctggtgtgcaaggcgggaccttggtggtgggcggtgatggccgctacggcaaccgccgcgccatcgatgtcatcacccggatggcggcagcccatggactggggcggattgtgctgaccaccggcggcatcctctccacccctgccgcttccaacttgatccgccaacgccaggccattggcggcatcatcctctcggccagccacaaccctggagggcccaaaggcgactttggcgtcaaggtcaatggcgccaacggcggccctgcccctgaatctcttaccgatgccatctacgcctgcagccagcagctcgatggctaccgcatcgcaagtggaaccgcactgcccctcgacgccccagccgagcatcaaatcggtgcgttgaacgtggaggtgatcgacggcgtcgacgactacctgcaactgatgcagcacttgttcgacttcgatctgatcagcgatttgctcaagggctcatggccaatggcctttgacgccatgcatgccgtcactggtccctacgccagcaaactctttgagcagctcctaggagccccaagcgggaccgtgcgcaacgggcgctgcctcgaagactttggtggcggccatcccgatcccaacctcacctacgccaaagagctggcgacgctgctgctggatggtgatgactatcgctttggcgcggcctgtgatggcgatggcgaccgcaacatgattttggggcagcgctgctttgtgaaccccagcgacagcctcgctgtcttaacggcgaacgccaccttggtgaagggctatgcctccggcctggccggcgttgctcgctcgatgcccaccagtgccgcagtggatgtggtggccaagcagctggggatcaattgctttgagacccccaccggttggaaatttttcggcaacctgctcgatgccggacgcatcaccctttgcggggaagagagctttggaacaggcagtgatcacatccgcgaaaaagatggcctctgggctgtgttgttttggctctcgatcctggccaagcgccaatgctctgttgcggaggtgatgcagcagcactggagcacctacgggcgtcattactactcgcgccatgactacgaaggtgtcgaaaccgatcgggcccatgggctctacaacggcctgcgcgatcggcttggcgagctgactggaaccagctttgccgatagccgcatcgccaatgctgacgacttcgcctacagcgaccccgtcgatggctcactgacccagaagcaaggcctacgtctgctcctggaggacggcagccgcatcatcctgcggctctcgggaaccggcaccaaaggagccacgctgcggctctatctcgagcgctatgtcgccactggcggcaacctcgatcaaaatccccagcaagccttagccggcatgattgcggccgccgatgccctcgccggcatccggtcaaccaccggcatggatgtccccacggtgatcacctga

SEQ ID NO: 79 - protein, 553 aa, Synechococcus sp. RCC 307
MTLSSPSTEFSVQQIKLPEAFQDQKPGTSGLRKSTQQFEQPHYLESFIEAIFRTLPGVQGGTLVVGGDGRYGNRRAIDVITRMAAAHGLGRIVLTTGGILSTPAASNLIRQRQAIGGIILSASHNPGGPKGDFGVKVNGANGGPAPESLTDAIYACSQQLDGYRIASGTALPLDAPAEHQIGALNVEVIDGVDDYLQLMQHLFDFDLISDLLKGSWPMAFDAMHAVTGPYASKLFEQLLGAPSGTVRNGRCLEDFGGGHPDPNLTYAKELATLLLDGDDYRFGAACDGDGDRNMILGQRCFVNPSDSLAVLTANATLVKGYASGLAGVARSMPTSAAVDVVAKQLGINCFETPTGWKFFGNLLDAGRITLCGEESFGTGSDHIREKDGLWAVLFWLSILAKRQCSVAEVMQQHWSTYGRHYYSRHDYEGVETDRAHGLYNGLRDRLGELTGTSFADSRIANADDFAYSDPVDGSLTQKQGLRLLLEDGSRIILRLSGTGTKGATLRLYLERYVATGGNLDQNPQQALAGMIAAADALAGIRSTTGMDVPTVIT

SEQ ID NO: 80 - DNA, 1467 bp, Synechococcus sp PCC 7002
gtgttggcgtttgggaatcaacagccgattcggttcggcacagacggttggcgtggcattattgcggcggattttacctttgaacgggtgcaacgggtggcgatcgccacagcccatgttttaaaagaaaatttcgcaaaccaagccattgataacacgataatcgtcggctacgaccggcggtttctcgcagatgaatttgcccttgctgccgccgaagcgatccagggggaaggatttcacgtacttctagccaatagttttgcgccaaccccagccctgagctatgccgcccaccaccacaaggctctgggggcgatcgccttaacggccagccataatccagcgggttatttaggattaaaagtgaaaggggctttcggcggctcggtttccgaagaaattacggctcagattgaagcgcgactggaagccgggattgatcctcaacattcaacgacgggccgtttagattattttgatccctggcaggactattgcgccggattacagcaactggttgatttagaaaaaattcgccaggcgatcgccgctggtcgtctccaggtctttgccgatgtaatgtatggcgcagcggcgggcggtttgacccaactgctcaatgcggcgatccaagaaatccattgtgaaccagatcctttgttcggcggccgcccaccagagcctttagaaaaacatttgtctcaactgcaacgcaccattcgcgccgcccataatcaagatttagaggcaattcaggtgggatttgtctttgatggtgatggcgatcgcattgctgctgtggctggggatggtgagtttctcagttcccaaaagctaatcccgattttgctggcccatttgtcccaaaatcgccaatatcaaggggaagtggtaaaaactgtcagcggctctgatttaatcccccgtttgagcgaatactacggtttgccagtctttgaaacacccatcggctacaaatacattgccgaacgaatgcaacagacccaggtgcttcttggtggcgaagaatccggcggcattggctacggccaccacattcccgaacgggatgcgctgctggcggcattgtatctcctagaggcgatcgccatttttgatcaagacctcggcgagatttaccagagtcttcaaagcaaagctaatttttatggcgcctacgaccgcattgatttacatttgcgggatttctccagccgcgatcgcctattaaaaatcctcgcgacaaatccccccaaggcgatctccaaccatgacgtaattcacagcgaccccaaagatggctataaattccgccttgctgatcaaagttggttgctgattcgcttcagtggtaccgagcctgtactgcggttatatagtgaagcggtcaatcctaaagccgtacaagaaatcctcgcctgggcgcaaacctgggctgaggctgccgaccaagccgaaggttag

SEQ ID NO: 81 - protein, 488 aa, Synechococcus sp PCC 7002
MLAFGNQQPIRFGTDGWRGIIAADFTFERVQRVAIATAHVLKENFANQAIDNTIIVGYDRRFLADEFALAAAEAIQGEGFHVLLANSFAPTPALSYAAHHHKALGAIALTASHNPAGYLGLKVKGAFGGSVSEEITAQIEARLEAGIDPQHSTTGRLDYFDPWQDYCAGLQQLVDLEKIRQAIAAGRLQVFADVMYGAAAGGLTQLLNAAIQEIHCEPDPLFGGRPPEPLEKHLSQLQRTIRAAHNQDLEAIQVGFVFDGDGDRIAAVAGDGEFLSSQKLIPILLAHLSQNRQYQGEVVKTVSGSDLIPRLSEYYGLPVFETPIGYKYIAERMQQTQVLLGGEESGGIGYGHHIPERDALLAALYLLEAIAIFDQDLGEIYQSLQSKANFYGAYDRIDLHLRDFSSRDRLLKILATNPPKAISNHDVIHSDPKDGYKFRLADQSWLLIRFSGTEPVLRLYSEAVNPKAVQEILAWAQTWAEAADQAEG

SEQ ID NO: 82 - DNA, 1038 bp, Synechococcus elongatus PCC 7942
atgaccttgctattggccggggatatcggcggaaccaaaacgaatttaatgttggcgatcgcctctgattgcgatcgtttagaaccgctccatcaggccagttttgccagtgcggcctaccctgatttagtgccgatggtgcaggagtttttggctgccgcaccctccgccgaggtgcgatcgccagttgtggcttgttttggcattgccggccccgttgtccatggaaccgcgaagctgacgaacctgccttggcagctctctgaagcgcggctggcgaaggaattgggcattgcgcaggtggcgttgatcaatgattttgctgcgatcgcctacggcctacccggcttgaccgccgaagatcaagtcgttgtgcaagtcggtgaagccgatccggcggctccgatcgccattctgggggcaggaactggcttgggcgaaggcttcatcattcccacagcccaaggccgccaagtgtttggcagcgaaggttctcacgctgactttgcgccgcaaaccgaactggagtccgagttactgcattttctacgcaatttttacgcaatcgagcatatctcggtcgagcgagtggtctccggccaagggattgcagccatctacgccttcctgcgcgatcgccatcccgaccaagaaaatccagcccttggggcgattgcctcggcttggcaaacgggcggcgaccaagcccctgatctggcagcagccgtatcccaagcagccttgagcgatcgcgatccgctggccctacaagccatgcagatatttgtcagtgcttacggggcggaagccggcaacctcgcgttgaaattgctctcctacggcggggtctacgtcgccggcgggattgcgggcaaaatcctgccgctcttgactgatggcacttttctgcaagccttccaagccaagggacgggtgaaggggctgctgacgcggatgcctatcacgatcgtcacgaaccacgaagtcgggctgatcggggctggactgcgggcggctgcgatcgctactcaaccatga

SEQ ID NO: 83 - protein, 345 aa, Synechococcus elongatus PCC 7942
MTLLLAGDIGGTKTNLMLAIASDCDRLEPLHQASFASAAYPDLVPMVQEFLAAAPSAEVRSPVVACFGIAGPVVHGTAKLTNLPWQLSEARLAKELGIAQVALINDFAAIAYGLPGLTAEDQVVVQVGEADPAAPIAILGAGTGLGEGFIIPTAQGRQVFGSEGSHADFAPQTELESELLHFLRNFYAIEHISVERVVSGQGIAAIYAFLRDRHPDQENPALGAIASAWQTGGDQAPDLAAAVSQAALSDRDPLALQAMQIFVSAYGAEAGNLALKLLSYGGVYVAGGIAGKILPLLTDGTFLQAFQAKGRVKGLLTRMPITIVTNHEVGLIGAGLRAAAIATQP

SEQ ID NO: 84 - DNA, 1587 bp, Synechococcus elongatus PCC 7942
atgaccgcccagcagctctggcaacgctacctcgattggctctactacgatccctcgctggagttttacctcgacatcagccgcatgggattcgatgacgctttcgttactagcatgcagcccaagttccagcacgcctttgcggcgatggcagagctcgaggccggagcgatcgccaaccccgatgaacagcggatggtcggccactactggctgcgcgatcctgagctggcacccacaccggagctgcagacccaaattcgcgacacgctggccgcgatccaagacttcgccctcaaagtacacagtggcgtgttgcggccacccaccggctcccgcttcaccgacattctctcaattggcattggcgggtcggccctagggccgcagtttgtctcagaagccctccggcctcaagcggcactgctccagattcacttctttgacaacaccgatccagctggcttcgatcgcgttttagctgatctcggcgatcgccttgcttccaccttagtaatcgttatttccaaatctggcggcactcccgaaacccgcaacggcatgctggaggttcagtccgcctttgcccagcgagggattgcctttgcgccccaagctgtcgccgtcacaggggtggggagccatctcgatcatgtagcgatcacagaaagatggctggcccgtttccccatggaagactgggtgggcggccgcacctctgaactatctgcagtcggtctactctcggcagccctactgggcatcgacatcaccgccatgctggccggggcgcggcaaatggacgccctgacccgccattccgatttgcgacaaaatccggcagcgctcttggctttgagctggtactgggccggcaatgggcaaggcaaaaaagacatggtcatcctgccctacaaggacagcctgctgctgtttagccgctatctgcagcagttgatcatggagtcactgggcaaggagcgcgatctgctcggcaaggtagttcaccaaggcatcgccgtttacggcaacaaaggctcgaccgatcaacatgcctacgtccagcaactgcgcgagggcattcctaacttctttgccacgtttatcgaggtgctcgaagaccgacaggggccgtcgccagtcgtggagcctggcatcaccagtggcgactatctcagcgggctgcttcaaggcacccgcgcggcgctttacgaaaatgggcgtgagtcgatcacgattacggtgccgcgcgttgatgcacaacaggtgggggccttgatcgcgctgtatgaacgggcggtgggactctatgccagcttggttggcatcaatgcctatcaccagccgggggtggaagccggcaaaaaggctgctgccggtgttctcgagatccagcgccagattgtggagttgctccaacagggacaaccactctcgatcgcagcgatcgcagacgatttaggtcagagtgagcagattgaaacgatctacaaaatcctgcgccatctcgaagccaatcaacgcggcgttcagttaaccggcgatcgccataatcccctcagtctgattgcgagttggcaacgataa

SEQ ID NO: 85 - protein, 528 aa, Synechococcus elongatus PCC 7942
MTAQQLWQRYLDWLYYDPSLEFYLDISRMGFDDAFVTSMQPKFQHAFAAMAELEAGAIANPDEQRMVGHYWLRDPELAPTPELQTQIRDTLAAIQDFALKVHSGVLRPPTGSRFTDILSIGIGGSALGPQFVSEALRPQAALLQIHFFDNTDPAGFDRVLADLGDRLASTLVIVISKSGGTPETRNGMLEVQSAFAQRGIAFAPQAVAVTGVGSHLDHVAITERWLARFPMEDWVGGRTSELSAVGLLSAALLGIDITAMLAGARQMDALTRHSDLRQNPAALLALSWYWAGNGQGKKDMVILPYKDSLLLFSRYLQQLIMESLGKERDLLGKVVHQGIAVYGNKGSTDQHAYVQQLREGIPNFFATFIEVLEDRQGPSPVVEPGITSGDYLSGLLQGTRAALYENGRESITITVPRVDAQQVGALIALYERAVGLYASLVGINAYHQPGVEAGKKAAAGVLEIQRQIVELLQQGQPLSIAAIADDLGQSEQIETIYKILRHLEANQRGVQLTGDRHNPLSLIASWQR

SEQ ID NO: 86 - protein, 439 aa, Synechocystis sp. PCC 6803
MCCWQSRGLLVKRVLAIILGGGAGTRLYPLTKLRAKPAVPLAGKYRLIDIPVSNCINSEIVKIYVLTQFNSASLNRHISRAYNFSGFQEGFVEVLAAQQTKDNPDWFQGTADAVRQYLWLFREWDVDEYLILSGDHLYRMDYAQFVKRHRETNADITLSVVPVDDRKAPELGLMKIDAQGRITDFSEKPQGEALRAMQVDTSVLGLSAEKAKLNPYIASMGIYVFKKEVLHNLLEKYEGATDFGKEIIPDSASDHNLQAYLFDDYWEDIGTIEAFYEANLALTKQPSPDFSFYNEKAPIYTRGRYLPPTKMLNSTVTESMIGEGCMIKQCRIHHSVLGIRSRIESDCTIEDTLVMGNDFYESSSERDTLKARGEIAAGIGSGTTIRRAIIDKNARIGKNVMIVNKENVQEANREELGFYIRNGIVVVIKNVTIADGTVI

SEQ ID NO: 87 - protein, 429 aa, Nostoc sp. PCC 7120
MKKVLAIILGGGAGTRLYPLTKLRAKPAVPVAGKYRLIDIPVSNCINSEIFKIYVLTQFNSASLNRHIARTYNFSGFSEGFVEVLAAQQTPENPNWFQGTADAVRQYLWMLQEWDVDEFLILSGDHLYRMDYRLFIQRHRETNADITLSVIPIDDRRASDFGLMKIDNSGRVIDFSEKPKGEALTKMRVDTTVLGLTPEQAASQPYIASMGIYVFKKDVLIKLLKEALERTDFGKEIIPDAAKDHNVQAYLFDDYWEDIGTIEAFYNANLALTQQPMPPFSFYDEEAPIYTRARYLPPTKLLDCHVTESIIGEGCILKNCRIQHSVLGVRSRIETGCMIEESLLMGADFYQASVERQCSIDKGDIPVGIGPDTIIRRAIIDKNARIGHDVKIINKDNVQEADRESQGFYIRSGIVVVLKNAVITDGTII

SEQ ID NO: 88 - protein, 429 aa, Anabaena variabilis
MKKVLAIILGGGAGTRLYPLTKLRAKPAVPVAGKYRLIDIPVSNCINSEIFKIYVLTQFNSASLNRHIARTYNFSGFSEGFVEVLAAQQTPENPNWFQGTADAVRQYLWMLQEWDVDEFLILSGDHLYRMDYRLFIQRHRETNADITLSVIPIDDRRASDFGLMKIDNSGRVIDFSEKPKGEALTKMRVDTTVLGLTPEQAASQPYIASMGIYVFKKDVLIKLLKESLERTDFGKEIIPDASKDHNVQAYLFDDYWEDIGTIEAFYNANLALTQQPMPPFSFYDEEAPIYTRARYLPPTKLLDCHVTESIIGEGCILKNCRIQHSVLGVRSRIETGCVIEESLLMGADFYQASVERQCSIDKGDIPVGIGPDTIIRRAIIDKNARIGHDVKIINKDNVQEADRESQGFYIRSGIVVVLKNAVITDGTII

SEQ ID NO: 89 - protein, 428 aa, Trichodesmium erythraeum IMS 101
MKNVLSIILGGGAGTRLYPLTKLRAKPAVPLAGKYRLIDIPISNCINSEIQKIYVLTQFNSASLNRHITRTYNFSGFSDGFVEVLAAQQTKDNPEWFQGTADAVRKYIWLFKEWDIDYYLILSGDHLYRMDYRDFVQRHIDTKADITLSVLPIDEARASEFGVMKIDNSGRIVEFSEKPKGNALKAMAVDTSILGVSPEIATKQPYIASMGIYVFNKDAMIKLIEDSEDTDFGKEILPKSAQSYNLQAYPFQGYWEDIGTIKSFYEANLALTQQPQPPFSFYDEQAPIYTRSRYLPPSKLLDCEITESIVGEGCILKKCRIDHCVLGVRSRIEANCIIQDSLLMGSDFYESPTERRYGLKKGSVPLGIGAETKIRGAIIDKNARIGCNVQIINKDNVEEAQREEEGFIIRSGIVVVLKNATIPDGTVI

SEQ ID NO: 90 - protein, 430 aa, Synechococcus elongatus PCC 7942
MKNVLAIILGGGAGSRLYPLTKQRAKPAVPLAGKYRLIDIPVSNCINADINKIYVLTQFNSASLNRHLSQTYNLSSGFGNGFVEVLAAQITPENPNWFQGTADAVRQYLWLIKEWDVDEYLILSGDHLYRMDYSQFIQRHRDTNADITLSVLPIDEKRASDFGLMKLDGSGRVVEFSEKPKGDELRAMQVDTTILGLDPVAAAAQPFIASMGIYVFKRDVLIDLLSHHPEQTDFGKEVIPAAATRYNTQAFLFNDYWEDIGTIASFYEANLALTQQPSPPFSFYDEQAPIYTRARYLPPTKLLDCQVTQSIIGEGCILKQCTVQNSVLGIRSRIEADCVIQDALLMGADFYETSELRHQNRANGKVPMGIGSGSTIRRAIVDKNAHIGQNVQIVNKDHVEEADREDLGFMIRSGIVVVVKGAVIPDNTVI

SEQ ID NO: 91 - protein, 431 aa, Synechococcus sp. WH8102
MKRVLAIILGGGAGTRLYPLTKMRAKPAVPLAGKYRLIDIPISNCINSNINKMYVMTQFNSASLNRHLSQTFNLSASFGQGFVEVLAAQQTPDSPSWFEGTADAVRKYQWLFQEWDVDEYLILSGDQLYRMDYSLFVEHHRSTGADLTVAALPVDPKQAEAFGLMRTDGDGDIKEFREKPKGDSLLEMAVDTSRFGLSANSAKERPYLASMGIYVFSRDTLFDLLDSNPGYKDFGKEVIPEALKRGDKLKSYVFDDYWEDIGTIGAFYEANLALTQQPTPPFSFYDEKFPIYTRPRYLPPSKLVDAQITNSIVGEGSILKSCSIHHCVLGVRSRIETDVVLQDTLVMGADFFESSDERAVLRERGGIPVGVGQGTTVKRAILDKNARIGSNVTIVNKDHVEEADRSDQGFYIRNGIVVVVKNATIQDGTVI

SEQ ID NO: 92 - protein, 431 aa, Synechococcus sp. RCC 307
MKRVLAIILGGGAGTRLYPLTKMRAKPAVPLAGKYRLIDIPVSNCINSGINKIYVLTQFNSASLNRHIAQTFNLSSGFDQGFVEVLAAQQTPDSPSWFEGTADAVRKYEWLLQEWDIDEVLILSGDQLYRMDYAHFVAQHRASGADLTVAALPVDREQAQSFGLMHTGAEASITKFREKPKGEALDEMSCDTASMGLSAEEAHRRPFLASMGIYVFKRDVLFRLLAENPGATDFGKEIIPKALDDGFKLRSYLFDDYWEDIGTIRAFYEANLALTTQPRPPFSFYDKRFPIYTRHRYLPPSKLQDAQVTDSIVGEGSILKACSIHHCVLGVRSRIEDEVALQDTLVMGNDFYESGEERAILRERGGIPMGVGRGTTVKKAILDKNVRIGSNVSIINKDNVEEADRAEQGFYIRGGIVVITKNASIPDGMVI

SEQ ID NO: 93 - protein, 429 aa, Synechococcus sp. PCC 7002
MKRVLGIILGGGAGTRLYPLTKLRAKPAVPLAGKYRLIDIPVSNCINSEIHKIYILTQFNSASLNRHISRTYNFTGFTEGFTEVLAAQQTKENPDWFQGTADAVRQYSWLLEDWDVDEYIILSGDHLYRMDYREFIQRHRDTGADITLSVVPVGEKVAPAFGLMKIDANGRVVDFSEKPTGEALKAMQVDTQSLGLDPEQAKEKPYIASMGIYVFKKQVLLDLLKEGKDKTDFGKEIIPDAAKDYNVQAYLFDDYWADIGTIEAFYEANLGLTKQPIPPFSFYDEKAPIYTRARYLPPTKVLNADVTESMISEGCIIKNCRIHHSVLGIRTRVEADCTIEDTMIMGADYYQPYEKRQDCLRRGKPPIGIGEGTTIRRAIIDKNARIGKNVMIVNKENVEESNREELGYYIRSGITVVLKNAVIPDGTVI

SEQ ID NO: 94 - protein, 477 aa, Synechocystis sp. PCC 6803
MKILFVAAEVSPLAKVGGMGDVVGSLPKVLHQLGHDVRVFMPYYGFIGDKIDVPKEPVWKGEAMFQQFAVYQSYLPDTKIPLYLFGHPAFDSRRIYGGDDEAWRFTFFSNGAAEFAWNHWKPEIIHCHDWHTGMIPVWMHQSPDIATVFTIHNLAYQGPWRGLLETMTWCPWYMQGDNVMAAAIQFANRVTTVSPTYAQQIQTPAYGEKLEGLLSYLSGNLVGILNGIDTEIYNPAEDRFISNVFDADSLDKRVKNKIAIQEETGLEINRNAMVVGIVARLVEQKGIDLVIQILDRFMSYTDSQLIILGTGDRHYETQLWQMASRFPGRMAVQLLHNDALSRRVYAGADVFLMPSRFEPCGLSQLMAMRYGCIPIVRRTGGLVDTVSFYDPINEAGTGYCFDRYEPLDCFTAMVRAWEGFRFKADWQKLQQRAMRADFSWYRSAGEYIKVYKGVVGKPEELSPMEEEKIAELTASYR

SEQ ID NO: 95 - protein, 472 aa, Nostoc sp. PCC 7120
MRILFVAAEAAPIAKVGGMGDVVGALPKVLRKMGHDVRIFLPYYGFLPDKMEIPKDPIWKGYAMFQDFTVHEAVLPGTDVPLYLFGHPAFTPRRIYSGDDEDWRFTLFSNGAAEFCWNYWKPDIIHCHDWHTGMIPVWMNQSPDITTVFTIHNLAYQGPWRWYLDKITWCPWYMQGHNTMAAAVQFADRVNTVSPTYAEQIKTPAYGEKIEGLLSFISGKLSGIVNGIDTEVYDPANDKYIAQTFTADTLDKRKANKIALQEEVGLEVNSNAFLIGMVTRLVEQKGLDLVIQMLDRFMAYTDAQFVLLGTGDRYYETQMWQLASRYPGRMATYLLYNDALSRRIYAGTDAFLMPSRFEPCGISQMMALRYGSIPIVRRTGGLVDTVSHHDPINEAGTGYCFDRYEPLDLFTCMIRAWEGFRYKPQWQELQKRGMSQDFSWYKSAKEYDKLYRSMYGLPDPEETQPELILTNQ

SEQ ID NO: 96 - protein, 472 aa, Anabaena variabilis
MRILFVAAEAAPIAKVGGMGDVVGALPKVLRKMGHDVRIFLPYYGFLPDKMEIPKDPIWKGYAMFQDFTVHEAVLPGTDVPLYLFGHPAFNPRRIYSGDDEDWRFTLFSNGAAEFCWNYWKPEIIHCHDWHTGMIPVWMNQSPDITTVFTIHNLAYQGPWRWYLDKITWCPWYMQGHNTMAAAVQFADRVNTVSPTYAEQIKTPAYGEKIEGLLSFISGKLSGIVNGIDTEVYDPANDKFIAQTFTADTLDKRKANKIALQEEVGLEVNSNAFLIGMVTRLVEQKGLDLVIQMLDRFMAYTDAQFVLLGTGDRYYETQMWQLASRYPGRMATYLLYNDALSRRIYAGSDAFLMPSRFEPCGISQMMALRYGSIPIVRRTGGLVDTVSHHDPVNEAGTGYCFDRYEPLDLFTCMIRAWEGFRYKPQWQELQKRGMSQDFSWYKSAKEYDRLYRSIYGLPEAEETQPELILANQ

SEQ ID NO: 97 - protein, 460 aa, Trichodesmium erythraeum IMS 101
MRILFVSAEATPLAKVGGMADVVGALPKVLRKMGHDVRIFMPYYGFLGDKMEVPEEPIWEGTAMYQNFKIYETVLPKSDVPLYLFGHPAFWPRHIYYGDDEDWRFTLFANGAAEFCWNGWKPEIVHCNDWHTGMIPVWMHETPDIKTVFTIHNLAYQGPWRWYLERITWCPWYMEGHNTMAAAVQFADRVTTVSPTYASQIQTPAYGENLDGLMSFITGKLHGILNGIDMNFYNPANDRYIPQTYDVNTLEKRVDNKIALQEEVGFEVNKNSFLMGMVSRLVEQKGLDLMLQVLDRFMAYTDTQFILLGTGDRFYETQMWQIASRYPGRMSVQLLHNDALSRRIYAGTDAFLMPSRFEPCGISQLLAMRYGSIPIVRRTGGLVDTVSFYDPINNVGTGYSFDRYEPLDLLTAMVRAYEGFRFKDQWQELQKRGMRENFSWDKSAQGYIKMYKSMLGLPEE

SEQ ID NO: 98 - protein, 465 aa, Synechococcus elongatus PCC 7942
MRILFVAAECAPFAKVGGMGDVVGSLPKVLKALGHDVRIFMPYYGFLNSKLDIPAEPIWWGYAMFNHFAVYETQLPGSDVPLYLMGHPAFDPHRIYSGEDEDWRFTFFANGAAEFSWNYWKPQVIHCHDWHTGMIPVWMHQSPDISTVFTIHNLAYQGPWRWKLEKITWCPWYMQGDSTMAAALLYADRVNTVSPTYAQQIQTPTYGEKLEGLLSFISGKLSGILNGIDVDSYNPATDTRIVANYDRDTLDKRLNNKLALQKEMGLEVNPDRFLIGFVARLVEQKGIDLLLQILDRFLSYSDAQFVVLGTGERYYETQLWELATRYPGRMSTYLMYDEGLSRRIYAGSDAFLVPSRFEPCGITQMLALRYGSVPIVRRTGGLVDTVFHHDPRHAEGNGYCFDRYEPLDLYTCLVRAWESYQYQPQWQKLQQRGMAVDLSWKQSAIAYEQLYAEAIGLPIDVLQEA

SEQ ID NO: 99 - protein, 513 aa, Synechococcus sp. WH8102
MRILFAAAECAPMIKVGGMGDVVGSLPPALAKLGHDVRLIMPGYSKLWTKLTISDEPIWRAQTMGTEFAVYETKHPGNGMTIYLVGHPVFDPERIYGGEDEDWRFTFFASAAAEFAWNVWKPNVLHCHDWHTGMIPVWMHQDPEISTVFTIHNLKYQGPWRWKLDRITWCPWYMQGDHTMAAALLYADRVNAVSPTYAEEIRTAEYGEKLDGLLNFVSGKLRGILNGIDLEAWNPQTDGALPATFSADDLSGKAVCKRVLQERMGLEVRDDAFVLGMVSRLVDQKGVDLLLQVADRLLAYTDTQIVVLGTGDRGLESGLWQLASRHAGRCAVFLTYDDDLSRLIYAGSDAFLMPSRFEPCGISQLYAMRYGSVPVVRKVGGLVDTVPPHSPADASGTGFCFDRFEPVDFYTALVRAWEAYRHRDSWQELQKRGMQQDYSWDRSAIDYDVMYRDVCGLKEPTPDAAMVEQFSQGQAADPSRPEDDAINAAPEAVTAPSGPSRNPLNRLFGRRAD

SEQ ID NO: 100 - protein, 507 aa, Synechococcus sp RCC 307
MRILFAAAECAPMVKVGGMGDVVGSLPPALAELGHDVRVIMPGYGKLWSQLDVPSEPIWRAQTMGTDFAVYETRHPKTGLTIYLVGHPVFDGERIYGGEDEDWRFTFFASATSEFAWNAWKPQVLHCHDWHTGMIPVWMHQDPEISTVFTIHNLKYQGPWRWKLERMTWCPWYMQGDHTMAAALLYADRVNAVSPTYAQEIRTPEYGEQLEGLLNYISGKLRGILNGIDVEAWNPATDSRIPATYSTADLSGKAVCKRALQERMGLQVNPDTFVIGLVSRLVDQKGVDLLLQVAERFLAYTDTQIVVLGTGDRHLESGLWQMASQHSGRFASFLTYDDDLSRLIYAGSDAFLMPSRFEPCGISQLLSMRYGTIPVVRRVGGLVDTVPPYVPATQEGNGFCFDRYEAIDLYTALVRAWEAYRHQDSWQQLMKRVMQVDFSWARSALEYDRMYRDVCGMKEPTPEADAVAAFSIPQPPEQQAARAAAEAADPNPQRRFNPLGLLRRNGG

SEQ ID NO: 101 - protein, 478 aa, Synechococcus sp. PCC 7002
MRILFVSAEAAPIAKAGGMGDVVGSLPKVLRQLGHDARIFLPYYGFLNDKLDIPAEPVWWGSAMFNTFAVYETVLPNTDVPLYLFGHPAFDGRHIYGGQDEFWRFTFFANGAAEFMWNHWKPQIAHCHDWHTGMIPVWMHQSPDISTVFTIHNLAYQGPWRGFLERNTWCPWYMDGDNVMASALMFADQVNTVSPTYAQQIQTKVYGEKLEGLLSWISGKSRGIVNGIDVELYNPSNDQALVKQFSTTNLEDRAANKVIIQEETGLEVNSKAFLMAMVTRLVEQKGIDLLLNILEQFMAYTDAQLIILGTGDRHYETQLWQTAYRFKGRMSVQLLYNDALSRRIYAGSDVFLMPSRFEPCGISQMMAMRYGSVPIVRRTGGLVDTVSFHDPIHQTGTGFSFDRYEPLDMYTCMVRAWESFRYKKDWAELQRRGMSHDFSWYKSAGEYLKMYRQSIKEAPELTTDEAEKITYLVKKHAI

SEQ ID NO: 102 - DNA, 1380 bp, Synechococcus elongatus PCC 7942
atgactgctgtcgttctccctgctgccgctgaaacgctggctgctttacaagcaacctttgatcggggggatacacgcacgctcgccttccgactggcgcgattacaggatctggccaagctagttgctgacaatgaagcggagctattgcaagccttggcgtcagacctccgcaaaccagcactggaagcctacgccagtgagatttatttcgtgcgcgaccaaatcaaactgacctgcaagcatctgcggcgctggatgcaacccgagaagcagtcgatttccttgatgcagcagcctggccaggcctatcgccaagcagaaccgctcggagtcgtgctgatcattggcccctggaactatccctttcagctgctcatcacgccgttgattggggcgatcgcggcgggaaattgtgccgtactcaaaccatcggaactggctcccgcgacttccagcctgattcagcgactgatcagcgatcgctttgaccctgattacatccgcgttttagaaggcgatgctagcgttagccaagccctgattactcagcccttcgatcacatcttcttcactggcggcacggcgatcgggcgaaaagtgatggctgctgcggccgaaaacctgacgcccgtcaccctcgagttgggcggtaagtcaccctgcattgttgataccgatatcgacctcgatgtggccgcccgtcgcatcgcctggggcaaattcttcaacgccggtcaaacctgcattgcgcctgactatttgttggtgcaacgcacggtcgcagagccgttcattgaagcgctgatcgacaacatccagcagttctatggcgaggatccgcaacagagtgctgactacgcccgcattgtcagcgatcgccactggcaaaggctaaatagcctgttggttgatggcacgattcgccatggtggtcaggtggataggagcgatcgctacatcgcaccgactttaattacggacgtcaactggcgcgatcccatcctgcaagaggagatttttgggcccctcttgccgattttgatttacgaccaattggatgaggcgatcgcccaaattcgtgcccagcccaagcccctcgcgctctatctattcagccgcgatcgccaagtgcaagagcgcgtcctagcggaaaccagcgccggtagcgtctgcctcaacgacacgatcctgcaggtcggcgtccccgatgctgcttttggtggggtcggccccagcggcatgggcggctatcacggcaaagccagtttcgaaaccttcagtcactacaagctggtgctcaagcgaccgttttggctcgatctggccctgcgctatccgccctacggcgacaagatcaacctcttccgcaagctctag

SEQ ID NO: 103 - protein, 459 aa, Synechococcus elongatus PCC 7942
MTAVVLPAAAETLAALQATFDRGDTRTLAFRLARLQDLAKLVADNEAELLQALASDLRKPALEAYASEIYFVRDQIKLTCKHLRRWMQPEKQSISLMQQPGQAYRQAEPLGVVLIIGPWNYPFQLLITPLIGAIAAGNCAVLKPSELAPATSSLIQRLISDRFDPDYIRVLEGDASVSQALITQPFDHIFFTGGTAIGRKVMAAAAENLTPVTLELGGKSPCIVDTDIDLDVAARRIAWGKFFNAGQTCIAPDYLLVQRTVAEPFIEALIDNIQQFYGEDPQQSADYARIVSDRHWQRLNSLLVDGTIRHGGQVDRSDRYIAPTLITDVNWRDPILQEEIFGPLLPILIYDQLDEAIAQIRAQPKPLALYLFSRDRQVQERVLAETSAGSVCLNDTILQVGVPDAAFGGVGPSGMGGYHGKASFETFSHYKLVLKRPFWLDLALRYPPYGDKINLFRKL

SEQ ID NO: 104 - DNA, 1011 bp, Synechycystis sp. PCC6083
atgattaaagcctacgctgccctggaagccaacggaaaactccaaccctttgaatacgaccccggtgccctgggtgctaatgaggtggagattgaggtgcagtattgtggggtgtgccacagtgatttgtccatgattaataacgaatggggcatttccaattaccccctagtgccgggtcatgaggtggtgggtactgtggccgccatgggcgaaggggtgaaccatgttgaggtgggggatttagtggggctgggttggcattcgggctactgcatgacctgccatagttgtttatctggctaccacaacctttgtgccacggcggaatcgaccattgtgggccactacggtggctttggcgatcgggttcgggccaagggagtcagcgtggtgaaattacctaaaggcattgacctagccagtgccgggccccttttctgtggaggaattaccgttttcagtcctatggtggaactgagtttaaagcccactgcaaaagtggcagtgatcggcattgggggcttgggccatttagcggtgcaatttctccgggcctggggctgtgaagtgactgcctttacctccagtgccaggaagcaaacggaagtgttggaattgggcgctcaccacatactagattccaccaatccagaggcgatcgccagtgcggaaggcaaatttgactatattatctccactgtgaacctgaagcttgactggaacttatacatcagcaccctggcgccccagggacatttccactttgttggggtggtgttggagcctttggatctaaatctttttccccttttgatgggacaacgctccgtttctgcctccccagtgggtagtcccgccaccattgccaccatgttggactttgctgtgcgccatgacattaaacccgtggtggaacaatttagctttgatcagatcaacgaggcgatcgcccatctagaaagcggcaaagcccattatcgggtagtgctcagccatagtaaaaattag

SEQ ID NO: 105 - protein, 336 aa, Synechycystis sp. PCC6083
MIKAYAALEANGKLQPFEYDPGALGANEVEIEVQYCGVCHSDLSMINNEWGISNYPLVPGHEVVGTVAAMGEGVNHVEVGDLVGLGWHSGYCMTCHSCLSGYHNLCATAESTIVGHYGGFGDRVRAKGVSVVKLPKGIDLASAGPLFCGGITVFSPMVELSLKPTAKVAVIGIGGLGHLAVQFLRAWGCEVTAFTSSARKQTEVLELGAHHILDSTNPEAIASAEGKFDYIISTVNLKLDWNLYISTLAPQGHFHFVGVVLEPLDLNLFPLLMGQRSVSASPVGSPATIATMLDFAVRHDIKPVVEQFSFDQINEAIAHLESGKAHYRVVLSHSKN

SEQ ID NO: 106 - DNA, 1023 bp, Acinetobacter baylyi
atgacaactaatgtgattcatgcttatgctgcaatgcaggcaggtgaagcactcgtgccttattcgtttgatgcaggcgaactgcaaccacatcaggttgaagttaaagtcgaatattgtgggctgtgccattccgatgtctcggtactcaacaacgaatggcattcttcggtttatccagtcgtggcaggtcatgaagtgattggtacgattacccaactgggaagtgaagccaaaggactaaaaattggtcaacgtgttggtattggctggacggcagaaagctgtcaggcctgtgaccaatgcatcagtggtcagcaggtattgtgcacgggcgaaaataccgcaactattattggtcatgctggtggctttgcagataaggttcgtgcaggctggcaatgggtcattcccctgcccgacgaactcgatccgaccagtgctggtcctttgctgtgtggcggaatcacagtatttgatccaattttaaaacatcagattcaggctattcatcatgttgctgtgattggtatcggtggtttgggacatatggccatcaagctacttaaagcatggggctgtgaaattactgcgtttagttcaaatccaaacaaaaccgatgagctcaaagccatgggggccgatcacgtggtcaatagccgtgatgatgccgaaattaaatcgcaacagggtaaatttgatttactgctgagtacagttaatgtgcctttaaactggaatgcgtatctaaacacactggcacccaatggcactttccattttttgggcgtggtgatggaaccaatccctgtacctgtcggtgcgctgctaggaggtgccaaatcgctaacagcatcaccaactggctcgcctgctgccttacgtaagctgctcgaatttgcggcacgtaagaatatcgcacctcaaatcgagatgtatcctatgtcggagctgaatgaggccatcgaacgcttacattcgggtcaagcacgttatcggattgtacttaaagccgatttttaa

SEQ ID NO: 107 - protein, 340 aa, Acinetobacter baylyi
MTTNVIHAYAAMQAGEALVPYSFDAGELQPHQVEVKVEYCGLCHSDVSVLNNEWHSSVYPVVAGHEVIGTITQLGSEAKGLKIGQRVGIGWTAESCQACDQCISGQQVLCTGENTATIIGHAGGFADKVRAGWQWVIPLPDELDPTSAGPLLCGGITVFDPILKHQIQAIHHVAVIGIGGLGHMAIKLLKAWGCEITAFSSNPNKTDELKAMGADHVVNSRDDAEIKSQQGKFDLLLSTVNVPLNWNAYLNTLAPNGTFHFLGVVMEPIPVPVGALLGGAKSLTASPTGSPAALRKLLEFAARKNIAPQIEMYPMSELNEAIERLHSGQARYRIVLKADF

(19) United States
(12) Patent Application Publication
(10) Pub. No.: US 2014/0004580 A1
(43) Pub. Date: Jan. 2, 2014

(54) MODIFIED PHOTOSYNTHETIC MICROORGANISMS FOR PRODUCING LIPIDS

(71) Applicants: James Roberts, Seattle, WA (US); Fred Cross, New York, NY (US); Brett K. Kaiser, Seattle, WA (US)

(72) Inventors: James Roberts, Seattle, WA (US); Fred Cross, New York, NY (US); Brett K. Kaiser, Seattle, WA (US)

(73) Assignee: Matrix Genetics, LLC, Seattle, WA (US)

(21) Appl. No.: 13/995,912

(22) PCT Filed: Dec. 19, 2011
(86) PCT No.: PCT/US2011/065896

§ 371 Date: Sep. 6, 2013

Related U.S. Application Data
(60) Provisional application No. 61/425,181, filed on Dec. 20, 2010.
(60) Provisional application No. 61/452,525, filed on Mar. 14, 2011.
(60) Provisional application No. 61/477,773, filed on Apr. 21, 2011.

(51) Int. Cl. C12P 7/64; C12N 15/82

(52) U.S. Cl. CPC C12P 7/6436; C12N 15/8242
USPC 435/134; 435/257.2; 435/471

(57) ABSTRACT

This disclosure describes genetically modified photosynthetic microorganisms, e.g., Cyanobacteria, that overexpress an acyl-acyl carrier protein reductase (acyl-ACP reductase). These microorganisms may optionally overexpress one or more fatty acid synthesis proteins such as ACP and ACCase, and/or one or more polypeptides associated with glycogen breakdown. Also included are photosynthetic microorganisms comprising mutations or deletions in a glycogen biosynthesis or storage pathway, which accumulate a reduced amount of glycogen under reduced nitrogen conditions as compared to a wild type photosynthetic microorganism. The modified photosynthetic microorganisms provided herein are capable of producing increased amounts of lipids such as fatty acids or wax esters and/or synthesizing triglycerides.

### CROSS REFERENCE TO RELATED APPLICATIONS

This application claims the benefit under 35 U.S.C. §119(e) of U.S. Provisional Application No. 61/425,181, filed Dec. 20, 2010, U.S. Provisional Application No. 61/452,525 filed Mar. 14, 2011, and U.S. Provisional Application No. 61/477,773, filed Apr. 21, 2011, each of which is incorporated by reference in its entirety. This application claims priority to PCT Patent Application PCT/US2011/065896, filed on Dec. 19, 2011, which is incorporated by reference in its entirety.

### SEQUENCE LISTING

The Sequence Listing associated with this application is provided in text format in lieu of a paper copy, and is hereby incorporated by reference into the specification. The name of the text file containing the Sequence Listing is TARG_{—}021_{—}03WO_ST25.txt. The text file is about 328 KB, was created on Dec. 19, 2011, and is being submitted electronically via EFS-Web.

### BACKGROUND

1. Technical Field

The present invention relates generally to modified photosynthetic microorganisms (e.g., Cyanobacteria) that overexpress acyl-acyl carrier protein reductase (acyl-ACP reductase), or an active fragment or variant thereof, to produce increased levels of lipids such as fatty acids. Also included are related methods of using these modified photosynthetic microorganisms as a feedstock, e.g., for producing biofuels and other specialty chemicals.

2. Description of the Related Art

Fatty acids are carboxylic acids with an unbranched aliphatic tail or chain, the latter ranging from about four to about 28 carbon atoms in length. Triglycerides are neutral polar molecules consisting of glycerol esterified with three fatty acid molecules. Triglycerides and fatty acids can be utilized as carbon and energy storage molecules by most eukaryotic organisms, including plants and algae, and by certain prokaryotic organisms, including certain species of actinomycetes and members of the genus Acinetobacter.

Triglycerides and fatty acids may also be utilized as a feedstock in the production of biofuels and/or various specialty chemicals. For example, triglycerides and free fatty acids may be subject to a transesterification reaction, in which an alcohol reacts with triglyceride oils or fatty acid molecules, such as those contained in vegetable oils, animal fats, recycled greases, to produce biodiesels such as fatty acid alkyl esters. When triglycerides are included in the starting material, such reactions also produce glycerin as a by-product, which can be purified for use in the pharmaceutical and cosmetic industries

Certain organisms can be utilized as a source of triglycerides or free fatty acids in the production of biofuels. For example, algae naturally produce triglycerides as energy storage molecules, and certain biofuel-related technologies are presently focused on the use of algae as a feedstock for biofuels. Algae are photosynthetic organisms, and the use of triglyceride-producing organisms such as algae provides the ability to produce biodiesel from sunlight, water, CO₂, macronutrients, and micronutrients. Algae, however, cannot be readily genetically manipulated, and produce much less oil (i.e., triglycerides, fatty acids) under culture conditions than in the wild.

Like algae, Cyanobacteria obtain energy from photosynthesis, utilizing chlorophyll A and water to reduce CO₂. Certain Cyanobacteria can produce metabolites, such as carbohydrates, proteins, and fatty acids, from just sunlight, water, CO₂, water, and inorganic salts. Unlike algae, Cyanobacteria can be genetically manipulated. For example, Synechococcus is a genetically manipulable, oligotrophic Cyanobacterium that thrives in low nutrient level conditions, and in the wild accumulates fatty acids in the form of lipid membranes to about 10% by dry weight. Cyanobacteria such as Synechococcus, however, produce no triglyceride energy storage molecules, since Cyanobacteria typically lack the essential enzymes involved in triglyceride synthesis. Instead, Synechococcus in the wild typically accumulates glycogen as its primary carbon storage form.

Clearly, therefore, there is a need in the art for modified photosynthetic microorganisms, including Cyanobacteria, capable of producing lipids such as triglycerides and fatty acids, e.g., to be used as feed stock in the production of biofuels and/or various specialty chemicals.

### BRIEF SUMMARY

In various embodiments, the present invention provides modified photosynthetic microorganisms, as well as methods of producing and using the same. Certain embodiments include modified photosynthetic microorganisms, comprising an overexpressed acyl-acyl carrier protein (ACP) reductase polypeptide, wherein said modified photosynthetic microorganism produces an increased amount of free fatty acid (FFA) as compared to an unmodified photosynthetic microorganism of the same species. Some embodiments include modified photosynthetic microorganisms, comprising an overexpressed acyl-acyl carrier protein (ACP) reductase polypeptide, wherein said modified photosynthetic microorganism produces lipids in an amount of at least about 25-35 μg/mg/day. In certain embodiments, said microorganism is a Cyanobacterium. In certain embodiments, said lipid is a free fatty acid (FFA).

In certain embodiments, said overexpressed acyl-ACP reductase polypeptide is encoded by (i) an endogenous polynucleotide which is operably linked to one or more introduced regulatory elements, or (ii) an introduced polynucleotide. In certain embodiments, one or more introduced regulatory elements are derived from the same genus as said modified photosynthetic microorganism. In specific embodiments, said one or more introduced regulatory elements are derived from the same species as said modified photosynthetic microorganism. In some embodiments, said one or more introduced regulatory elements are derived from a different genus or species relative to said modified photosynthetic microorganism. In particular embodiments, said one or more introduced regulatory elements are selected from at least one of a promoter, enhancer, repressor, ribosome binding site, and a transcription termination site.

In certain embodiments, said one or more introduced regulatory elements comprises an inducible promoter. In some embodiments, said inducible promoter is a weak promoter under non-induced conditions. In certain embodiments, said one or more introduced regulatory elements comprises a constitutive promoter.

In certain embodiments, said overexpressed acyl-ACP reductase polypeptide is from Synechococcus elongatus PCC7942 (orf1594) or Synechocystis sp. PCC6803 (orfsII0209), or a fragment or variant thereof. In specific embodiments, said overexpressed acyl-ACP reductase polypeptide has the amino acid sequence set forth in SEQ ID NO:2, or a fragment or variant thereof.

In certain embodiments, the modified microorganism further comprises one or more of the following: (i) one or more overexpressed (e.g., introduced) polynucleotides encoding (a) an acyl carrier protein (ACP), (b) an acetyl coenzyme A carboxylase (ACCase), (c) a diacylglycerol acyltransferase (DGAT) optionally in combination with a fatty acyl Co-A synthetase, (d) an aldehyde dehydrogenase, (e) an alcohol dehydrogenase that is capable of converting a fatty aldehyde into a fatty alcohol optionally in combination with a wax ester synthase (e.g., DGAT having wax ester synthase activity), or (f) any combination of (a)-(e); (ii) reduced expression of one or more genes of a glycogen biosynthesis or storage pathway as compared to a wild-type photosynthetic microorganism; (iii) one or more introduced polynucleotides encoding a protein of a glycogen breakdown pathway; (iv) reduced expression of one or more genes encoding an endogenous aldehyde decarbonylase; (v) reduced expression of one or more genes encoding an acyl-ACP synthetase (Aas), or (vi) any combination of (i)-(v).

In certain embodiments, said ACP is a bacterial or a plant ACP. In particular embodiments, said ACP is from Synechococcus, Spinacia oleracea, Acinetobacter, Streptomyces, or Alcanivorax. In certain embodiments, said ACP has the amino acid sequence of any one of SEQ ID NOS:6, 8, 10, 12, or 14, or a fragment or variant thereof. In certain embodiments, said ACCase is from Synechococcus, Saccharomyces cerevisiae, or Triticum aestivum. In particular embodiments, said DGAT is an Acinetobacter DGAT, a Streptomyces DGAT, or an Alcanivorax DGAT.

In certain embodiments, said fatty acyl Co-A synthetase is from E. coli (FadD) or S. cerevisiae, or a fragment or variant thereof. In specific embodiments, said fatty acyl Co-A synthetase from S. cerevisiae is Faa1p, Faa2p, or Faa3p, or a fragment or variant thereof. In certain embodiments, the aldehyde dehydrogenase is from Synechococcus elongatus PCC7942. In some embodiments, the aldehyde dehydrogenase is encoded by orf0489 of Synechococcus elongatus PCC7942, or a homolog or paralog thereof, or a fragment or variant thereof. In specific embodiments, the aldehyde dehydrogenase has the amino acid sequence of SEQ ID NO:103, or a fragment or variant thereof. In particular embodiments, photosynthetic microorganisms comprising the introduced (or overexpressed) aldehyde dehydrogenase in addition to the overexpressed acyl-ACP reductase have increased cell growth, increased production of free fatty acids, or both, relative to photosynthetic microorganisms comprising the overexpressed acyl-ACP reductase without the introduced (or overexpressed) aldehyde dehydrogenase.

In certain embodiments, said one or more genes of a glycogen biosynthesis or storage pathway are selected from a glucose-1-phosphate adenyltransferase (glgC) gene and a phosphoglucomutase (pgm) gene. Some embodiments comprise a full or partial deletion of said one or more genes of a glycogen breakdown pathway. In certain embodiments, said one or more proteins of a glycogen breakdown pathway are selected from glycogen phosphorylase (GlgP), glycogen isoamylase (GlgX), glucanotransferase (MalQ), phosphoglucomutase (Pgm), glucokinase (Glk), and phosphoglucose isomerase (Pgi).

Certain embodiments comprise a full or partial deletion of the endogenous aldehyde decarbonylase. In particular embodiments, said aldehyde decarbonylase is encoded by orf1593 of S. elongatus PCC7942, orfsII0208 of Synechocystis sp. PCC6803, or a homolog or paralog thereof, or a fragment or variant thereof. Certain embodiments comprise a full or partial deletion of the endogenous Aas. Certain embodiments combine the acyl-ACP reductase and the aldehyde dehydrogenase.

In certain embodiments, one or more of said introduced polynucleotides is present in one or more expression constructs. In certain embodiments, said expression construct is stably integrated into the genome of said modified photosynthetic microorganism. In certain embodiments, said expression construct comprises an inducible promoter. In some embodiments, one or more of said introduced polynucleotides are present in an expression construct comprising a weak promoter under non-induced conditions.

In certain embodiments, one or more of said introduced polynucleotides are codon-optimized for expression in a Cyanobacterium. In particular embodiments, one or more of said codon-optimized polynucleotides are codon-optimized for expression in a Synechococcus elongatus. In certain embodiments, said photosynthetic microorganism is a Cyanobacterium and said Cyanobacterium is a Synechococcus elongatus. In specific embodiments, said Synechococcus elongatus is strain PCC7942. In certain embodiments, said Cyanobacterium is a salt tolerant variant of Synechococcus elongatus strain PCC7942. In other embodiments, said photosynthetic microorganism is a Cyanobacterium and said Cyanobacterium is Synechococcus sp. PCC7002. In certain embodiments, said photosynthetic microorganism is a Cyanobacterium and said Cyanobacterium is Synechocystis sp. PCC6803.

Also included is a modified Synechococcus elongatus PCC7942, comprising an overexpressed acyl-acyl carrier protein (ACP) reductase polypeptide, wherein said overexpressed polypeptide is encoded by (i) an endogenous polynucleotide which is operably linked to one or more introduced regulatory elements, or (ii) an introduced polynucleotide, and wherein said modified Synechococcus elongatus PCC7942 produces or accumulates an increased amount of free fatty acid as compared to a corresponding wild-type or unmodified Synechococcus elongatus PCC7942.

In certain embodiments, such as DGAT-expressing strains, said microorganism produces an increased amount of triglycerides as compared to a DGAT only-expressing microorganism of the same species, or a DGAT-expressing microorganism of the same species which does not overexpress an acyl-ACP reductase.

Also included are methods of producing a modified photosynthetic microorganism that produces or accumulates an increased amount of lipid as compared to a corresponding wild-type photosynthetic microorganism, comprising over-expressing an acyl-acyl carrier protein (ACP) reductase polypeptide in said modified photosynthetic microorganism. In certain embodiments, said modified photosynthetic microorganism is a Cyanobacterium. In particular embodiments, said modified photosynthetic microorganism produces one or more lipids in an amount of at least about 25-35 μg/mg/day. In some embodiments, said lipid is a free fatty acid (FFA).

Certain methods comprise (i) introducing one or more regulatory elements which are operably linked to an endogenous polynucleotide that encodes said acyl-ACP reductase, and/or (ii) introducing a polynucleotide that encodes said acyl-ACP reductase. In certain embodiments, said one or more introduced regulatory elements are derived from the same genus as said modified photosynthetic microorganism. In certain embodiments, said one or more introduced regulatory elements are derived from the same species as said modified photosynthetic microorganism. In certain embodiments, said one or more introduced regulatory elements are derived from a different genus or species relative to said modified photosynthetic microorganism. In some embodiments, said one or more introduced regulatory elements are selected from at least one of a promoter, enhancer, repressor, ribosome binding site, and a transcription termination site.

In certain embodiments, said one or more introduced regulatory elements comprises an inducible promoter. In particular embodiments, said inducible promoter is a weak promoter under non-induced conditions. In certain embodiments, said one or more introduced regulatory elements comprises a constitutive promoter. In certain embodiments, said overexpressed acyl-ACP reductase polypeptide is from Synechococcus elongatus PCC7942 (orf1594) or Synechocystis sp. PCC6803 (orfsII0209), or a fragment or variant thereof. In specific embodiments, said overexpressed acyl-ACP reductase polypeptide has the amino acid sequence set forth in SEQ ID NO:2, or a fragment or variant thereof.

Certain methods further comprise one or more of the following: (i) introducing one or more polynucleotides encoding (a) an acyl carrier protein (ACP), (b) an acetyl coenzyme A carboxylase (ACCase), (c) a diacylglycerol acyltransferase (DGAT) optionally in combination with a fatty acyl Co-A synthetase, (d) an aldehyde dehydrogenase, (e) an alcohol dehydrogenase that is capable of converting a fatty aldehyde into a fatty alcohol optionally in combination with a wax ester synthase (e.g., DGAT having wax ester synthase activity), or (f) any combination of (a)-(e); (ii) modifying the microorganism to reduce expression of one or more genes of a glycogen biosynthesis or storage pathway as compared to a wild-type photosynthetic microorganism; (iii) introducing one or more polynucleotides encoding a protein of a glycogen breakdown pathway; (iv) modifying the microorganism to reduce expression of one or more genes encoding an endogenous aldehyde decarbonylase; (v) modifying the microorganism to reduce expression of one or more genes encoding an acyl-ACP synthetase (Aas), or (vi) any combination of (i)-(v). In certain embodiments, said ACP is a bacterial or a plant ACP. In certain embodiments, said ACP is from Synechococcus, Spinacia oleracea, Acinetobacter, Streptomyces, or Alcanivorax. In particular embodiments, said ACP has the amino acid sequence of any one of SEQ ID NOS:6, 8, 10, 12, or 14, or a fragment or variant thereof.

In certain embodiments, said ACCase is from Synechococcus, Saccharomyces cerevisiae, or Triticum aestivum. In some embodiments, said DGAT is an Acinetobacter DGAT, a Streptomyces DGAT, or an Alcanivorax DGAT. In certain embodiments, said fatty acyl Co-A synthetase is from E. coli (FadD) or S. cerevisiae, or a fragment or variant thereof. In certain embodiments, said fatty acyl Co-A synthetase from S. cerevisiae is Faa1p, Faa2p, or Faa3p, or a fragment or variant thereof.

In certain embodiments, the aldehyde dehydrogenase is from Synechococcus elongatus PCC7942. In some embodiments, the aldehyde dehydrogenase is encoded by orf0489 of Synechococcus elongatus PCC7942, or a homolog or paralog thereof, or a fragment or variant thereof. In specific embodiments, the aldehyde dehydrogenase has the amino acid sequence of SEQ ID NO:103, or a fragment or variant thereof. In particular embodiments, the introduced (overexpressed) aldehyde dehydrogenase increases cell growth, increases production of free fatty acids, or both, relative to overexpression of the acyl-ACP reductase without the introduced aldehyde dehydrogenase.

In certain embodiments, said one or more genes of a glycogen biosynthesis or storage pathway are selected from a glucose-1-phosphate adenyltransferase (glgC) gene and a phosphoglucomutase (pgm) gene. Some embodiments comprise a full or partial deletion of said one or more genes of a glycogen breakdown pathway. In certain embodiments, said one or more proteins of a glycogen breakdown pathway are selected from glycogen phosphorylase (GlgP), glycogen isoamylase (GlgX), glucanotransferase (MalQ), phosphoglucomutase (Pgm), glucokinase (Glk), and phosphoglucose isomerase (Pgi).

Particular embodiments comprise a full or partial deletion of the endogenous aldehyde decarbonylase. In certain embodiments, said aldehyde decarbonylase is encoded by orf1593 of S. elongatus PCC7942, orfsII0208 of Synechocystis sp. PCC6803, or a homolog or paralog thereof, or a fragment or variant thereof. Some embodiments comprise a full or partial deletion of the endogenous Aas. Certain embodiments combine the acyl-ACP reductase and the aldehyde dehydrogenase.

In certain embodiments, one or more of said introduced polynucleotides is present in one or more expression constructs. In certain embodiments, said expression construct is stably integrated into the genome of said modified photosynthetic microorganism. In certain embodiments, said expression construct comprises an inducible promoter. In some embodiments, one or more of said introduced polynucleotides are present in an expression construct comprising a weak promoter under non-induced conditions. In certain embodiments, one or more of said introduced polynucleotides are codon-optimized for expression in a Cyanobacterium. In particular embodiments, one or more of said codon-optimized polynucleotides are codon-optimized for expression in a Synechococcus elongatus. In certain embodiments, said photosynthetic microorganism is a Cyanobacterium and said Cyanobacterium is a Synechococcus elongatus. In specific embodiments, said Synechococcus elongatus is strain PCC7942. In certain embodiments, said Cyanobacterium is a salt tolerant variant of Synechococcus elongatus strain PCC7942. In other embodiments, said photosynthetic microorganism is a Cyanobacterium and said Cyanobacterium is Synechococcus sp. PCC7002. In some embodiments, said photosynthetic microorganism is a Cyanobacterium and said Cyanobacterium is Synechocystis sp. PCC6803.

Also included are methods for the production of lipids, comprising culturing a modified photosynthetic microorganism described herein, wherein said modified photosynthetic microorganism accumulates an increased amount of lipid as compared to a corresponding wild-type photosynthetic microorganism. In certain embodiments, said culturing comprises inducing expression of one or more of said introduced polynucleotides. In some embodiments, said culturing comprises culturing under static growth conditions. In certain embodiments, said inducing occurs under static growth conditions.

In particular embodiments, said culturing comprises culturing in media supplemented with bicarbonate. In specific embodiments, the concentration of bicarbonate is selected from about 5, 10, 20, 50, 75, 100, 200, 300, 400, 500, 600, 700, 800, 900, and 1000 mM bicarbonate. In certain embodiments, the bicarbonate is present prior to inducing expressing of the introduced polynucleotide. In some embodiments, the bicarbonate is present during induction of the introduced polynucleotide.

In certain embodiments, said lipid is a free fatty acid. In specific embodiments, said free fatty acid is a C16:0 fatty acid. In some embodiments, said lipid is a wax ester.

Also included are modified photosynthetic microorganisms, comprising an overexpressed acyl-acyl carrier protein (ACP) reductase polypeptide; and an aldehyde dehydrogenase polypeptide that converts a fatty (acyl) aldehyde to a fatty acid, wherein the modified photosynthetic microorganism produces an increased amount of free fatty acid (FFA) as compared to an unmodified photosynthetic microorganism of the same species, or as compared to a modified photosynthetic microorganism of the same species that overexpresses the acyl-ACP reductase without expressing the aldehyde dehydrogenase (e.g., a deletion mutant or a species of microorganism that does not naturally express the aldehyde dehydrogenase). In certain instances, the aldehyde dehydrogenase can be characterized by its ability to utilize an acyl aldehyde such as nonyl-aldehyde or C16 fatty aldehydes as a substrate, and convert the acyl aldehyde to a fatty acid. Certain of these and related embodiments are combined with reduced expression of one or more Aas genes.

In some embodiments, the aldehyde dehydrogenase is encoded by an unmodified, endogenous polynucleotide (i.e., it is a naturally-occurring aldehyde dehydrogenase, expressed at naturally-occurring levels). In particular embodiments, the aldehyde dehydrogenase is encoded by an endogenous polynucleotide, and is overexpressed by operably linking the endogenous polynucleotide to one or more introduced regulatory elements. In specific embodiments, the microorganism is Synechococcus elongatus PCC7942, and the aldehyde dehydrogenase is encoded by orf0489 of S. elongatus PCC7942. In certain embodiments, the aldehyde dehydrogenase is overexpressed and encoded by an introduced polynucleotide. In certain embodiments, the introduced polynucleotide is orf0489 of Synechococcus elongatus PCC7942. In specific embodiments, the aldehyde dehydrogenase has the amino acid sequence of SEQ ID NO:103, or a fragment or variant thereof.

In some embodiments, the aldehyde dehydrogenase increases cell growth, increases production of free fatty acids, or both, compared to overexpression of the acyl-ACP reductase without expression of the aldehyde dehydrogenase. In particular embodiments, the overexpressed aldehyde dehydrogenase increases cell growth, increases production of free fatty acids, or both, compared to overexpression of the acyl-ACP reductase with naturally-occurring levels of the aldehyde dehydrogenase.

In certain embodiments, a modified photosynthetic microorganism of the present invention (comprising an overexpressed acyl-ACP reductase) further comprises an overexpressed alcohol dehydrogenase (e.g., long-chain alcohol dehydrogenase) in optional combination with an overexpressed wax ester synthase (e.g., DGAT having wax ester synthase activity), and produces an increased amount of wax ester(s) as compared to a wild-type or unmodified microorganism of the same species. In some embodiments, the alcohol dehydrogenase is from Synechocystis sp. PCC6803 or Acinetobacter baylyi. In some embodiments, the alcohol dehydrogenase has the amino acid sequence of SEQ ID NO:105 (slr1192) or 107 (ACIAD3612), or an active fragment or variant thereof. In specific embodiments, the DGAT is aDGAT.

Further to an acyl-ACP reductase, alcohol dehydrogenase, and wax ester synthase, certain modified photosynthetic microorganisms may also comprise (i) reduced expression of an endogenous aldehyde dehydrogenase, (i) reduced expression of an aldehyde decarbonylase, (iii) an overexpressed acyl carrier protein (ACP) optionally in combination with an over expressed acyl-ACP synthetase (Aas), or (iv) any combination of (i)-(iii). Specific modified microorganisms have reduced expression of the aldehyde dehydrogenase encoded by orf0489, for example, by deletion of all or a portion of the orf0489 coding sequence, or a regulatory sequence thereof. Some modified microorganisms have reduced expression of the aldehyde decarbonylase encoded by orf1593. Certain of these modified photosynthetic microorganisms produce an increased amount of wax ester(s) as compared to a modified microorganism without any of (i)-(iv). Certain microorganisms having reduced expression of orf0489 produce an increased amount of wax ester(s) as compared to modified or unmodified microorganisms without reduced expression of orf1489. Also included are methods of producing wax esters, comprising culturing one or more of these and related modified photosynthetic microorganisms under conditions suitable for wax ester formation.

### BRIEF DESCRIPTION OF THE SEVERAL VIEWS OF THE DRAWINGS

FIGS. 1A-1D show that overexpression of orf1594 (acyl-ACP reductase) in S. elongatus PCC7942 leads to a significant increase in free fatty acids. Cells were subcultured to achieve an OD₇₅₀ of 0.1 the day before induction and induced with 1 mM IPTG (0 hr) (FIG. 1A). At 24 hours post-induction, 0.5 OD equivalents of whole lysate were separated by thin layer chromatograph (TLC) using a mobile phase for polar lipids (70 mL chloroform/22 mL methanol/3 mL water); 5 μg of a palmitic acid standard was included (FIG. 1B). Samples on TLC include WT (lane 1); Ald1 (lanes 2 and 3); orf1594 (lanes 4 and 5); and orf1594/ACP (lanes 6 and 7). Samples of wild-type, uninduced orf1594, and induced orf1594 were also analyzed at 24 h and 48 h post-induction by gas chromatography (GC) for total fatty acid methyl esters (FAMES) (FIG. 1C). Quantitation by GC of constituent FAMES (C14:0, C14:1, C16:0, C16:1n9 and C18:0) (FIG. 1D); the increase in total FAMES is primarily due to an increase in C16:0.

FIG. 2 illustrates a pathway in which acyl-ACP is first converted to an acyl aldehyde by acyl-ACP reductase, and is then converted to an alkane by an aldehyde decarbonylase. FIG. 2 also illustrates a pathway in which acyl aldehyde is converted to a free fatty acid by an aldehyde dehydrogenase, such as orf0489. FIG. 2 further illustrates a pathway in which acyl aldehyde is converted to a fatty alcohol by an alcohol dehydrogenase, which is then converted into a wax ester by an enzyme having wax ester synthase activity (e.g., aDGAT).

FIG. 3 shows the role of aldehyde dehydrogenase (orf0489) in orf1594-induced FFA production. Strains (1594; Δ0489 (or D0489); and 1594/Δ0489#1 and #2 were diluted to an OD₇₅₀ of 0.1 the day before inductions and induced with 1 mM IPTG at t=0. Samples were collected for TLC and GC analysis 24 and 48 hr post-induction (FIGS. 3A and 3B). For TLC, 0.50D-equivalents were separated using non-polar solvents; 5 μg or 10 μg of palmitic acid (“PA”) standard was included. The free fatty acid produced in the orf1594-expressing strain is indicated by an asterisk (FIG. 3B). Colony forming units (CFU) were assessed at 48 hours post-induction (FIG. 3C). The lipid content from the strains was measured 24 and 48 hours post-induction by GC (FIG. 3D).

FIG. 4 shows that purified h6-orf0489 utilizes nonyl-aldehyde as a substrate. FIG. 4A shows the reaction scheme for measuring orf0489 aldehyde dehydrogenase activity. The reaction was started by mixing together a fatty aldehyde substrate, purified h6-orf0489 and NAD(P)+. The progress of the reaction was assessed by measuring the production of NAD(P)H at 340 nm using the SpectraMax M5. FIG. 4B shows the SDS-PAGE of metal affinity-purified h₆-0489. FIG. 4C shows the results of the enzyme assay with varying concentrations of h₆-0489 (0.3, 1.5 or 6 μM final concentration). Nonyl-aldehyde and either NAD+ or NAD(P)+ were used at 1 mM. Measurements were taken every 30 seconds for 30 minutes.

FIGS. 5A-5D show the results of co-overexpression of acyl-ACP reductase (orf1594) with aldehyde dehydrogenase (orf0489). Strains (WT; orf1594(2×); (orf15942X)/0489#3 and #4) were grown in BG11 media (plus antibiotic), diluted to an OD₇₅₀ of 0.1 the day before induction in BG11 (without antibiotic), and induced the following day with 1 mM IPTG. Samples were then collected at 48 and 96 hours for growth and CFU (FIGS. 5A and 5B), 24 hours for qPCR (FIG. 5C, testing with primer/probes sets specific for orf0489 and orf1594); and 48, 96 and 144 hours for GC (FIGS. 5D and 5E). The total FAMES are represented either as μg/OD*mL (FIG. 5D) or as μg/mL (FIG. 5E).

FIGS. 6A-6D show the production of wax esters by Cyanobacteria modified to co-express acyl-ACP reductase (orf1594), an alcohol dehydrogenase (slr1192 or ACIAD3612), and wax ester synthase (aDGAT). FIG. 6A shows growth curves (induction started at 0 hours) as measured by colony-forming units (CFU). FIGS. 6A and 6B show thin layer chromatograpy (TLC) of samples 24 hours post-induction. 0.5 OD-equivalents were separated using nonpolar solvents (90% hexane/10% diethyl ether; FIG. 6A) to show TAG and WE formation, or polar solvents (FIG. 6B) to show fatty acid formation. 5 μg of WE and TAG standards were included for analysis the non-polar plate; and 5 μg of palmitic acid (PA) was included for analysis of the polar plate. FIG. 6D shows total FAMES (expressed as μg/OD*mL) from samples collected 24 h post-induction.

### DETAILED DESCRIPTION

The present invention is based upon the discovery that photosynthetic microorganisms, e.g., Cyanobacteria, modified to overexpress an acyl-acyl carrier protein reductase (acyl-ACP reductase), or a fragment or variant thereof, produce increased amounts of lipids, e.g., free fatty acids, and/or wax esters, and often demonstrate an increase in total cellular lipid content, which is advantageous for the production of carbon-based products, including biofuels.

As described in the accompanying Examples, a modified Cyanobacterium overexpressing an acyl-ACP reductase gene (orf1594) from Synechococcus elongatus (ACP) produced a significantly increased amount of fatty acids compared to the unmodified strain. The orf1594-expressing strain not only displayed no growth defects or toxicity, but also showed constant production of fatty acids throughout the time course, with a preferential increase in C16:0 fatty acids, thus yielding an attractive strain for continuous production of fatty acids.

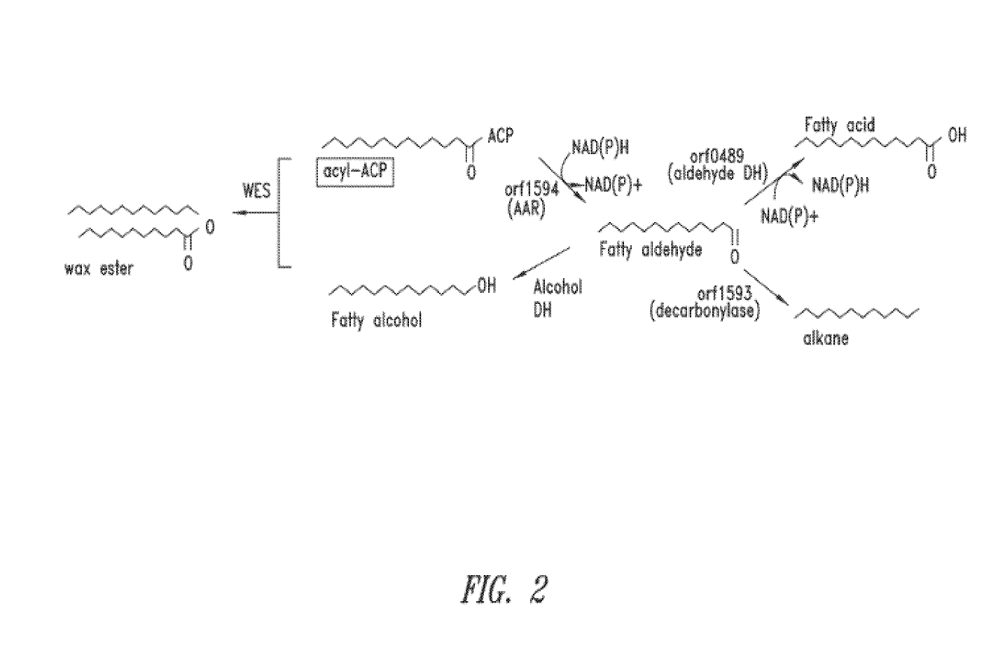

Increased production of fatty acids from this orf1594 (acyl-ACP reductase)-expressing strain is surprising and unexpected. Without wishing to be bound by any one theory, it is understood that the acyl-ACP reductase encoded by orf1594 of S. elongatus converts acyl-ACP to an acyl aldehyde. An aldehyde decarbonylase encoded by orf1593 then converts this acyl aldehyde to an alkane or an alkene (see Schirmer et al., Science. 329:559-562, 2010; and FIG. 2 for an illustration of this model). Overexpression of orf1594 by itself in E. coli has been shown to increase production of acyl aldehyde (hexadecanal) and acyl alcohol (hexadecanol) (see Schirmer et al., supra). The high levels of acyl alcohol (hexadecanol) were explained by the possible presence of endogenous E. coli alcohol dehydrogenase(s), which converts acyl aldehydes to acyl alcohol. However, without more, there would have been little or no reason to expect overexpression of orf1594 by itself to increase production of free fatty acids in photosynthetic microorganisms such as Cyanobacteria. If anything, based on the above results, overexpression of orf1594 by itself might have been expected to increase levels of acyl aldehydes, acyl alcohols, and/or possibly alkanes/alkenes. Despite this expectation, the accompanying Examples show that overexpression of orf1594 by itself is sufficient to significantly increase levels of fatty acids in Cyanobacteria.

According to one non-limiting theory, this unexpected result is due to the presence of a previously unidentified Cyanobacterial aldehyde dehydrogenase, which rapidly converts acyl aldehydes to free fatty acids. In the complete absence of such an enzyme, the overexpression of acyl-ACP reductase might otherwise result in increased alkane production (by activity of aldehyde decarbonylase), or increased accumulation of acyl aldehydes with potentially negative effects on cell viability. Studies provided herein have shown that one such exemplary aldehyde dehydrogenase is encoded by orf0489 of Synechococcus elongatus PCC7942 (see SEQ ID NOS:102 and 103). Among other characteristics, this aldehyde dehydrogenase and its functional equivalents may be characterized, for example, by the ability to utilize certain acyl aldehydes (e.g., nonyl-aldehyde, C₁₄, C₁₆, C₁₈ fatty aldehyde) as a substrate, and convert them into fatty acids.

Hence, in certain embodiments, overexpression of orf0489 or a functionally equivalent aldehyde dehydrogenase may further increase the production of fatty acids or other lipids by an acyl-ACP-overexpressing microorganism, improve its growth characteristics (e.g., by reducing accumulation of potentially toxic acyl aldehydes), or both, relative to a modified microorganism that expresses only naturally-occurring levels of the aldehyde dehydrogenase. As one example, these embodiments can be useful in a modified microorganism that highly overexpresses an acyl-ACP reductase, e.g., a microorganism having two or more introduced polynucleotides that encode an acyl-ACP reductase.

Expression of orf0489 or a functionally equivalent aldehyde dehydrogenase may also be utilized in a modified strain that does not naturally express such an enzyme; in these instances, the combination of acyl-ACP reductase overexpression (to produce acyl aldehydes) and orf0489 (over)expression would then lead to increased production of fatty acids, instead of excess accumulation of acyl aldehydes or production of other products such as alkanes. Regardless, it has been shown that for certain Cyanobacteria, overexpression of orf1594 by itself increases production of free fatty acids without inducing significant toxicity or growth inhibition, and that naturally-occurring levels of orf0489 expression contribute to this result.

The present invention, therefore, relates generally to modified photosynthetic microorganisms, including modified Cyanobacteria, that overexpress one or more acyl-ACP reductases, or fragments or variants thereof, as well as methods of producing such modified photosynthetic microorganisms and methods of using them for the production of fatty acids and lipids, e.g., for use in the production of carbon-based products. Because the genome of certain Cyanobacterium such as Synechococcus elongatus contains an endogenous or naturally-occurring acyl-ACP reductase (e.g., orf1594), certain embodiments relate to overexpressing these endogenous genes without introducing a foreign copy of the gene, such as by stably introducing one or more promoters or other operatively linked regulatory elements into a genomic region surrounding (i.e., upstream or downstream) an endogenous acyl-ACP reductase gene. Such promoters or other regulatory elements (e.g., promoters, enhancers, repressors, ribosome binding sites, transcription termination sites) can be derived from any suitable source; exemplary regulatory elements are described elsewhere herein. In certain aspects, the one or more regulatory elements are all derived from the same species of microorganism being modified. Even though these and related microorganisms are modified by recombinant techniques, they do not necessarily contain any foreign nucleic acid sequences (i.e., sequences from other microorganisms), and thus are not “genetically modified organisms (GMOs)” in the traditional sense of that term.

As one example, certain embodiments include the introduction of inducible and/or constitutive promoters, which can be derived from the same or a different genus/species of photosynthetic microorganism relative to the microorganism being modified. Specific embodiments include, for instance, the introduction of a Synechococcus (elongatus) promoter upstream of orf1594 (encoding acyl-ACP reductase) in Synechococcus elongatus PCC7942. Related exemplary embodiments include the introduction of a Synechocystis promoter upstream of orfsII0209 (encoding acyl-ACP reductase) in Synechocystis sp. PCC6803.

Acyl-ACP reductase polypeptides can also be overexpressed by recombinantly introducing one or more polynucleotides encoding said polypeptide(s), whether derived from the same or a different genus/species of microorganism relative to the microorganism being modified. In certain embodiments, the overexpression of acyl-ACP reductase can be combined with the overexpression of one or more selected lipid biosynthesis genes, such as acyl carrier protein (ACP), acetyl coenzyme A carboxylase (ACCase), an aldehyde dehydrogenase, and/or diacylglycerol acyltransferase (DGAT) when optionally combined with a fatty acyl-CoA synthetase, to further increase production of lipids such as fatty acids and/or triglycerides. Separately or in combination with strains having overexpressed lipid biosynthesis proteins, the overexpression of acyl-ACP reductase can also be combined with strains having reduced expression of one or more genes of a glycogen biosynthesis or storage pathway as compared to a wild-type photosynthetic microorganism, and/or strains having overexpressed proteins involved in a glycogen breakdown pathway. Certain of these embodiments are detailed elsewhere herein.

Aspects of the present invention can also be combined with the discovery that photosynthetic microorganisms such as Cyanobacteria can be genetically modified in other ways to increase the production of fatty acids, as described herein and in International Patent Application US2009/061936 and U.S. patent application Ser. No. 12/605,204. Since fatty acids provide the starting material for triglycerides, increasing the production of fatty acids in genetically modified photosynthetic microorganisms may be utilized to increase the production of triglycerides, as described herein and in International Patent Application PCT/US2009/061936. In addition to diverting carbon usage away from glycogen synthesis and towards lipid production, photosynthetic microorganisms of the present invention can also be modified to increase the production of fatty acids by introducing one or more exogenous polynucleotide sequences that encode one or more enzymes associated with fatty acid synthesis. In certain aspects, the exogenous polynucleotide sequence encodes an enzyme that comprises an acyl-CoA carboxylase (ACCase) activity, typically allowing increased ACCase expression, and, thus, increased intracellular ACCase activity. Increased intracellular ACCase activity contributes to the increased production of fatty acids because this enzyme catalyzes the “commitment step” of fatty acid synthesis. Specifically, ACCase catalyzes the production of a fatty acid synthesis precursor molecule, malonyl-CoA. In certain embodiments, the polynucleotide sequence encoding the ACCase is not native the photosynthetic microorganisms's genome.

Embodiments of the present invention are also useful in combination with the related discovery that photosynthetic microorganisms, including Cyanobacteria, such as Synechococcus, which do not naturally produce triglycerides, can be genetically modified to synthesize triglycerides, as described herein and in International Patent Application US20091061936 and U.S. patent application Ser. No. 12/605,204, filed Oct. 23, 2009, titled Modified Photosynthetic Microorganisms for Producing Triglycerides. For instance, the addition of one or more polynucleotide sequences that encode one or more enzymes associated with triglyceride synthesis renders Cyanobacteria capable of converting their naturally-occurring fatty acids into triglyceride energy storage molecules. Examples of enzymes associated with triglyceride synthesis include enzymes having a phosphatidate phosphatase activity and enzymes having a diacylglycerol acyltransferase activity (DGAT). Specifically, phosphatidate phosphatase enzymes catalyze the production of diacylglycerol molecules, an immediate pre-cursor to triglycerides, and DGAT enzymes catalyze the final step of triglyceride synthesis by converting the diacylglycerol precursors to triglycerides.

Aspects of the present invention may also be combined with the discovery that the functional removal of certain genes involved in glycogen synthesis, such as by mutation or deletion, leads to reduced glycogen accumulation and/or storage in photosynthetic microorganisms, such as Cyanobacteria, as described in PCT Application No. US2009/069285 and U.S. patent application Ser. No. 12/645,228. For instance, Cyanobacteria, such as Synechococcus, which contain deletions of the glucose-1-phosphate adenylyltransferase gene (glgC), the phosphoglucomutase gene (pgm), and/or the glycogen synthase gene (glgA), individually or in various combinations, may produce and accumulate significantly reduced levels of glycogen as compared to wild-type Cyanobacteria. The reduction of glycogen accumulation may be especially pronounced under stress conditions, including the reduction of nitrogen. Aspects of the present invention may be further combined with the discovery that overexpression of genes or proteins involved in glycogen breakdown in photosynthetic microorganisms, such as Cyanobacteria, also leads to reduced glycogen and/or storage.

Embodiments of the present invention may also be combined with the discovery that the co-expression of an alcohol dehydrogenase and a wax ester synthase results in wax ester formation, via the acyl-ACP=>fatty aldehyde pathway. For instance, as shown in the accompanying Examples, Cyanobacteria over-expressing an acyl-ACP reductase (e.g., orf1594), a long chain alcohol dehydrogenase, and the bi-functional aDGAT enzyme not only produce fewer triglycerides, but also produce wax esters. Because the accompanying Examples also show that these modified Cyanobacteria produce free fatty acids, and thus suggest that endogenous aldehyde dehydrogenase encoded by orf0489 competes with alcohol dehydrogenase for acyl aldehyde substrate, reduced expression (e.g., deletion) of orf0489 may increase wax ester synthesis in these and related microorganisms, relative to modified microorganisms having no reduced expression of orf0489. Further, because aldehyde decarbonylase encoded by orf1593 may also compete with alcohol dehydrogenase for acyl aldehyde substrate, reduced expression (e.g., deletion) of orf1593 may independently increase wax ester synthesis, and when combined with reduced expression of orf0489 may even further increase wax ester synthesis. Increased wax ester formation may also be achieved by combining any one of these or related embodiments with overexpression of other genes related to fatty aldehyde synthesis, including acyl carrier protein (ACP), Aas, or both.

### DEFINITIONS

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length.

The term “biologically active fragment”, as applied to fragments of a reference polynucleotide or polypeptide sequence, refers to a fragment that has at least about 0.1, 0.5, 1, 2, 5, 10, 12, 14, 16, 18, 20, 22, 24, 26, 28, 30, 35, 40, 45, 50, 55, 60, 65, 70, 75, 80, 85, 90, 95, 96, 97, 98, 99, 100, 110, 120, 150, 200, 300, 400, 500, 600, 700, 800, 900, 1000% or more of the activity of a reference sequence. The term “reference sequence” refers generally to a nucleic acid coding sequence, or amino acid sequence, to which another sequence is being compared. All sequences provided in the Sequence Listing are also included as reference sequences.

The term “biologically active variant”, as applied to variants of a reference polynucleotide or polypeptide sequence, refers to a variant that has at least about 0.1, 0.5, 1, 2, 5, 10, 12, 14, 16, 18, 20, 22, 24, 26, 28, 30, 35, 40, 45, 50, 55, 60, 65, 70, 75, 80, 85, 90, 95, 96, 97, 98, 99, 100, 110, 120, 150, 200, 300, 400, 500, 600, 700, 800, 900, 1000% or more of the activity (e.g., an enzymatic activity) of a reference sequence. The term “reference sequence” refers generally to a nucleic acid coding sequence, or amino acid sequence, to which another sequence is being compared. The term “variant” encompasses biologically active variants, which may also be referred to as functional variants.

Included within the scope of the present invention are biologically active fragments of at least about 18, 19, 20, 21, 22, 23, 24, 25, 26, 27, 28, 29, 30, 40, 50, 60, 70, 80, 90, 100, 120, 140, 160, 180, 200, 220, 240, 260, 280, 300, 320, 340, 360, 380, 400, 500, 600 or more contiguous nucleotides or amino acid residues in length, including all integers in between, which comprise or encode a polypeptide having an activity of a reference polynucleotide or polypeptide. Representative biologically active fragments and variants generally participate in an interaction, e.g., an intra-molecular or an inter-molecular interaction. An inter-molecular interaction can be a specific binding interaction or an enzymatic interaction. Examples of enzymatic interactions or activities include, without limitation, acyl-acyl carrier protein reductase activity, acyl carrier protein activity, glycogen breakdown activity, and/or acetyl-CoA carboxylase activity, aldehyde dehydrogenase activity, alcohol dehydrogenase activity, and others described herein.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present.

By “consisting essentially of is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids” bases are matched according to the base pairing rules. Or, there may be “complete” or “total” complementarity between the nucleic acids. The degree of complementarity between nucleic acid strands has significant effects on the efficiency and strength of hybridization between nucleic acid strands.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

By “derivative” is meant a polypeptide that has been derived from the basic sequence by modification, for example by conjugation or complexing with other chemical moieties (e.g., pegylation) or by post-translational modification techniques as would be understood in the art. The term “derivative” also includes within its scope alterations that have been made to a parent sequence including additions or deletions that provide for functionally equivalent molecules.

By “enzyme reactive conditions” it is meant that any necessary conditions are available in an environment (i.e., such factors as temperature, pH, lack of inhibiting substances) which will permit the enzyme to function. Enzyme reactive conditions can be either in vitro, such as in a test tube, or in vivo, such as within a cell.

As used herein, an “acyl-acyl carrier protein reductase” (or “acyl-ACP reductase”) includes an enzyme that converts acyl-ACP to acyl-aldehyde.

As used herein, the terms “function” and “functional” and the like refer to a biological, enzymatic, or therapeutic function.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

“Homology” refers to the percentage number of amino acids that are identical or constitute conservative substitutions. Homology may be determined using sequence comparison programs such as GAP (Deveraux et al., 1984, Nucleic Acids Research 12, 387-395) which is incorporated herein by reference. In this way sequences of a similar or substantially different length to those cited herein could be compared by insertion of gaps into the alignment, such gaps being determined, for example, by the comparison algorithm used by GAP.

The term “host cell” includes an individual cell or cell culture which can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell which comprises a recombinant vector of the invention is a recombinant host cell.

By “isolated” is meant material that is substantially or essentially free from components that normally accompany it in its native state. For example, an “isolated polynucleotide”, as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

By “increased” or “increasing” is meant the ability of one or more modified photosynthetic microorganisms, e.g., Cyanobacteria, to produce or store a greater amount of a given fatty acid, lipid molecule, or triglyceride as compared to a control photosynthetic microorganism, typically of the same species, such as an unmodified Cyanobacteria or a differently modified Cyanobacteria. Also included are increases in total lipids, total fatty acids, total free fatty acids, total intracellular fatty acids, and/or total secreted fatty acids, separately or together. For instance, in certain embodiments, total lipids may increase, with either corresponding increases in all types of lipids, or relative increases in one or more specific types of lipid (e.g., fatty acids, free fatty acids, secreted fatty acids, triglycerides, wax esters). In certain embodiments, total lipids may increase or they may stay the same (i.e., total lipids are not significantly increased compared to an unmodified microorganism of the same type), and the production or storage of fatty acids (e.g., free fatty acids, secreted fatty acids) may increase relative to other lipids. In particular embodiments, the production or storage of one or more selected types of fatty acids (e.g., secreted fatty acids, free fatty acids, intracellular fatty acids, specific fatty acids such as C14:0, C14:1, C16:0, C16:1n9, and C18:0 fatty acids) may increase relative to other types of fatty acids (e.g., secreted fatty acids, free fatty acids, intracellular fatty acids, specific fatty acids such as C14:0, C14:1, C16:0, C16:1n9, and C18:0 fatty acids).

An “increased” or “enhanced” amount is typically a “statistically significant” amount, and may include an increase that is 1.1, 1.2, 1.5, 1.7, 2, 2.5, 3, 3.5, 4, 4.5, 5, 6, 7, 8, 9, 10, 15, 20, 30 or more times (e.g., 100, 500, 1000 times) (including all integers and decimal points in between and above 1, e.g., 1.5, 1.6, 1.7. 1.8, etc.) the amount produced by an unmodified microorganism or a differently modified microorganism, typically of the same species. In some embodiments, production or storage of total lipids, total triglycerides, total fatty acids, total free fatty acids, selected fatty acids (e.g., C16:0) total intracellular fatty acids, total secreted fatty acids, and/or total wax esters is increased relative to an unmodified or differently modified microorganism (e.g., for triglycerides, a DGAT-only expressing strain, or a DGAT-expressing strain that does not overexpress an acyl-ACP reductase), as described above, or by at least 10%, at least 15%, at least 20%, at least 25%, at least 30%, at least 40%, at least 50%, at least 60%, at least 70%, at least 80%, at least 90%, at least 100%, at least 150%, at least 200%, at least 300%, at least 400%, at least 500%, or at least 1000%. In certain embodiments, production or storage of total lipids, total triglycerides, total fatty acids, total free fatty acids, total intracellular fatty acids, total secreted fatty acids, and/or total wax esters is increased by 50% to 200%.

Production of lipids such as fatty acids can be measured according to techniques known in the art, such as Nile Red staining, thin layer chromatography and gas chromatography. Production of triglycerides can be measured, for example, using commercially available enzymatic tests, including colorimetric enzymatic tests using glycerol-3-phosphate-oxidase. Production of free fatty acids can be measured in absolute units such as overall accumulation of FAMES (e.g., OD/ml, μg/ml) or in units that reflect the production of FAMES over time, i.e., the rate of FAMES production (e.g., OD/ml/day, μg/ml/day). For example, certain modified microorganisms described herein may produce at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 μg/mL/day; and/or in the range of at least about 20-30, 20-35, 20-40, 20-45, 20-50, 25-30, 25-35, 25-40, 25-45, 25-50, 30-35, 30-40, 30-45, 30-50, 35-40, 35-45, 35-50, 40-45, or 40-50 μg/mL/day. Production of TAGs can be measured similarly.

In certain instances, by “decreased” or “reduced” is meant the ability of one or more modified photosynthetic microorganisms, e.g., Cyanobacteria, to produce or accumulate a lesser amount (e.g., a statistically significant amount) of a given carbon-based product, such as glycogen, as compared to a control photosynthetic microorganism, such as an unmodified Cyanobacteria or a differently modified Cyanobacteria. Production of glycogen and related molecules can be measured according to techniques known in the art, as exemplified herein (see Example 6; and Suzuki et al., Biochimica et Biophysica Acta 1770:763-773, 2007). In certain instances, by “decreased” or “reduced” is meant a lesser level of expression (e.g., a statistically significant amount), by a modified photosynthetic microorganism, e.g., Cyanobacteria, of one or more genes associated with a glycogen biosynthesis or storage pathway, as compared to the level of expression in a control photosynthetic microorganism, such as an unmodified Cyanobacteria or a differently modified Cyanobacteria. In particular embodiments, production or accumulation of a carbon-based product, or expression of one or more genes associated with glycogen biosynthesis or storage is reduced by at least 10%, at least 20%, at least 30%, at least 40%, at least 50%, at least 60%, at least 70%, at least 80%, at least 90%, or 100%. In particular embodiments, production or accumulation of a carbon-based product, or expression of one or more genes associated with glycogen biosynthesis or storage is reduced by 50-100%.

“Stress conditions” refers to any condition that imposes stress upon the Cyanobacteria, including both environmental and physical stresses. Examples of stresses include but not limited to: reduced or increased temperature as compared to standard; nutrient deprivation; reduced or increased light exposure, e.g., intensity or duration, as compared to standard; exposure to reduced or increased nitrogen, iron, sulfur, phosphorus, and/or copper as compared to standard; altered pH, e.g., more or less acidic or basic, as compared to standard; altered salt conditions as compared to standard; exposure to an agent that causes DNA synthesis inhibitor or protein synthesis inhibition; and increased or decreased culture density as compared to standard. Standard growth and culture conditions for various Cyanobacteria are known in the art.

“Reduced nitrogen conditions,” or conditions of “nitrogen limitation,” refer generally to culture conditions in which a certain fraction or percentage of a standard nitrogen concentration is present in the culture media. Such fractions typically include, but are not limited to, about 1/50, 1/40, 1/30, 1/10, ⅕, ¼, or about ½ the standard nitrogen conditions. Such percentages typically include, but are not limited to, less than about 1%, 2%, 3%, 4%, 5%, 6%, 7%, 8%, 9%, 10%, 15%, 20%, 30%, 40%, or 50% the standard nitrogen conditions. “Standard” nitrogen conditions can be estimated, for example, by the amount of nitrogen present in BG11 media, as exemplified herein and known in the art. For instance, BG11 media usually contains nitrogen in the form of NaNO₃ at a concentration of about 1.5 grams/liter (see, e.g., Rippka et al., J. Gen Microbiol. 111:1-61, 1979).

By “obtained from” is meant that a sample such as, for example, a polynucleotide or polypeptide is isolated from, or derived from, a particular source, such as a desired organism or a specific tissue within a desired organism. “Obtained from” can also refer to the situation in which a polynucleotide or polypeptide sequence is isolated from, or derived from, a particular organism or tissue within an organism. For example, a polynucleotide sequence encoding an acyl-ACP reductase, ACP, diacylglycerol acyltransferase, acyl-CoA synthetase, glycogen breakdown protein, aldehyde dehydrogenase, alcohol dehydrogenase, and/or acetyl-CoA carboxylase enzyme, among others described herein, may be isolated from a variety of prokaryotic or eukaryotic organisms, or from particular tissues or cells within certain eukaryotic organism.

The term “operably linked” as used herein means placing a gene under the regulatory control of a promoter, which then controls the transcription and optionally the translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the gene from which it is derived. “Constitutive promoters” are typically active, i.e., promote transcription, under most conditions. “Inducible promoters” are typically active only under certain conditions, such as in the presence of a given molecule factor (e.g., IPTG) or a given environmental condition (e.g., particular CO₂ concentration, nutrient levels, light, heat). In the absence of that condition, inducible promoters typically do not allow significant or measurable levels of transcriptional activity. For example, inducible promoters may be induced according to temperature, pH, a hormone, a metabolite (e.g., lactose, mannitol, an amino acid), light (e.g., wavelength specific), osmotic potential (e.g., salt induced), a heavy metal, or an antibiotic. Numerous standard inducible promoters will be known to one of skill in the art.

The recitation “polynucleotide” or “nucleic acid” as used herein designates mRNA, RNA, cRNA, rRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA and RNA.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide, or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence that encodes an acyl-ACP reductase, ACP, glycogen breakdown protein, aldehyde dehydrogenase, alcohol dehydrogenase, and/or an acetyl-CoA carboxylase enzyme, among others described herein. The terms “polynucleotide variant” and “variant” also include naturally-occurring allelic variants and orthologs that encode these enzymes.

With regard to polynucleotides, the term “exogenous” refers to a polynucleotide sequence that does not naturally-occur in a wild type cell or organism, but is typically introduced into the cell by molecular biological techniques. Examples of exogenous polynucleotides include vectors, plasmids, and/or man-made nucleic acid constructs encoding a desired protein. With regard to polynucleotides, the term “endogenous” or “native” refers to naturally-occurring polynucleotide sequences that may be found in a given wild type cell or organism. For example, certain Cyanobacterial species do not typically contain a DGAT gene, and, therefore, do not comprise an “endogenous” polynucleotide sequence that encodes a DGAT polypeptide. Also, a particular polynucleotide sequence that is isolated from a first organism and transferred to second organism by molecular biological techniques is typically considered an “exogenous” polynucleotide with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The recitations “mutation” or “deletion,” in relation to the genes of a “glycogen biosynthesis or storage pathway,” refer generally to those changes or alterations in a photosynthetic microorganism, e.g., a Cyanobacterium, that render the product of that gene non-functional or having reduced function with respect to the synthesis and/or storage of glycogen. Examples of such changes or alterations include nucleotide substitutions, deletions, or additions to the coding or regulatory sequences of a targeted gene (e.g., glgA, glgC, and pgm), in whole or in part, which disrupt, eliminate, down-regulate, or significantly reduce the expression of the polypeptide encoded by that gene, whether at the level of transcription or translation. Techniques for producing such alterations or changes, such as by recombination with a vector having a selectable marker, are exemplified herein and known in the molecular biological art. In particular embodiments, one or more alleles of a gene, e.g., two or all alleles, may be mutated or deleted within a photosynthetic microorganism. In particular embodiments, modified photosynthetic microorganisms, e.g., Cyanobacteria, of the present invention are merodiploids or partial diploids.

The “deletion” of a targeted gene may also be accomplished by targeting the mRNA of that gene, such as by using various antisense technologies (e.g., antisense oligonucleotides and siRNA) known in the art. Accordingly, targeted genes may be considered “non-functional” when the polypeptide or enzyme encoded by that gene is not expressed by the modified photosynthetic microorganism, or is expressed in negligible amounts, such that the modified photosynthetic microorganism produces or accumulates less glycogen than an unmodified or differently modified photosynthetic microorganism.

In certain aspects, a targeted gene may be rendered “non-functional” by changes or mutations at the nucleotide level that alter the amino acid sequence of the encoded polypeptide, such that a modified polypeptide is expressed, but which has reduced function or activity with respect to glycogen biosynthesis or storage, whether by modifying that polypeptide's active site, its cellular localization, its stability, or other functional features apparent to a person skilled in the art. Such modifications to the coding sequence of a polypeptide involved in glycogen biosynthesis or storage may be accomplished according to known techniques in the art, such as site directed mutagenesis at the genomic level and/or natural selection (i.e., directed evolution) of a given photosynthetic microorganism.

“Polypeptide,” “polypeptide fragment,” “peptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions.

The recitation polypeptide “variant” refers to polypeptides that are distinguished from a reference polypeptide sequence by the addition, deletion or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

The present invention contemplates the use in the methods described herein of variants of full-length enzymes having acyl-ACP reductase activity, ACP activity, glycogen breakdown activity, diacylglyecerol transferase activity (DGAT), fatty acyl-CoA synthetase activity, aldehyde dehydrogenase activity, alcohol dehydrogenase activity, and/or acetyl-CoA carboxylase activity, truncated fragments of these full-length enzymes and polypeptides, variants of truncated fragments, as well as their related biologically active fragments. Typically, biologically active fragments of a polypeptide may participate in an interaction, for example, an intra-molecular or an inter-molecular interaction. An inter-molecular interaction can be a specific binding interaction or an enzymatic interaction (e.g., the interaction can be transient and a covalent bond is formed or broken).

Biologically active fragments of a polypeptide/enzyme having a selected activity include peptides comprising amino acid sequences sufficiently similar to, or derived from, the amino acid sequences of a (putative) full-length reference polypeptide sequence. Typically, biologically active fragments comprise a domain or motif with at least one activity of an acyl-ACP reductase, aldehyde decarbonylase, aldehyde dehydrogenase, alcohol dehydrogenase, ACP polypeptide, DGAT polypeptide, fatty acyl-CoA synthetase polypeptide, acetyl-CoA carboxylase polypeptide, or polypeptide associated with a glycogen breakdown pathway, and may include one or more (and in some cases all) of the various active domains. A biologically active fragment of such polypeptides can be a polypeptide fragment which is, for example, 10, 11, 12, 13, 14, 15, 16, 17, 18, 19, 20, 21, 22, 23, 24, 25, 26, 27, 28, 29, 30, 40, 50, 60, 70, 80, 90, 100, 110, 120, 130, 140, 150, 160, 170, 180, 190, 200, 220, 240, 260, 280, 300, 320, 340, 360, 380, 400, 450, 500, 600 or more contiguous amino acids, including all integers in between, of a reference polypeptide sequence. In certain embodiments, a biologically active fragment comprises a conserved enzymatic sequence, domain, or motif, as described elsewhere herein and known in the art. Suitably, the biologically-active fragment has no less than about 1%, 10%, 25%, 50% of an activity of the wild-type polypeptide from which it is derived.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 97%, 98%, 99% or 100% sequence identity to any of the reference sequences described herein (see, e.g., Sequence Listing), typically where the polypeptide variant maintains at least one biological activity of the reference polypeptide.

Terms used to describe sequence relationships between two or more polynucleotides or polypeptides include “reference sequence”, “comparison window”, “sequence identity”, “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

As used herein, the term “triglyceride” (triacylglycerol or neutral fat) refers to a fatty acid triester of glycerol. Triglycerides are typically non-polar and water-insoluble.

“Phosphoglycerides” (or glycerophospholipids) are major lipid components of biological membranes, and include, for example, any derivative of sn-glycero-3-phosphoric acid that contains at least one O-acyl, or O-alkyl or O-alk-1′-enyl residue attached to the glycerol moiety and a polar head made of a nitrogenous base, a glycerol, or an inositol unit. Phosphoglycerides can also be characterized as amphipathic lipids formed by esters of acylglycerols with phosphate and another hydroxylated compound.

“Transformation” refers to the permanent, heritable alteration in a cell resulting from the uptake and incorporation of foreign DNA into the host-cell genome; also, the transfer of an exogenous gene from one organism into the genome of another organism.

By “vector” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. Such a vector may comprise specific sequences that allow recombination into a particular, desired site of the host chromosome. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably one which is operably functional in a photosynthetic microorganism cell, such as a Cyanobacterial cell. The vector can include a reporter gene, such as a green fluorescent protein (GFP), which can be either fused in frame to one or more of the encoded polypeptides, or expressed separately. The vector can also include a selection marker such as an antibiotic resistance gene that can be used for selection of suitable transformants.

The term “wild-type” refers to a gene or gene product that has the characteristics of that gene or gene product when isolated from a naturally-occurring source. A wild type gene or gene product (e.g., a polypeptide) is that which is most frequently observed in a population and is thus arbitrarily designed the “normal” or “wild-type” form of the gene.

### Modified Photosynthetic Microorganisms

Certain embodiments of the present invention relate to modified photosynthetic microorganisms, including Cyanobacteria, and methods of use thereof, wherein the modified photosynthetic microorganisms comprise one or more over-expressed, exogenous, or introduced polynucleotides encoding an acyl-ACP reductase polypeptide, or a fragment or variant thereof. In particular embodiments, the fragment or variant thereof retains at least 50% of one or more activities of the wild-type acyl-ACP reductase polypeptide. An overexpressed acyl-ACP reductase polypeptide can be encoded by an endogenous or naturally-occurring polynucleotide which is operably linked to an introduced promoter, typically upstream of the microorganism's natural acyl-ACP reductase coding region, and/or it can be encoded by an introduced polynucleotide.

In certain embodiments, the introduced promoter is inducible, and in some embodiments it is constitutive. Included are weak promoters under non-induced conditions. Exemplary promoters are described elsewhere herein and known in the art. In particular embodiments, the introduced promoter is exogenous or foreign to the photosynthetic microorganism, i.e., it is derived from a genus/species that differs from the microorganism being modified. In other embodiments, the introduced promoter is a recombinantly introduced copy of an otherwise endogenous or naturally-occurring promoter sequence, i.e., it is derived from the same species of microorganism being modified.

Similar principles can apply to the introduced polynucleotide which encodes the acyl-ACP reductase or other overexpressed polypeptide (e.g., aldehyde dehydrogenase). For instance, in particular embodiments, the introduced polynucleotide encoding the acyl-ACP reductase or other polypeptide is exogenous or foreign to the photosynthetic microorganism, i.e., it is derived from a genus/species that differs from the microorganism being modified. In other embodiments, the introduced polynucleotide is a recombinantly introduced copy of an otherwise endogenous or naturally-occurring sequence, i.e., it is derived from the same species of microorganism being modified.

Acyl-ACP reductase polypeptides, and fragments and variants thereof, that may be used according to the compositions and methods of the present invention are described in further detail infra. The present invention contemplates the use of naturally-occurring and non-naturally-occurring variants of these acyl-ACP reductase and lipid biosynthesis proteins (e.g., ACP, ACCase, DGAT, acyl-CoA synthetase, aldehyde dehydrogenase), as well as variants of their encoding polynucleotides. These enzyme encoding sequences may be derived from any organism (e.g., plants, bacteria) having a suitable sequence, and may also include any man-made variants thereof, such as any optimized coding sequences (i.e., codon-optimized polynucleotides) or optimized polypeptide sequences.

The modified photosynthetic microorganism described herein preferably produce increased levels of lipids such as free fatty acids relative to unmodified microorganisms of the same genus/species. Acyl-ACP reductase polypeptides may also be optionally overexpressed in strains of photosynthetic microorganisms that have been modified to overexpress selected lipid biosynthesis proteins (e.g., selected fatty acid biosynthesis proteins, triacylglycerol biosynthesis proteins), such as ACP, ACCase, aldehyde dehydrogenases, DGAT when optionally combined with a fatty acyl-CoA synthetase, or any combination thereof, often to further increase production of lipids such as free fatty acids and/or triglycerides, relative to acyl-ACP reductase-only expressing strains, or relative to unmodified strains.

Separately or in combination with the presence of exogenous or overexpressed lipid biosynthesis proteins, acyl-ACP reductase polypeptides may be overexpressed in strains of photosynthetic microorganisms having reduced expression of one or more genes of a glycogen biosynthesis or storage pathway, typically as compared to a wild-type photosynthetic microorganism. In some embodiments, a modified photosynthetic microorganism may comprise one or more overexpressed acyl-ACP reductases in combination with one or more introduced polynucleotides encoding a protein involved in a glycogen breakdown pathway. These latter embodiments can be combined with those strains having reduced expression of glycogen biosynthesis or storage pathways and/or strains having one or more exogenously or overexpressed lipid biosynthesis proteins. For instance, a specific modified photosynthetic microorganism could comprise an overexpressed acyl-ACP reductase, combined with a full or partial deletion of the glgC gene and/or the pgm gene, optionally combined with an overexpressed ACP, ACCase, or both.

Other combinations include, for example, a modified photosynthetic microorganism comprising an overexpressed acyl-ACP reductase in combination with an overexpressed ACP; an acyl-ACP reductase on combination with an ACCase; an acyl-ACP reductase on combination with an ACP and an ACCase; an overexpressed acyl-ACP reductase in combination with an overexpressed DGAT and optionally an overexpressed acyl-CoA synthetase (e.g., a DGAT/acyl-CoA synthetase combination); an overexpressed acyl-ACP reductase with an overexpressed ACP and an overexpressed DGAT, optionally combined with an overexpressed acyl-CoA synthetase; an overexpressed acyl-ACP reductase with an overexpressed ACCase and an overexpressed DGAT, optionally in combination with an overexpressed acyl-CoA synthetase; and an overexpressed acyl-ACP reductase with an overexpressed ACP, ACCase, and an overexpressed DGAT, optionally in combination with an overexpressed acyl-CoA synthetase. Acyl-ACP reductase and DGAT-overexpressing strains, optionally in combination with an overexpressed acyl-CoA synthetase, typically produce increased triglycerides relative to DGAT-only overexpressing strains. Any one of these embodiments can be combined with one or more introduced polynucleotides encoding a protein involved in a glycogen breakdown pathway, and/or with a strain having reduced expression of glycogen biosynthesis or storage pathways (e.g., full or partial deletion of glucose-1-phosphate adenyltransferase (glgC) gene and/or a phosphoglucomutase (pgm) gene). The present invention contemplates the use of any type of polynucleotide encoding a protein or enzyme associated with glycogen breakdown, removal, and/or elimination, as long as the modified photosynthetic microorganism accumulates a reduced amount of glycogen as compared to the wild type photosynthetic microorganism (e.g., under stress conditions).

Any one of these embodiments can also be combined with a strain having reduced expression of an aldehyde decarbonylase. In certain embodiments, such as Cyanobacteria including S. elongatus PCC7942, orf1593 resides directly upstream of orf1594 (acyl-ACP reductase coding region) and encodes an aldehyde decarbonylase. According to one non-limiting theory, because the aldehyde decarbonylase encoded by orf1593 utilizes acyl aldehyde as a substrate for alkane production, reducing expression of this protein may further increase yields of free fatty acids by shunting acyl aldehydes (produced by acyl-ACP reductase) away from an alkane-producing pathway, and towards a fatty acid-producing and storage pathway. PCC7942_orf1593 orthologs can be found, for example, in Synechocystis sp. PCC6803 (encoded by orfsII0208), N. punctiforme PCC 73102, Thermosynechococcus elongatus BP-1, Synechococcus sp. Ja-3-3AB, P. marinus MIT9313, P. marinus NATL2A, and Synechococcus sp. RS 9117, the latter having at least two paralogs (RS 9117-1 and -2). Included are strains having mutations or full or partial deletions of one or more genes encoding these and other aldehyde decarbonylases, such as S. elongatus PCC7942 having a full or partial deletion of orf1593, and Synechocystis sp. PCC6803 having a full or partial deletion of orfsII0208). For instance, a specific modified photosynthetic microorganism could comprise an overexpressed acyl-ACP reductase, combined with a full or partial deletion of the glgC gene and/or the pgm gene, optionally combined with an overexpressed ACP, ACCase, DGAT/acyl-CoA synthetase, or all of the foregoing, and optionally combined with a full or partial deletion of a gene encoding an aldehyde decarbonylase (e.g., PCC7942_orf1593, PCC6803_orfsII0208).

Any one of these embodiments can also be combined with a strain having reduced expression of an acyl-ACP synthetase (Aas). Without wishing to be bound by any one theory, an endogenous aldehyde dehydrogenase is acting on the acyl-aldehydes generated by orf1594 and converting them to free fatty acids. The normal role of such a dehydrogenase might involve removing or otherwise dealing with damaged lipids. In this scenario, it is then likely that the Aas gene product recycles these free fatty acids by ligating them to ACP. Accordingly, reducing or eliminating expression of the Aas gene product might ultimately increase production of fatty acids, by reducing or preventing their transfer to ACP. Included are mutations and full or partial deletions of one or more Aas genes, such as the Aas gene of Synechococcus elongatus PCC 7942. As one example, a specific modified photosynthetic microorganism could comprise an overexpressed acyl-ACP reductase, combined with a full or partial deletion of the glgC gene and/or the pgm gene, optionally combined with an overexpressed ACP, ACCase, DGAT/acyl-CoA synthetase, or all of the foregoing, optionally combined with a full or partial deletion of a gene encoding an aldehyde decarbonylase (e.g., PCC7942_orf1593, PCC6803_orfsII0208), and optionally combined with a full or partial deletion of an Aas gene encoding an acyl-ACP synthetase.

Any one or more of these embodiments can also be combined with a strain having increased expression of an aldehyde dehydrogenase. One exemplary aldehyde dehydrogenase is encoded by orf0489 of Synechococcus elongatus PCC7942. Also included are homologs or paralogs thereof, functional equivalents thereof, and fragments or variants thereofs. Functional equivalents can include aldehyde dehydrogenases with the ability to convert acyl aldehydes (e.g., nonyl-aldehyde) into fatty acids. In specific embodiments, the aldehyde dehydrogenase has the amino acid sequence of SEQ ID NO:103 (encoded by the polynucleotide sequence of SEQ ID NO:102), or an active fragment or variant of this sequence.

Any one or more of these embodiments can also be combined with a strain having increased expression of an alcohol dehydrogenase, such as a long-chain alcohol dehydrogenase, optionally in combination with increased expression of a wax ester synthase (e.g., an enzyme such as aDGAT having wax ester synthase activity). Exemplary alcohol dehydrogenases include slr1192 from Synechycystis sp. PCC6083 and ACIAD3612 from Acinetobacter baylyi (see SEQ ID NOS:104-107). Also included are homologs or paralogs thereof, functional equivalents thereof, and fragments or variants thereofs. Functional equivalents can include alcohol dehydrogenases with the ability to convert acyl aldehydes (e.g., nonyl-aldehyde, C₁₂, C₁₄, C₁₆, C₁₈, C₂₀ fatty aldehydes) into fatty alcohols, which can then be converted into wax esters by the wax ester synthase. In specific embodiments, the alcohol dehydrogenase has the amino acid sequence of SEQ ID NO:105 (slr1192; encoded by the polynucleotide sequence of SEQ ID NO:104), or an active fragment or variant of this sequence. In some embodiments, the alcohol dehydrogenase has the amino acid sequence of SEQ ID NO:107 (ACIAD3612; encoded by the polynucleotide sequence of SEQ ID NO:106), or an active fragment or variant of this sequence. Certain of these and related embodiment can be combined with any one or more of reduced expression of an endogenous aldehyde dehydrogenase (e.g., orf0489 deletion), reduced expression of an aldehyde decarbonylase (e.g., orf1593 deletion), or an overexpressed acyl carrier protein (ACP), optionally in combination with an overexpressed acyl-ACP synthetase (Aas).

Increased expression can be achieved a variety of ways, for example, by introducing a polynucleotide into the microorganism, modifying an endogenous gene to overexpress the polypeptide, or both. For instance, one or more copies of an otherwise endogenous polynucleotide sequence can be introduced by recombinant techniques to increase expression.

Modified photosynthetic microorganisms of the present invention may be produced using any type of photosynthetic microorganism. These include, but are not limited to photosynthetic bacteria, green algae, and cyanobacteria. The photosynthetic microorganism can be, for example, a naturally photosynthetic microorganism, such as a Cyanobacterium, or an engineered photosynthetic microorganism, such as an artificially photosynthetic bacterium. Exemplary microorganisms that are either naturally photosynthetic or can be engineered to be photosynthetic include, but are not limited to, bacteria; fungi; archaea; protists; eukaryotes, such as a green algae; and animals such as plankton, planarian, and amoeba. Examples of naturally occurring photosynthetic microorganisms include, but are not limited to, Spirulina maximum, Spirulina platensis, Dunaliella salina, Botrycoccus braunii, Chlorella vulgaris, Chlorella pyrenoidosa, Serenastrum capricomutum, Scenedesmus auadricauda, Porphyridium cruentum, Scenedesmus acutus, Dunaliella sp., Scenedesmus obliquus, Anabaenopsis, Aulosira, Cylindrospermum, Synechococcus sp., Synechocystis sp., and/or Tolypothrix.

A modified Cyanobacteria of the present invention may be from any genera or species of Cyanobacteria that is genetically manipulable, i.e., permissible to the introduction and expression of exogenous genetic material. Examples of Cyanobacteria that can be engineered according to the methods of the present invention include, but are not limited to, the genus Synechocystis, Synechococcus, Thermosynechococcus, Nostoc, Prochlorococcu, Microcystis, Anabaena, Spirulina, and Gloeobacter.

Cyanobacteria, also known as blue-green algae, blue-green bacteria, or Cyanophyta, is a phylum of bacteria that obtain their energy through photosynthesis. Cyanobacteria can produce metabolites, such as carbohydrates, proteins, lipids and nucleic acids, from CO₂, water, inorganic salts and light. Any Cyanobacteria may be used according to the present invention.

Cyanobacteria include both unicellular and colonial species. Colonies may form filaments, sheets or even hollow balls. Some filamentous colonies show the ability to differentiate into several different cell types, such as vegetative cells, the normal, photosynthetic cells that are formed under favorable growing conditions; akinetes, the climate-resistant spores that may form when environmental conditions become harsh; and thick-walled heterocysts, which contain the enzyme nitrogenase, vital for nitrogen fixation.

Heterocysts may also form under the appropriate environmental conditions (e.g., anoxic) whenever nitrogen is necessary. Heterocyst-forming species are specialized for nitrogen fixation and are able to fix nitrogen gas, which cannot be used by plants, into ammonia (NH₃), nitrites (NO₂⁻), or nitrates (NO₃⁻), which can be absorbed by plants and converted to protein and nucleic acids.

Many Cyanobacteria also form motile filaments, called hormogonia, which travel away from the main biomass to bud and form new colonies elsewhere. The cells in a hormogonium are often thinner than in the vegetative state, and the cells on either end of the motile chain may be tapered. In order to break away from the parent colony, a hormogonium often must tear apart a weaker cell in a filament, called a necridium.

Each individual Cyanobacterial cell typically has a thick, gelatinous cell wall. Cyanobacteria differ from other gram-negative bacteria in that the quorum sensing molecules autoinducer-2 and acyl-homoserine lactones are absent. They lack flagella, but hormogonia and some unicellular species may move about by gliding along surfaces. In water columns, some Cyanobacteria float by forming gas vesicles, like in archaea.

Cyanobacteria have an elaborate and highly organized system of internal membranes that function in photosynthesis. Photosynthesis in Cyanobacteria generally uses water as an electron donor and produces oxygen as a by-product, though some Cyanobacteria may also use hydrogen sulfide, similar to other photosynthetic bacteria. Carbon dioxide is reduced to form carbohydrates via the Calvin cycle. In most forms, the photosynthetic machinery is embedded into folds of the cell membrane, called thylakoids. Due to their ability to fix nitrogen in aerobic conditions, Cyanobacteria are often found as symbionts with a number of other groups of organisms such as fungi (e.g., lichens), corals, pteridophytes (e.g., Azolla), and angiosperms (e.g., Gunnera), among others.

Cyanobacteria are the only group of organisms that are able to reduce nitrogen and carbon in aerobic conditions. The water-oxidizing photosynthesis is accomplished by coupling the activity of photosystem (PS) II and I (Z-scheme). In anaerobic conditions, Cyanobacteria are also able to use only PS I (i.e., cyclic photophosphorylation) with electron donors other than water (e.g., hydrogen sulfide, thiosulphate, or molecular hydrogen), similar to purple photosynthetic bacteria. Furthermore, Cyanobacteria share an archaeal property; the ability to reduce elemental sulfur by anaerobic respiration in the dark. The Cyanobacterial photosynthetic electron transport system shares the same compartment as the components of respiratory electron transport. Typically, the plasma membrane contains only components of the respiratory chain, while the thylakoid membrane hosts both respiratory and photosynthetic electron transport.

Phycobilisomes, attached to the thylakoid membrane, act as light harvesting antennae for the photosystems of Cyanobacteria. The phycobilisome components (phycobiliproteins) are responsible for the blue-green pigmentation of most Cyanobacteria. Color variations are mainly due to carotenoids and phycoerythrins, which may provide the cells with a red-brownish coloration. In some Cyanobacteria, the color of light influences the composition of phycobilisomes. In green light, the cells accumulate more phycoerythrin, whereas in red light they produce more phycocyanin. Thus, the bacteria appear green in red light and red in green light. This process is known as complementary chromatic adaptation and represents a way for the cells to maximize the use of available light for photosynthesis.

In particular embodiments, the Cyanobacteria may be, e.g., a marine form of Cyanobacteria or a fresh water form of Cyanobacteria. Examples of marine forms of Cyanobacteria include, but are not limited to Synechococcus WH8102, Synechococcus RCC307, Synechococcus NKBG 15041c, and Trichodesmium. Examples of fresh water forms of Cyanobacteria include, but are not limited to, S. elongatus PCC7942, Synechocystis PCC6803, Plectonema boryanum, and Anabaena sp. Exogenous genetic material encoding the desired enzymes or polypeptides may be introduced either transiently, such as in certain self-replicating vectors, or stably, such as by integration (e.g., recombination) into the Cyanobacterium's native genome.

In other embodiments, a genetically modified Cyanobacteria of the present invention may be capable of growing in brackish or salt water. When using a fresh water form of Cyanobacteria, the overall net cost for production of triglycerides will depend on both the nutrients required to grow the culture and the price for freshwater. One can foresee freshwater being a limited resource in the future, and in that case it would be more cost effective to find an alternative to freshwater. Two such alternatives include: (1) the use of waste water from treatment plants; and (2) the use of salt or brackish water.

Salt water in the oceans can range in salinity between 3.1% and 3.8%, the average being 3.5%, and this is mostly, but not entirely, made up of sodium chloride (NaCl) ions. Brackish water, on the other hand, has more salinity than freshwater, but not as much as seawater. Brackish water contains between 0.5% and 3% salinity, and thus includes a large range of salinity regimes and is therefore not precisely defined. Waste water is any water that has undergone human influence. It consists of liquid waste released from domestic and commercial properties, industry, and/or agriculture and can encompass a wide range of possible contaminants at varying concentrations.

There is a broad distribution of Cyanobacteria in the oceans, with Synechococcus filling just one niche. Specifically, Synechococcus sp. PCC 7002 (formerly known as Agmenellum quadruplicatum strain PR-6) grows in brackish water, is unicellular and has an optimal growing temperature of 38° C. While this strain is well suited to grow in conditions of high salt, it will grow slowly in freshwater. In particular embodiments, the present invention contemplates the use of a Cyanobacteria S. elongatus PCC7942, altered in a way that allows for growth in either waste water or salt/brackish water. A S. elongatus PCC7942 mutant resistant to sodium chloride stress has been described (Bagchi, S. N. et al., Photosynth Res. 2007, 92:87-101), and a genetically modified S. elongatus PCC7942 tolerant of growth in salt water has been described (Waditee, R. et al., PNAS 2002, 99:4109-4114). According to the present invention, a salt water tolerant strain is capable of growing in water or media having a salinity in the range of 0.5% to 4.0% salinity, although it is not necessarily capable of growing in all salinities encompassed by this range. In one embodiment, a salt tolerant strain is capable of growth in water or media having a salinity in the range of 1.0% to 2.0% salinity. In another embodiment, a salt water tolerant strain is capable of growth in water or media having a salinity in the range of 2.0% to 3.0% salinity.

Examples of Cyanobacteria that may be utilized and/or genetically modified according to the methods described herein include, but are not limited to, Chroococcales Cyanobacteria from the genera Aphanocapsa, Aphanothece, Chamaesiphon, Chroococcus, Chroogloeocystis, Coelosphaerium, Crocosphaera, Cyanobacterium, Cyanobium, Cyanodictyon, Cyanosarcina, Cyanothece, Dactylococcopsis, Gloecapsa, Gloeothece, Merismopedia, Microcystis, Radiocystis, Rhabdoderma, Snowella, Synychococcus, Synechocystis, Thermosenechococcus, and Woronichinia; Nostacales Cyanobacteria from the genera Anabaena, Anabaenopsis, Aphanizomenon, Aulosira, Calothrix, Coleodesmium, Cyanospira, Cylindrospermosis, Cylindrospermum, Fremyella, Gleotrichia, Microchaete, Nodularia, Nostoc, Rexia, Richelia, Scytonema, Sprirestis, and Toypothrix; Oscillatoriales Cyanobacteria from the genera Arthrospira, Geitlerinema, Halomicronema, Halospirulina, Katagnymene, Leptolyngbya, Limnothrix, Lyngbya, Microcoleus, Oscillatoria, Phormidium, Planktothricoides, Planktothrix, Plectonema, Pseudoanabaena/Limnothrix, Schizothrix, Spirulina, Symploca, Trichodesmium, Tychonema; Pleurocapsales cyanobacterium from the genera Chroococcidiopsis, Dermocarpa, Dermocarpella, Myxosarcina, Pleurocapsa, Stanieria, Xenococcus; Prochlorophytes Cyanobacterium from the genera Prochloron, Prochlorococcus, Prochlorothrix; and Stigonematales cyanobacterium from the genera Capsosira, Chlorogeoepsis, Fischerella, Hapalosiphon, Mastigocladopsis, Nostochopsis, Stigonema, Symphyonema, Symphonemopsis, Umezakia, and Westiellopsis. In certain embodiments, the Cyanobacterium is from the genus Synechococcus, including, but not limited to Synechococcus bigranulatus, Synechococcus elongatus, Synechococcus leopoliensis, Synechococcus lividus, Synechococcus nidulans, and Synechococcus rubescens.

In certain embodiments, the Cyanobacterium is Anabaena sp. strain PCC 7120, Synechocystis sp. strain PCC6803, Nostoc muscorum, Nostoc ellipsosporum, or Nostoc sp. strain PCC 7120. In certain preferred embodiments, the Cyanobacterium is S. elongatus sp. strain PCC7942.

Additional examples of Cyanobacteria that may be utilized in the methods provided herein include, but are not limited to, Synechococcus sp. strains WH7803, WH8102, WH8103 (typically genetically modified by conjugation), Baeocyte-forming Chroococcidiopsis spp. (typically modified by conjugation/electroporation), non-heterocyst-forming filamentous strains Planktothrix sp., Plectonema boryanum M101 (typically modified by electroporation), and Heterocyst-forming strains Anabaena sp. strains ATCC 29413 (typically modified by conjugation), Tolypothrix sp. strain PCC 7601 (typically modified by conjugation/electroporation) and Nostoc punctiforme strain ATCC 29133 (typically modified by conjugation/electroporation).

In certain preferred embodiments, the Cyanobacterium may be S. elongatus sp. strain PCC7942 or Synechococcus sp. PCC 7002 (originally known as Agmenellum quadruplicatum).

In particular embodiments, the genetically modified, photosynthetic microorganism, e.g., Cyanobacteria, of the present invention may be used to produce triglycerides and/or other carbon-based products from just sunlight, water, air, and minimal nutrients, using routine culture techniques of any reasonably desired scale. In particular embodiments, the present invention contemplates using spontaneous mutants of photosynthetic microorganisms that demonstrate a growth advantage under a defined growth condition. Among other benefits, the ability to produce large amounts of triglycerides from minimal energy and nutrient input makes the modified photosynthetic microorganism, e.g., Cyanobacteria, of the present invention a readily manageable and efficient source of feedstock in the subsequent production of both biofuels, such as biodiesel, as well as specialty chemicals, such as glycerin.

### Methods of Producing Modified Photosynthetic Microorganisms

Embodiments of the present invention also include methods of producing the modified photosynthetic microorganisms (e.g., Cyanobacterium) described herein.

In one embodiments, the present invention comprises a method of modifying a photosynthetic microorganism to produce a modified photosynthetic microorganism that produces an increased amount of lipids, e.g., free fatty acids, as compared to a corresponding unmodified or wild-type photosynthetic microorganism, comprising introducing into said microorganism one or more operatively linked promoters or other regulatory elements surrounding a region encoding a naturally-occurring or endogenous acyl-ACP reductase. In other embodiments, the present invention comprises a method of modifying a photosynthetic microorganism to produce a modified photosynthetic microorganism that produces an increased amount of lipids, e.g., free fatty acids, as compared to a corresponding wild type photosynthetic microorganism, comprising introducing into said microorganism one or more polynucleotides encoding an acyl-ACP reductase, including active fragments or variants thereof. The method may further comprise a step of selecting for photosynthetic microorganisms in which the one or more desired promoters or polynucleotides were successfully introduced, where the polynucleotides were, e.g., present in a vector the expressed a selectable marker, such as an antibiotic resistance gene. As one example, selection and isolation may include the use of antibiotic resistant markers known in the art (e.g., kanamycin, spectinomycin, and streptomycin).

In certain embodiments, methods of the present invention comprise both: (1) introducing into said photosynthetic microorganism one or more operatively linked promoters (e.g., inducible or regulable promoters) into a region upstream of an endogenous acyl-ACP reductase coding sequence, and/or introducing one or more polynucleotides encoding an acyl-ACP reductase, or a fragment or variant thereof; and (2) introducing into said photosynthetic microorganism one or more polynucleotides encoding one or more lipid biosynthesis proteins, e.g., enzymes associated with fatty acid biosynthesis. In certain embodiments, the one or more enzymes associated with fatty acid biosynthesis have an acyl carrier protein (ACP) activity, an ACCase activity, or any combination thereof.

Thus, in one particular embodiment, the present invention includes a method of producing a modified photosynthetic microorganism, e.g., a Cyanobacteria, comprising: (1) introducing into said photosynthetic microorganism one or more operatively linked promoters (e.g., inducible or otherwise regulable promoters), enhancers, or other regulatory elements into a region surrounding an endogenous acyl-ACP reductase coding sequence, and/or introducing one or more polynucleotides encoding an acyl-ACP reductase, or a fragment or variant thereof; and (2) introducing into said photosynthetic microorganism one or more polynucleotides encoding an ACP, or a fragment or variant thereof. In one particular embodiment, the present invention includes a method of producing a modified photosynthetic microorganism, e.g., a Cyanobacteria, comprising: (1) introducing into said photosynthetic microorganism one or more operatively linked promoters (e.g., inducible or regulable promoters), enhancers, or other regulatory elements into a region surrounding an endogenous acyl-ACP reductase coding sequence, and/or introducing one or more polynucleotides encoding an acyl-ACP reductase, or a fragment or variant thereof; and (2) introducing into said photosynthetic microorganism one or more polynucleotides encoding an ACCase, or a fragment or variant thereof.

In one particular embodiment, the present invention includes a method of producing a modified photosynthetic microorganism, e.g., a Cyanobacteria, comprising: (1) introducing into said photosynthetic microorganism one or more operatively linked promoters (e.g., inducible or regulable promoters), enhancers, or other regulatory elements into a region surrounding an endogenous acyl-ACP reductase coding sequence, and/or introducing one or more polynucleotides encoding an acyl-ACP reductase, or a fragment or variant thereof; and (2) introducing into said photosynthetic microorganism one or more polynucleotides encoding a DGAT optionally combined with one or more polynucleotides encoding a fatty acyl-CoA synthetase, or a fragment or variant thereof. Specific embodiments further include 3) introducing into said photosynthetic microorganism one or more polynucleotides encoding an ACP and/or an ACCase. Because of the presence of a DGAT and an optionally fatty acyl-CoA synthetase (the fatty acyl-CoA synthetase converts the free fatty acids to acyl-CoA, which is a substrate for DGAT), these and related embodiments typically produce increased levels of fatty acids and/or triglycerides.

In certain embodiments, methods of the present invention comprise both: (1) introducing into said photosynthetic microorganism one or more operatively linked promoters (e.g., inducible or regulable promoters), enhancers, or other regulatory elements into a region surrounding an endogenous acyl-ACP reductase coding sequence, and/or introducing one or more polynucleotides encoding an acyl-ACP reductase, or a fragment or variant thereof; and (2) modifying the photosynthetic microorganism so that it expresses a reduced amount of one or more genes associated with a glycogen biosynthesis or storage pathway and/or an increased amount of one or more polynucleotides encoding a polypeptide associated with a glycogen breakdown pathway. Thus, in one particular embodiment, the present invention includes a method of producing a modified photosynthetic microorganism, e.g., a Cyanobacteria, comprising: (1) introducing into said photosynthetic microorganism one or more operatively linked promoters (e.g., inducible or regulable promoters), enhancers, or other regulatory elements into a region surrounding an endogenous acyl-ACP reductase coding sequence, and/or introducing one or more polynucleotides encoding an acyl-ACP reductase, or a fragment or variant thereof; and (2) modifying the photosynthetic microorganism so that it has a reduced level of expression of one or more genes of a glycogen biosynthesis or storage pathway. In particular embodiments, expression or activity is reduced by mutating or deleting a portion or all of said one or more genes. In particular embodiments, expression or activity is reduced by knocking out or knocking down one or more alleles of said one or more genes. In particular embodiments, expression or activity of the one or more genes is reduced by contacting the photosynthetic microorganism with an antisense oligonucleotide or interfering RNA, e.g., an siRNA, that targets said one or more genes. In particular embodiments, a vector that expresses a polynucleotide that hybridizes to said one or more genes, e.g., an antisense oligonucleotide or an siRNA is introduced into said photosynthetic microorganism.

In certain embodiments, methods of the present invention comprise both: (1) introducing into said photosynthetic microorganism one or more operatively linked promoters (e.g., inducible or regulable promoters), enhancers, or other regulatory elements into a region surrounding an endogenous acyl-ACP reductase coding sequence, and/or introducing one or more polynucleotides encoding an acyl-ACP reductase, or a fragment or variant thereof; (2) introducing into said photosynthetic microorganism one or more polynucleotides encoding one or more lipid biosynthesis proteins (e.g., enzymes associated with fatty acid and/or triglyceride biosynthesis); and (3) modifying the photosynthetic microorganism so that it expresses a reduced amount of one or more genes associated with a glycogen biosynthesis or storage pathway and/or an increased amount of one or more polynucleotides encoding a polypeptide associated with a glycogen breakdown pathway.

In certain embodiments, methods of the present invention comprise both: (1) introducing into said photosynthetic microorganism one or more operatively linked promoters (e.g., inducible or regulable promoters), enhancers, or other regulatory elements into a region surrounding an endogenous acyl-ACP reductase coding sequence, and/or introducing one or more polynucleotides encoding an acyl-ACP reductase, or a fragment or variant thereof; and (2) modifying the photosynthetic microorganism so that it has reduced levels of expression of one or more genes encoding an aldehyde decarbonylase, such as orf1593 in certain Cyanobacteria (e.g., S. elongatus PCC7942), and/or modifying the microorganism so that it has reduced levels of expression of one or more genes encoding an acyl-ACP synthetase (Aas). Because the aldehyde decarbonylase encoded by genes such as orf1593 converts acyl aldehydes to alkanes (see FIG. 2), potentially shunting said acyl aldehydes away from fatty acid related pathways, its reduction or deletion may increase the availability of acyl aldehydes and thereby further increase production of free fatty acids. Also, because Aas may recycle excess free fatty acids by ligating them to ACP, its reduction or deletion may increase levels of fatty acids. These and related embodiments can be combined with any of the other methods described herein.

In certain embodiments, methods of the present invention comprise: (1) introducing into said photosynthetic microorganism one or more operatively linked promoters (e.g., inducible or regulable promoters), enhancers, or other regulatory elements into a region surrounding an endogenous acyl-ACP reductase coding sequence, and/or introducing one or more polynucleotides encoding an acyl-ACP reductase, or a fragment or variant thereof; (2) introducing into said photosynthetic microorganism one or more operatively linked promoters (e.g., inducible or regulable promoters), enhancers, or other regulatory elements into a region surrounding an endogenous (long-chain) alcohol dehydrogenase coding sequence, and/or introducing one or more polynucleotides encoding an alcohol dehydrogenase, or a fragment or variant thereof; and (3) or introducing one or more polynucleotides encoding a wax ester synthase, such as DGAT (e.g., aDGAT) having wax ester synthase activity, or a fragment or variant thereof. The acyl-ACP reductase increases production of fatty aldehydes, the alcohol dehydrogenase converts the fatty aldehydes into fatty alcohols, and the wax ester synthase then converts the fatty alcohols into wax esters.

Certain of these and related embodiments further comprise: (4) modifying the photosynthetic microorganism so that it has reduced levels of expression of one or more genes encoding an aldehyde decarbonylase, such as orf1593 in certain Cyanobacteria (e.g., S. elongatus PCC7942), and/or (5) modifying the microorganism so that it has reduced levels of expression of one or more genes encoding an aldehyde dehydrogenase, such as orf0489 in certain Cyanobacteria. Because the aldehyde decarbonylase encoded by genes such as orf1593, and the aldehyde dehydrogenase encoded by genes such as orf0489, respectively convert acyl aldehydes to alkanes and free fatty acids (see FIG. 2), potentially shunting said acyl aldehydes away from fatty alcohol and wax ester related pathways, their reduction or deletion, either alone or in combination, may increase the availability of acyl aldehydes and thereby further increase production of wax esters.

Alternatively to or in combination with (4) and/or (5), certain of these and related embodiments may further comprise (6) introducing into said photosynthetic microorganism one or more operatively linked promoters (e.g., inducible or regulable promoters), enhancers, or other regulatory elements into a region surrounding an endogenous ACP coding sequence, and/or introducing one or more polynucleotides encoding an ACP, or a fragment or variant thereof; and/or (7) introducing into said photosynthetic microorganism one or more operatively linked promoters (e.g., inducible or regulable promoters), enhancers, or other regulatory elements into a region surrounding an endogenous Aas coding sequence, and/or introducing one or more polynucleotides encoding an Aas polypeptide, or a fragment or variant thereof.

Photosynthetic microorganisms, e.g., Cyanobacteria, may be genetically modified according to techniques known in the art, e.g., to delete a portion or all of a gene or to introduce a polynucleotide that expresses a functional polypeptide. As noted above, in certain aspects, genetic manipulation in photosynthetic microorganisms, e.g., Cyanobacteria, can be performed by the introduction of non-replicating vectors which contain native photosynthetic microorganism sequences, exogenous genes of interest, and selectable markers or drug resistance genes. Upon introduction into the photosynthetic microorganism, the vectors may be integrated into the photosynthetic microorganism's genome through homologous recombination. In this way, an exogenous gene of interest and the drug resistance gene are stably integrated into the photosynthetic microorganism's genome. Such recombinants cells can then be isolated from non-recombinant cells by drug selection. Cell transformation methods and selectable markers for Cyanobacteria are also well known in the art (see, e.g., Wirth, Mol Gen Genet 216:175-7, 1989; and Koksharova, Appl Microbiol Biotechnol 58:123-37, 2002; and THE CYANOBACTERIA: MOLECULAR BIOLOGY, GENETICS, AND EVOLUTION (eds. Antonio Herrera and Enrique Flores) Caister Academic Press, 2008, each of which is incorporated by reference for their description on gene transfer into Cyanobacteria, and other information on Cyanobacteria).

In certain embodiments, an endogenous version of a protein (e.g., acyl-ACP reductase, ACP, glycogen breakdown protein, DGAT, acyl-CoA synthetase, ACCase, aldehyde dehydrogenase, alcohol dehydrogenase), if naturally present in the modified photosynthetic microorganism, can be overexpressed by introducing one or more operatively linked regulatory element(s) in a region surrounding the endogenous gene encoding that protein, i.e., the naturally-occurring version of that gene. Regulatory elements can be stably and operatively introduced upstream and/or downstream of the genomic region of the endogenous gene. Examples of regulatory elements include promoters, enhancers, repressors, ribosome binding sites, and transcription termination sites. Such promoters or regulatory elements may be constitutive or inducible. Such promoters or regulatory elements may be derived from the same or a different genus/species relative to the microorganism being modified. In specific embodiments, all of the one or more regulatory elements are derived from the same species of microorganism that is being modified.

In certain embodiments, an exogenous (or introduced) version of a protein (e.g., acyl-ACP reductase, ACP, glycogen breakdown protein, DGAT, acyl-CoA synthetase, ACCase, aldehyde dehydrogenase, alcohol dehydrogenase) is introduced into the photosynthetic microorganism using recombinant techniques known in the art and described elsewhere herein. For instance, introduced polynucleotides encoding a desired polypeptide may be operably linked to one or more regulatory elements (e.g., promoters, enhancers, repressors, ribosome binding sites, transcription termination sites) as part of an expression construct or vector. Included are self-replicating or episomal vectors, in addition to integrative vectors, which integrate into the genome of the modified microorganism. The sequence of the introduced polynucleotide can be derived from or the same as the sequence of an otherwise endogenous/naturally-occurring sequence.

Generation of deletions or mutations of any of the one or more genes associated with the biosynthesis or storage of glycogen can be accomplished according to a variety of methods known in the art, including the use of a non-replicating, selectable vector system that is targeted to the upstream and downstream flanking regions of a given gene (e.g., glgC, pgm, orf1593, Aas), and which recombines with the Cyanobacterial genome at those flanking regions to replace the endogenous coding sequence with the vector sequence. Given the presence of a selectable marker in the vector sequence, such as a drug selectable marker, Cyanobacterial cells containing the gene deletion can be readily isolated, identified and characterized. Such selectable vector-based recombination methods need not be limited to targeting upstream and downstream flanking regions, but may also be targeted to internal sequences within a given gene, as long as that gene is rendered “non-functional,” as described herein.

The generation of deletions or mutations can also be accomplished using antisense-based technology. For instance, Cyanobacteria have been shown to contain natural regulatory events that rely on antisense regulation, such as a 177-nt ncRNA that is transcribed in antisense to the central portion of an iron-regulated transcript and blocks its accumulation through extensive base pairing (see, e.g., Dühring, et al., Proc. Natl. Acad. Sci. USA 103:7054-7058, 2006), as well as a alr1690 mRNA that overlaps with, and is complementary to, the complete furA gene, which acts as an antisense RNA (α-furA RNA) interfering with furA transcript translation (see, e.g., Hernandez et al., Journal of Molecular Biology 355:325-334, 2006). Thus, the incorporation of antisense molecules targeted to genes involved in glycogen biosynthesis or storage would be similarly expected to negatively regulate the expression of these genes, rendering them “non-functional,” as described herein.

As used herein, antisense molecules encompass both single and double-stranded polynucleotides comprising a strand having a sequence that is complementary to a target coding strand of a gene or mRNA. Thus, antisense molecules include both single-stranded antisense oligonucleotides and double-stranded siRNA molecules.

Photosynthetic microorganisms may be cultured according to techniques known in the art. For example, Cyanobacteria may be cultured or cultivated according to techniques known in the art, such as those described in Acreman et al. (Journal of Industrial Microbiology and Biotechnology 13:193-194, 1994), in addition to photobioreactor based techniques, such as those described in Nedbal et al. (Biotechnol Bioeng. 100:902-10, 2008). One example of typical laboratory culture conditions for Cyanobacterium is growth in BG-11 medium (ATCC Medium 616) at 30° C. in a vented culture flask with constant agitation and constant illumination at 30-100 μmole photons m⁻² sec⁻¹.

A wide variety of mediums are available for culturing Cyanobacteria, including, for example, Aiba and Ogawa (AO) Medium, Allen and Arnon Medium plus Nitrate (ATCC Medium 1142), Antia's (ANT) Medium, Aquil Medium, Ashbey's Nitrogen-free Agar, ASN-III Medium, ASP 2 Medium, ASW Medium (Artificial Seawater and derivatives), ATCC Medium 617 (BG-11 for Marine Blue-Green Algae; Modified ATCC Medium 616 [BG-11 medium]), ATCC Medium 819 (Blue-green Nitrogen-fixing Medium; ATCC Medium 616 [BG-11 medium] without NO₃), ATCC Medium 854 (ATCC Medium 616 [BG-11 medium] with Vitamin B₁₂), ATCC Medium 1047 (ATCC Medium 957 [MN marine medium] with Vitamin B₁₂), ATCC Medium 1077 (Nitrogen-fixing marine medium; ATCC Medium 957 [MN marine medium] without NO₃), ATCC Medium 1234 (BG-11 Uracil medium; ATCC Medium 616 [BG-11 medium] with uracil), Beggiatoa Medium (ATCC Medium 138), Beggiatoa Medium 2 (ATCC Medium 1193), BG-11 Medium for Blue Green Algae (ATCC Medium 616), Blue-Green (BG) Medium, Bold's Basal (BB) Medium, Castenholtz D Medium, Castenholtz D Medium Modified (Halophilic cyanobacteria), Castenholtz DG Medium, Castenholtz DGN Medium, Castenholtz ND Medium, Chloroflexus Broth, Chloroflexus Medium (ATCC Medium 920), Chu's #10 Medium (ATCC Medium 341), Chu's #10 Medium Modified, Chu's #11 Medium Modified, DCM Medium, DYIV Medium, E27 Medium, E31 Medium and Derivatives, f/2 Medium, f/2 Medium Derivatives, Fraquil Medium (Freshwater Trace Metal-Buffered Medium), Gorham's Medium for Algae (ATCC Medium 625), h/2 Medium, Jaworski's (JM) Medium, K Medium, L1 Medium and Derivatives, MN Marine Medium (ATCC Medium 957), Plymouth Erdschreiber (PE) Medium, Prochlorococcus PC Medium, Proteose Peptone (PP) Medium, Prov Medium, Prov Medium Derivatives, S77 plus Vitamins Medium, S88 plus Vitamins Medium, Saltwater Nutrient Agar (SNA) Medium and Derivatives, SES Medium, SN Medium, Modified SN Medium, SNAX Medium, Soil/Water Biphasic (S/W) Medium and Derivatives, SOT Medium for Spirulina: ATCC Medium 1679, Spirulina (SP) Medium, van Rijn and Cohen (RC) Medium, Walsby's Medium, Yopp Medium, and Z8 Medium, among others.

### Methods of Producing Lipids

The modified photosynthetic microorganisms of the present invention may be used to produce lipids, such as fatty acids, triglycerides, and/or wax esters. Accordingly, the present invention provides methods of producing lipids such as fatty acids comprising culturing any of the modified photosynthetic microorganisms of the present invention (described elsewhere herein) under conditions wherein the modified photosynthetic microorganism produces and/or accumulates (e.g., stores, secretes) an increased amount of cellular lipid as compared to a corresponding wild-type or unmodified photosynthetic microorganism.

In one embodiment, the modified photosynthetic microorganism is a Cyanobacterium that produces or accumulates increased fatty acids relative to an unmodified or wild-type Cyanobacterium of the same species. In specific embodiments, the modified photosynthetic microorganism such as Cyanobacteria produces increased levels of particular fatty acids, such as C16:0 fatty acids. In certain embodiments, the modified photosynthetic microorganism is a Cyanobacterium that produces or accumulates increased wax esters relative to an unmodified or wild-type Cyanobacterium of the same species

In certain embodiments, a naturally-occurring or endogenous gene (e.g., encoding an acyl-ACP reductase, ACP) is overexpressed by introducing an operatively linked heterologous promoter or other regulatory element surrounding its coding region. In particular embodiments, the promoter is an inducible promoter. In some embodiments, the introduced promoter is a weak promoter under non-induced conditions.

In certain embodiments, the one or more introduced polynucleotides are present in one or more expression constructs. In particular embodiments, the one or more expression constructs comprises one or more inducible promoters. In certain embodiments, the one or more expression constructs are stably integrated into the genome of said modified photosynthetic microorganism. In certain embodiments, the introduced polynucleotide encoding an introduced protein is present in an expression construct comprising a weak promoter under non-induced conditions. In certain embodiments, one or more of the introduced polynucleotides are codon-optimized for expression in a Cyanobacterium, e.g., a Synechococcus elongatus.

In particular embodiments, the photosynthetic microorganism is a Synechococcus elongatus, such as Synechococcus elongatus strain PCC7942 or a salt tolerant variant of Synechococcus elongatus strain PCC7942.

In particular embodiments, the photosynthetic microorganism is a Synechococcus sp. PCC 7002 or a Synechocystis sp. PCC6803.

In particular embodiments, the modified photosynthetic microorganisms are cultured under conditions suitable for inducing expression of the introduced polynucleotide(s), e.g., wherein the introduced polynucleotide(s) comprise an inducible promoter. Conditions and reagents suitable for inducing inducible promoters are known and available in the art. Also included are the use of auto-inductive systems, for example, where a metabolite represses expression of the introduced polynucleotide, and the use of that metabolite by the microorganism over time decreases its concentration and thus its repressive activities, thereby allowing increased expression of the polynucleotide sequence.

In certain embodiments, modified photosynthetic microorganisms, e.g., Cyanobacteria, are grown under conditions favorable for producing lipids, triglycerides and/or fatty acids. In particular embodiments, light intensity is between 100 and 2000 uE/m2/s, or between 200 and 1000 uE/m2/s. In particular embodiments, the pH range of culture media is between 7.0 and 10.0. In certain embodiments, CO₂ is injected into the culture apparatus to a level in the range of 1% to 10%. In particular embodiments, the range of CO₂ is between 2.5% and 5%. In certain embodiments, nutrient supplementation is performed during the linear phase of growth. Each of these conditions may be desirable for triglyceride production.

In certain embodiments, the modified photosynthetic microorganisms are cultured, at least for some time, under static growth conditions as opposed to shaking conditions. For example, the modified photosynthetic microorganisms may be cultured under static conditions prior to inducing expression of an introduced polynucleotide (e.g., acyl-ACP reductase, ACP, glycogen breakdown protein, ACCase, DGAT, fatty acyl-CoA synthetase, aldehyde dehydrogenase, alcohol dehydrogenase) and/or the modified photosynthetic microorganism may be cultured under static conditions while expression of an introduced polynucleotide is being induced, or during a portion of the time period during which expression on an introduced polynucleotide is being induced. Static growth conditions may be defined, for example, as growth without shaking or growth wherein the cells are shaken at less than or equal to 30 rpm or less than or equal to 50 rpm.

In certain embodiments, the modified photosynthetic microorganisms are cultured, at least for some time, in media supplemented with varying amounts of bicarbonate. For example, the modified photosynthetic microorganisms may be cultured with bicarbonate at 5, 10, 20, 50, 75, 100, 200, 300, 400, 500, 600, 700, 800, 900, 1000 mM bicarbonate prior to inducing expression of an introduced polynucleotide (e.g., acyl-ACP reductase, aldehyde dehydrogenase, ACP, glycogen breakdown protein, ACCase, DGAT, fatty acyl-CoA synthetase, alcohol dehydrogenase) and/or the modified photosynthetic microorganism may be cultured with aforementioned bicarbonate concentrations while expression of an introduced polynucleotide is being induced, or during a portion of the time period during which expression on an introduced polynucleotide is being induced.

In related embodiments, modified photosynthetic microorganisms and methods of the present invention may be used in the production of a biofuel and/or a specialty chemical, such as glycerin or a wax ester. Thus, in particular embodiments, a method of producing a biofuel comprises culturing any of the modified photosynthetic microorganisms of the present invention under conditions wherein the modified photosynthetic microorganism accumulates an increased amount of total cellular lipid, fatty acid, wax ester, and/or triglyceride, as compared to a corresponding wild-type photosynthetic microorganism, obtaining cellular lipid, fatty acid, wax ester, and/or triglyceride from said microorganism, and processing the obtained cellular lipid, fatty acid, wax ester, and/or triglyceride to produce a biofuel. In another embodiment, a method of producing a biofuel comprises processing lipids, fatty acids, wax esters, and/or triglycerides produced by a modified photosynthetic microorganism of the present invention to produce a biofuel. In a further embodiment, a method of producing a biofuel comprises obtaining lipid, fatty acid, wax esters, and/or triglyceride produced by a modified photosynthetic microorganism of the present invention, and processing the obtained cellular lipid, fatty acid, wax ester, and/or triglyceride to produce a biofuel.

Methods of processing lipids from microorganisms to produce a biofuel or specialty chemical, e.g., biodiesel, are known and available in the art. For example, triglycerides may be transesterified to produce biodiesel. Transesterification may be carried out by any one of the methods known in the art, such as alkali-, acid-, or lipase-catalysis (see, e.g., Singh et al. Recent Pat Biotechnol. 2008, 2(2):130-143). Various methods of transesterification utilize, for example, use of a batch reactor, a supercritical alcohol, an ultrasonic reactor, or microwave irradiation (Such methods are described, e.g., in Jeong and Park. Appl Biochem Biotechnol. 2006, 131(1-3):668-679; Fukuda et al. Journal of Bioscience and Engineering. 2001, 92(5):405-416; Shah and Gupta. Chemistry Central Journal. 2008, 2(1):1-9; and Carrillo-Munoz et al. J Org Chem. 1996, 61(22):7746-7749). The biodiesel may be further processed or purified, e.g., by distillation, and/or a biodiesel stabilizer may be added to the biodiesel, as described in U.S. patent application publication No. 2008/0282606.

### Nucleic Acids and Polypeptides

Modified photosynthetic microorganisms of the present invention comprise one or more overexpressed acyl-ACP reductase polypeptides. These modified photosynthetic microorganism can optionally comprise one or more overexpressed lipid biosynthesis proteins, e.g., one or more proteins associated with fatty acid synthesis such as ACP and ACCase, and/or one or more overexpressed proteins associated with glycogen breakdown. It is further understood that the compositions and methods of the present invention may be practiced using biologically active fragments and/or variants of any of these or other introduced or overexpressed polypeptides. Also, these modified microorganisms (e.g., those that comprise an overexpressed acyl-ACP reductase) may optionally comprise a mutation or deletion in one or more genes associated with glycogen biosynthesis or storage, and/or a mutation or deletion in or more genes encoding an aldehyde decarbonylase, either alone or in combination with the presence of overexpressed proteins associated with fatty biosynthesis proteins and/or glycogen breakdown. As will be apparent, modified photosynthetic microorganisms of the present invention may comprise any combination of one or more of the additional modifications noted herein, typically as long as they have an overexpressed acyl-ACP reductase.

### Acyl-ACP Reductases

Acyl-ACP reductases (or acyl-ACP dehydrogenases) are members of the reductase or short-chain dehydrogenase family, and are key enzymes of the type II fatty acid synthesis (FAS) system. Among other potential catalytic activities, an “acyl-ACP reductase” or “acyl-ACP dehydrogenase” as used herein is capable of catalyzing the conversion (reduction) of acyl-ACP to an acyl aldehyde (see Schirmer et al., supra; and FIG. 2) and the concomitant oxidation of NAD(P)H to NADP⁺. In some embodiments, the acyl-ACP reductase preferentially interacts with acyl-ACP, and does not interact significantly with acyl-CoA, i.e., it does not significantly catalyze the conversion of acyl-CoA to acyl aldehyde. Acyl-ACP reductases can be derived from a variety of plants and bacteria, included photosynthetic microorganisms such as Cyanobacteria. One exemplary acyl-ACP reductase is encoded by orf1594 of Synechococcus elongatus PCC7942 (see SEQ ID NOs:1 and 2 for the polynucleotide and polypeptide sequences, respectively). Another exemplary acyl-ACP reductase is encoded by orfsII0209 of Synechocystis sp. PCC6803 (SEQ ID NOs:3 and 4 for the polynucleotide and polypeptide sequences, respectively).

### Lipid Biosynthesis Proteins

In various embodiments, modified photosynthetic microorganisms, e.g., Cyanobacteria, of the present invention further comprise one or more exogenous (i.e., introduced) or overexpressed nucleic acids that encode a lipid biosynthesis protein, e.g., a polypeptide having an activity associated with fatty acid biosynthesis, including but not limited to any of those described herein. Specific examples of lipid or fatty acid biosynthesis proteins include acyl carrier protein (ACP), ACCase, DGAT, and fatty acyl-CoA synthetase.

In particular embodiments, the exogenous nucleic acid does not comprise a nucleic acid sequence that is native to the microorganism's genome. In particular embodiments, the exogenous nucleic acid comprises a nucleic acid sequence that is native to the microorganism's genome, but it has been introduced into the microorganism, e.g., in a vector or by molecular biology techniques, for example, to increase expression of the nucleic acid and/or its encoded polypeptide in the microorganism. In certain embodiments, the expression of a native or endogenous nucleic acid and its corresponding protein can be increased by introducing a heterologous promoter upstream of the native gene. As noted herein, lipid biosynthesis proteins can be involved in triglyceride biosynthesis, fatty acid synthesis, or both.

Triglyceride Biosynthesis.

Triglycerides, or triacylglycerols (TAGs), consist primarily of glycerol esterified with three fatty acids, and yield more energy upon oxidation than either carbohydrates or proteins. Triglycerides provide an important mechanism of energy storage for most eukaryotic organisms. In mammals, TAGs are synthesized and stored in several cell types, including adipocytes and hepatocytes (Bell et al. Annu. Rev. Biochem. 49:459-487, 1980) (herein incorporated by reference). In plants, TAG production is mainly important for the generation of seed oils.

In contrast to eukaryotes, the observation of triglyceride production in prokaryotes has been limited to certain actinomycetes, such as members of the genera Mycobacterium, Nocardia, Rhodococcus and Streptomyces, in addition to certain members of the genus Acinetobacter. In certain Actinomycetes species, triglycerides may accumulate to nearly 80% of the dry cell weight, but accumulate to only about 15% of the dry cell weight in Acinetobacter. In general, triglycerides are stored in spherical lipid bodies, with quantities and diameters depending on the respective species, growth stage, and cultivation conditions. For example, cells of Rhodococcus opacus and Streptomyces lividans contain only few TAGs when cultivated in complex media with a high content of carbon and nitrogen; however, the lipid content and the number of TAG bodies increase drastically when the cells are cultivated in mineral salt medium with a low nitrogen-to-carbon ratio, yielding a maximum in the late stationary growth phase. At this stage, cells can be almost completely filled with lipid bodies exhibiting diameters ranging from 50 to 400 nm. One example is R. opacus PD630, in which lipids can reach more than 70% of the total cellular dry weight.

In bacteria, TAG formation typically starts with the docking of a diacylglycerol acyltransferase enzyme to the plasma membrane, followed by formation of small lipid droplets (SLDs). These SLDs are only some nanometers in diameter and remain associated with the membrane-docked enzyme. In this phase of lipid accumulation, SLDs typically form an emulsive, oleogenous layer at the plasma membrane. During prolonged lipid synthesis, SLDs leave the membrane-associated acyltransferase and conglomerate to membrane-bound lipid prebodies. These lipid prebodies reach distinct sizes, e.g., about 200 nm in A. calcoaceticus and about 300 nm in R. opacus, before they lose contact with the membrane and are released into the cytoplasm. Free and membrane-bound lipid prebodies correspond to the lipid domains occurring in the cytoplasm and at the cell wall, as observed in M. smegmatis during fluorescence microscopy and also confirmed in R. opacus PD630 and A. calcoaceticus ADP1 (see, e.g., Christensen et al., Mol. Microbiol. 31:1561-1572, 1999; and Wältermann et al., Mol. Microbiol. 55:750-763, 2005). Inside the lipid prebodies, SLDs coalesce with each other to form the homogenous lipid core found in mature lipid bodies, which often appear opaque in electron microscopy.

The compositions and structures of bacterial TAGs vary considerably depending on the microorganism and on the carbon source. In addition, unusual acyl moieties, such as phenyldecanoic acid and 4,8,12 trimethyl tridecanoic acid, may also contribute to the structural diversity of bacterial TAGs (see, e.g., Alvarez et al., Appl Microbiol Biotechnol. 60:367-76, 2002).

As with eukaryotes, the main function of TAGs in prokaryotes is to serve as a storage compound for energy and carbon. TAGs, however, may provide other functions in prokaryotes. For example, lipid bodies may act as a deposit for toxic or useless fatty acids formed during growth on recalcitrant carbon sources, which must be excluded from the plasma membrane and phospholipid (PL) biosynthesis. Furthermore, many TAG-accumulating bacteria are ubiquitous in soil, and in this habitat, water deficiency causing dehydration is a frequent environmental stress. Storage of evaporation-resistant lipids might be a strategy to maintain a basic water supply, since oxidation of the hydrocarbon chains of the lipids under conditions of dehydration would generate considerable amounts of water. Cyanobacteria such as Synechococcus, however, do not produce triglycerides, because these organisms lack the enzymes necessary for triglyceride biosynthesis.

Triglycerides are synthesized from fatty acids and glycerol. As one mechanism of triglyceride (TAG) synthesis, sequential acylation of glycerol-3-phosphate via the “Kennedy Pathway” leads to the formation of phosphatidate. Phosphatidate is then dephosphorylated by the enzyme phosphatidate phosphatase to yield 1,2 diacylglycerol (DAG). Using DAG as a substrate, at least three different classes of enzymes are capable of mediating TAG formation. As one example, an enzyme having diacylglycerol acyltransferase (DGAT) activity catalyzes the acylation of DAG using acyl-CoA as a substrate. Essentially, DGAT enzymes combine acyl-CoA with 1,2 diacylglycerol molecule to form a TAG. As an alternative, Acyl-CoA-independent TAG synthesis may be mediated by a phospholipid:DAG acyltransferase found in yeast and plants, which uses phospholipids as acyl donors for DAG esterification. Third, TAG synthesis in animals and plants may be mediated by a DAG-DAG-transacylase, which uses DAG as both an acyl donor and acceptor, yielding TAG and monoacylglycerol.

Modified photosynthetic microorganisms, e.g., Cyanobacteria, of the present invention may comprise one or more exogenous polynucleotides encoding polypeptides comprising one or more of the polypeptides and enzymes described herein. In particular embodiments, the one or more exogenous polynucleotides encode a diacylglycerol acyltransferase and a fatty acyl-CoA synthetase, or a variant or function fragment thereof.

Since wild type Cyanobacteria do not typically encode the enzymes necessary for triglyceride synthesis, such as the enzymes having diacylglycerol acyltransferase activity, embodiments of the present invention include genetically modified Cyanobacteria that comprise polynucleotides encoding one or more enzymes having a diacylglycerol acyltransferase activity, typically in combination with one or more enzymes having a fatty acyl-CoA synthetase activity.

Moreover, since triglycerides are typically formed from fatty acids, the level of fatty acid biosynthesis in a cell may limit the production of triglycerides. Increasing the level of fatty acid biosynthesis may, therefore, allow increased production of triglycerides. As discussed below, acetyl-CoA carboxylase catalyzes the commitment step to fatty acid biosynthesis. Thus, certain embodiments of the present invention include Cyanobacterium, and methods of use thereof, comprising polynucleotides that encode one or more enzymes having Acetyl-CoA carboxylase activity to increase fatty acid biosynthesis and lipid production, in addition to one or more enzymes having diacylglycerol acyltransferase activity and one or more enzymes having fatty acyl-CoA synthetase activity, to catalyze triglyceride production. These and related embodiments are detailed below.

Fatty Acid Biosynthesis.

Fatty acids are a group of negatively charged, linear hydrocarbon chains of various length and various degrees of oxidation states. The negative charge is located at a carboxyl end group and is typically deprotonated at physiological pH values (pK ˜2-3). The length of the fatty acid ‘tail’ determines its water solubility (or rather insolubility) and amphipathic characteristics. Fatty acids are components of phospholipids and sphingolipids, which form part of biological membranes, as well as triglycerides, which are primarily used as energy storage molecules inside cells.

Fatty acids are formed from acetyl-CoA and malonyl-CoA precursors. Malonyl-CoA is a carboxylated form of acetyl-CoA, and contains a 3-carbon dicarboxylic acid, malonate, bound to Coenzyme A. Acetyl-CoA carboxylase catalyzes the 2-step reaction by which acetyl-CoA is carboxylated to form malonyl-CoA. In particular, malonate is formed from acetyl-CoA by the addition of CO₂ using the biotin cofactor of the enzyme acetyl-CoA carboxylase.

Fatty acid synthase (FAS) carries out the chain elongation steps of fatty acid biosynthesis. FAS is a large multienzyme complex. In mammals, FAS contains two subunits, each containing multiple enzyme activities. In bacteria and plants, individual proteins, which associate into a large complex, catalyze the individual steps of the synthesis scheme. For example, in bacteria and plants, the acyl carrier protein is a smaller, independent protein.

Fatty acid synthesis starts with acetyl-CoA, and the chain grows from the “tail end” so that carbon 1 and the alpha-carbon of the complete fatty acid are added last. The first reaction is the transfer of an acetyl group to a pantothenate group of acyl carrier protein (ACP), a region of the large mammalian fatty acid synthase (FAS) protein. In this reaction, acetyl CoA is added to a cysteine —SH group of the condensing enzyme (CE) domain: acetyl CoA+CE-cys-SH->acetyl-cys-CE+CoASH. Mechanistically, this is a two step process, in which the group is first transferred to the ACP (acyl carrier peptide), and then to the cysteine —SH group of the condensing enzyme domain.

In the second reaction, malonyl CoA is added to the ACP sulfhydryl group: malonyl CoA+ACP-SH->malonyl ACP+CoASH. This —SH group is part of a phosphopantethenic acid prosthetic group of the ACP.

In the third reaction, the acetyl group is transferred to the malonyl group with the release of carbon dioxide: malonyl ACP+acetyl-cys-CE->beta-ketobutyryl-ACP+CO₂.

In the fourth reaction, the keto group is reduced to a hydroxyl group by the beta-ketoacyl reductase activity: beta-ketobutyryl-ACP+NADPH+H⁺->beta-hydroxybutyryl-ACP+NAD⁺.

In the fifth reaction, the beta-hydroxybutyryl-ACP is dehydrated to form a trans-monounsaturated fatty acyl group by the beta-hydroxyacyl dehydratase activity: beta-hydroxybutyryl-ACP->2-butenoyl-ACP+H₂O.

In the sixth reaction, the double bond is reduced by NADPH, yielding a saturated fatty acyl group two carbons longer than the initial one (an acetyl group was converted to a butyryl group in this case): 2-butenoyl-ACP+NADPH+H⁺->butyryl-ACP+NADP⁺. The butyryl group is then transferred from the ACP sulfhydryl group to the CE sulfhydryl: butyryl-ACP+CE-cys-SH->ACP-SH+butyryl-cys-CE. This step is catalyzed by the same transferase activity utilized previously for the original acetyl group. The butyryl group is now ready to condense with a new malonyl group (third reaction above) to repeat the process. When the fatty acyl group becomes 16 carbons long, a thioesterase activity hydrolyses it, forming free palmitate: palmitoyl-ACP+H₂O->palmitate+ACP-SH. Fatty acid molecules can undergo further modification, such as elongation and/or desaturation.

Modified photosynthetic microorganisms, e.g., Cyanobacteria, may comprise one or more exogenous polynucleotides encoding any of the above polypeptides or enzymes involved in fatty acid synthesis. In particular embodiments, the enzyme is an acetyl-CoA carboxylase or a variant or functional fragment thereof. Certain exemplary lipid biosynthesis proteins are described below.

Wax Ester Synthesis.

Wax esters are esters of a fatty acid and a long-chain alcohol. These neutral lipids are composed of aliphatic alcohols and acids, with both moieties usually long-chain (e.g., C₁₆ and C₁₈) or very-long-chain (C₂₀ and longer) carbon structures, though medium-chain-containing wax esters are included (e.g., C₁₀, C₁₂ and C₁₄). Wax esters have diverse biological functions in bacteria, insects, mammals, and terrestrial plants and are also important substrates for a variety of industrial applications. Various types of wax ester are widely used in the manufacture of fine chemicals such as cosmetics, candles, printing inks, lubricants, coating stuffs, and others.

In certain organisms, such as Acinetobacter, the pathway for wax ester synthesis of Acinetobacter spp. has been assumed to start from acyl coenzyme A (acyl-CoA), which is then reduced to the corresponding alcohol via acyl-CoA reductase and aldehyde reductase. In other organisms, for example, wax ester biosynthesis involves elongation of saturated C₁₆ and C₁₈ fatty acyl-CoAs to very-long-chain fatty acid wax precursors between 24 and 34 carbons in length, and their subsequent modification by either the alkane-forming (decarbonylation) or the alcohol-forming (acyl reduction) pathway (see Li et al., Plant Physiology 148:97-107, 2008).

Here, the accompanying Examples have shown that in certain Cyanobacteria, wax ester synthesis can occur via the acyl-ACP=>acyl aldehyde pathway. In this pathway, acyl-ACP reductase overexpression increases conversion of acyl-ACP into acyl aldehydes, alcohol dehydrogenase overexpression then increases conversion of acyl aldehydes into fatty alcohols, and DGAT overexpression cooperatively increases conversion of the fatty alcohols into their corresponding wax esters. Modified photosynthetic microorganisms, e.g., Cyanobacteria, may therefore comprise one or more exogenous polynucleotides encoding any of the above polypeptides or enzymes involved in wax ester synthesis.

Acyl-Carrier Proteins (ACP)

Embodiments of the present invention optionally include one or more exogenous (e.g., recombinantly introduced) or overexpressed ACP proteins. These proteins play crucial roles in fatty acid synthesis. Fatty acid synthesis in bacteria, including Cyanobacteria, is carried out by highly conserved enzymes of the type II fatty acid synthase system (FAS II; consisting of about 19 genes) in a sequential, regulated manner. Acyl carrier protein (ACP) plays a central role in this process by carrying all the intermediates as thioesters attached to the terminus of its 4′-phosphopantetheine prosthetic group (ACP-thioesters). Apo-ACP, the product of acp gene, is typically activated by a phosphopantetheinyl transferease (PPT) such as the acyl carrier protein synthase (AcpS) type found in E. coli or the Sfp (surfactin type) PTT as characterized in Bacillus subtilis. Cyanobacteria posses an Sfp-like PPT, which is understood to act in both primary and secondary metabolism. Embodiments of the present invention therefore include overexpression of PPTs such as AcpS and/or Sfp-type PPTs in combination with overexpression of cognate ACP encoding genes, such as ACP.

The ACP-thioesters are substrates for all of the enzymes of the FAS II system. The end product of fatty acid synthesis is a long acyl chain typically consisting of about 14-18 carbons attached to ACP by a thioester bond.

At least three enzymes of the FAS II system in other bacteria can be subject to feedback inhibition by acyl-ACPs: 1) the ACCase complex—a heterotetramer of the AccABCD genes that catalyzes the production of malonyl-coA, the first step in the pathway; 2) the product of the FabH gene (β-ketoacyl-ACP synthase III), which catalyzes the condensation of acetyl-CoA with malonyl-ACP; and 3) the product of the Fabl gene (enoyl-ACP reductase), which catalyzes the final elongation step in each round of elongation. Certain proteins such as acyl-ACP reductase are capable of increasing fatty acid production in photosynthetic bacteria such as Cyanobacteria, and it is believed that overexpression of ACP in combination with this protein and possibly other biosynthesis proteins will further increases fatty acid production in such strains.

An ACP can be derived from a variety of eukaryotic organisms, microorganisms (e.g., bacteria, fungi), or plants. In certain embodiments, an ACP polynucleotide sequence and its corresponding polypeptide sequence are derived from Cyanobacteria such as Synechococcus. In certain embodiments, ACPs can be derived from plants such as spinach. SEQ ID NOS:5-12 provide the nucleotide and polypeptide sequences of exemplary bacterial ACPs from Synechococcus and Acinetobacter, and SEQ ID NOS:13-14 provide the same for an exemplary plant ACP from Spinacia oleracea (spinach). SEQ ID NOS:5 and 6 derive from Synechococcus elongatus PCC7942, and SEQ ID NOS:7-12 derive from Acinetobacter sp. ADP1.

Examples of prokaryotic organisms having an ACP include certain actinomycetes, a group of Gram-positive bacteria with high G+C ratio, such as those from the representative genera Actinomyces, Arthrobacter, Corynebacterium, Frankia, Micrococcus, Mocrimonospora, Mycobacterium, Nocardia, Propionibacterium, Rhodococcus and Streptomyces. Particular examples of actinomycetes that have one or more genes encoding an ACP activity include, for example, Mycobacterium tuberculosis, M. avium, M. smegmatis, Micromonospora echinospora, Rhodococcus opacus, R. ruber, and Streptomyces lividans. Additional examples of prokaryotic organisms that encode one or more enzymes having an ACP activity include members of the genera Acinetobacter, such as A. calcoaceticus, A. baumanii, A. baylii, and members of the generua Alcanivorax. In certain embodiments, an ACP gene or enzyme is isolated from Acinetobacter baylii sp. ADP1, a gram-negative triglyceride forming prokaryote.

Acetyl CoA Carboxylases (ACCase)

Embodiments of the present invention optionally include one or more exogenous (e.g., recombinantly introduced) or overexpressed ACCase proteins. As used herein, an “acetyl CoA carboxylase” gene of the present invention includes any polynucleotide sequence encoding amino acids, such as protein, polypeptide or peptide, obtainable from any cell source, which demonstrates the ability to catalyze the carboxylation of acetyl-CoA to produce malonyl-CoA under enzyme reactive conditions, and further includes any naturally-occurring or non-naturally occurring variants of an acetyl-CoA carboxylase sequence having such ability.

Acetyl-CoA carboxylase (ACCase) is a biotin-dependent enzyme that catalyses the irreversible carboxylation of acetyl-CoA to produce malonyl-CoA through its two catalytic activities, biotin carboxylase (BC) and carboxyltransferase (CT). The biotin carboxylase (BC) domain catalyzes the first step of the reaction: the carboxylation of the biotin prosthetic group that is covalently linked to the biotin carboxyl carrier protein (BCCP) domain. In the second step of the reaction, the carboxyltransferase (CT) domain catalyzes the transfer of the carboxyl group from (carboxy) biotin to acetyl-CoA. Formation of malonyl-CoA by acetyl-CoA carboxylase (ACCase) represents the commitment step for fatty acid synthesis, because malonyl-CoA has no metabolic role other than serving as a precursor to fatty acids. Because of this reason, acetyl-CoA carboxylase represents a pivotal enzyme in the synthesis of fatty acids.

In most prokaryotes, ACCase is a multi-subunit enzyme, whereas in most eukaryotes it is a large, multi-domain enzyme. In yeast, the crystal structure of the CT domain of yeast ACCase has been determined at 2.7 A resolution (Zhang et al., Science, 299:2064-2067 (2003). This structure contains two domains, which share the same backbone fold. This fold belongs to the crotonase/ClpP family of proteins, with a b-b-a superhelix. The CT domain contains many insertions on its surface, which are important for the dimerization of ACCase. The active site of the enzyme is located at the dimer interface.

Although Cyanobacteria, such as Synechococcus, express a native ACCase enzyme, these bacteria typically do not produce or accumulate significant amounts of fatty acids. For example, Synechococcus in the wild accumulates fatty acids in the form of lipid membranes to a total of about 4% by dry weight.

Given the role of ACCase in the commitment step of fatty acid biosynthesis, embodiments of the present invention include methods of increasing the production of fatty acid biosynthesis, and, thus, lipid production, in Cyanobacteria by introducing one or more polynucleotides that encode an ACCase enzyme that is exogenous to the Cyanobacterium's native genome. Embodiments of the present invention also include a modified Cyanobacterium, and compositions comprising said Cyanobacterium, comprising one or more polynucleotides that encode an ACCase enzyme that is exogenous to the Cyanobacterium's native genome.

A polynucleotide encoding an ACCase enzyme may be isolated or obtained from any organism, such as any prokaryotic or eukaryotic organism that contains an endogenous ACCase gene. Examples of eukaryotic organisms having an ACCase gene are well-known in the art, and include various animals (e.g., mammals, fruit flies, nematodes), plants, parasites, and fungi (e.g., yeast such as S. cerevisiae and Schizosaccharomyces pombe). In certain embodiments, the ACCase encoding polynucleotide sequences are obtained from Synechococcus sp. PCC7002.

Examples of prokaryotic organisms that may be utilized to obtain a polynucleotide encoding an enzyme having ACCase activity include, but are not limited to, Escherichia coli, Legionella pneumophila, Listeria monocytogenes, Streptococcus pneumoniae, Bacillus subtilis, Ruminococcus obeum ATCC 29174, marine gamma proteobacterium HTCC2080, Roseovarius sp. HTCC2601, Oceanicola granulosus HTCC2516, Bacteroides caccae ATCC 43185, Vibrio alginolyticus 12G01, Pseudoalteromonas tunicata D2, Marinobacter sp. ELB17, marine gamma proteobacterium HTCC2143, Roseobacter sp. SK209-2-6, Oceanicola batsensis HTCC2597, Rhizobium leguminosarum bv. trifolii WSM1325, Nitrobacter sp. Nb-311A, Chloroflexus aggregans DSM 9485, Chlorobaculum parvum, Chloroherpeton thalassium, Acinetobacter baumannii, Geobacillus, and Stenotrophomonas maltophilia, among others.

Diacylglycerol Acyltransferases (DGAT)

As used herein, a “diacylglycerol acyltransferase” (DGAT) gene of the present invention includes any polynucleotide sequence encoding amino acids, such as protein, polypeptide or peptide, obtainable from any cell source, which demonstrates the ability to catalyze the production of triacylglycerol from 1,2-diacylglycerol and fatty acyl substrates under enzyme reactive conditions, in addition to any naturally-occurring (e.g., allelic variants, orthologs) or non-naturally occurring variants of a diacylglycerol acyltransferase sequence having such ability. DGAT genes of the present invention also include polynucleotide sequences that encode bi-functional proteins, such as those bi-functional proteins that exhibit a DGAT activity as well as a CoA:fatty alcohol acyltransferase activity, e.g., a wax ester synthesis (WS) activity, as often found in many TAG producing bacteria.

Diacylglycerol acyltransferases (DGATs) are members of the O-acyltransferase superfamily, which esterify either sterols or diacyglycerols in an oleoyl-CoA-dependent manner. DGAT in particular esterifies diacylglycerols, which reaction represents the final enzymatic step in the production of triacylglycerols in plants, fungi and mammals. Specifically, DGAT is responsible for transferring an acyl group from acyl-coenzyme-A to the sn-3 position of 1,2-diacylglycerol (DAG) to form triacylglycerol (TAG). DGAT is an integral membrane protein that has been generally described in Harwood (Biochem. Biophysics. Acta, 1301:7-56, 1996), Daum et al. (Yeast 16:1471-1510, 1998), and Coleman et al. (Annu. Rev. Nutr. 20:77-103, 2000) (each of which are herein incorporated by reference).

In plants and fungi, DGAT is associated with the membrane and lipid body fractions. In catalyzing TAGs, DGAT contributes mainly to the storage of carbon used as energy reserves. In animals, however, the role of DGAT is more complex. DGAT not only plays a role in lipoprotein assembly and the regulation of plasma triacylglycerol concentration (Bell, R. M., et al.), but participates as well in the regulation of diacylglycerol levels (Brindley, Biochemistry of Lipids, Lipoproteins and Membranes, eds. Vance, D. E. & Vance, J. E. (Elsevier, Amsterdam), 171-203; and Nishizuka, Science 258:607-614 (1992) (each of which are herein incorporated by reference)).

In eukaryotes, at least three independent DGAT gene families (DGAT1, DGAT2, and PDAT) have been described that encode proteins with the capacity to form TAG. Yeast contain all three of DGAT1, DGAT2, and PDAT, but the expression levels of these gene families varies during different phases of the life cycle (Dahlqvst, A., et al. Proc. Natl. Acad. Sci. USA 97:6487-6492 (2000) (herein incorporated by reference).

In prokaryotes, WS/DGAT from Acinetobacter calcoaceticus ADP1 represents the first identified member of a widespread class of bacterial wax ester and TAG biosynthesis enzymes. This enzyme comprises a putative membrane-spanning region but shows no sequence homology to the DGAT1 and DGAT2 families from eukaryotes. Under in vitro conditions, WS/DGAT shows a broad capability of utilizing a large variety of fatty alcohols, and even thiols as acceptors of the acyl moieties of various acyl-CoA thioesters. WS/DGAT acyltransferase enzymes exhibit extraordinarily broad substrate specificity. Genes for homologous acyltransferases have been found in almost all bacteria capable of accumulating neutral lipids, including, for example, Acinetobacter baylii, A. baumanii, and M. avium, and M. tuberculosis CDC1551, in which about 15 functional homologues are present (see, e.g., Daniel et al., J. Bacteriol. 186:5017-5030, 2004; and Kalscheuer et al., J. Biol. Chem. 287:8075-8082, 2003).

DGAT proteins may utilize a variety of acyl substrates in a host cell, including fatty acyl-CoA and fatty acyl-ACP molecules. In addition, the acyl substrates acted upon by DGAT enzymes may have varying carbon chain lengths and degrees of saturation, although DGAT may demonstrate preferential activity towards certain molecules.

Like other members of the eukaryotic O-acyltransferase superfamily, eukaryotic DGAT polypeptides typically contain a FYxDWWN (SEQ ID NO:15) heptapeptide retention motif, as well as a histidine (or tyrosine)-serine-phenylalanine (H/YSF) tripeptide motif, as described in Zhongmin et al. (Journal of Lipid Research, 42:1282-1291, 2001) (herein incorporated by reference). The highly conserved FYxDWWN (SEQ ID NO:15) is believed to be involved in fatty Acyl-CoA binding.

DGAT enzymes utilized according to the present invention may be isolated from any organism, including eukaryotic and prokaryotic organisms. Eukaryotic organisms having a DGAT gene are well-known in the art, and include various animals (e.g., mammals, fruit flies, nematodes), plants, parasites, and fungi (e.g., yeast such as S. cerevisiae and Schizosaccharomyces pombe). Examples of prokaryotic organisms include certain actinomycetes, a group of Gram-positive bacteria with high G+C ratio, such as those from the representative genera Actinomyces, Arthrobacter, Corynebacterium, Frankia, Micrococcus, Mocrimonospora, Mycobacterium, Nocardia, Propionibacterium, Rhodococcus and Streptomyces. Particular examples of actinomycetes that have one or more genes encoding a DGAT activity include, for example, Mycobacterium tuberculosis, M. avium, M. smegmatis, Micromonospora echinospora, Rhodococcus opacus, R. ruber, and Streptomyces lividans. Additional examples of prokaryotic organisms that encode one or more enzymes having a DGAT activity include members of the genera Acinetobacter, such as A. calcoaceticus, A. baumanii, A. baylii, and members of the generua Alcanivorax. In certain embodiments, a DGAT gene or enzyme is isolated from Acinetobacter baylii sp. ADP1, a gram-negative triglyceride forming prokaryote, which contains a well-characterized DGAT (AtfA).

In certain embodiments, the modified photosynthetic microorganisms of the present invention may comprise two or more polynucleotides that encode DGAT or a variant or fragment thereof. In particular embodiments, the two or more polynucleotides are identical or express the same DGAT. In certain embodiments, these two or more polynucleotides may be different or may encode two different DGAT polypeptides. For example, in one embodiment, one of the polynucleotides may encode ADGATd, while another polynucleotide may encode ScoDGAT. In particular embodiments, the following DGATs are coexpressed in modified photosynthetic microorganisms, e.g., Cyanobacteria, using one of the following double DGAT strains: ADGATd(NS1)::ADGATd(NS2); ADGATn(NS1)::ADGATn(NS2); ADGATn(NS1)::SDGAT(NS2); SDGAT(NS1)::ADGATn(NS2); SDGAT(NS1)::SDGAT(NS2). For the NS1 vector, pAM2291, EcoRI follows ATG and is part of the open reading frame (ORF). For the NS2 vector, pAM1579, EcoRI follows ATG and is part of the ORF. A DGAT having EcoRI nucleotides following ATG may be cloned in either pAM2291 or pAM1579; such a DGAT is referred to as ADGATd. Other embodiments utilize the vector, pAM2314FTrc3, which is an NS1 vector with Nde/BglII sites, or the vector, pAM1579FTrc3, which is the NS2 vector with Nde/BglII sites. A DGAT without EcoRI nucleotides may be cloned into either of these last two vectors. Such a DGAT is referred to as ADGATn. Modified photosynthetic microorganisms expressing different DGATs express TAGs having different fatty acid compositions. Accordingly, certain embodiments of the present invention contemplate expressing two or more different DGATs, in order to produce TAGs having varied fatty acid compositions.

Fatty Acyl-CoA Synthetases

Certain embodiments relate to the use of overexpressed fatty acyl-CoA synthetases to increase activation of fatty acids, and thereby increase production of TAGs in a TAG-producing strain (e.g., a DGAT-expressing strain). For instance, specific embodiments may utilize an acyl-ACP reductase in combination with a fatty acyl-CoA synthetase and a DGAT. These embodiments may then further utilize an ACP, an ACCase, or both, and/or any of the modifications to glycogen production and storage or glycogen breakdown described herein.

Fatty acyl-CoA synthetases activate fatty acids for metabolism by catalyzing the formation of fatty acyl-CoA thioesters. Fatty acyl-CoA thioesters can then serve not only as substrates for beta-oxidation, at least in bacteria capable of growing on fatty acids as a sole source of carbon (e.g., E. coli, Salmonella), but also as acyl donors in phospholipid biosynthesis. Many fatty acyl-CoA synthetases are characterized by two highly conserved sequence elements, an ATP/AMP binding motif, which is common to enzymes that form an adenylated intermediate, and a fatty acid binding motif.

According to one non-limiting theory, certain embodiments may employ fatty acyl-CoA synthetases to increase activation of free fatty acids, which can then be incorporated into TAGs, mainly by the DGAT-expressing (and thus TAG-producing) photosynthetic microorganisms described herein. Hence, fatty acyl-CoA synthetases can be used in any of the embodiments described herein, such as those that produce increased levels of free fatty acids, where it is desirable to turn free fatty acids into TAGs. As noted above, these free fatty acids can then be activated by fatty acyl-CoA synthetases to generate acyl-CoA thioesters, which can then serve as substrates by DGAT to produce increased levels of TAGs.

One exemplary fatty acyl-CoA synthetase includes the FadD gene from E. coli (SEQ ID NOS:16 and 17 for nucleotide and polypeptide sequence, respectively), which encodes a fatty acyl-CoA synthetase having substrate specificity for medium and long chain fatty acids. Other exemplary fatty acyl-CoA synthetases include those derived from S. cerevisiae; Faalp can use C12-C16 acyl-chains in vitro (see SEQ ID NOS:18 and 19 for nucleotide and polypeptide sequence, respectively), Faa2p shows a less restricted specificity ranging from C7-C17 (see SEQ ID NOS:20 and 21 for nucleotide and polypeptide sequence, respectively), and Faa3p, together with that of DGAT1, enhances lipid accumulation in the presence of exogenous fatty acids in S. cerevisiae (see SEQ ID NO:22 and 23 for nucleotide and polypeptide sequence, respectively). SEQ ID NO:22 is codon-optimized for expression in S. elongatus PCC7942.

### Glycogen Synthesis, Storage, and Breakdown

In particular embodiments, a modified photosynthetic microorganism further comprises additional modifications, such that it has reduced expression of one or more genes associated with a glycogen synthesis or storage pathway and/or increased expression of one or more polynucleotides that encode a protein associated with a glycogen breakdown pathway, or a functional variant of fragment thereof.

In various embodiments, modified photosynthetic microorganisms, e.g., Cyanobacteria, of the present invention have reduced expression of one or more genes associated with glycogen synthesis and/or storage. In particular embodiments, these modified photosynthetic microorganisms have a mutated or deleted gene associated with glycogen synthesis and/or storage. In particular embodiments, these modified photosynthetic microorganisms comprise a vector that includes a portion of a mutated or deleted gene, e.g., a targeting vector used to generate a knockout or knockdown of one or more alleles of the mutated or deleted gene. In certain embodiments, these modified photosynthetic microorganisms comprise an antisense RNA or siRNA that binds to an mRNA expressed by a gene associated with glycogen synthesis and/or storage.

In certain embodiments, modified photosynthetic microorganisms, e.g., Cyanobacteria, of the present invention comprise one or more exogenous or introduced nucleic acids that encode a polypeptide having an activity associated with a glycogen breakdown or triglyceride or fatty acid biosynthesis, including but not limited to any of those described herein. In particular embodiments, the exogenous nucleic acid does not comprise a nucleic acid sequence that is native to the microorganism's genome. In particular embodiments, the exogenous nucleic acid comprises a nucleic acid sequence that is native to the microorganism's genome, but it has been introduced into the microorganism, e.g., in a vector or by molecular biology techniques, for example, to increase expression of the nucleic acid and/or its encoded polypeptide in the microorganism.

Glycogen Biosynthesis and Storage.

Glycogen is a polysaccharide of glucose, which functions as a means of carbon and energy storage in most cells, including animal and bacterial cells. More specifically, glycogen is a very large branched glucose homopolymer containing about 90% α-1,4-glucosidic linkages and 10% α-1,6 linkages. For bacteria in particular, the biosynthesis and storage of glycogen in the form of α-1,4-polyglucans represents an important strategy to cope with transient starvation conditions in the environment.

Glycogen biosynthesis involves the action of several enzymes. For instance, bacterial glycogen biosynthesis occurs generally through the following general steps: (1) formation of glucose-1-phosphate, catalyzed by phosphoglucomutase (Pgm), followed by (2) ADP-glucose synthesis from ATP and glucose 1-phosphate, catalyzed by glucose-1-phosphate adenylyltransferase (GlgC), followed by (3) transfer of the glucosyl moiety from ADP-glucose to a pre-existing α-1,4 glucan primer, catalyzed by glycogen synthase (GlgA). This latter step of glycogen synthesis typically occurs by utilizing ADP-glucose as the glucosyl donor for elongation of the α-1,4-glucosidic chain.

In bacteria, the main regulatory step in glycogen synthesis takes place at the level of ADP-glucose synthesis, or step (2) above, the reaction catalyzed by glucose-1-phosphate adenylyltransferase (GlgC), also known as ADP-glucose pyrophosphorylase (see, e.g., Ballicora et al., Microbiology and Molecular Biology Reviews 6:213-225, 2003). In contrast, the main regulatory step in mammalian glycogen synthesis occurs at the level of glycogen synthase. As shown herein, by altering the regulatory and/or other active components in the glycogen synthesis pathway of photosynthetic microorganisms such as Cyanobacteria, and thereby reducing the biosynthesis and storage of glycogen, the carbon that would have otherwise been stored as glycogen can be utilized by said photosynthetic microorganism to synthesize other carbon-based storage molecules, such as lipids, fatty acids, and triglycerides.

Therefore, certain modified photosynthetic microorganisms, e.g., Cyanobacteria, of the present invention may comprise a mutation, deletion, or any other alteration that disrupts one or more of these steps (i.e., renders the one or more steps “non-functional” with respect to glycogen biosynthesis and/or storage), or alters any one or more of the enzymes directly involved in these steps, or the genes encoding them. As noted above, such modified photosynthetic microorganisms, e.g., Cyanobacteria, are typically capable of producing and/or accumulating an increased amount of lipids, such as fatty acids, as compared to a wild type photosynthetic microorganism. Certain exemplary glycogen biosynthesis genes are described below.

i. Phosphoglucomutase Gene (Pgm)

In one embodiment, a modified photosynthetic microorganism, e.g., a Cyanobacteria, expresses a reduced amount of the phosphoglucomutase gene. In particular embodiments, it may comprise a mutation or deletion in the phosphoglucomutase gene, including any of its regulatory elements (e.g., promoters, enhancers, transcription factors, positive or negative regulatory proteins, etc.). Phosphoglucomutase (Pgm), encoded by the gene pgm, catalyzes the reversible transformation of glucose 1-phosphate into glucose 6-phosphate, typically via the enzyme-bound intermediate, glucose 1,6-biphosphate (see, e.g., Lu et al., Journal of Bacteriology 176:5847-5851, 1994). Although this reaction is reversible, the formation of glucose-6-phosphate is markedly favored.

However, typically when a large amount of glucose-6-phosphate is present, Pgm catalyzes the phosphorylation of the 1-carbon and the dephosphorylation of the c-carbon, resulting in glucose-1-phosphate. The resulting glucose-1-phosphate is then converted to UDP-glucose by a number of intermediate steps, including the catalytic activity of GlgC, which can then be added to a glycogen storage molecule by the activity of glycogen synthase, described below. Thus, under certain conditions, the Pgm enzyme plays an intermediary role in the biosynthesis and storage of glycogen.

The pgm gene is expressed in a wide variety of organisms, including most, if not all, Cyanobacteria. The pgm gene is also fairly conserved among Cyanobacteria, as can be appreciated upon comparison of SEQ ID NOs:24 (S. elongatus PCC7942), 25 (Synechocystis sp. PCC6803), and 26 (Synechococcus sp. WH8102), which provide the polynucleotide sequences of various pgm genes from Cyanobacteria.

Deletion of the pgm gene in Cyanobacteria, such as Synechococcus, has been demonstrated herein for the first time to reduce the accumulation of glycogen in said Cyanobacteria, and also to increase the production of other carbon-based products, such as lipids and fatty acids.

ii. Glucose-1-Phosphate Adenylyltransferase (glgC)

In one embodiment, a modified photosynthetic microorganism, e.g., a Cyanobacteria, expresses a reduced amount of a glucose-1-phosphate adenylyltransferase (glgC) gene. In certain embodiments, it may comprise a mutation or deletion in the glgC gene, including any of its regulatory elements. The enzyme encoded by the glgC gene (e.g., EC 2.7.7.27) participates generally in starch, glycogen and sucrose metabolism by catalyzing the following chemical reaction:

ATP+alpha-D-glucose 1-phosphate [symbol] diphosphate+ADP-glucose

Thus, the two substrates of this enzyme are ATP and alpha-D-glucose 1-phosphate, whereas its two products are diphosphate and ADP-glucose. The glgC-encoded enzyme catalyzes the first committed and rate-limiting step in starch biosynthesis in plants and glycogen biosynthesis in bacteria. It is the enzymatic site for regulation of storage polysaccharide accumulation in plants and bacteria, being allosterically activated or inhibited by metabolites of energy flux.

The enzyme encoded by the glgC gene belongs to a family of transferases, specifically those transferases that transfer phosphorus-containing nucleotide groups nucleotidyl-transferases). The systematic name of this enzyme class is typically referred to as ATP:alpha-D-glucose-1-phosphate adenylyltransferase. Other names in common use include ADP glucose pyrophosphorylase, glucose 1-phosphate adenylyltransferase, adenosine diphosphate glucose pyrophosphorylase, adenosine diphosphoglucose pyrophosphorylase, ADP-glucose pyrophosphorylase, ADP-glucose synthase, ADP-glucose synthetase, ADPG pyrophosphorylase, and ADP:alpha-D-glucose-1-phosphate adenylyltransferase.

The glgC gene is expressed in a wide variety of plants and bacteria, including most, if not all, Cyanobacteria. The glgC gene is also fairly conserved among Cyanobacteria, as can be appreciated upon comparison of SEQ ID NOs:27 (S. elongatus PCC7942), 28 (Synechocystis sp. PCC6803), 29 (Synechococcus sp. PCC 7002), 30 (Synechococcus sp. WH8102), 31 (Synechococcus sp. RCC 307), 32 (Trichodesmium erythraeum IMS 101), 33 (Anabaena varibilis), and 31 (Nostoc sp. PCC 7120), which describe the polynucleotide sequences of various glgC genes from Cyanobacteria.

Deletion of the glgC gene in Cyanobacteria, such as Synechococcus, has been demonstrated herein for the first time to reduce the accumulation of glycogen in said Cyanobacteria, and also to increase the production of other carbon-based products, such as lipids and fatty acids.

iii. Glycogen Synthase (glgA)

In one embodiment, a modified photosynthetic microorganism, e.g., a Cyanobacteria, expresses a reduced amount of a glycogen synthase gene. In particular embodiments, it may comprise a deletion or mutation in the glycogen synthase gene, including any of is regulatory elements. Glycogen synthase (GlgA), also known as UDP-glucose-glycogen glucosyltransferase, is a glycosyltransferase enzyme that catalyses the reaction of UDP-glucose and (1,4-α-D-glucosyl)ₙ to yield UDP and (1,4-α-D-glucosyl)ₙ₊₁. Glycogen synthase is an α-retaining glucosyltransferase that uses ADP-glucose to incorporate additional glucose monomers onto the growing glycogen polymer. Essentially, GlgA catalyzes the final step of converting excess glucose residues one by one into a polymeric chain for storage as glycogen.

Classically, glycogen synthases, or α-1,4-glucan synthases, have been divided into two families, animal/fungal glycogen synthases and bacterial/plant starch synthases, according to differences in sequence, sugar donor specificity and regulatory mechanisms. However, detailed sequence analysis, predicted secondary structure comparisons, and threading analysis show that these two families are structurally related and that some domains of animal/fungal synthases were acquired to meet the particular regulatory requirements of those cell types.

Crystal structures have been established for certain bacterial glycogen synthases (see, e.g., Buschiazzo et al., The EMBO Journal 23, 3196-3205, 2004). These structures show that reported glycogen synthase folds into two Rossmann-fold domains organized as in glycogen phosphorlyase and other glycosyltransferases of the glycosyltransferases superfamily, with a deep fissure between both domains that includes the catalytic center. The core of the N-terminal domain of this glycogen synthase consists of a nine-stranded, predominantly parallel, central β-sheet flanked on both sides by seven α-helices. The C-terminal domain (residues 271-456) shows a similar fold with a six-stranded parallel β-sheet and nine α-helices. The last α-helix of this domain undergoes a kink at position 457-460, with the final 17 residues of the protein (461-477) crossing over to the N-terminal domain and continuing as α-helix, a typical feature of glycosyltransferase enzymes.

These structures also show that the overall fold and the active site architecture of glycogen synthase are remarkably similar to those of glycogen phosphorylase, the latter playing a central role in the mobilization of carbohydrate reserves, indicating a common catalytic mechanism and comparable substrate-binding properties. In contrast to glycogen phosphorylase, however, glycogen synthase has a much wider catalytic cleft, which is predicted to undergo an important interdomain ‘closure’ movement during the catalytic cycle.

Crystal structures have been established for certain GlgA enzymes (see, e.g., Jin et al., EMBO J 24:694-704, 2005, incorporated by reference). These studies show that the N-terminal catalytic domain of GlgA resembles a dinucleotide-binding Rossmann fold and the C-terminal domain adopts a left-handed parallel beta helix that is involved in cooperative allosteric regulation and a unique oligomerization. Also, communication between the regulator-binding sites and the active site involves several distinct regions of the enzyme, including the N-terminus, the glucose-1-phosphate-binding site, and the ATP-binding site.

The glgA gene is expressed in a wide variety of cells, including animal, plant, fungal, and bacterial cells, including most, if not all, Cyanobacteria. The glgA gene is also fairly conserved among Cyanobacteria, as can be appreciated upon comparison of SEQ ID NOs:35 (S. elongatus PCC7942), 36 (Synechocystis sp. PCC6803), 37 (Synechococcus sp. PCC 7002), 38 (Synechococcus sp. WH8102), 39 (Synechococcus sp. RCC 307), 40 (Trichodesmium erythraeum IMS 101), 41 (Anabaena variabilis), and 42 (Nostoc sp. PCC 7120), which describe the polynucleotide sequences of various glgA genes from Cyanobacteria.

Glycogen Breakdown.

In certain embodiments, a modified photosynthetic microorganism of the present invention expresses an increased amount of one or more genes associated with a glycogen breakdown pathway. In particular embodiments, said one or more polynucleotides encode glycogen phosphorylase (GlgP), glycogen isoamylase (GlgX), glucanotransferase (MalQ), phosphoglucomutase (Pgm), glucokinase (Glk), and/or phosphoglucose isomerase (Pgi), or a functional fragment or variant thereof. Pgm, Glk, and Pgi are bidirectional enzymes that can promote glycogen synthesis or breakdown depending on conditions.

### Aldehyde Decarbonylases

Certain embodiments include photosynthetic microorganism having reduced expression of one or more aldehyde decarbonylases. As used herein, an “aldehyde decarbonylase” is capable of catalyzing the conversion of an acyl aldehyde (or fatty aldehyde) to an alkane or alkene (see FIG. 2). Included are members of the ferritin-like or ribonucleotide reductase-like family of nonheme diiron enzymes (see, e.g., Stubbe et al., Trends Biochem Sci. 23:438-43, 1998). According to one non-limiting theory, because the aldehyde decarbonylase encoded by PCC7942_orf1593 or PCC6803_orfsII0208 (from Synechostis sp. PCC6803) utilizes acyl aldehyde as a substrate for alkane or alkene production, reducing expression of this protein may further increase yields of free fatty acids by shunting acyl aldehydes (produced by acyl-ACP reductase) away from an alkane-producing pathway, and towards a fatty acid-producing pathway. PCC7942_orf1593 and PCC6803_orfsII0208 orthologs can be found, for example, in N. punctiforme PCC73102, Thermosynechococcus elongatus BP-1, Synechococcus sp. Ja-3-3AB, P. marinus MIT9313, P. marinus NATL2A, and Synechococcus sp. RS 9117, the latter having at least two paralogs (RS 9117-1 and -2). Included are mutations (e.g., genomic) that reduce or eliminate the enzymatic activity of one or more endogenous aldehyde decarbonylases. Also included are full or partial deletions of an endogenous gene encoding an aldehyde decarbonylase.

### Acyl-ACP Synthetases (Aas)

Acyl-ACP synthetases (Aas) catalyze the ATP-dependent acylation of the thiol of acyl carrier protein (ACP) with fatty acids, including those fatty acids having chain lengths from about C4 to C18. In Cyanobacteria, among other functions, Aas enzymes not only directly incorporate exogenous fatty acids from the culture medium into other lipids, but also play a role in the recycling of acyl chains from lipid membranes. Deletion of Aas in cyanobacteria can lead to secretion of free fatty acids into the culture medium. See, e.g., Kaczmarzyk and Fulda, Plant Physiology 152:1598-1610, 2010.

According to one non-limiting theory, an endogenous aldhehyde dehydrogenase may be acting on the excess acyl-aldehydes generated by overexpressed orf1594 and converting them to free fatty acids. The normal role of such a dehydrogenase might involve removing or otherwise dealing with damaged lipids. In this scenario, it is then likely that the Aas gene product recycles these free fatty acids by ligating them to ACP. Accordingly, reducing or eliminating expression of the Aas gene product might ultimately increase production of fatty acids, by reducing or preventing their transfer to ACP.

Included are mutations (e.g., genomic) that reduce or eliminate the enzymatic activity of one or more endogenous acyl-ACP synthetases (or synthases). Also included are full or partial deletions of an endogenous gene encoding an acyl-ACP synthetase. SEQ ID NOS:43 and 44, respectively, provide the nucleotide and polypeptide sequences of an exemplary Aas from Synechococcus elongatus PCC 7942.

Other embodiments may overexpress one or more Aas polypeptides described herein and known in the art. According to one non-limiting theory, overexpression of Aas in combination with overexpression of ACP leads to increased TAG production in DGAT-expressing strains, for example, by boosting acyl-ACP levels. Overexpression of Aas in optional combination with overexpression of ACP may likewise increase wax ester formation, for example, when combined with overexpression of one or more alcohol dehydrogenase(s) and wax ester synthase(s). Certain embodiments therefore include modified photosynthetic microorganisms comprising overexpressed Aas polypeptide(s), optionally in combination with overexpressed ACP polypeptide(s), especially when combined with overexpression of alcohol dehydrogenase, acyl-ACP reductase (e.g., orf1594), and wax ester synthase (e.g., aDGAT).

### Aldehyde Dehydrogenases

Embodiments of the present invention optionally include one or more aldehyde dehydrogenases. Examples of aldehyde dehydrogenases include enzymes capable of using acyl aldehydes (e.g., nonyl-aldehyde, C16 fatty aldehyde) as a substrate, and converting them into fatty acids. In certain embodiments, the aldehyde dehydrogenase is naturally-occurring or endogenous to the modified microorganism, and is sufficient to convert increased acyl aldehydes (produced by acyl-ACP reductase) into fatty acids, and thereby contribute to increased fatty acid production and overall satisfactory growth characteristics.

In certain embodiments, the aldehyde dehydrogenase can be overexpressed, for example, by recombinantly introducing a polynucleotide that encodes the enzyme, increasing expression of an endogenous enzyme, or both. An aldehyde dehydrogenase can be overexpressed in a strain that already expresses a naturally-occurring or endogenous enzyme, to further increase fatty acid production of an acyl-ACP reductase over-expressing strain and/or improve its growth characteristics, relative, for example, to an acyl-ACP reductase-overexpressing strain that only expresses endogenous aldehyde dehydrogenase. An aldehyde dehydrogenase can also be expressed or overexpressed in a strain that does not have a naturally occurring aldehyde dehydrogenase of that type, e.g., it does not naturally express an enzyme that is capable of efficiently converting acyl aldehydes such as nonyl-aldehyde into fatty acids.

In these and related embodiments, expression or overexpression of an aldehyde dehydrogenase may increase shunting of acyl aldehydes towards production of fatty acids, and away from production of other products such as alkanes. It may also reduce accumulation of potentially toxic acyl aldehydes, and thereby improve growth characteristics of a modified microorganism.

One exemplary aldehyde dehydrogenase is encoded by orf0489 of Synechococcus elongatus PCC7942. Also included are homologs or paralogs thereof, functional equivalents thereof, and fragments or variants thereofs. Functional equivalents can include aldehyde dehydrogenases with the ability to efficiently convert acyl aldehydes (e.g., nonyl-aldehyde) into fatty acids. In specific embodiments, the aldehyde dehydrogenase has the amino acid sequence of SEQ ID NO:103 (encoded by the polynucleotide sequence of SEQ ID NO:102), or an active fragment or variant of this sequence.

### Alcohol Dehydrogenases

Embodiments of the present invention optionally include one or more alcohol dehydrogenases. Examples of alcohol dehydrogenases include those capable of using acyl or fatty aldehydes (e.g., one or more of nonyl-aldehyde, C₁₂, C₁₄, C₁₆, C₁₈, C₂₀ fatty aldehyde) as a substrate, and converting them into fatty alcohols. Specific examples include long-chain alcohol dehydrogenases, capable of using long-chain aldehydes (e.g., C₁₆, C₁₈, C₂₀) as substrates. In certain embodiments, the alcohol dehydrogenase is naturally-occurring or endogenous to the modified microorganism, and is sufficient to convert increased acyl aldehydes (produced by acyl-ACP reductase) into fatty alcohols, and thereby contribute to increased wax ester production and overall satisfactory growth characteristics. In certain embodiments, the alcohol dehydrogenase is derived from a microorganism that differs from the one being modified.

In these and related embodiments, expression or overexpression of an alcohol dehydrogenase may increase shunting of acyl aldehydes towards production of fatty alcohols, and away from production of other products such as alkanes, fatty acids, or triglycerides. When combined with one or more wax ester synthases, such as DGAT or other enzyme having wax ester synthase activity (e.g., the ability to convert fatty alcohols into wax esters), alcohol dehydrogenases may contribute to production of wax esters. They may also reduce accumulation of potentially toxic acyl aldehydes, and thereby improve growth characteristics of a modified microorganism.

Non-limiting examples of alcohol dehydrogenases include those encoded by slr1192 of Synechocystis sp. PCC6803 (SEQ ID NOS:104-105) and ACIAD3612 of Acinetobacter baylyi (SEQ ID NOS:106-107). Also included are homologs or paralogs thereof, functional equivalents thereof, and fragments or variants thereofs. Functional equivalents can include alcohol dehydrogenases with the ability to efficiently convert acyl aldehydes (e.g., C₆, C₈, C₁₀, C₁₂, C₁₄, C₁₆, C₁₈, C₂₀ aldehydes) into fatty alcohols. Specific examples of functional equivalents include long-chain alcohol dehydrogenases, having the ability to utilize long-chain aldehydes (e.g., C₁₆, C₁₈, C₂₀) as substrates. In particular embodiments, the alcohol dehydrogenase has the amino acid sequence of SEQ ID NO:105 (encoded by the polynucleotide sequence of SEQ ID NO:104), or an active fragment or variant of this sequence. In some embodiments, the alcohol dehydrogenase has the amino acid sequence of SEQ ID NO:107 (encoded by the polynucleotide sequence of SEQ ID NO:106), or an active fragment or variant of this sequence.

### Polynucleotides and Vectors

Certain modified photosynthetic microorganisms (e.g., Cyanobacteria) of the present invention comprise one or more introduced polynucleotides encoding an acyl-ACP reductase. These and related modified microorganisms (e.g., those containing only an introduced promoter upstream of an endogenous acyl-ACP reductase gene) may optionally comprise one or more introduced polynucleotides encoding a lipid or fatty acid biosynthesis protein such as an ACP or an ACCase, and/or one or more introduced polynucleotides encoding a polypeptide associated with glycogen breakdown, including functional variants and fragment thereof. Accordingly, the present invention utilizes isolated polynucleotides that encode acyl-ACP reductases, ACPs, ACCases, DGATs, acyl-CoA synthetases, and the various glycogen breakdown pathway proteins, in addition to nucleotide sequences that encode any functional naturally-occurring variants or fragments (i.e., allelic variants, orthologs, splice variants) or non-naturally occurring variants or fragments of these native enzymes (i.e., optimized by engineering), as well as compositions comprising such polynucleotides, including, e.g., cloning and expression vectors.

As used herein, the terms “DNA” and “polynucleotide” and “nucleic acid” refer to a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

As will be understood by those skilled in the art, the polynucleotide sequences of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

As will be recognized by the skilled artisan, polynucleotides may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials.

Polynucleotides may comprise a native sequence (e.g., an endogenous sequence that encodes an acyl-ACP reductase, an ACP, a diacylglycerol acyltransferase, a fatty acyl-CoA synthetase, a glycogen breakdown protein, an acetyl-CoA carboxylase, aldehyde dehydrogenase, or a portion thereof) or may comprise a variant, or a biological functional equivalent of such a sequence. Polynucleotide variants may contain one or more substitutions, additions, deletions and/or insertions, as further described below, preferably such that the enzymatic activity of the encoded polypeptide is not substantially diminished relative to the unmodified polypeptide. The effect on the enzymatic activity of the encoded polypeptide may generally be assessed as described herein.

In certain embodiments, a modified photosynthetic microorganism comprises one or more polynucleotides encoding one or more acyl-ACP reductase polypeptides. Exemplary acyl-ACP reductase nucleotide sequences include orf1954 from Synechococcus elongatus PCC7942 (SEQ ID NO:1), and orfsII0209 from Synechocystis sp. PCC6803 (SEQ ID NO:3).

In certain embodiments, a modified photosynthetic microorganism comprises one or more polynucleotides encoding one or more acyl carrier proteins (ACP). Exemplary ACP nucleotide sequences include SEQ ID NO:5 from Synechococcus elongatus PCC7942, SEQ ID NOS:7, 9, and 11 from Acinetobacter sp. ADP1, and SEQ ID NO:13 from Spinacia oleracea.

In certain embodiments of the present invention, a polynucleotide encodes an acetyl-CoA carboxylase (ACCase) comprising or consisting of a polypeptide sequence set forth in any of SEQ ID NOs:55, 45, 46, 47, 48 or 49, or a fragment or variant thereof. In particular embodiments, a ACCase polynucleotide comprises or consists of a polynucleotide sequence set forth in any of SEQ ID NOs:56, 57, 50, 51, 52, 53 or 54, or a fragment or variant thereof. SEQ ID NO:55 is the sequence of Saccharomyces cerevisiae acetyl-CoA carboxylase (yAcc1); and SEQ ID NO:56 is a codon-optimized for expression in Cyanobacteria sequence that encodes yAcc1. SEQ ID NO:45 is Synechococcus sp. PCC 7002 AccA; SEQ ID NO:46 is Synechococcus sp. PCC 7002 AccB; SEQ ID NO:47 is Synechococcus sp. PCC 7002 AccC; and SEQ ID NO:48 is Synechococcus sp. PCC 7002 AccD. SEQ ID NO:50 encodes Synechococcus sp. PCC 7002 AccA; SEQ ID NO:51 encodes Synechococcus sp. PCC 7002 AccB; SEQ ID NO:52 encodes Synechococcus sp. PCC 7002 AccC; and SEQ ID NO:53 encodes Synechococcus sp. PCC 7002 AccD. SEQ ID NO:49 is a Triticum aestivum ACCase; and SEQ ID NO:54 encodes this Triticum aestivum ACCase.

In certain embodiments, a modified photosynthetic microorganism comprises one or more polynucleotides encoding one or more DGAT enzymes. In certain embodiments of the present invention, a polynucleotide encodes a DGAT comprising of consisting of a polypeptide sequence set forth in any one of SEQ ID NOs:58, 59, 60 or 61, or a fragment or variant thereof. SEQ ID NO:58 is the sequence of DGATn; SEQ ID NO: 59 is the sequence of Streptomyces coelicolor DGAT (ScoDGAT or SDGAT); SEQ ID NO:60 is the sequence of Alcanivorax borkumensis DGAT (AboDGAT); and SEQ ID NO:61 is the sequence of DGATd (Acinetobacter baylii sp.). In certain embodiments of the present invention, a DGAT polynucleotide comprises or consists of a polynucleotide sequence set forth in any one of SEQ ID NOs:62, 63, 64, 65 or 66, or a fragment or variant thereof. SEQ ID NO:62 is a codon-optimized for expression in Cyanbacteria sequence that encodes DGATn; SEQ ID NO: 63 has homology to SEQ ID NO:62; SEQ ID NO:64 is a codon-optimized for expression in Cyanobacteria sequence that encodes ScoDGAT; SEQ ID NO:65 is a codon-optimized for expression in Cyanobacteria sequence that encodes AboDGAT; and SEQ ID NO:66 is a codon-optimized for expression in Cyanobacteria sequence that encodes DGATd. DGATn and DGATd correspond to Acinetobacter baylii DGAT and a modified form thereof, which includes two additional amino acid residues immediately following the initiator methionine.

Certain embodiments employ one or more fatty acyl-CoA synthetase encoding polynucleotide sequences. One exemplary fatty acyl-CoA synthetase includes the FadD gene from E. coli (SEQ ID NO:16) which encodes a fatty acyl-CoA synthetase having substrate specificity for medium and long chain fatty acids. Other exemplary fatty acyl-CoA synthetases include those derived from S. cerevisiae; for example, the Faa1p coding sequence is set forth in SEQ ID NO:18, the Faa2p coding sequence is set forth in SEQ ID NO:20, and the Faa3p is set forth in SEQ ID NO:22. SEQ ID NO:22 is codon-optimized for expression in S. elongatus PCC7942.

Certain embodiments may employ one or more aldehyde dehydrogenase encoding polynucleotide sequences. One exemplary aldehyde dehydrogenase is orf0489 of Synechococcus elongatus PCC7942 (SEQ ID NO:102). Also included are active fragments or variants of this sequence.

Certain embodiments may employ one or more alcohol dehydrogenase encoding polynucleotide sequences. Exemplary alcohol dehydrogenases include slr1192 of Synechocystis sp. PCC6803 (SEQ ID NO:104) and ACIAD3612 from Acinetobacter baylyi (SEQ ID NO:106).

In certain embodiments of the present invention, a modified photosynthetic microorganism comprise one or more polynucleotides encoding one or more polypeptides associated with a glycogen breakdown, or a fragment or variant thereof. In particular embodiments, the one or more polypeptides are glycogen phosphorylase (GlgP), glycogen isoamylase (GlgX), glucanotransferase (MalQ), phosphoglucomutase (Pgm), glucokinase (Glk), and/or phosphoglucose isomerase (Pgi), or a functional fragment or variant thereof. A representative glgP polynucleotide sequence is provided in SEQ ID NO:67, and a representative GlgP polypeptide sequence is provided in SEQ ID NO:68. A representative glgX polynucleotide sequence is provided in SEQ ID NO:69, and a representative GlgX polypeptide sequence is provided in SEQ ID NO:70. A representative malQ polynucleotide sequence is provided in SEQ ID NO:71, and a representative MalQ polypeptide sequence is provide in SEQ ID NO:72. A representative phosphoglucomutase (pgm) polynucleotide sequence is provided in SEQ ID NO:24, and a representative phosphoglucomutase (Pgm) polypeptide sequence is provided in SEQ ID NO:73, with others provided infra (SEQ ID NOs:25, 26, 74-81). A representative glk polynucleotide sequence is provided in SEQ ID NO:82, and a representative Glk polypeptide sequence is provided in SEQ ID NO:83. A representative pgi polynucleotide sequence is provided in SEQ ID NO:84, and a representative Pgi polypeptide sequence is provided in SEQ ID NO:85. In particular embodiments of the present invention, a polynucleotide comprises one of these polynucleotide sequences, or a fragment or variant thereof, or encodes one of these polypeptide sequences, or a fragment or variant thereof.

In certain embodiments, the present invention provides isolated polynucleotides comprising various lengths of contiguous stretches of sequence identical to or complementary to an acyl-ACP reductase, acyl carrier protein (ACP), acetyl-CoA carboxylase (ACCase), glycogen breakdown protein, diacylglycerol acyltransferase (DGAT), aldehyde dehydrogenase, or fatty acyl-CoA synthetase, wherein the isolated polynucleotides encode a biologically active, truncated enzyme.

Exemplary nucleotide sequences that encode the proteins and enzymes of the application encompass full-length acyl-ACP reductases, ACPs, glycyogen breakdown proteins, ACCases, DGATs, fatty acyl-CoA synthetases, aldehyde dehydrogenases, alcohol dehydrogenases, as well as portions of the full-length or substantially full-length nucleotide sequences of these genes or their transcripts or DNA copies of these transcripts. Portions of a nucleotide sequence may encode polypeptide portions or segments that retain the biological activity of the reference polypeptide. A portion of a nucleotide sequence that encodes a biologically active fragment of an enzyme provided herein may encode at least about 20, 21, 22, 23, 24, 25, 30, 40, 50, 60, 70, 80, 90, 100, 120, 150, 200, 300, 400, 500, 600, or more contiguous amino acid residues, almost up to the total number of amino acids present in a full-length enzyme. It will be readily understood that “intermediate lengths,” in this context and in all other contexts used herein, means any length between the quoted values, such as 101, 102, 103, etc.; 151, 152, 153, etc.; 201, 202, 203, etc.

The polynucleotides of the present invention, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a polynucleotide fragment of almost any length may be employed, with the total length preferably being limited by the ease of preparation and use in the intended recombinant DNA protocol.

The invention also contemplates variants of the nucleotide sequences of the acyl-ACP reductases, ACPs, DGATs, glycogen breakdown proteins, fatty acyl-CoA synthetases, aldehyde dehydrogenases, alcohol dehydrogenases, and ACCases utilized according to methods and compositions provided herein. Nucleic acid variants can be naturally-occurring, such as allelic variants (same locus), homologs (different locus), and orthologs (different organism) or can be non naturally-occurring. Naturally occurring variants such as these can be identified and isolated using well-known molecular biology techniques including, for example, various polymerase chain reaction (PCR) and hybridization-based techniques as known in the art. Naturally occurring variants can be isolated from any organism that encodes one or more genes having an acyl-ACP reductase activity, an ACP activity, glycogen breakdown protein, DGAT, fatty acyl-CoA synthetase, aldehyde dehydrogenase, and/or an acetyl-CoA carboxylase activity. Embodiments of the present invention, therefore, encompass Cyanobacteria comprising such naturally occurring polynucleotide variants.

Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. In certain aspects, non-naturally occurring variants may have been optimized for use in Cyanobacteria, such as by engineering and screening the enzymes for increased activity, stability, or any other desirable feature. The variations can produce both conservative and non-conservative amino acid substitutions (as compared to the originally encoded product). For nucleotide sequences, conservative variants include those sequences that, because of the degeneracy of the genetic code, encode the amino acid sequence of a reference polypeptide. Variant nucleotide sequences also include synthetically derived nucleotide sequences, such as those generated, for example, by using site-directed mutagenesis but which still encode a biologically active polypeptide, such as a polypeptide having an acyl-ACP reductase activity, an ACP activity, glycogen breakdown activity, DGAT activity, fatty acyl-CoA synthetase activity, aldehyde dehydrogenase activity, alcohol dehydrogenase, and/or an acetyl-CoA carboxylase activity. Generally, variants of a particular reference nucleotide sequence will have at least about 30%, 40% 50%, 55%, 60%, 65%, 70%, generally at least about 75%, 80%, 85%, 90%, 95% or 98% or more sequence identity to that particular nucleotide sequence as determined by sequence alignment programs described elsewhere herein using default parameters.

Known acyl-ACP reductase, ACP, glycogen breakdown protein, DGAT, fatty acyl-CoA synthetase, aldehyde dehydrogenase, alcohol dehydrogenase, and/or a acetyl-CoA carboxylase nucleotide sequences can be used to isolate corresponding sequences and alleles from other organisms, particularly other microorganisms. Methods are readily available in the art for the hybridization of nucleic acid sequences. Coding sequences from other organisms may be isolated according to well known techniques based on their sequence identity with the coding sequences set forth herein. In these techniques all or part of the known coding sequence is used as a probe which selectively hybridizes to other reference coding sequences present in a population of cloned genomic DNA fragments or cDNA fragments (i.e., genomic or cDNA libraries) from a chosen organism.

Accordingly, the present invention also contemplates polynucleotides that hybridize to reference nucleotide sequences, or to their complements, under stringency conditions described below. As used herein, the term “hybridizes under low stringency, medium stringency, high stringency, or very high stringency conditions” describes conditions for hybridization and washing. Guidance for performing hybridization reactions can be found in Ausubel et al., (1998, supra), Sections 6.3.1-6.3.6. Aqueous and non-aqueous methods are described in that reference and either can be used.

Reference herein to “low stringency” conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6× sodium chloride/sodium citrate (SSC) at about 45° C., followed by two washes in 0.2×SSC, 0.1% SDS at least at 50° C. (the temperature of the washes can be increased to 55° C. for low stringency conditions).

“Medium stringency” conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C.

“High stringency” conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In certain embodiments, a acyl-ACP reductase, ACP, glycogen breakdown protein, aldehyde dehydrogenase, alcohol dehydrogenase, alcohol dehydrogenase, and/or acetyl-CoA carboxylase enzyme (or other enzyme described herein) is encoded by a polynucleotide that hybridizes to a disclosed nucleotide sequence under very high stringency conditions. One embodiment of very high stringency conditions includes hybridizing in 0.5 M sodium phosphate, 7% SDS at 65° C., followed by one or more washes in 0.2×SSC, 1% SDS at 65° C.

Other stringency conditions are well known in the art and the skilled artisan will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8).

In general, the Tₘ of a perfectly matched duplex of DNA may be predicted as an approximation by the formula: Tₘ=81.5+16.6 (log₁₀ M)+0.41 (% G+C)−0.63 (% formamide)−(600/length) wherein: M is the concentration of Na⁺, preferably in the range of 0.01 molar to 0.4 molar; % G+C is the sum of guano sine and cytosine bases as a percentage of the total number of bases, within the range between 30% and 75% G+C; % formamide is the percent formamide concentration by volume; length is the number of base pairs in the DNA duplex. The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In one example of a hybridization procedure, a membrane (e.g., a nitrocellulose membrane or a nylon membrane) containing immobilized DNA is hybridized overnight at 42° C. in a hybridization buffer (50% deionizer formamide, 5×SSC, 5×Reinhardt's solution (0.1% fecal, 0.1% polyvinylpyrollidone and 0.1% bovine serum albumin), 0.1% SDS and 200 mg/mL denatured salmon sperm DNA) containing a labeled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.

Polynucleotides and fusions thereof may be prepared, manipulated and/or expressed using any of a variety of well established techniques known and available in the art. For example, polynucleotide sequences which encode polypeptides of the invention, or fusion proteins or functional equivalents thereof, may be used in recombinant DNA molecules to direct expression of a triglyceride or lipid biosynthesis enzyme in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence. Such nucleotides are typically referred to as “codon-optimized.”

Moreover, the polynucleotide sequences of the present invention can be engineered using methods generally known in the art in order to alter polypeptide encoding sequences for a variety of reasons, including but not limited to, alterations which modify the cloning, processing, expression and/or activity of the gene product.

In order to express a desired polypeptide, a nucleotide sequence encoding the polypeptide, or a functional equivalent, may be inserted into appropriate expression vector, i.e., a vector that contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. In certain embodiments, the polynucleotides of the present invention may be introduced and expressed in Cyanobacterial systems. As such, the present invention contemplates the use of vector and plasmid systems having regulatory sequences (e.g., promoters and enhancers) that are suitable for use in various Cyanobacteria (see, e.g., Koksharova et al. Applied Microbiol Biotechnol 58:123-37, 2002). For example, the promiscuous RSF1010 plasmid provides autonomous replication in several Cyanobacteria of the genera Synechocystis and Synechococcus (see, e.g., Mermet-Bouvier et al., Curr Microbiol 26:323-327, 1993). As another example, the pFC1 expression vector is based on the promiscuous plasmid RSF1010. pFC1 harbors the lambda cl857 repressor-encoding gene and pR promoter, followed by the lambda cro ribosome-binding site and ATG translation initiation codon (see, e.g., Mermet-Bouvier et al., Curr Microbiol 28:145-148, 1994). The latter is located within the unique NdeI restriction site (CATATG) of pFC1 and can be exposed after cleavage with this enzyme for in-frame fusion with the protein-coding sequence to be expressed.

The “control elements” or “regulatory sequences” present in an expression vector (or employed separately) are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. Generally, it is well-known that strong E. coli promoters work well in Cyanobacteria. Also, when cloning in Cyanobacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. Other vectors containing IPTG inducible promoters, such as pAM1579 and pAM2991trc, may be utilized according to the present invention.

Certain embodiments may employ a temperature inducible system or temperature inducible regulatory sequences (e.g., promoters, enhancers, repressors). As one example, an operon with the bacterial phage left-ward promoter (P_{L}) and a temperature sensitive repressor gene C1857 may be employed to produce a temperature inducible system for producing fatty acids and/or triglycerides in Cyanobacteria (see, e.g., U.S. Pat. No. 6,306,639, herein incorporated by reference). It is believed that at a non-permissible temperature (low temperature, 30 degrees Celsius), the repressor binds to the operator sequence, and thus prevents RNA polymerase from initiating transcription at the P_{L} promoter. Therefore, the expression of encoded gene or genes is repressed. When the cell culture is transferred to a permissible temperature (37-42 degrees Celsius), the repressor cannot bind to the operator. Under these conditions, RNA polymerase can initiate the transcription of the encoded gene or genes.

In Cyanobacterial systems, a number of expression vectors or regulatory sequences may be selected depending upon the use intended for the expressed polypeptide. When large quantities are needed, vectors or regulatory sequences which direct high level expression of encoded proteins may be used. For example, overexpression of ACCase enzymes may be utilized to increase fatty acid biosynthesis. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST).

Certain embodiments may employ Cyanobacterial promoters or regulatory operons. In certain embodiments, a promoter may comprise an rbcLS operon of Synechococcus, as described, for example, in Ronen-Tarazi et al. (Plant Physiology 18:1461-1469, 1995), or a cpc operon of Synechocystis sp. strain PCC 6714, as described, for example, in Imashimizu et al. (J Bacteriol. 185:6477-80, 2003). In certain embodiments, the tRNApro gene from Synechococcus may also be utilized as a promoter, as described in Chungjatupornchai et al. (Curr Microbiol. 38:210-216, 1999). Certain embodiments may employ the nirA promoter from Synechococcus sp. strain PCC7942, which is repressed by ammonium and induced by nitrite (see, e.g., Maeda et al., J. Bacteriol. 180:4080-4088, 1998; and Qi et al., Applied and Environmental Microbiology 71:5678-5684, 2005). The efficiency of expression may be enhanced by the inclusion of enhancers which are appropriate for the particular Cyanobacterial cell system which is used, such as those described in the literature.

In certain embodiments, expression vectors or introduced promoters utilized to overexpress an exogenous or endogenous acyl-ACP reductase, ACP, DGAT, fatty acyl-CoA synthetase, glycogen breakdown protein, aldehyde dehydrogenase, alcohol dehydrogenase, and/or acetyl-CoA carboxylase, or fragment or variant thereof, comprise a weak promoter under non-inducible conditions, e.g., to avoid toxic effects of long-term overexpression of any of these polypeptides. One example of such a vector for use in Cyanobacteria is the pBAD vector system. Expression levels from any given promoter may be determined, e.g., by performing quantitative polymerase chain reaction (qPCR) to determine the amount of transcript or mRNA produced by a promoter, e.g., before and after induction. In certain instances, a weak promoter is defined as a promoter that has a basal level of expression of a gene or transcript of interest, in the absence of inducer, that is ≦2.0% of the expression level produced by the promoter of the rnpB gene in S. elongatus PCC7942. In other embodiments, a weak promoter is defined as a promoter that has a basal level of expression of a gene or transcript of interest, in the absence of inducer, that is ≦5.0% of the expression level produced by the promoter of the rnpB gene in S. elongatus PCC7942.

It will be apparent that further to their use in vectors, any of the regulatory elements described herein (e.g., promoters, enhancers, repressors, ribosome binding sites, transcription termination sites) may be introduced directly into the genome of a photosynthetic microorganism (e.g., Cyanobacterium), typically in a region surrounding (e.g., upstream or downstream of) an endogenous or naturally-occurring gene such as an acyl-ACP reductase (e.g., orf1594 in Synechococcus elongatus), an ACP, or an ACCase, to regulate expression (e.g., facilitate overexpression) of that gene.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983). The presence of a desired polynucleotide, such as an acyl-ACP reductase, ACP, glycogen breakdown protein, and/or an acetyl-CoA carboxylase encoding polypeptide, may also be confirmed by PCR.

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Cyanobacterial host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct localization of the encoded polypeptide to a desired site within the cell. Other recombinant constructions may be used to join sequences encoding a polypeptide of interest to nucleotide sequence encoding a polypeptide domain which will direct secretion of the encoded protein.

In particular embodiments of the present invention, a modified photosynthetic microorganism of the present invention has reduced expression of one or more genes selected from glucose-1-phosphate adenyltransferase (glgC), phosphoglucomutase (pgm), and/or glycogen synthase (glgA). In particular embodiments, the modified photosynthetic microorganism comprises a mutation of one or more of these genes. Specific glgC, pgm, and glgA sequences may be mutated or modified, or targeted to reduce expression.

Examples of such glgC polynucleotide sequences are provided in SEQ ID NOs:28 (Synechocystis sp. PCC6803), 34 (Nostoc sp. PCC 7120), 33 (Anabaena variabilis), 32 (Trichodesmium erythraeum IMS 101), 27 (Synechococcus elongatus PCC7942), 30 (Synechococcus sp. WH8102), 31 (Synechococcus sp. RCC 307), and 29 (Synechococcus sp. PCC 7002), which respectively encode GlgC polypeptides having sequences set forth in SEQ ID NOs: 86, 87, 88, 89, 90, 91, 92, and 93.

Examples of such pgm polynucleotide sequences are provided in SEQ ID NOs: 25 (Synechocystis sp. PCC6803), 75 (Synechococcus elongatus PCC7942), 26 (Synechococcus sp. WH8102), 78 (Synechococcus RCC307), and 80 (Synechococcus 7002), which respectively encode Pgm polypeptides having sequences set forth in SEQ ID NOs:74, 76, 77, 79 and 81.

Examples of such glgA polynucleotide sequences are provided in SEQ ID NOs:36 (Synechocystis sp. PCC6803), 42 (Nostoc sp. PCC 7120), 41 (Anabaena variabilis), 40 (Trichodesmium erythraeum IMS 101), 35 (Synechococcus elongatus PCC7942), 38 (Synechococcus sp. WH8102), 39 (Synechococcus sp. RCC 307), and 37 (Synechococcus sp. PCC 7002), which respectively encode GlgA polypeptides having sequences set forth in SEQ ID NOs:94, 95, 96, 97, 98, 99, 100 and 101.

In particular embodiments of the present invention, a modified photosynthetic microorganism of the present invention has reduced expression of one or more aldehyde decarbonylases. One example of an aldehyde decarbonylase is encoded by orf1953 in S. elongatus PCC7942. Another example is an aldehyde decarbonylase encoded by orfsII0208 in Synechocystis sp. PCC6803. In particular embodiments, a modified photosynthetic microorganism of the present invention has reduced expression of one or more acyl-ACP synthetases (Aas). One example is encoded by Aas of S. elongatus PCC7942.

### Polypeptides

The present invention contemplates the use of modified photosynthetic microorganisms, e.g., Cyanobacteria, comprising one or more overexpressed acyl-ACP reductase polypeptides. Specific embodiments of the present invention contemplate the use of modified photosynthetic microorganisms, e.g., Cyanobacteria, comprising an overexpressed acyl-ACP reductase in combination one or more additional introduced or overexpressed polypeptides, including those associated with fatty acid or triglyceride biosynthesis (e.g., ACP, ACCase, DGAT, aldehyde dehydrogenase, alcohol dehydrogenase, fatty acyl-CoA synthetase) and/or glycogen breakdown pathways. Also included are truncated, variant and/or modified polypeptides thereof, for increasing lipid or fatty acid production in said modified photosynthetic microorganism.

In certain embodiments, an acyl-ACP reductase comprises or consists of the exemplary polypeptide sequence of SEQ ID NO:2, encoded by orf1594 from Synechococcus elongatus PCC7942, including active variants or fragments thereof. In some embodiments, an acyl-ACP reductase comprises or consists of the exemplary polypeptide sequence of SEQ ID NO:4, encoded by orfsII0209 from Synechocystis sp. PCC6803, including active variants or fragments thereof.

In certain embodiments, an acyl carrier protein (ACP) comprises or consists of the exemplary ACP polypeptide sequences include SEQ ID NO:6 from Synechococcus elongatus PCC7942, SEQ ID NOS:8, 10, and 12 from Acinetobacter sp. ADP1, or SEQ ID NO:14 from Spinacia oleracea.

In certain embodiments of the present invention, an acetyl-CoA carboxylase (ACCase) polypeptide comprises or consists of a polypeptide sequence set forth in any of SEQ ID NOs:55, 45, 46, 48 or 49, or a fragment or variant thereof. In particular embodiments, an ACCase polypeptide is encoded by a polynucleotide sequence set forth in any of SEQ ID NOs:56, 57, 50, 51, 52, 53 or 54, or a fragment or variant thereof. SEQ ID NO:55 is the sequence of Saccharomyces cerevisiae acetyl-CoA carboxylase (yAcc1); and SEQ ID NO:56 is a codon-optimized for expression in Cyanobacteria sequence that encodes yAcc1. SEQ ID NO:45 is Synechococcus sp. PCC 7002 AccA; SEQ ID NO:46 is Synechococcus sp. PCC 7002 AccB; SEQ ID NO:47 is Synechococcus sp. PCC 7002 AccC; and SEQ ID NO:48 is Synechococcus sp. PCC 7002 AccD. SEQ ID NO:50 encodes Synechococcus sp. PCC 7002 AccA; SEQ ID NO:51 encodes Synechococcus sp. PCC 7002 AccB; SEQ ID NO:52 encodes Synechococcus sp. PCC 7002 AccC; and SEQ ID NO:53 encodes Synechococcus sp. PCC 7002 AccD. SEQ ID NO:49 is a T. aestivum ACCase; and SEQ ID NO:54 encodes this Triticum aestivum ACCase.

In certain embodiments of the present invention, a DGAT polypeptide comprises or consists of a polypeptide sequence set forth in any one of SEQ ID NOs:58, 59, 60 Or 61, or a fragment or variant thereof. SEQ ID NO:58 is the sequence of DGATn; SEQ ID NO:59 is the sequence of Streptomyces coelicolor DGAT (ScoDGAT or SDGAT); SEQ ID NO:60 is the sequence of Alcanivorax borkumensis DGAT (AboDGAT); and SEQ ID NO:61 is the sequence of DGATd. In certain embodiments of the present invention, a DGAT polypeptide is encoded by a polynucleotide sequence set forth in any one of SEQ ID NOs:62, 63, 64, 65 or 66, or a fragment or variant thereof. SEQ ID NO:62 is a codon-optimized for expression in Cyanbacteria sequence that encodes DGATn; SEQ ID NO:63 has homology to SEQ ID NO:62; SEQ ID NO:64 is a codon-optimized for expression in Cyanobacteria sequence that encodes ScoDGAT; SEQ ID NO:65 is a codon-optimized for expression in Cyanobacteria sequence that encodes AboDGAT; and SEQ ID NO:66 is a codon-optimized for expression in Cyanobacteria sequence that encodes DGATd.

Certain embodiments employ one or more fatty acyl-CoA synthetase polypeptides. One exemplary fatty acyl-CoA synthetase includes the polypeptide sequence of the FadD gene from E. coli (SEQ ID NO:17), a fatty acyl-CoA synthetase having substrate specificity for medium and long chain fatty acids. Other exemplary fatty acyl-CoA synthetases include those derived from S. cerevisiae; for example, the Faa1p polypeptide sequence is set forth in SEQ ID NO:19, the Faa2p polypeptide sequence is set forth in SEQ ID NO:21, and the Faa3p polypeptide sequence is set forth in SEQ ID NO:23.

In certain embodiments, the aldehyde dehydrogenase is encoded by orf0489 of Synechococcus elongatus PCC7942, and has the amino acid sequence of SEQ ID NO:103. Also included are active fragments or variants of this sequence, and functional equivalents.

In certain embodiments, the alcohol dehydrogenase is encoded by slr1192 of Synechocystis sp. PCC6803, and has the amino acid sequence of SEQ ID NO:105. In some embodiments, the alcohol dehydrogenase is encoded by ACIAD3612 of Acinetobacter baylyi, and has the amino acid sequence of SEQ ID NO:107. Also included are active fragments or variants of this sequence, and functional equivalents.

Variant proteins encompassed by the present application are biologically active, that is, they continue to possess the enzymatic activity of a reference polypeptide. Such variants may result from, for example, genetic polymorphism or from human manipulation. Biologically active variants of a reference polypeptide will have at least 40%, 50%, 60%, 70%, generally at least 75%, 80%, 85%, usually about 90% to 95% or more, and typically about 97% or 98% or more sequence similarity or identity to the amino acid sequence for a reference protein as determined by sequence alignment programs described elsewhere herein using default parameters. A biologically active variant of a reference polypeptide may differ from that protein generally by as much 200, 100, 50 or 20 amino acid residues or suitably by as few as 1-15 amino acid residues, as few as 1-10, such as 6-10, as few as 5, as few as 4, 3, 2, or even 1 amino acid residue. In some embodiments, a variant polypeptide differs from the reference sequences referred to herein (see, e.g., the Sequence Listing) by at least one but by less than 15, 10 or 5 amino acid residues. In other embodiments, it differs from the reference sequences by at least one residue but less than 20%, 15%, 10% or 5% of the residues.

A reference polypeptide may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (“Molecular Biology of the Gene”, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of reference polypeptides. Recursive ensemble mutagenesis (REM), a technique which enhances the frequency of functional mutants in the libraries, can be used in combination with the screening assays to identify polypeptide variants (Arkin and Yourvan (1992) Proc. Natl. Acad. Sci. USA 89: 7811-7815; Delgrave et al., (1993) Protein Engineering, 6: 327-331). Conservative substitutions, such as exchanging one amino acid with another having similar properties, may be desirable as discussed in more detail below.

Polypeptide variants may contain conservative amino acid substitutions at various locations along their sequence, as compared to a reference amino acid sequence. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al., (1978), A model of evolutionary change in proteins. Matrices for determining distance relationships In M. O. Dayhoff, (ed.), Atlas of protein sequence and structure, Vol. 5, pp. 345-358, National Biomedical Research Foundation, Washington D.C.; and by Gonnet et al., (Science, 256: 14430-1445, 1992), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behaviour.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always non-aromatic. Dependent on their structural properties, amino acid residues may fall in two or more classes. For the naturally-occurring protein amino acids, sub-classification according to this scheme is presented in Table A.

TABLE A
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine, |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional truncated and/or variant polypeptide can readily be determined by assaying its enzymatic activity, as described herein. Conservative substitutions are shown in Table B under the heading of exemplary substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE B
Exemplary Amino Acid Substitutions

| | | Preferred |
|---|---|---|
| Original Residue | Exemplary Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, | Leu |
| | Norleu | |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, | Leu |
| | Norleu | |

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm.C. Brown Publishers (1993).

Thus, a predicted non-essential amino acid residue in reference polypeptide is typically replaced with another amino acid residue from the same side chain family. Alternatively, mutations can be introduced randomly along all or part of a coding sequence, such as by saturation mutagenesis, and the resultant mutants can be screened for an activity of the parent polypeptide to identify mutants which retain that activity. Following mutagenesis of the coding sequences, the encoded peptide can be expressed recombinantly and the activity of the peptide can be determined. A “non-essential” amino acid residue is a residue that can be altered from the wild-type sequence of an embodiment polypeptide without abolishing or substantially altering one or more of its activities. Suitably, the alteration does not substantially abolish one of these activities, for example, the activity is at least 20%, 40%, 60%, 70% or 80% 100%, 500%, 1000% or more of wild-type. An “essential” amino acid residue is a residue that, when altered from the wild-type sequence of a reference polypeptide, results in abolition of an activity of the parent molecule such that less than 20% of the wild-type activity is present. For example, such essential amino acid residues may include those that are conserved in acyl-ACP reductases, ACPs, glycogen breakdown polypeptides, DGATs, fatty acyl-CoA synthetases, aldehyde dehydrogenases, alcohol dehydrogenases, and/or acetyl-CoA carboxylase polypeptides across different species, including those sequences that are conserved in the enzymatic sites of polypeptides from various sources.

Accordingly, the present invention also contemplates variants of the naturally-occurring reference polypeptide sequences or their biologically-active fragments, wherein the variants are distinguished from the naturally-occurring sequence by the addition, deletion, or substitution of one or more amino acid residues. In general, variants will display at least about 30, 40, 50, 55, 60, 65, 70, 75, 80, 85, 90, 91, 92, 93, 94, 95, 96, 97, 98, 99% similarity or sequence identity to a reference polypeptide sequence. Moreover, sequences differing from the native or parent sequences by the addition, deletion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100 or more amino acids but which retain the properties of a parent or reference polypeptide sequence are contemplated.

In some embodiments, variant polypeptides differ from an acyl-ACP reductase, ACP, glycogen breakdown polypeptide, DGAT, fatty acyl-CoA synthetase, aldehyde dehydrogenase, alcohol dehydrogenase, and/or acetyl-CoA carboxylase polypeptide sequence by at least one but by less than 50, 40, 30, 20, 15, 10, 8, 6, 5, 4, 3 or 2 amino acid residue(s). In other embodiments, variant polypeptides differ from a reference sequence by at least 1% but less than 20%, 15%, 10% or 5% of the residues. (If this comparison requires alignment, the sequences should be aligned for maximum similarity. “Looped” out sequences from deletions or insertions, or mismatches, are considered differences.)

In certain embodiments, a variant polypeptide includes an amino acid sequence having at least about 50%, 55%, 60%, 65%, 70%, 75%, 80%, 85%, 90%, 91%, 92%, 93%, 94% 95%, 96%, 97%, 98% or more sequence identity or similarity to a corresponding sequence of an acyl-ACP reductase, ACP, glycogen breakdown polypeptide, DGAT, fatty acyl-CoA synthetase, aldehyde dehydrogenase, alcohol dehydrogenase, acetyl-CoA carboxylase reference polypeptide, or other polypeptide described herein, and retains the enzymatic activity of that reference polypeptide.

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch, (1970, J. Mol. Biol. 48: 444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al., (1997, Nucleic Acids Res, 25: 3389-3402). When utilizing BLAST and Gapped BLAST programs, the default parameters of the respective programs (e.g., XBLAST and NBLAST) can be used.

Variants of an acyl-ACP reductase, ACP, glycogen breakdown polypeptide, DGAT, fatty acyl-CoA synthetase, aldehyde dehydrogenase, alcohol dehydrogenase, and/or acetyl-CoA carboxylase reference polypeptide can be identified by screening combinatorial libraries of mutants of a reference polypeptide. Libraries or fragments e.g., N terminal, C terminal, or internal fragments, of protein coding sequence can be used to generate a variegated population of fragments for screening and subsequent selection of variants of a reference polypeptide.

Methods for screening gene products of combinatorial libraries made by point mutation or truncation, and for screening cDNA libraries for gene products having a selected property are known in the art. Such methods are adaptable for rapid screening of the gene libraries generated by combinatorial mutagenesis of polypeptides.

The present invention also contemplates the use of chimeric or fusion proteins for increasing lipid or fatty acid production and/or producing triglycerides. As used herein, a “chimeric protein” or “fusion protein” includes an acyl-ACP reductase, ACP, glycogen breakdown polypeptide, DGAT, fatty acyl-CoA synthetase, aldehyde dehydrogenase, alcohol dehydrogenase, and/or acetyl-CoA carboxylase reference polypeptide, or a polypeptide fragment linked to either another reference polypeptide (e.g., to create multiple fragments), to a non-reference polypeptide, or to both. A “non-reference polypeptide” refers to a “heterologous polypeptide” having an amino acid sequence corresponding to a protein which is different from the reference protein sequence, and which is derived from the same or a different organism. The reference polypeptide of the fusion protein can correspond to all or a portion of a biologically active amino acid sequence. In certain embodiments, a fusion protein includes at least one or two biologically active portions of an acyl-ACP reductase, ACP, glycogen breakdown polypeptide, DGAT, fatty acyl-CoA synthetase, aldehyde dehydrogenase, alcohol dehydrogenase, and/or acetyl-CoA carboxylase protein. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order.

The fusion partner may be designed and included for essentially any desired purpose provided they do not adversely affect the enzymatic activity of the polypeptide. For example, in one embodiment, a fusion partner may comprise a sequence that assists in expressing the protein (an expression enhancer) at higher yields than the native recombinant protein. Other fusion partners may be selected so as to increase the solubility or stability of the protein or to enable the protein to be targeted to desired intracellular compartments.

The fusion protein can include a moiety which has a high affinity for a ligand. For example, the fusion protein can be a GST-fusion protein in which the reference polypeptide sequences are fused to the C-terminus of the GST sequences. Such fusion proteins can facilitate the purification and/or identification of the resulting polypeptide. Alternatively, the fusion protein can be reference polypeptide containing a heterologous signal sequence at its N-terminus. In certain host cells, expression and/or secretion of such proteins can be increased through use of a heterologous signal sequence.

Fusion proteins may generally be prepared using standard techniques. For example, DNA sequences encoding the polypeptide components of a desired fusion may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures, if desired. Such a peptide linker sequence is incorporated into the fusion protein using standard techniques well known in the art. Certain peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39 46 (1985); Murphy et al., Proc. Natl. Acad. Sci. USA 83:8258 8262 (1986); U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences may be operably linked to suitable transcriptional or translational regulatory elements. Certain of the regulatory elements responsible for expression of DNA are located 5′ to the DNA sequence encoding the first polypeptides. Similarly, other regulatory elements such as stop codons required to end translation and transcription termination signals are present 3′ to the DNA sequence encoding the second polypeptide.

In general, polypeptides and fusion polypeptides (as well as their encoding polynucleotides) are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

### EXAMPLES

### Example 1

### Overexpression of Acyl-ACP Reductase Increases Fatty Acid Production in Cyanobacteria

Overexpression of orf1594 in S. elongatus PCC7942 was performed to examine its effects on production of lipids, such as free fatty acids. S. elongatus PCC7942 was transformed with a vector containing the orf1594 sequence under the control of an IPTG-inducible promoter, with or without a vector encoding ACP. Transformed and control (wild-type) cells were then subcultured to achieve an OD₇₅₀ of 0.1 the day before induction, and induced with 1 mM IPTG (0 hr). At 24 hours post-induction, 0.5 OD equivalents of whole lysate were separated by thin layer chromatography (TLC) using a mobile phase for polar lipids (70 mL chloroform/22 mL methanol/3 mL water). 5 μg of a palmitic acid standard was included. Samples of wild-type, uninduced orf1594, and induced orf1594 were also analyzed at 24 h and 48 hours post-induction by gas chromatography (GC) for total fatty acid methyl esters (FAMES). Quantitation of constituent FAMES (e.g., C14:0, C14:1, C16:0, C16:1n9 and C18:0) was also performed by GC.

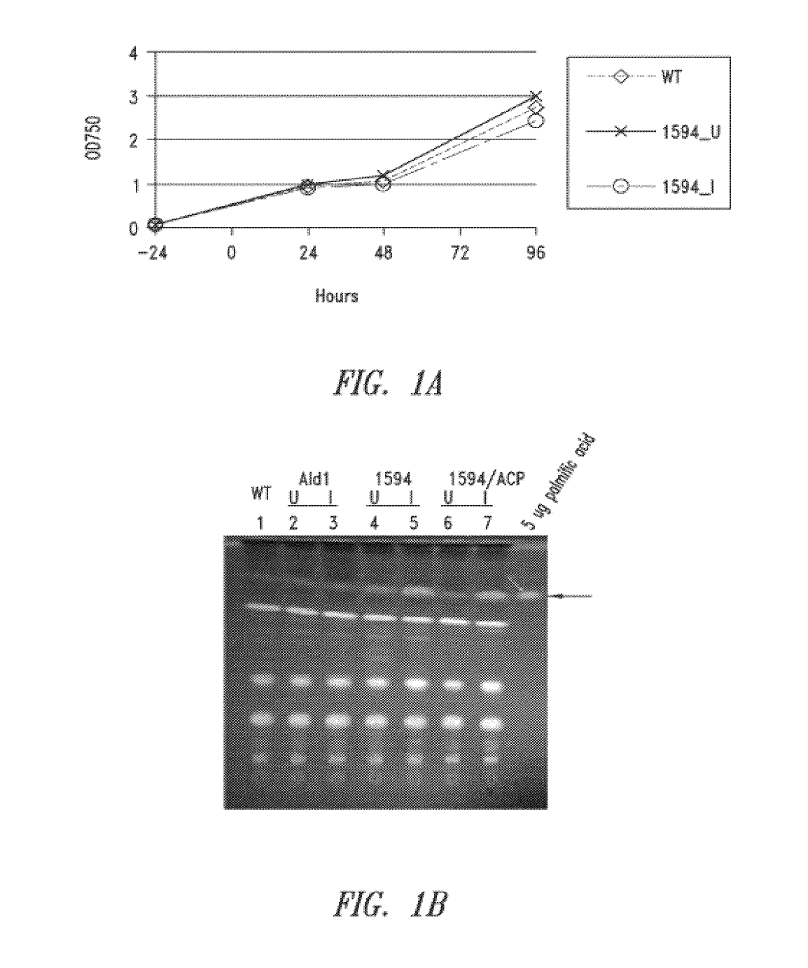

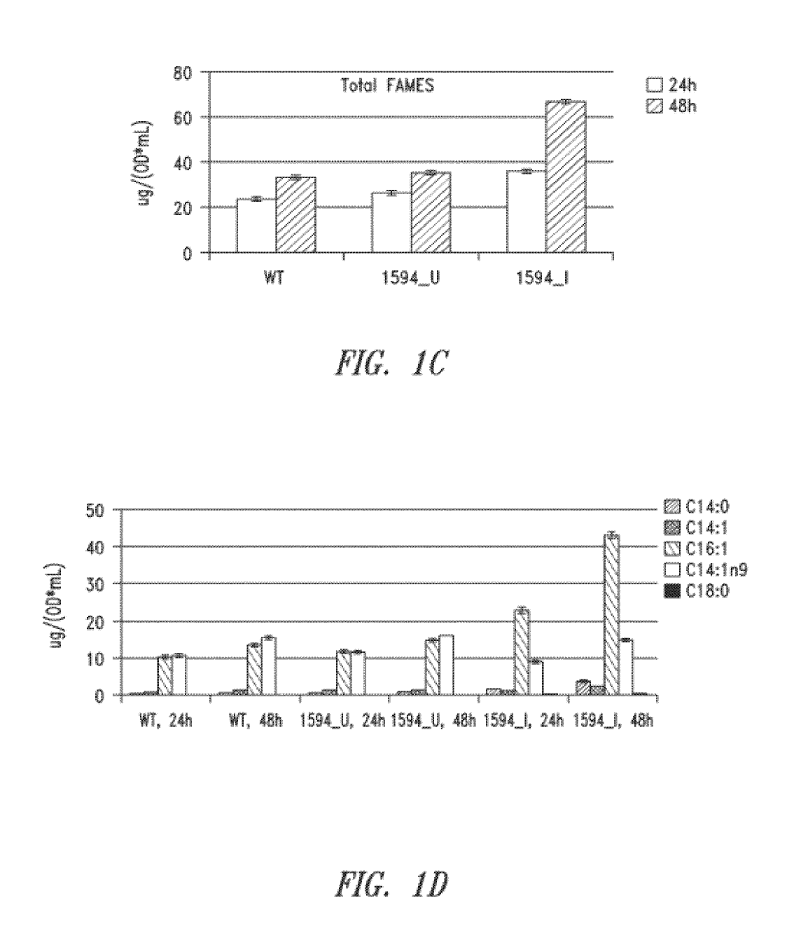

The results are shown in FIGS. 1A-1D. FIG. 1A shows that all cell cultures read an OD₇₅₀ of 0.1 the day before induction. FIG. 2A shows the TLC samples as WT (lane 1); Ald1 (lanes 2 and 3); orf1594 (lanes 4 and 5); and orf1594/ACP (lanes 6 and 7); increased fatty acid levels can be seen, for example, in lanes 5 and 7 (lane 5=induced orf1594 only, and lane 7=orf1594/ACP, respectively). FIG. 1C shows the samples of WT, uninduced orf1594 and induced orf1594 that were analyzed at 24 h and 48 h post-induction by GC for total; increased fatty acid levels can be seen in induced cells relative to controls, especially at 48 hours. FIG. 1D shows that the increase in total FAMES is primarily due to an increase in C16:0.

### Example 2

### Aldehyde Dehydrogenase Catalyzes Fatty Acid Production in Cyanobacteria that Overexpress Acyl-ACP Reductase

To evaluate the role of endogenous aldehyde dehydrogenase (orf0489) in free fatty acid production in an acyl-ACP reductase (orf1594) overexpressing strain of Cyanobacteria, the endogenous orf0489 gene was disrupted by transposon-mediated insertional mutagenesis. The orf0489 disruption was made in both wild-type Synechococcus elongatus PCC7942 and orf1594 (NS2_trc)-overexpressing backgrounds, by introducing a cosmid with a transposon disruption of the orf0489 gene.

Four different strains were diluted to an OD₇₅₀ of 0.1 the day before induction and then induced with 1 mM IPTG at t=0. One strain overexpressed acyl-ACP reductase (1594), another strain had a deletion in the aldehyde dehydrogenase gene (D0498), and two other strains overexpressed 1594 and had a deletion in orf0489 (15941D0489#1 and 15941D0489#2). Samples were collected for TLC and GC analysis 24 and 48 hr post-induction. Colony-forming-units were also measured to evaluate the effects on cell growth.

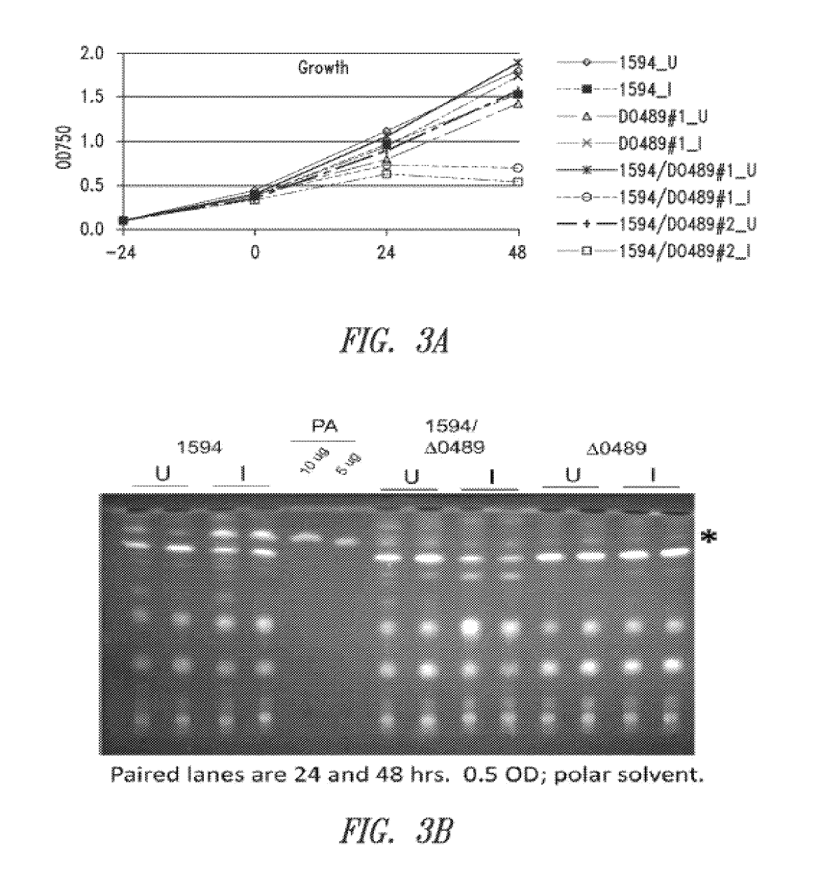

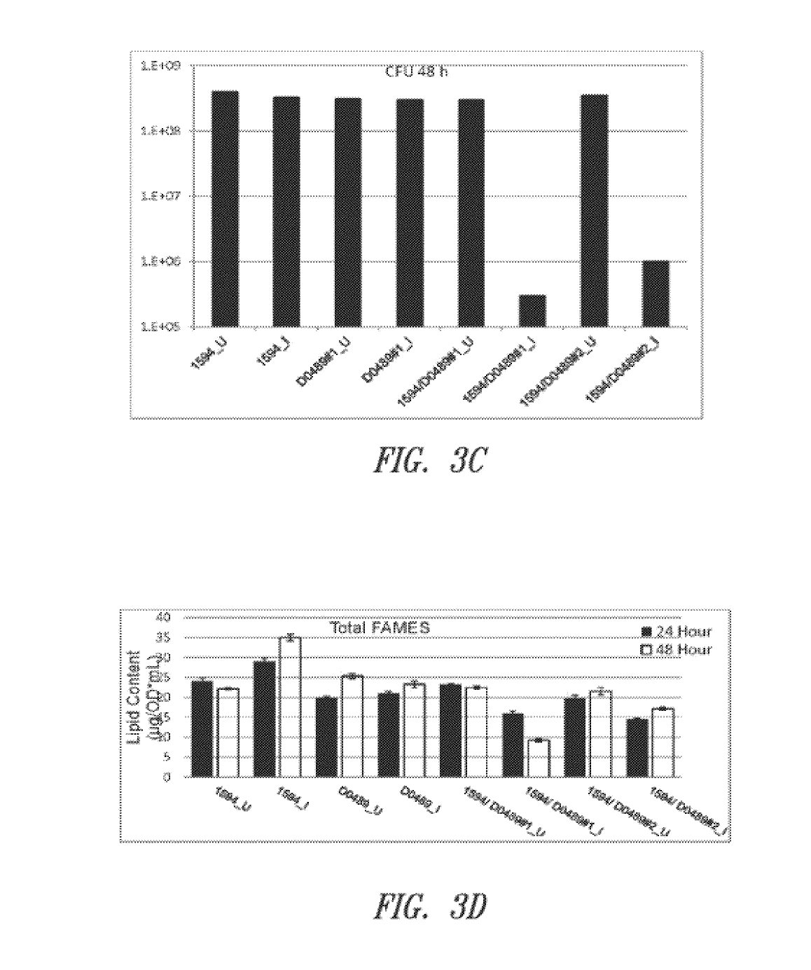

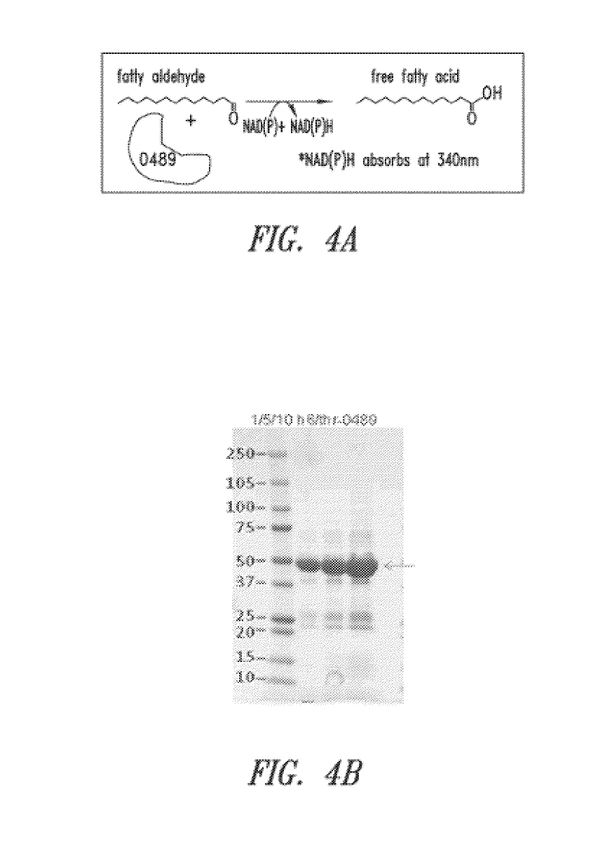

As shown in FIGS. 3A-3D, induction of orf1594 in a WT background resulted in a significant increase in free fatty acids (as seen by both TLC in FIG. 3B and GC in FIG. 3D), with minimal effect on growth or toxicity (see the growth curve in FIG. 3A and CFU's in FIG. 3C). Deletion of orf0489 in a WT background had no discernible effect on growth or toxicity; and no effect on free fatty acid production compared to a WT strain. However, deletion of orf0489 in the orf1594-expressing strains (1594/D0489 #1 and #2) caused a significant reduction in free fatty acid production (FIGS. 3B and 3D); and reduced cell growth (FIGS. 3A and 3C).

### Example 3

### Purified H6-orf0489 Utilizes Nonyl-Aldehyde as a Substrate

To directly test whether orf0489 is an aldehyde dehydrogenase, a histidine-tagged version of this protein (h₆-orf0489) was overexpressed in E. coli and purified using metal affinity chromatography. The purified protein was then employed in an in vitro reaction using a fatty aldehyde as a substrate.

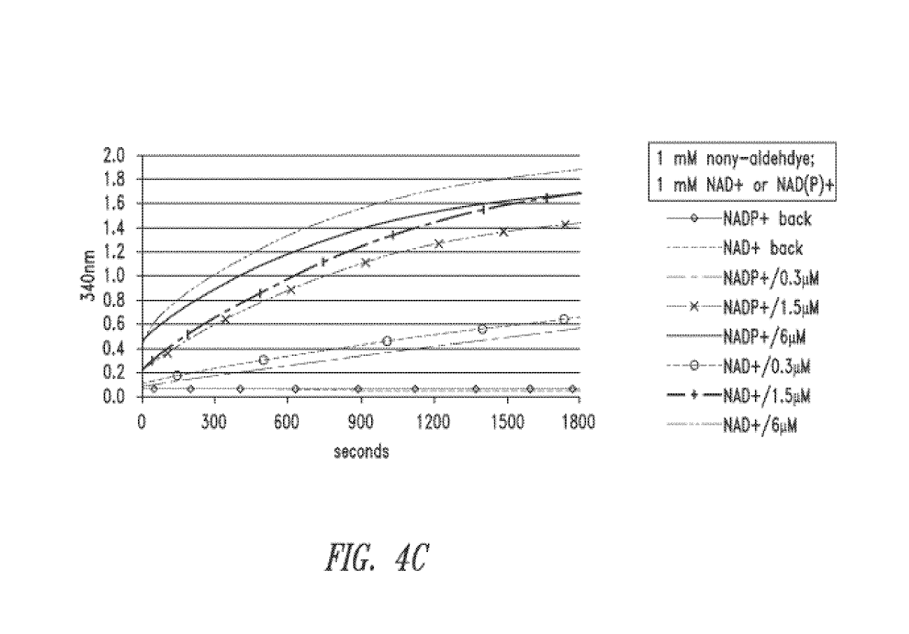

FIG. 4A shows the reaction scheme for measuring orf0489 aldehyde dehydrogenase activity, and FIG. 4B shows the SDS PAGE of metal affinity-purified h₆-0489. The reaction was started by mixing together a fatty aldehyde substrate (nonyl-aldehyde at 1 mM), various concentrations of purified h₆-orf0489 (0.3, 1.5 or 6 μM final concentration), and NAD+ or NAD(P)+ at 1 mM. The progress of the reaction was assessed by measuring the production of NAD(P)H at 340 nm using the SpectraMax M5; measurements were taken every 30 seconds for 30 minutes. FIG. 4C shows that the purified h₆-orf0489 polypeptide utilizes nonyl-aldehyde as a substrate, in the presence of either NAD+ or NAD(P)+, as evidenced by increased NAD(P)H production relative to controls (NAD+back and NADP+back).

To demonstrate that orf0489 is an aldehyde dehydrogenase of numerous substrates, a histidine-tagged version of this protein (h₆-orf0489) was overexpressed in E. coli and purified using metal affinity- and size exclusion chromatography (SEC). The protein eluted off of SEC as a single monodisperse peak. The enzymatic characteristics of purified protein were then measured in vitro using fatty aldehydes of varying chain length (4-16) as substrate. In this assay, h₆-0489 was incubated with 150 mM NaCl, 50 mM Tris, pH 7.5, 0.05% IGEPAL, 1 mM NAD+ and varying concentrations of the fatty aldehyde substrate being tested. The reaction was carried out in a cuvette with a 1 cm pathlength, and the progress was assessed by measuring the production of NADH at 340 nm using a SpectraMax M5 spectrophotometer. Measurements were taken every second for 2-5 minutes, and the initial velocities of each reaction were determined in the linear range. The A₃₄₀ was converted into amount of NADH formed using the molar extinction coefficient for NADH (6200 M⁻¹ cm⁻¹). The Michaelis constants Kₘ and Vₘₐₓ were determined by fitting the data using non-linear progression in Prism to the equation Y=(Vmax*X)/(Km+X), where X=substrate concentration and Y=velocity. K_{cat} was determined from the equation Vmax=Kcat*Eₜ, where Eₜ=the concentration of h₆-0489. Data are presented in Table C as averages of at least three different experiments, with errors presented as standard deviation.

TABLE C
Enzymatic characterization of h₆-0489.

| | AVE | | | STDEV | |
|---|---|---|---|---|---|
| | k_{cat} (min⁻¹) | (μM) | k_{cat}/Km | k_{cat} | Km |
| butyraldehyde | 459.2 | 189.4 | 2.4 | | |
| hexanal | 510.5 | 19.3 | 26.5 | 24.4 | 4.4 |
| octanal | 810.1 | 15.4 | 52.5 | 63.2 | 5.2 |
| decanal | 724.3 | 7.6 | 95.6 | 65.0 | 3.1 |
| dodecyl aldehyde | 557.5 | 11.4 | 49.1 | 74.9 | 2.4 |
| hexadecenal | 647.8 | 38.1 | 17.0 | 37.4 | 10.5 |

Consistent with in vivo results in cells over-expressing orf1594, in vitro h₆-0489 was able to oxidize hexadecenal to a free fatty acid. h₆-0489 demonstrated the highest efficiency (i.e., K_{cat}/Kₘ) towards aldehydes of medium chain length (8-12), although the enzyme was also able to utilize substrates of shorter chain length (4-6). The Kₘ of the enzyme ranged from 7.6 to 38.1 for chain lengths 6-16. These results demonstrate that h6-0489 is an aldehyde dehydrogenase that can utilize medium- to long-chain acyl aldehyde substrates.

### Example 4

### Co-Overexpression of orf0489 with Orf1594(2×) Increases Free Fatty Acid Production

To test whether overexpression of orf0489 further increases fatty acid production in acyl-ACP reductase (orf1594)-overexpressing strains of Cyanobacteria, strains containing either (i) two copies of orf1594 (1594(2×)), or (ii) two copies of orf1594 co-expressed with orf0489 (orf1594(2X)/0489 #3 or #4) were induced with IPTG at time 0 and evaluated for cell growth and fatty acid production. Samples were then taken for CFU at 48 and 96 hours; for qPCR at 24 hours; and for GC at 48, 96 and 144 hours.

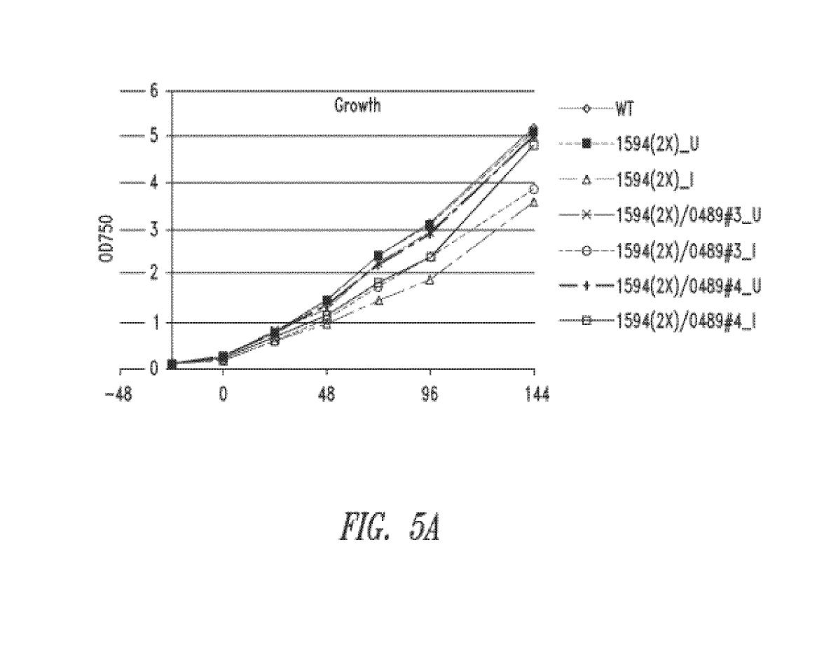

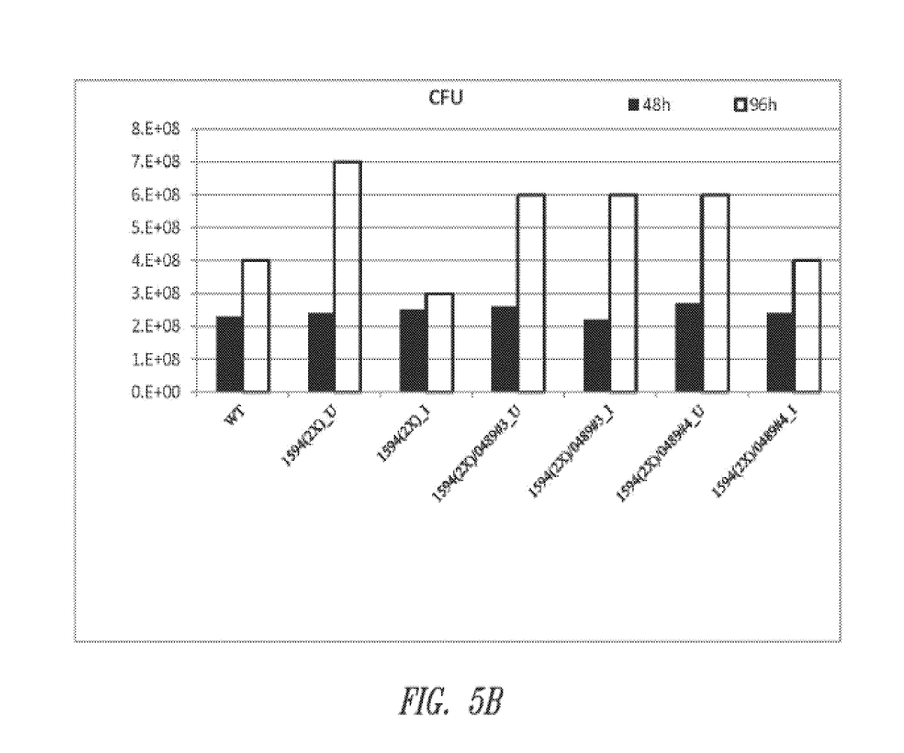

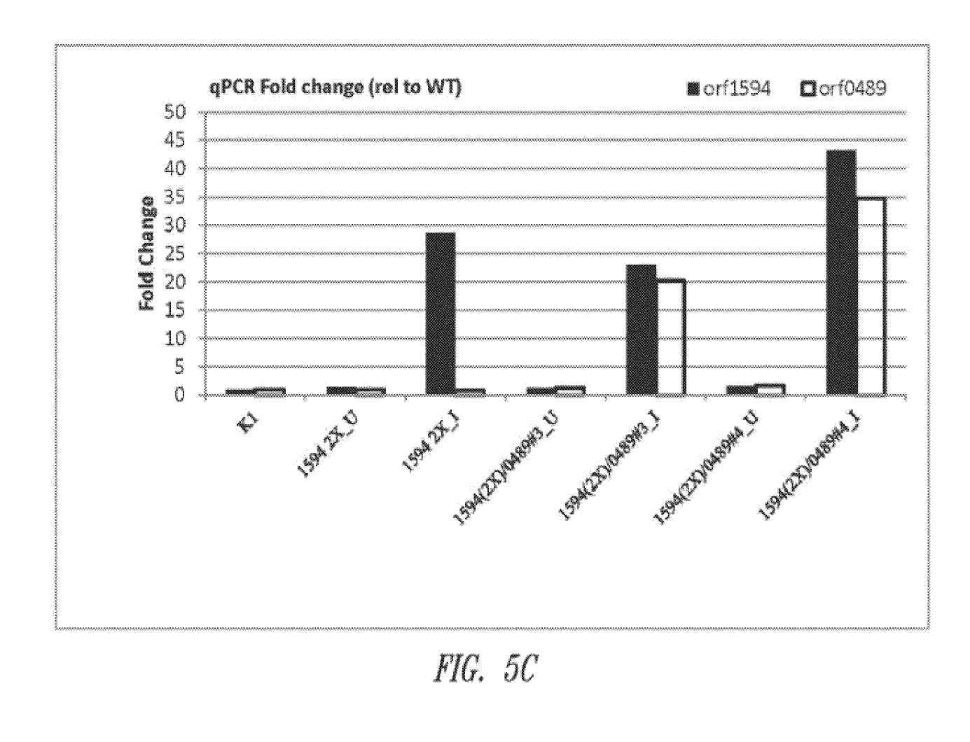

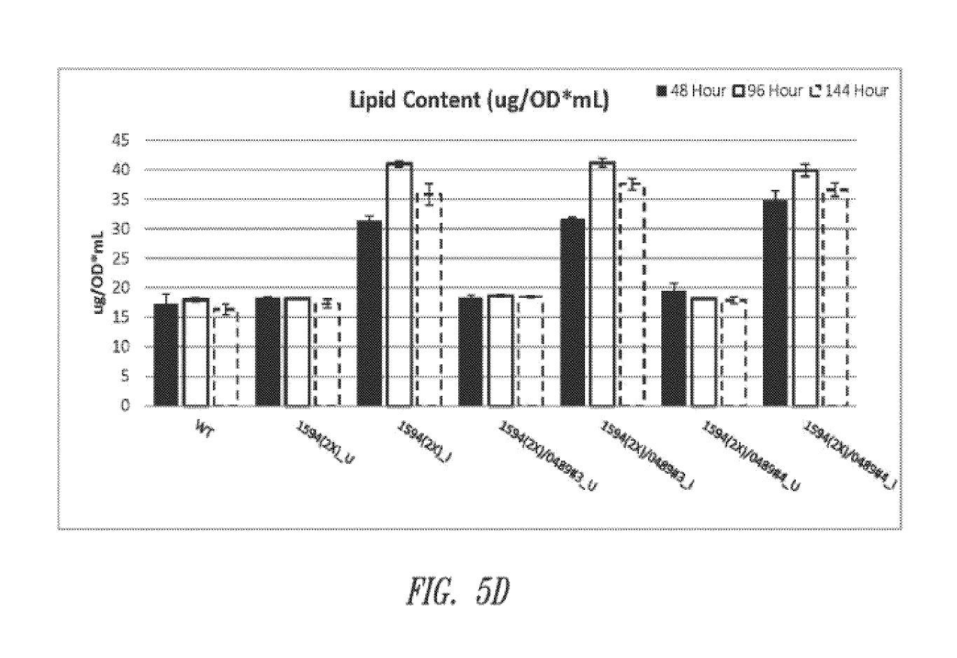

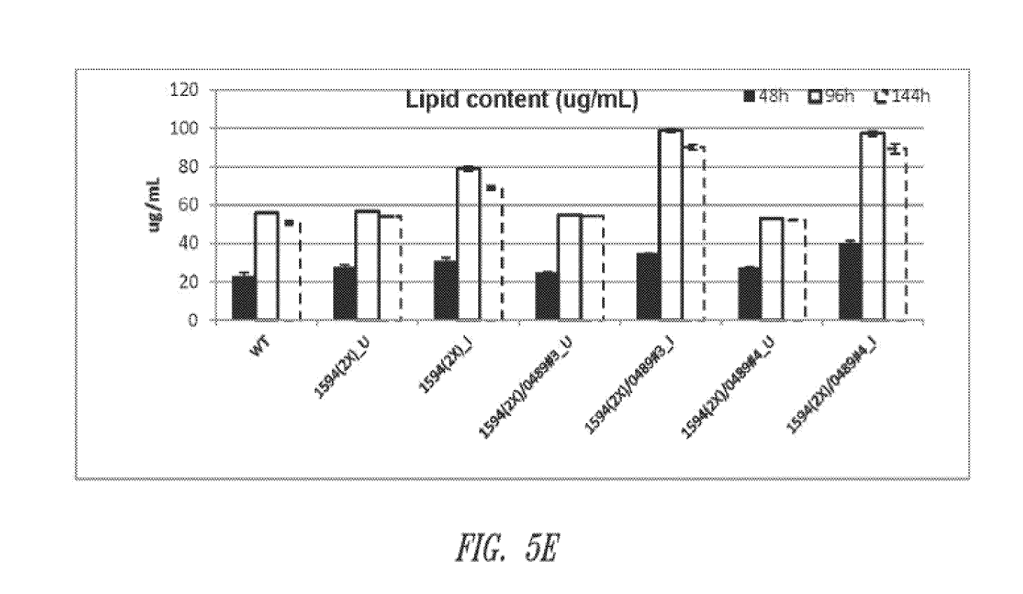

FIGS. 5A and 5B show the growth of the various strains over time, as measured by OD750 (FIG. 5A) and colony forming units (CFU) (FIG. 5B). FIG. 5C confirms the overexpression of orf0489 and orf1594. As shown in FIGS. 5D and 5E, the GC data for total lipid was plotted in two ways: either as a per density basis (μg/OD*mL in FIG. 5D) or a per volume basis (μg/mL in FIG. 5E). Induction and high-overexpression of orf1594(2X) causes a growth phenotype and a reduction in CFUs, and co-expression of orf0489 alleviates the growth phenotype (FIGS. 5A and 5B). With regards to total lipids, 1594(2X)/orf0489 produces equivalent amounts of total lipids as orf1594(2X) in terms of μg/OD*mL (FIG. 5D), but significantly more total lipids on a per volume basis (ug/mL) (FIG. 5E) due to its enhanced growth.

These results show that when acyl-ACP reductase (e.g., orf1594) is highly overexpressed in Cyanobacteria, the co-overexpression of aldehyde dehydrogenase (orf0489) can improve cell growth and/or increase overall fatty acid production (especially as measured on a per volume basis) relative to Cyanobacteria that highly overexpress acyl-ACP reductase and only have endogenous expression levels of aldehyde dehydrogenase.

### Example 5

### Production of Wax Esters by Cyanobacteria

As shown below, co-expression of orf1594, an alcohol dehydrogenase (slr1192 or ACIAD3612), and aDGAT not only induced wax ester (WE) formation, but also dramatically reduced TAG production. Alcohol dehydrogenases slr1192 from Synechocystis sp. PCC6803 and ACIAD3612 from Acinetobacter baylyi were amplified from genomic DNA, cloned into an NS1 vector, and transformed into an aDGAT(NS4)/orf1594(NS4) strain. All genes were expressed from the pTrc promoter.

Strains were grown in BG11 (plus antibiotic), diluted to an OD750 of 0.1 the day before induction, then induced with 1 mM IPTG. Samples were collected for GC and TLC at 24 hours, and for CFU at 24 and 48 hours.

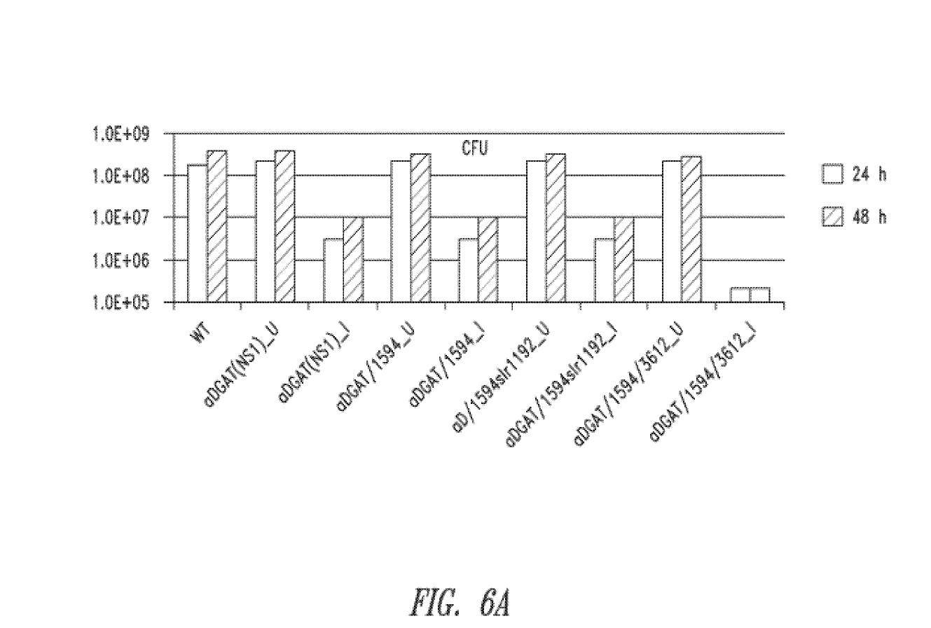

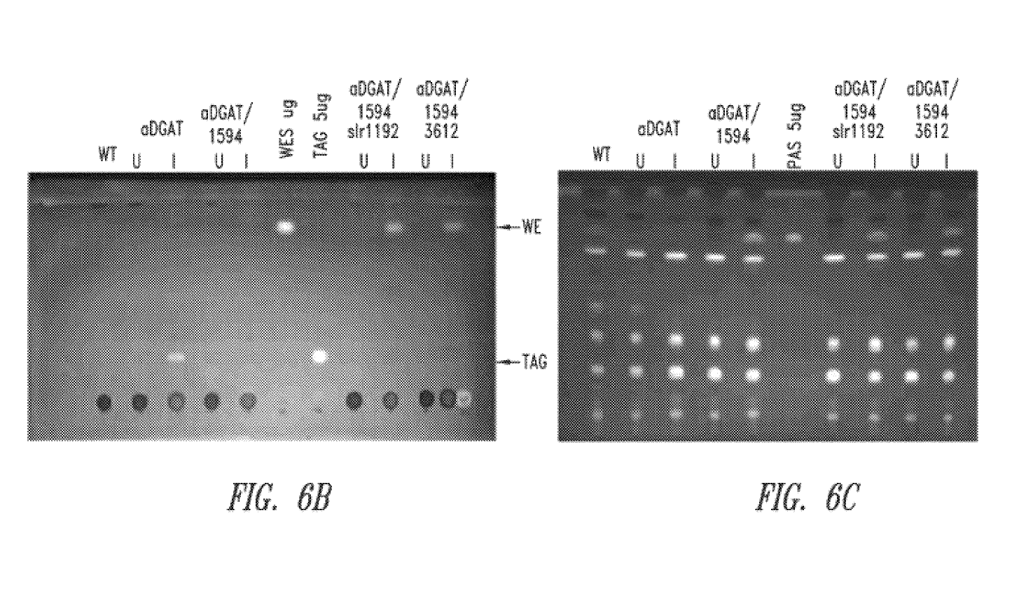

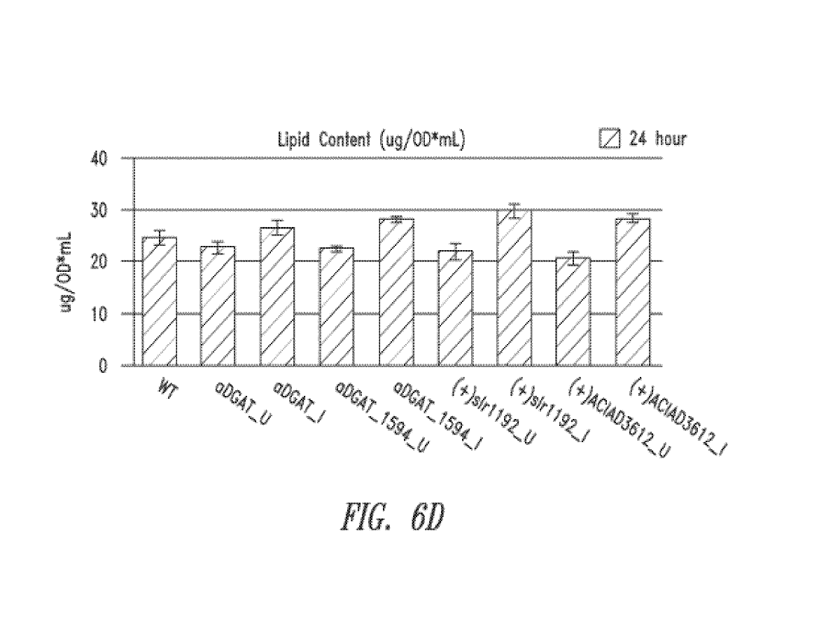

FIG. 6A shows growth curves (induction started at 0 hours) as measured by colony-forming units (CFU). FIGS. 6A and 6B show thin layer chromatograpy (TLC) of samples 24 hours post-induction. 0.50 D-equivalents were separated using nonpolar solvents (90% hexane/10% diethyl ether; FIG. 6A) to show TAG and WE formation, or polar solvents (FIG. 6B) to show fatty acid formation. 5 μg of WE and TAG standards were included for analysis the non-polar plate; and 5 μg of palmitic acid (PA) was included for analysis of the polar plate. FIG. 6D shows total FAMES (expressed as μg/OD*mL) from samples collected 24 h post-induction.

These results show that over-expressed alcohol dehydrogenases are able to convert fatty aldehydes into long chain fatty alcohols, and that DGATs having wax ester synthase activity (e.g., aDGAT) utilize these fatty alcohols to form wax esters. Also, the orf1594-containing strains produced free fatty acids (see FIG. 6C), suggesting that the endogenous orf0489 activity efficiently competes with the alcohol dehydrogenase transgenes for the fatty aldehyde substrate. Hence, deleting orf0489 (Δ0489) in these and related strains could lead to significant increases in wax ester formation. Wax ester formation could also be increased by deleting aldehyde carbonylase (Δ1593) to reduce competition between aldehyde dehydrogenase and aldehyde decarbonylase for fatty aldehyde substrate. Combinations of Δ0489 and Δ1593 could optimize shunting of fatty aldehydes towards aldehyde dehydrogenase/WE synthase pathways, and further increase wax ester formation.

### Alternative Embodiments

1. A modified photosynthetic microorganism, comprising an overexpressed acyl-acyl carrier protein (ACP) reductase polypeptide,

wherein said modified photosynthetic microorganism produces an increased amount of free fatty acid (FFA) as compared to an unmodified photosynthetic microorganism of the same species.

2. A modified photosynthetic microorganism, comprising an overexpressed acyl-acyl carrier protein (ACP) reductase polypeptide,

wherein said modified photosynthetic microorganism produces one or more lipids in an amount of at least about 25-35 μg/mg/day.

3. The modified photosynthetic microorganism of embodiment 2, wherein said lipid is a free fatty acid (FFA).

4. The modified photosynthetic microorganism of any one of embodiments 1-3, wherein said overexpressed acyl-ACP reductase polypeptide is encoded by (i) an endogenous polynucleotide which is operably linked to one or more introduced regulatory elements, or (ii) an introduced polynucleotide.

5. The modified photosynthetic microorganism of embodiment 4, wherein said one or more introduced regulatory elements are derived from the same genus as said modified photosynthetic microorganism.

6. The modified photosynthetic microorganism of embodiment 4, wherein said one or more introduced regulatory elements are derived from the same species as said modified photosynthetic microorganism.

7. The modified photosynthetic microorganism of embodiment 4, wherein said one or more introduced regulatory elements are derived from a different genus or species relative to said modified photosynthetic microorganism.

8. The modified photosynthetic microorganism of embodiment 4, wherein said one or more introduced regulatory elements are selected from at least one of a promoter, enhancer, repressor, ribosome binding site, and a transcription termination site.

9. The modified photosynthetic microorganism of embodiment 4, wherein said one or more introduced regulatory elements comprises an inducible promoter.

10. The modified photosynthetic microorganism of embodiment 9, wherein said inducible promoter is a weak promoter under non-induced conditions.

11. The modified photosynthetic microorganism of embodiment 4, wherein said one or more introduced regulatory elements comprises a constitutive promoter.

12. The modified photosynthetic microorganism of any one of embodiments 1-11, wherein said overexpressed acyl-ACP reductase polypeptide is from Synechococcus elongatus PCC7942 (orf1594) or Synechocystis sp. PCC6803 (orfsII0209), or a fragment or variant thereof.

13. The modified photosynthetic microorganism of any one of embodiments 1-12, wherein said overexpressed acyl-ACP reductase polypeptide has the amino acid sequence set forth in SEQ ID NO:2, or a fragment or variant thereof.

14. The modified photosynthetic microorganism of any one of embodiments 1-13, further comprising one or more of the following:

(i) one or more overexpressed or introduced polynucleotides encoding (a) an acyl carrier protein (ACP), (b) an acetyl coenzyme A carboxylase (ACCase), (c) a diacylglycerol acyltransferase (DGAT) optionally in combination with a fatty acyl Co-A synthetase, (d) an aldehyde dehydrogenase that is capable of converting an acyl aldehyde into a fatty acid, (e) an alcohol dehydrogenase that is capable of converting an acyl aldehyde into a fatty alcohol optionally in combination with a DGAT having wax ester synthase activity, or (f) any combination of (a)-(e);

(ii) reduced expression of one or more genes of a glycogen biosynthesis or storage pathway as compared to a wild-type photosynthetic microorganism;

(iii) one or more introduced polynucleotides encoding a protein of a glycogen breakdown pathway;

(iv) reduced expression of one or more genes encoding an endogenous aldehyde decarbonylase;

(v) reduced expression of one or more genes encoding an acyl-ACP synthetase (Aas), or

(vi) any combination of (i)-(v).

15. The modified photosynthetic microorganism of embodiment 14, wherein said ACP is a bacterial or a plant ACP.

16. The modified photosynthetic microorganism of embodiment 14, wherein said ACP is from Synechococcus, Spinacia oleracea, Acinetobacter, Streptomyces, or Alcanivorax.

17. The modified photosynthetic microorganism of embodiment 14, wherein said ACP has the amino acid sequence of any one of SEQ ID NOS:6, 8, 10, 12, or 14, or a fragment or variant thereof.

18. The modified photosynthetic microorganism of embodiment 14, wherein said ACCase is from Synechococcus, Saccharomyces cerevisiae, or Triticum aestivum.

19. The modified photosynthetic microorganism of embodiment 14, wherein said DGAT is an Acinetobacter DGAT, a Streptomyces DGAT, or an Alcanivorax DGAT.

20. The modified photosynthetic microorganism of embodiment 14, wherein said fatty acyl Co-A synthetase is from E. coli (FadD) or S. cerevisiae, or a fragment or variant thereof.

21. The modified photosynthetic microorganism of embodiment 20, wherein said fatty acyl Co-A synthetase from S. cerevisiae is Faa1p, Faa2p, or Faa3p, or a fragment or variant thereof.

22. The modified photosynthetic microorganism of embodiment 14, wherein said aldehyde dehydrogenase is from Synechococcus elongatus PCC7942.

23. The modified photosynthetic microorganism of embodiment 14, wherein said aldehyde dehydrogenase is encoded by orf0489 of Synechococcus elongatus PCC7942, or a homolog or paralog thereof, or a fragment or variant thereof.

24. The modified photosynthetic microorganism of embodiment 23, wherein said aldehyde dehydrogenase has the amino acid sequence of SEQ ID NO:103, or a fragment or variant thereof.

25. The modified photosynthetic microorganism of any one of embodiments 22-24, wherein the introduced aldehyde dehydrogenase increases cell growth, increases production of free fatty acids, or both, relative to overexpression of the acyl-ACP reductase without the introduced aldehyde dehydrogenase.

26. The modified photosynthetic microorganism of embodiment 14, wherein said one or more genes of a glycogen biosynthesis or storage pathway are selected from a glucose-1-phosphate adenyltransferase (glgC) gene and a phosphoglucomutase (pgm) gene.

27. The modified photosynthetic microorganism of embodiment 26, comprising a full or partial deletion of said one or more genes of a glycogen breakdown pathway.

28. The modified photosynthetic microorganism of embodiment 14, wherein said one or more proteins of a glycogen breakdown pathway are selected from glycogen phosphorylase (GlgP), glycogen isoamylase (GlgX), glucanotransferase (MalQ), phosphoglucomutase (Pgm), glucokinase (Glk), and phosphoglucose isomerase (Pgi).

29. The modified photosynthetic microorganism of embodiment 14, comprising a full or partial deletion of the endogenous aldehyde decarbonylase.

30. The modified photosynthetic microorganism of embodiment 29, wherein said aldehyde decarbonylase is encoded by orf1593 of S. elongatus PCC7942, orfsII0208 of Synechocystis sp. PCC6803, or a homolog or paralog thereof, or a fragment or variant thereof.

31. The modified photosynthetic microorganism of embodiment 14, comprising a full or partial deletion of the endogenous Aas.

32. The modified photosynthetic microorganism of embodiment 14, combining the acyl-ACP reductase and the aldehyde dehydrogenase.

33. The modified photosynthetic microorganism of any one of embodiments 4-32, wherein one or more of said introduced polynucleotides is present in one or more expression constructs.

34. The modified photosynthetic microorganism of embodiment 33, wherein said expression construct is stably integrated into the genome of said modified photosynthetic microorganism.

35. The modified photosynthetic microorganism of embodiment 33 or embodiment 34, wherein said expression construct comprises an inducible promoter.

36. The modified photosynthetic microorganism of any one of embodiments 33-35, wherein one or more of said introduced polynucleotides are present in an expression construct comprising a weak promoter under non-induced conditions.

37. The modified photosynthetic microorganism of any one of embodiments 4-36, wherein one or more of said introduced polynucleotides are codon-optimized for expression in a Cyanobacterium.

38. The modified photosynthetic microorganism of embodiment 37, wherein one or more of said codon-optimized polynucleotides are codon-optimized for expression in a Synechococcus elongatus.

39. The modified photosynthetic microorganism of any of embodiments 1-38, wherein said photosynthetic microorganism is a Cyanobacterium and said Cyanobacterium is a Synechococcus elongatus.

40. The modified photosynthetic microorganism of embodiment 39, wherein said Synechococcus elongatus is strain PCC7942.

41. The modified photosynthetic microorganism of embodiment 39, wherein said Cyanobacterium is a salt tolerant variant of Synechococcus elongatus strain PCC7942.

42. The modified photosynthetic microorganism of any of embodiments 1-38, wherein said photosynthetic microorganism is a Cyanobacterium and said Cyanobacterium is Synechococcus sp. PCC7002.

43. The modified photosynthetic microorganism of any of embodiments 1-38, wherein said photosynthetic microorganism is a Cyanobacterium and said Cyanobacterium is Synechocystis sp. PCC6803.

44. A modified Synechococcus elongatus PCC7942, comprising an overexpressed acyl-acyl carrier protein (ACP) reductase polypeptide, wherein said overexpressed polypeptide is encoded by (i) an endogenous polynucleotide which is operably linked to one or more introduced regulatory elements, or (ii) an introduced polynucleotide, and wherein said modified Synechococcus elongatus PCC7942 produces or accumulates an increased amount of free fatty acid as compared to a corresponding wild-type or unmodified Synechococcus elongatus PCC7942.

45. The modified photosynthetic microorganism of embodiment 14(i)(c), wherein said microorganism produces an increased amount of triglycerides as compared to a DGAT only-expressing microorganism of the same species, or a DGAT-expressing microorganism of the same species which does not overexpress an acyl-ACP reductase.

46. A method of producing a modified photosynthetic microorganism that produces or accumulates an increased amount of lipid as compared to a corresponding wild-type photosynthetic microorganism, comprising over-expressing an acyl-acyl carrier protein (ACP) reductase polypeptide in said modified photosynthetic microorganism.

47. The method of embodiment 46, wherein said modified photosynthetic microorganism is a Cyanobacterium.

48. The method of embodiment 46 or 47, wherein said modified photosynthetic microorganism produces one or more lipids in an amount of at least about 25-35 μg/mg/day.

49. The method of any one of embodiments 46-48, wherein said lipid is a free fatty acid (FFA).

50. The method of any one of embodiments 45-49, comprising (i) introducing one or more regulatory elements which are operably linked to an endogenous polynucleotide that encodes said acyl-ACP reductase, and/or (ii) introducing a polynucleotide that encodes said acyl-ACP reductase.

51. The method of embodiment 50, wherein said one or more introduced regulatory elements are derived from the same genus as said modified photosynthetic microorganism.

52. The method of embodiment 50, wherein said one or more introduced regulatory elements are derived from the same species as said modified photosynthetic microorganism.

53. The method of embodiment 50, wherein said one or more introduced regulatory elements are derived from a different genus or species relative to said modified photosynthetic microorganism.

54. The method of embodiment 50, wherein said one or more introduced regulatory elements are selected from at least one of a promoter, enhancer, repressor, ribosome binding site, and a transcription termination site.

55. The method of embodiment 50, wherein said one or more introduced regulatory elements comprises an inducible promoter.

56. The method of embodiment 55, wherein said inducible promoter is a weak promoter under non-induced conditions.

57. The method of embodiment 50, wherein said one or more introduced regulatory elements comprises a constitutive promoter.

58. The method of any one of embodiments 46-57, wherein said overexpressed acyl-ACP reductase polypeptide is from Synechococcus elongatus PCC7942 (orf1594) or Synechocystis sp. PCC6803 (orfsII0209), or a fragment or variant thereof.

59. The method of any one of embodiments 46-58, wherein said overexpressed acyl-ACP reductase polypeptide has the amino acid sequence set forth in SEQ ID NO:2, or a fragment or variant thereof.

60. The method of any one of embodiments 46-59, further comprising one or more of the following:

(i) overexpressing or introducing one or more polynucleotides encoding (a) an acyl carrier protein (ACP), (b) an acetyl coenzyme A carboxylase (ACCase), (c) a diacylglycerol acyltransferase (DGAT) optionally in combination with a fatty acyl Co-A synthetase, (d) an aldehyde dehydrogenase, (e) an alcohol dehydrogenase that is capable of converting an acyl aldehyde into a fatty alcohol optionally in combination with a DGAT having wax ester synthase activity, or (f) any combination of (a)-(e);

(ii) modifying the microorganism to reduce expression of one or more genes of a glycogen biosynthesis or storage pathway as compared to a wild-type photosynthetic microorganism;

(iii) introducing one or more polynucleotides encoding a protein of a glycogen breakdown pathway;

(iv) modifying the microorganism to reduce expression of one or more genes encoding an endogenous aldehyde decarbonylase;

(v) modifying the microorganism to reduce expression of one or more genes encoding an acyl-ACP synthetase (Aas), or

(vi) any combination of (i)-(v).

61. The method of embodiment 60, wherein said ACP is a bacterial or a plant ACP.

62. The method of embodiment 60, wherein said ACP is from Synechococcus, Spinacia oleracea, Acinetobacter, Streptomyces, or Alcanivorax.

63. The method of embodiment 60, wherein said ACP has the amino acid sequence of any one of SEQ ID NOS:6, 8, 10, 12, or 14, or a fragment or variant thereof.

64. The method of embodiment 60, wherein said ACCase is from Synechococcus, Saccharomyces cerevisiae, or Triticum aestivum.

65. The method of embodiment 60, wherein said DGAT is an Acinetobacter DGAT, a Streptomyces DGAT, or an Alcanivorax DGAT.

66. The method of embodiment 60, wherein said fatty acyl Co-A synthetase is from E. coli (FadD) or S. cerevisiae, or a fragment or variant thereof.

67. The method of embodiment 66, wherein said fatty acyl Co-A synthetase from S. cerevisiae is Faa1p, Faa2p, or Faa3p, or a fragment or variant thereof.

68. The method of embodiment 60, wherein said aldehyde dehydrogenase is from Synechococcus elongatus PCC7942.

69. The method of embodiment 60, wherein said aldehyde dehydrogenase is encoded by orf0489 of Synechococcus elongatus PCC7942, or a homolog or paralog thereof, or a fragment or variant thereof.

70. The method of embodiment 69, wherein said aldehyde dehydrogenase has the amino acid sequence of SEQ ID NO:103, or a fragment or variant thereof.

71. The method of any one of embodiments 68-70, wherein the introduced aldehyde dehydrogenase increases cell growth, increases production of free fatty acids, or both, relative to overexpression of the acyl-ACP reductase without the introduced aldehyde dehydrogenase.

72. The method of embodiment 60, wherein said one or more genes of a glycogen biosynthesis or storage pathway are selected from a glucose-1-phosphate adenyltransferase (glgC) gene and a phosphoglucomutase (pgm) gene.

73. The method of embodiment 72, comprising a full or partial deletion of said one or more genes of a glycogen breakdown pathway.

74. The method of embodiment 60, wherein said one or more proteins of a glycogen breakdown pathway are selected from glycogen phosphorylase (GlgP), glycogen isoamylase (GlgX), glucanotransferase (MalQ), phosphoglucomutase (Pgm), glucokinase (Glk), and phosphoglucose isomerase (Pgi).

75. The method of embodiment 60, comprising a full or partial deletion of the endogenous aldehyde decarbonylase.

76. The method of embodiment 75, wherein said aldehyde decarbonylase is encoded by orf1593 of S. elongatus PCC7942, orfsII0208 of Synechocystis sp. PCC6803, or a homolog or paralog thereof, or a fragment or variant thereof.

77. The method of embodiment 60, comprising a full or partial deletion of the endogenous Aas.

78. The method of embodiment 60, combining the acyl-ACP reductase and the aldehyde dehydrogenase.

79. The method of any one of embodiments 60-78, wherein one or more of said introduced polynucleotides is present in one or more expression constructs.

80. The method of embodiment 79, wherein said expression construct is stably integrated into the genome of said modified photosynthetic microorganism.

81. The method of embodiment 79 or embodiment 80, wherein said expression construct comprises an inducible promoter.

82. The method of any one of embodiments 79-81, wherein one or more of said introduced polynucleotides are present in an expression construct comprising a weak promoter under non-induced conditions.

83. The method of any one of embodiments 50-82, wherein one or more of said introduced polynucleotides are codon-optimized for expression in a Cyanobacterium.

84. The method of embodiment 83, wherein one or more of said codon-optimized polynucleotides are codon-optimized for expression in a Synechococcus elongatus.

85. The method of any of embodiments 46-82, wherein said photosynthetic microorganism is a Cyanobacterium and said Cyanobacterium is a Synechococcus elongatus.

86. The method of embodiment 85, wherein said Synechococcus elongatus is strain PCC7942.

87. The method of embodiment 85, wherein said Cyanobacterium is a salt tolerant variant of Synechococcus elongatus strain PCC7942.

88. The method of any of embodiments 46-83, wherein said photosynthetic microorganism is a Cyanobacterium and said Cyanobacterium is Synechococcus sp. PCC7002.

89. The method of any of embodiments 46-83, wherein said photosynthetic microorganism is a Cyanobacterium and said Cyanobacterium is Synechocystis sp. PCC6803.

90. A method for the production of lipids, comprising culturing a modified photosynthetic microorganism according to any one of embodiments 1-45 or 110-118, wherein said modified photosynthetic microorganism accumulates an increased amount of lipid as compared to a corresponding wild-type photosynthetic microorganism.

91. The method of embodiment 90, wherein said culturing comprises inducing expression of one or more of said introduced polynucleotides.

92. The method of embodiment 90 or 91, wherein said culturing comprises culturing under static growth conditions.

93. The method of embodiment 91, wherein said inducing occurs under static growth conditions.

94. The method of embodiment 90, wherein said culturing comprises culturing in media supplemented with bicarbonate.

95. The method of embodiment 94, wherein the concentration of bicarbonate is selected from about 5, 10, 20, 50, 75, 100, 200, 300, 400, 500, 600, 700, 800, 900, and 1000 mM bicarbonate.

96. The method of embodiment 94 or 95, wherein the bicarbonate is present prior to inducing expressing of the introduced polynucleotide.

97. The method of embodiment 94 or 95, wherein the bicarbonate is present during induction of the introduced polynucleotide.

98. The method of embodiment 90, wherein said lipid is a free fatty acid which is produced in an amount of at least about 25-35 μg/mg/day.

99. The method of embodiment 98, wherein said free fatty acid is a C16:0 fatty acid.

100. A modified photosynthetic microorganism, comprising

(i) an overexpressed acyl-acyl carrier protein (ACP) reductase polypeptide; and

(ii) an aldehyde dehydrogenase polypeptide that is capable of converting an acyl aldehyde into a fatty acid,

wherein said modified photosynthetic microorganism produces an increased amount of free fatty acid (FFA) as compared to an unmodified photosynthetic microorganism of the same species, or as compared to a modified photosynthetic microorganism of the same species that overexpresses the acyl-ACP reductase without expressing the aldehyde dehydrogenase.

101. The modified photosynthetic microorganism of embodiment 100, wherein said aldehyde dehydrogenase is encoded by an unmodified, endogenous polynucleotide.

102. The modified photosynthetic microorganism of embodiment 100, wherein said aldehyde dehydrogenase is encoded by an endogenous polynucleotide, and is overexpressed by operably linking the endogenous polynucleotide to one or more introduced regulatory elements.

103. The modified photosynthetic microorganism of any one of embodiments 100-102, wherein said microorganism is Synechococcus elongatus PCC7942, and said aldehyde dehydrogenase is encoded by orf0489 of S. elongatus PCC7942.

104. The modified photosynthetic microorganism of embodiment 100, wherein said aldehyde dehydrogenase is overexpressed and encoded by an introduced polynucleotide.

105. The modified photosynthetic microorganism of embodiment 104, wherein said introduced polynucleotide is orf0489 of Synechococcus elongatus PCC7942.

106. The method of any one of embodiments 100-105, wherein said aldehyde dehydrogenase has the amino acid sequence of SEQ ID NO:103, or a fragment or variant thereof.

107. The method of any one of embodiments 100-106, wherein said aldehyde dehydrogenase increases cell growth, increases production of free fatty acids, or both, compared to overexpression of the acyl-ACP reductase without expression of the aldehyde dehydrogenase.

108. The method of any one of embodiments 102 or 104-106, wherein the overexpressed aldehyde dehydrogenase increases cell growth, increases production of free fatty acids, or both, compared to overexpression of the acyl-ACP reductase with naturally-occurring levels of the aldehyde dehydrogenase.

109. The method of any one of embodiments 100 to 108, combined with reduced expression of one or more genes encoding an acyl-ACP synthetase (Aas).

110. The modified photosynthetic microorganism of embodiment 14, comprising the overexpressed alcohol dehydrogenase in combination with the overexpressed DGAT having wax ester synthase activity, wherein said microorganism produces an increased amount of wax ester(s) as compared to a wild-type or unmodified microorganism of the same species.

111. The modified photosynthetic microorganism of embodiment 110, wherein the alcohol dehydrogenase is from Synechocystis sp. PCC6803 or Acinetobacter baylyi.

112. The modified photosynthetic microorganism of embodiment 110, wherein the alcohol dehydrogenase has the amino acid sequence of SEQ ID NO:105 (slr1192) or 107 (ACIAD3612).

113. The modified photosynthetic microorganism of embodiment 110, wherein the DGAT is aDGAT.

114. The modified photosynthetic microorganism of any one of embodiments 110-113, further comprising (i) reduced expression of an endogenous aldehyde dehydrogenase, (i) reduced expression of an aldehyde decarbonylase, (iii) an overexpressed acyl carrier protein (ACP) optionally in combination with an overexpressed acyl-ACP synthetase (Aas), or (iv) any combination of (i)-(iii).

115. The modified photosynthetic microorganism of embodiment 114, comprising reduced expression of the aldehyde dehydrogenase encoded by orf0489.

116. The modified photosynthetic microorganism of embodiment 114, comprising reduced expression of the aldehyde decarbonylase encoded by orf1593.

117. The modified photosynthetic microorganism of any one of embodiments 114-116, wherein said microorganism produces an increased amount of wax ester(s) as compared to the modified microorganism of embodiment 110 without any of (i)-(iv).

118. The modified photosynthetic microorganism of embodiment 115, wherein said microorganism produces an increased amount of wax ester(s) as compared to the microorganism of embodiment 110 or 114 without reduced expression of orf1489.

119. The method of embodiment 90, comprising culturing a modified photosynthetic microorganism according to any one of embodiments 110-118, wherein said lipid is a wax ester.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not necessarily limited by the disclosure.

1.-119. (canceled)

120. A modified Cyanobacterium, comprising an overexpressed acyl-acyl carrier protein (ACP) reductase polypeptide,
wherein said modified Cyanobacterium produces an increased amount of lipid as compared to a wild-type Cyanobacterium.

121. The modified Cyanobacterium microorganism of claim 120, further comprising one or more of the following:
(i) an overexpressed aldehyde dehydrogenase;
(ii) reduced expression of one or more genes of a glycogen biosynthesis or storage pathway as compared to a wild-type Cyanobacterium;
(iii) one or more introduced polynucleotides encoding a protein of a glycogen breakdown pathway;
(iv) reduced expression of one or more genes encoding an endogenous aldehyde decarbonylase;
(v) reduced expression of one or more genes encoding an acyl-ACP synthetase (Aas);
(vi) an overexpressed acyl carrier protein (ACP);
(vii) an overexpressed acetyl coenzyme A carboxylase (ACCase); or
(viii) any combination of (i)-(vii).

122. The modified Cyanobacterium of claim 121, wherein said overexpressed acyl-ACP reductase polypeptide, said overexpressed aldehyde dehydrogenase, or both is/are encoded by (i) an endogenous polynucleotide which is operably linked to one or more introduced regulatory elements, or (ii) an introduced polynucleotide.

123. The modified Cyanobacterium of claim 120, further comprising an overexpressed alcohol dehydrogenase in combination with an overexpressed DGAT having wax ester synthase activity and one or more of the following:
(i) reduced expression of one or more genes of a glycogen biosynthesis or storage pathway as compared to a wild-type Cyanobacterium;
(ii) one or more introduced polynucleotides encoding a protein of a glycogen breakdown pathway;
(iii) reduced expression of one or more genes encoding an endogenous aldehyde decarbonylase;
(iv) reduced expression of one or more genes encoding an acyl-ACP synthetase (Aas);
(v) an overexpressed acyl carrier protein (ACP);
(vi) an overexpressed acetyl coenzyme A carboxylase (ACCase);
(vii) reduced expression of an endogenous aldehyde dehydrogenase; or
(viii) any combination of (i)-(vii),
wherein said Cyanobacterium produces an increased amount of wax ester(s) as compared to a wild-type Cyanobacterium or unmodified Cyanobacterium of the same species.

124. The modified Cyanobacterium of claim 120, wherein said lipid is a free fatty acid (FFA), triglyceride or wax ester.

125. The modified Cyanobacterium of claim 120, wherein said modified Cyanobacterium is a Synechococcus elongatus PCC 7942; a salt tolerant variant of Synechococcus elongatus sp. PCC 7942; a Synechococcus elongatus sp. PCC 7002; or a Synechocystis elongatus sp. PCC 6803.

126. A method of producing a modified Cyanobacterium that produces or accumulates an increased amount of lipid as compared to a corresponding wild-type Cyanobacterium, comprising overexpressing an acyl-acyl carrier protein (ACP) reductase polypeptide in said modified Cyanobacterium.

127. The method of claim 126, wherein said lipid is a free fatty acid (FFA), triglyceride or wax ester.

128. The method of claim 126, further comprising one or more of the following:
(i) overexpressing an aldehyde dehydrogenase;
(ii) underexpressing one or more genes of a glycogen biosynthesis or storage pathway as compared to a wild-type Cyanobacterium;
(iii) introducing one or more polynucleotides encoding a protein of a glycogen breakdown pathway;
(iv) underexpressing one or more genes encoding an endogenous aldehyde decarbonylase;
(v) underexpressing one or more genes encoding an acyl-ACP synthetase (Aas);
(vi) overexpressing an acyl carrier protein (ACP);
(vii) overexpressing an acetyl coenzyme A carboxylase (ACCase); or
(viii) any combination of (i)-(vii).

129. The method of claim 128, comprising (i) introducing one or more regulatory elements which are operably linked to an endogenous polynucleotide that encodes said acyl-ACP reductase, said aldehyde dehydrogenase, or both, and/or (ii) introducing a polynucleotide that encodes said acyl-ACP reductase, said aldehyde dehydrogenase, or both.

130. The method of claim 126, further comprising overexpressing an alcohol dehydrogenase in combination with an overexpressed DGAT having wax ester synthase activity and one or more of the following:
(i) underexpressing one or more genes of a glycogen biosynthesis or storage pathway as compared to a wild-type Cyanobacterium;
(ii) introducing one or more introduced polynucleotides encoding a protein of a glycogen breakdown pathway;
(iii) underexpressing one or more genes encoding an endogenous aldehyde decarbonylase;
(iv) underexpressing one or more genes encoding an acyl-ACP synthetase (Aas);
(v) overexpressing an acyl carrier protein (ACP);
(vi) overexpressing an acetyl coenzyme A carboxylase (ACCase);
(vii) underexpressing an endogenous aldehyde dehydrogenase; or
(viii) any combination of (i)-(vii),
wherein said Cyanobacterium produces an increased amount of wax ester(s) as compared to a wild-type Cyanobacterium or unmodified Cyanobacterium of the same species.

131. The method of claim 126, wherein said modified Cyanobacterium is a Synechococcus elongatus PCC 7942; a salt tolerant variant of Synechococcus elongatus sp. PCC 7942; a Synechococcus elongatus sp. PCC 7002; or a Synechocystis elongatus sp. PCC 6803.

132. A method for the production of lipids, comprising culturing a modified Cyanobacterium according to claim 120.

133. The method of claim 132, wherein said lipid is a free fatty acid, triglyceride or wax ester.

134. A method for the production of lipids comprising:
preparing a modified Cyanobacterium which overexpresses acyl-acyl carrier protein (ACP) reductase; and
culturing said modified Cyanobacterium to produce lipids.
